SEQUENCE LISTING (28 sequences)

SEQ ID NO: 1 - DNA, 18 bp, synthetic construct
  source 1..18: mol_type = other DNA
gaggagccagcagaaggt

SEQ ID NO: 2 - DNA, 21 bp, synthetic construct
  source 1..21: mol_type = other DNA
tcccaccctacactcactaca

SEQ ID NO: 3 - DNA, 21 bp, synthetic construct
  source 1..21: mol_type = other DNA
tcaagtcgcttccaacctcaa

SEQ ID NO: 4 - DNA, 19 bp, synthetic construct
  source 1..19: mol_type = other DNA
tgtcacagtgagccgggtg

SEQ ID NO: 5 - DNA, 25 bp, synthetic construct
  source 1..25: mol_type = other DNA
gtggaagcagagaggagagcatttg

SEQ ID NO: 6 - DNA, 19 bp, synthetic construct
  source 1..19: mol_type = other DNA
ggtcggggtagcggctgaa

SEQ ID NO: 7 - DNA, 22 bp, synthetic construct
  source 1..22: mol_type = other DNA
gccaccatgagccaggcctact

SEQ ID NO: 8 - DNA, 21 bp, synthetic construct
  source 1..21: mol_type = other DNA
tgctcgagggaacacgggaga

SEQ ID NO: 9 - DNA, 24 bp, synthetic construct
  source 1..24: mol_type = other DNA
actacatctccaaaggcagcacct

SEQ ID NO: 10 - DNA, 24 bp, synthetic construct
  source 1..24: mol_type = other DNA
tgtagaactggtcgttcatgggca

SEQ ID NO: 11 - DNA, 20 bp, synthetic construct
  source 1..20: mol_type = other DNA
gtctggagggtgctatgcta

SEQ ID NO: 12 - DNA, 20 bp, synthetic construct
  source 1..20: mol_type = other DNA
caggggcagaaactccagaa

SEQ ID NO: 13 - DNA, 20 bp, synthetic construct
  source 1..20: mol_type = other DNA
caatgaccccttcattgacc

SEQ ID NO: 14 - DNA, 20 bp, synthetic construct
  source 1..20: mol_type = other DNA
gatctcgctcctggaagatg

SEQ ID NO: 15 - DNA, 21 bp, synthetic construct
  source 1..21: mol_type = other DNA
cattccaagtttggcatgaaa

SEQ ID NO: 16 - DNA, 21 bp, synthetic construct
  source 1..21: mol_type = other DNA
cagcaacctgcggcacataaa

SEQ ID NO: 17 - DNA, 17 bp, synthetic construct
  source 1..17: mol_type = other DNA
ggggcctcggtcctagt

SEQ ID NO: 18 - DNA, 18 bp, synthetic construct
  source 1..18: mol_type = other DNA
gatggcaggggtacgaag

SEQ ID NO: 19 - DNA, 20 bp, synthetic construct
  source 1..20: mol_type = other DNA
cacgcctttggcacaatgaa

SEQ ID NO: 20 - DNA, 20 bp, synthetic construct
  source 1..20: mol_type = other DNA
atctcgacgaaacacacccc

SEQ ID NO: 21 - DNA, 20 bp, synthetic construct
  source 1..20: mol_type = other DNA
gaaggatgctgaagcctggt

SEQ ID NO: 22 - DNA, 20 bp, synthetic construct
  source 1..20: mol_type = other DNA
gtcagtaacctcggacctgc

SEQ ID NO: 23 - DNA, 21 bp, synthetic construct
  source 1..21: mol_type = other DNA
gacttctgaacgagagcgaga

SEQ ID NO: 24 - DNA, 21 bp, synthetic construct
  source 1..21: mol_type = other DNA
ccgttcttcaccgacttcctc

SEQ ID NO: 25 - DNA, 20 bp, synthetic construct
  source 1..20: mol_type = other DNA
tgcgactctccaaaaccctc

SEQ ID NO: 26 - DNA, 20 bp, synthetic construct
  source 1..20: mol_type = other DNA
attgcccatcgtcaacacct

SEQ ID NO: 27 - DNA, 20 bp, synthetic construct
  source 1..20: mol_type = other DNA
gcatcactgtttcttggcgt

SEQ ID NO: 28 - DNA, 20 bp, synthetic construct
  source 1..20: mol_type = other DNA
agagcatctccagcacactc

(19) United States
(12) Patent Application Publication
(10) Pub. No.: US 2025/0346643 A1
(43) Pub. Date: Nov. 13, 2025

(54) TROPOMYOSIN KINASE RECEPTOR (TRK) LIGANDS AS THERAPEUTICS TO SUPPORT LIVER REGENERATION

(71) Applicant: Vanderbilt University, Nashville, TN (US)

(72) Inventors: Youngmin A. Lee, Nashville, TN (US); Kevin C. Ray, Nashville, TN (US); Ting-Fang Lee, Nashville, TN (US); Kemal M. Akat, Nashville, TN (US); Vincent Q. Trinh, Nashville, TN (US); Seth J. Karp, Nashville, TN (US)

(21) Appl. No.: 19/203,015

(22) Filed: May 8, 2025

Related U.S. Application Data
(60) Provisional application No. 63/644,381, filed on May 8, 2024.

(51) Int. Cl. C07K 14/48; A61K 38/00

(52) U.S. Cl. CPC C07K 14/48; A61K 38/00

(57) ABSTRACT

Disclosed herein are methods for stimulating liver cell proliferation or liver regeneration in a subject. The methods include administering to the subject a therapeutically effective amount of a compound for activating a tropomyosin kinase receptor (TRK).

### CROSS-REFERENCE TO RELATED APPLICATIONS

This application claims priority to U.S. Provisional Patent Application No. 63/644,381, filed May 8, 2024, which is incorporated herein by reference in its entirety.

### STATEMENT REGARDING FEDERALLY SPONSORED RESEARCH

This invention was made with government support under grant 1R01DK133512 awarded by the National Institutes of Health. The government has certain rights in the invention.

### REFERENCE TO SEQUENCE LISTING

This application was filed with a Sequence Listing XML in ST.26 XML format in accordance with 37 C.F.R. § 1.821. The Sequence Listing XML file submitted in the USPTO Patent Center, “093386-0053-US02 Sequence Listing.xml,” was created on May 6, 2025, contains 28 sequences, has a file size of 25,720 bytes, and is hereby incorporated by reference in its entirety.

### FIELD

This disclosure relates to neurotrophin, melatonin precursor, synthetic, and flavone compounds and methods for increasing liver cell proliferation and liver regeneration.

### INTRODUCTION

Despite the significant public health impact of end-stage liver disease no regenerative therapies are available yet. In contrast to other organs, such as dialysis for chronic kidney disease, LVAD (left ventricular assisted device) in heart failure, or extra-corporal membrane oxygenation for pulmonary failure, there is no temporary replacement therapy for liver function available. Liver transplantation is the only curative therapy for liver cirrhosis and acute liver failure, but the therapy is very costly (about $850,000) and limited due to scarcity of donor organs. Approximately ten thousand liver transplantations were performed in the US in 2023, yet patients may need to wait up to 5 years for a deceased donor organ. Split liver transplantations, in which one organ is split between two recipients, or living donor transplantations have grown considerably to increase donor graft supply, with approximately 660 living donor liver transplants performed in 2023. These procedures rely on the liver's extraordinary ability to regenerate. But complications such as post-hepatectomy liver failure, or ‘small-for-size’ syndrome, which is a consequence of insufficient hepatic regeneration, are severe complications which occur in up to 32% cases 1-3 and contribute to mortality rates of up to 5%. As mentioned above, no regenerative or temporary replacement therapy for liver exists. The therapy is limited to supportive care and a second liver transplantation as a last resort.

Several growth factors that promote hepatocyte proliferation are known. Hepatocyte growth factor (HGF) and epidermal growth factor (EGF) are the only known ‘complete’ hepatocyte mitogens, which in contrast to ‘auxiliary’ hepatocyte mitogens can induce proliferation of primary hepatocytes and liver mass increase if injected into rodents. Extensive preclinical data from different animal models document beneficial effects of rhHGF or HGF-mimetics to promote liver regeneration, but their very low biodistribution and instability in patients have precluded any clinical application.

Thus, there is a need for novel compounds and methods for stimulating liver cell proliferation or liver regeneration.

### SUMMARY

In an aspect, the disclosure relates to a method of stimulating liver cell proliferation in a subject in need thereof. The method may comprise administering to the subject a tropomyosin kinase receptor (TRK) ligand in an amount effective to stimulate liver cell proliferation.

In a further aspect, the disclosure relates to a method of stimulating liver regeneration in a subject in need thereof. The method may comprise administering to the subject a TRK ligand in an amount effective to stimulate liver regeneration.

Another aspect of the disclosure provides a method of stimulating proliferation of a liver cell. The method may comprise contacting the liver cell with a TRK ligand in an amount effective to stimulate proliferation of the liver cell.

Another aspect of the disclosure provides a cell culture medium composition. The cell culture medium may comprise: a base cell culture medium comprising one or more amino acids, salts, buffers, trace minerals, lipids, nucleic acids, additives, and proteins; one or more TRK ligands; one or more growth factors other than a neurotrophin; and one or more additional additives.

The disclosure provides for other aspects and embodiments that will be apparent in light of the following detailed description and accompanying figures.

### BRIEF DESCRIPTION OF THE DRAWINGS

The application file contains at least one drawing executed in color. Copies of this patent application publication with color drawings will be provided by the Office upon request and payment of the necessary fee.

FIG. 1 is a representation of a map showing that Pdgfrb is highly expressed by HSCs. Analysis of a published mouse liver scRNA-seq dataset from uninjured and acetaminophen-treated livers (Ben-Moshe et al., Cell Stem Cell. 2022; 29(6): 973-989.e10) for Pdgfrb using a web interface for querying the dataset (itzkovitzapapp.weizmann.ac.il/apap/?gene1=Col1a1).

FIGS. 2A-L show Jedi T cells deplete HSCs. FIG. 2A is representative immunohistochemical staining for GFP of Pdgfrb-GFP adult mouse liver. PV, portal vein; CV, central vein. Scale bar, 50 μm. n=10 mice. FIG. 2B is representative immunofluorescence microscopy of liver section from Pdgfrb-GFP transgenic mice for GFP and desmin. Scale bar, 20 μm. n=10 mice. FIG. 2C is a diagram showing an experimental scheme. FIG. 2D is representative macroscopic liver images of control and HSC-depleted mice. Scale bar, 5 mm. FIG. 2E is a graph showing liver-to-body weight ratios of control and HSC-depleted mice. Mean+SD. n=8 mice per group. FIG. 2F and FIG. 2G are representative immunofluorescence microscopy of liver sections from control and HSC-depleted mice for desmin (FIG. 2F) and GFP (FIG. 2G). Nuclei are stained with DAPI. Squares indicate areas of enlargement. Scale bars, 100 μm (lower magnification) and 20 μm (enlargements in FIG. 2F) and 50 μm (enlargements in FIG. 2G). n=10 mice per group. FIG. 2H is a graph showing quantification of desmin+ tissue area in control and HSC-depleted mice. Mean+SD. n=5 mice per group. FIG. 2I and FIG. 2J are graphs showing qRT-PCR for Des (FIG. 2I) and Pdgfrb (FIG. 2J) expression in whole-liver RNA. Mean+SD. n=5 mice per group. FIG. 2K is representative H&E staining of sections of control and HSC-depleted livers. Scale bar, 50 μm. Arrows indicate examples of sinusoids. FIG. 2L is a graph showing serum ALT in control and HSC-depleted mice. Mean+SD. n=10 control and 7 HSC-depleted mice. * p<0.05, ** p<0.01, by unpaired two-tailed Mann-Whitney t-test.

FIGS. 3A-E show that Pdgfrb-GFP transgenic mice selectively express GFP in HSCs within liver. FIG. 3A is representative fluorescence microscopy of liver sections from Pdgfrb-GFP mice showing endogenous GFP fluorescence and immunofluorescence staining for GFP and PDGFRB. Nuclei are stained with DAPI. Scale bar, 20 μm. FIG. 3B is representative GFP fluorescence and immunofluorescence staining for desmin in primary HSCs isolated from Pdgfrb-GFP mice. Scale bar, 20 μm. FIG. 3C is representative Immunofluorescence staining for GFP and F4/80, CD31, and HNF4a in liver sections from Pdgfrb-GFP mice. Scale bars, 20 μm. FIG. 3D and FIG. 3E are representative immunofluorescence microscopy of liver sections from control and HSC-depleted livers 10 days after adoptive transfer for reelin (FIG. 3D) and GFAP (FIG. 3E). Squares indicate areas of enlargements (inset). Scale bars, 200 μm. All images are representative of 3 independent experiments.

FIGS. 4A-E show serum analysis and time course of hepatic Jedi T cell expansion and clearance after adoptive transfer. FIG. 4A is a graph showing serum total bilirubin in control and HSC-depleted mice 10 days after adoptive transfer. Mean+SD. * p<0.05 unpaired two-tailed Mann-Whitney test. n=10 (control) and 6 (HSC-depleted). FIG. 4B is a graph showing serum alkaline phosphatase (ALP) in control and HSC-depleted mice 10 days after adoptive transfer. Mean+SD. n=10 (control) and 8 (HSC-depleted). FIG. 4C is a graph showing serum albumin in control and HSC-depleted mice 10 days after adoptive transfer. Mean+SD. **** p<0.0001 by unpaired two-tailed Mann-Whitney test. n=10 (control) and 8 (HSC-depleted). FIG. 4D is plot showing whole liver RNA-seq analysis of control and HSC-depleted mice 10 days after adoptive transfer for Alb, Pros1, Proc, Serpinc1, n=6 (control) and 4 (HSC-depleted). FC, fold change, FDR, false discovery rate. FIG. 4E is representative immunohistochemical staining for granzyme B in liver sections from control and Pdgfrb-GFP mice 3 days, 10 days, and 6 weeks after adoptive transfer. Dashed rectangles indicate areas of enlargement. Scale bars, 100 μm (upper row) and 20 μm (lower row). n=3 controls and HSC-depleted mice (3 days after adoptive transfer), n=5 controls and HSC-depleted mice (10 days after adoptive transfer), n=5 controls and HSC-depleted mice (6 weeks after adoptive transfer).

FIGS. 5A-N show that HSC depletion decreases liver mass and diminishes midlobular hepatocyte numbers. FIG. 5A is a diagram showing an experimental scheme. FIG. 5B is representative immunohistochemical staining for desmin in control and HSC-depleted mice 6 weeks after adoptive transfer. Scale bar, 20 μm. n=5. FIG. 5C is representative macroscopic images of control and HSC-depleted mouse livers in situ. Scale bar, 5 mm. FIG. 5D is a graph showing liver-to-body weight ratios of control and HSC-depleted mice at 6 weeks. N=5 control and 4 HSC-depleted animals. * p<0.05 by unpaired two-tailed Mann-Whitney test.

FIG. 5E and FIG. 5F are representative high- and low-magnification of H&E staining of control and HSC-depleted livers 6 weeks after adoptive transfer. Scale bars, 50 μm (FIG. 5E) and 1 mm (FIG. 5F). n=5 control and 5 HSC-depleted mice. FIG. 5G is representative immunohistochemical staining for CD31 in an HSC-depleted liver section. Arrows indicate endothelial lining of vessels. PV, portal vein; CV central vein. Stars indicate acellular cavities with absent endothelium. Square indicates area of enlargement. Scale bars, 100 μm. Image is representative of 5 mice. FIG. 5H is representative H&E staining of HSC-depleted liver section at 6 weeks. In the annotated image, green dots indicate portal vein (PV), yellow dots central vein (CV), and blue dots acellular cavities. Scale bar, 500 μm. n=5. FIG. 5I is representative immunohistochemical staining for CCND1 in control and HSC-depleted livers at 10 days and 6 weeks after adoptive transfer. Squares indicate areas of enlargement in inserts. Scale bar, 50 μm. n=6 control and 11 HSC-depleted mice at 10 days after adoptive transfer. n=5 control and n=5 HSC-depleted mice 6 weeks after adoptive transfer. FIG. 5J is a representative western blot showing desmin, CCND1, and IGFBP2 in whole-liver lysates from control and HSC-depleted livers 10 days after adoptive transfer. Gapdh is a loading control. Each lane represents 1 mouse. n=3 control and 3 HSC-depleted mice. FIG. 5K is a graph showing whole-liver RNA-seq analysis of control and HSC-depleted livers 10 days after adoptive transfer. n=6 controls and 4 HSC-depleted mice. #, non-differentially expressed genes (FDR>0.34). FIG. 5L is representative immunohistochemical staining of Ki67 and cleaved caspase 3 in control and HSC-depleted livers at 10 days and 6 weeks after adoptive transfer. Scale bar, 30 μm. Dashed rectangles indicate areas of enlargement in inserts. PV, portal vein. CV, central vein. FIG. 5M is a graph showing quantification of Ki67 in control and HSC-depleted livers at 10 days and 6 weeks after adoptive transfer. FIG. 5N is a graph showing quantification of cleaved caspase 3 in control and HSC-depleted livers at 10 days and 6 weeks after adoptive transfer. Ki67+ hepatocytes per mm² in control and HSC-depleted livers at 10 days and 6 weeks after adoptive transfer. n=6 control and 11 HSC-depleted animals at 10 days after adoptive transfer. n=5 control and 5 HSC-depleted animals at 6 weeks after adoptive transfer. Mean+SD. Kruskal-Wallis with post hoc Dunn's test. ** p<0.005.

FIGS. 6A-H show sustained HSC depletion in Pdgfrb-GFP mice 10 weeks after adoptive transfer. FIG. 6A is a diagram showing an experimental outline. FIG. 6B is a graph showing liver-to-body weight ratios of control and HSC-depleted mice 10 weeks after adoptive transfer. Mean+SD. * p<0.0001 by unpaired two-tailed Mann-Whitney test. n=6 (control) and 3 (HSC-depleted) mice. FIG. 6C is a graph showing liver weights of control and HSC-depleted mice 10 weeks after adoptive transfer. Mean+SD. * p<0.05 by unpaired two-tailed Mann-Whitney test. n=6 (control) and 3 (HSC-depleted) mice. FIG. 6D is a graph showing body weights of control and HSC-depleted mice 10 weeks after adoptive transfer. Mean+SD. n=6 (control) and 3 (HSC-depleted) mice. p=0.4513 by unpaired two-tailed Mann-Whitney test. FIG. 6E is a graph showing serum ALT in control and HSC-depleted mice 10 weeks after adoptive transfer. Mean+SD. p=0.2857 by unpaired two-tailed Mann-Whitney test. n=5 (control) and 3 (HSC-depleted) mice. FIG. 6F is representative immunostaining for desmin of control and HSC-depleted livers 10 weeks after adoptive transfer. Scale bar, 50 μm. n=6 (control) and 3 (HSC-depleted) mice. FIG. 6G is representative immunostaining for GFP of control and HSC-depleted livers 10 weeks after adoptive transfer. Scale bar, 50 μm. n=6 (control) and 3 (HSC-depleted) mice. FIG. 6H is representative immunostaining for E-cadherin, CCND1, and glutamine synthetase in control and HSC-depleted livers 10 weeks after adoptive transfer. Scale bar, 100 μm. n=6 (control) and 3 (HSC-depleted) mice. CV, central vein. PV, portal vein.

FIGS. 7A-J show a diminished number of midlobular hepatocytes after sustained HSC depletion. FIG. 7A is representative immunohistochemical staining for GFP in control and HSC-depleted livers 6 weeks after adoptive transfer. Scale bar, 20 μm. n=5 mice. FIG. 7B is a graph showing body weights of control and HSC-depleted mice 6 weeks after adoptive transfer. Mean+SD. n=5 (control) and 4 (HSC-depleted) animals. FIG. 7C is a graph showing liver weights of control and HSC-depleted mice 6 weeks after adoptive transfer. Mean+SD. p=0.0635 by unpaired two-tailed Mann-Whitney test. n=5 (control) and 4 (HSC-depleted) animals. FIG. 7D is a heatmap showing gene expression analysis of whole-liver RNA from HSC-depleted mice vs. controls 10 days after adoptive transfer. FC, fold change; FDR, false discovery rate. n=6 controls and 4 HSC-depleted mice. FIG. 7E is representative low magnification and annotation of H&E staining of control and HSC-depleted livers 6 weeks after adoptive transfer. Scale bar, 1 mm. Dashed rectangle indicates area shown in FIG. 5H. n=5 mice. FIG. 7F and FIG. 7G are representative immunostaining for glutamine synthetase and E-cadherin in control and HSC-depleted livers 10 days (FIG. 7F) and 6 weeks (FIG. 7G) after adoptive transfer. Scale bars, 100 μm. CV, central vein. PV, portal vein. n=5 (control) and n=5 (HSC-depleted) mice. FIG. 7H-J are graphs showing densitometry analysis of immunoblots from liver lysates of control and HSC-depleted mice 10 days after adoptive transfer for desmin (FIG. 7H), CCND1 (FIG. 7I), IGFBP2 (FIG. 7J), normalized to loading control GAPDH. AU, arbitrary units. n=3 per group from 3 independent experiments. Mean+SD. **** p<0.0001, *** p<0.001 by unpaired two-tailed Mann-Whitney test.

FIGS. 8A-J show that NTF3 is an HSC-derived hepatocyte mitogen that increases CCND1. FIGS. 8A-C are graphs showing Olink® protein biomarker analysis (FIG. 8A), multidimensional scaling of Olink® biomarker analysis (FIG. 8B), and quantification of NTF3 (FIG. 8C) in lysates from control and HSC-depleted livers 10 days after adoptive transfer. NPX, normalized protein expression. n=9 control and 6 HSC-depleted mice. FIG. 8D is representative immunofluorescence microscopy for NTF3 and desmin in wild-type adult mouse liver. Nuclei are stained with DAPI. Dashed rectangle indicates area of enlargement. Scale bar, 100 μm and 20 μm in enlargement. PV, portal vein. CV central vein. n=5 mice. FIG. 8E is a representative Western blot of mouse and human HSC cell lines for NTF3. ACTB is a loading control. n=3 independent experiments per cell line. FIG. 8F is a graph showing proliferation of primary wild-type mouse hepatocytes after incubation with NTF3 (20 ng/ml) or vehicle in serum-free media for 5 days. Data from 3 independent experiments. Mean+SD. *** p=0.0002 by unpaired two-tailed Mann-Whitney test. FIG. 8G is a graph showing cell proliferation analysis of HepG2 cells incubated with NTF3, NTF3+Loxo-195, Loxo-195, or vehicle only (control). n=3 independent experiments. Mean+SD. FIG. 8H is a graph showing proliferation of HepG2 cells treated with NTF3, NTF3+Loxo-195, Loxo-195, or vehicle only for 5 days. n=3 independent experiments. Mean+SD. ** p<0.005 by unpaired two-tailed Mann-Whitney test. FIG. 8I is a representative Western blot of CCND1 in lysates from HepG2 cells treated with NTF3 and Loxo-195 as indicated or vehicle only. FIG. 8J is a graph showing Western blot analysis and quantification of CCND1 in lysates from HepG2 cells treated with NTF3 and Loxo-195 as indicated or vehicle only. ACTB is a loading control. n=5 independent experiments. Mean+SD. Kruskal Wallis with post hoc Dunn's test. * p<0.05, ** p<0.01.

FIGS. 9A-N show that NTF3 is an HSC-derived hepatocyte mitogen. FIG. 9A is graph showing analysis of a published scRNA-seq dataset (Ramachandran et al., Nature. 2019; 575(7783): 512-518) of uninjured and cirrhotic human livers for NTF3. FIG. 9B is a graph showing analysis of a published scRNA-seq dataset (Dobie et al., Cell Rep. 2019; 29(7): 1832-1847.e8) of uninjured and CCI4-treated mouse livers for NTF3. FIG. 9C is a graph showing analysis of a published scRNA-seq dataset (Massalha et al., Mol Syst Biol. 2020; 16(12): e9682) of normal human liver and hepatocellular carcinoma for NTF3. FIG. 9D is a graph showing cell proliferation of primary wild-type mouse hepatocytes after incubation with NTF3 (20 ng/ml) or vehicle only in DMEM/5% FBS. n=3 independent experiments. Mean+SD. **** p<0.0001 by unpaired two-tailed Mann-Whitney test. FIG. 9E is a graph showing cell proliferation analysis of murine AML12 cells incubated with NTF3, NTF3+Loxo-195, Loxo-195, or vehicle only. Mean+SD. n=3 independent experiments. FIG. 9F is a graph showing cell proliferation of AML12 cells incubated with NTF3, and Loxo-195 as indicated for 5 days. Mean+SD. **** p<0.0001, ** p<0.005 by Kruskal-Wallis, post hoc Dunn's test. n=3 independent experiments. FIG. 9G and FIG. 9H are a representative Western blot analysis (FIG. 9G) and a graph showing Western blot quantification (FIG. 9H) of phosphorylated TRK (pTRKAY674/675, pTRKBY706/707), total TRK, in lysates of HepG2 cells treated with NTF3 and Loxo-195 as indicated. n=5 independent experiments. * p<0.05 by Kruskal-Wallis, post hoc Dunn's test. FIG. 9I and FIG. 9J are a representative Western blot (FIG. 9I) and a graph showing Western blot quantification (FIG. 9J) for CCND1 and ACTB of AML12 cells incubated with recombinant NTF3 from three different vendors. n=3 independent experiments. * p<0.05 by Kruskal-Wallis, post hoc Dunn's test. FIG. 9K and FIG. 9L are a representative Western blot (FIG. 9K) and a graph showing Western blot quantification (FIG. 9L) for CCND1 and ACTB in cell lysates from HepG2 cells incubated with recombinant NTF3 from three different vendors. n=3 independent experiments. * p<0.05 by Kruskal-Wallis, post hoc Dunn's test. FIG. 9M is a graph showing cell proliferation of primary wild-type mouse hepatocytes after incubation with NTF3 (20 ng/ml), HGF (5 ng/ml), or vehicle only in serum-free medium. Mean+SD. n=3 independent experiments. *p<0.05, **** p<0.0001 by Kruskal-Wallis, post hoc Dunn's test. FIG. 9N is a graph showing cell proliferation of primary wild-type mouse hepatocytes after incubation with NTF3 (20 ng/ml), HGF (5 ng/ml), or NTF+HGF in serum-free medium. Mean+SD. n=3 independent experiments. p=0.6981 by Kruskal-Wallis, post hoc Dunn's test.

FIGS. 10A-I show that recombinant NTF3 increases hepatocyte CCND1 in HSC-depleted mice. FIG. 10A is a diagram showing an experimental scheme. FIG. 10B is representative macroscopic images of livers in control and HSC-depleted mice treated with vehicle or NTF3. Scale bar, 1 cm. FIG. 10C is a graph showing liver-to-body weight ratios of control and HSC-depleted mice treated with vehicle or the indicated concentrations of NTF3. *p<0.05 by Kruskal-Wallis, post hoc Dunn's test. n=8-13 animals per group. FIG. 10D is representative H&E and CCND1 staining of liver sections from control and HSC-depleted mice treated with vehicle or NTF3. Arrowheads indicate positive staining. Dashed rectangles indicate areas shown in insets. FIG. 10E is representative multiplex immunohistochemistry for CCND1 and HNF4a in liver sections from control and HSC-depleted mice treated with vehicle or NTF3. Dashed rectangles indicate areas of enlargement. Scale bars, 20 μm. Arrowheads indicate positive-staining nuclei. FIG. 10F is a graph showing quantification of CCND1+HNF4α+ nuclei per high power field (HPF). Grey bars indicate mean. n=3-5 animals per group. *p<0.05, ** p<0.005, **** p<0.0001 by Kruskal-Wallis, post hoc Dunn's test. FIG. 10G is a graph showing quantification of Ki67+ hepatocytes in control and HSC-depleted mice treated with vehicle or NTF3. Mean+SD. * p<0.05 by Kruskal-Wallis, post hoc Dunn's test. n=4-6 animals per group. FIG. 10H is representative Ki67 staining of liver sections from control and HSC-depleted mice treated with vehicle or NTF3. Arrowheads indicate positive staining. Dashed rectangles indicate areas shown in insets. FIG. 10I is a representative Western blot for desmin, CCND1, IGFBP2, and PCNA in whole-liver lysates from control and HSC-depleted mice treated with vehicle or recombinant NTF3. GAPDH is a loading control. N=3 animals per group.

FIGS. 11A-D show pan-lobular expression of HNF4a in hepatocytes in control and HSC-depleted mice. FIG. 11A is representative immunohistochemical staining for HNF4a in control and HSC-depleted livers 6 weeks after adoptive transfer. Scale bar, 50 μm. n=5 mice. FIG. 11B is a graph showing quantification of Western blots for PCNA and loading control GAPDH from liver lysates of control and HSC-depleted mice treated with NTF3 or vehicle. n=3 independent experiments. * p<0.05 by Kruskal-Wallis, post hoc Dunn's test. FIG. 11C is a graph showing quantification of Western blots for CCND1 and loading control GAPDH from liver lysates of control and HSC-depleted mice treated with NTF3 or vehicle. n=3 independent experiments. ** p=0.002 by Kruskal-Wallis, post hoc Dunn's test. FIG. 11D is representative immunofluorescence microscopy for TRKB in adult mouse liver. Nuclei are stained with DAPI. n=5 mice. PV, portal vein. CV, central vein. Scale bar represents 100 μm.

FIGS. 12A-H show that NTF3-induced cell proliferation is mediated by TRKB in primary mouse hepatocytes. FIG. 12A is a graph showing analysis of CCND1, NTRK1, NTRK2, and NTRK3 expression in a published mouse liver gene expression dataset (Halpern et al., Nature. 2017; 542(7641): 352-356) that enables gene expression analysis according to the hepatocyte's position in the lobule layers. FIG. 12B is representative immunofluorescence microscopy for TRKA and TRKB in normal adult mouse liver at 4 months of age. Dashed rectangle indicates area of enlargement. CV, central vein. PV, portal vein. Scale bar, 100 μm. n=3 mice. FIG. 12C is representative immunofluorescence microscopy for TRKB in adult mouse liver also stained with phalloidin to label F-actin. Nuclei are stained with DAPI. PV, portal vein. CV, central vein. Scale bar, 100 μm, 20 μm in enlargement. FIG. 12D is a graph showing proliferation of primary wild-type mouse hepatocytes after incubation with NTF3 (20 ng/ml), TRKB antagonist ANA-12 (10 nM), NTF3+ANA-12, or ANA-12 only in serum-free medium. Mean+SD. Data from n=3 independent experiments. **** p<0.0001, ** p<0.005 by Kruskal-Wallis, post hoc Dunn's. FIG. 12E is a graph showing qRT-PCR for Ntrk2 expression in primary mouse hepatocytes infected with lentivirus carrying non-targeting control shRNA (shNT), or Ntrk2-targeting shRNAs (shNtrk2_1 or shNtrk2_2). Mean+SD. Data from n=3 independent experiments. * p<0.05, **** p<0.0001 by Kruskal-Wallis, post hoc Dunn's. FIG. 12F is a graph showing proliferation analysis of primary mouse hepatocytes infected with shNT, shNtrk2_1, or shNtrk2_2 lentivirus and incubated with NTF3 (20 ng/ml) or vehicle only. Analysis by two-way ANOVA, post hoc Sidak test comparing vehicle-only-treated vs. NTF3-treated cells. Mean+SD. Data from n=3 independent experiments. FIG. 12G is a graph showing bulk RNA-seq analyses for Ntf3 in livers 72 hours after partial hepatectomy and controls. n=5 control and 3 hepatectomized mice per group. FC, fold change; FDR, false discovery rate. FIG. 12H is a graphical summary of the main findings. The diagram depicts the metabolic zonation of hepatocytes between the central vein (CV) and the portal triad, which includes branches of the portal vein and hepatic artery and a bile duct. Under normal conditions, HSCs produce NTF3 that activates TRKB on midlobular (zone 2) hepatocytes, leading to CCND1-dependent proliferation. Upon HSC ablation by Jedi T cells, midlobular hepatocytes stop proliferating, leading to a gradual reduction in liver mass as dead hepatocytes are not replaced.

FIGS. 13A-N show that hepatic stellate cells are required for liver regeneration after partial hepatectomy. FIG. 13A is a diagram showing an experimental outline. FIG. 13B is a Kaplan-Meier plot showing survival 1 h after partial hepatectomy. FIG. 13C is a Kaplan-Meier plot showing survival 24 h after partial hepatectomy. FIG. 13D is a Kaplan-Meier plot showing survival 72 h after partial hepatectomy. n=5 per group and time point. FIG. 13E is a graph showing body weight in control and HSC-depleted mice 24 h after partial hepatectomy. FIG. 13F is a graph showing liver weight in control and HSC-depleted mice 24 h after partial hepatectomy. FIG. 13G is a graph showing liver to body weight ratio 24 h after partial hepatectomy. FIG. 13H is a graph showing serum ALT levels in control and HSC-depleted animals. FIG. 13I is representative IHC images of Ki67 in control and HSC depleted mice 72 h after partial hepatectomy. FIG. 13J is a diagram showing an experimental outline. FIG. 13K is a Kaplan-Meier plot showing survival 24 h after partial hepatectomy in HSC-depleted mice (Gfap-Tk) and controls. FIG. 13L is a graph showing liver to body weight ratio 24 h after partial hepatectomy. FIG. 13M is a graph showing quantification of PCNA+ hepatocytes per high power field. FIG. 13N is representative images of PCNA immunostaining in control and HSC-depleted mice (Gfap-Tk) 24 h after partial hepatectomy. N=4-6 animals per group. Mean+SD.

FIGS. 14A-K show that NTF3 administration increases number of proliferating hepatocytes 2 days and 4 days after partial hepatectomy. FIG. 14A is a diagram showing an experimental outline. FIG. 14B is a graph showing liver/body weight ratio 2 days after partial hepatectomy in control and NTF3-treated animals. FIG. 14C is a graph showing quantification of Ki67+ hepatocytes per high power field (HPF) in vehicle- or NTF3-treated mice. **** p<0.001 by unpaired Mann-Whitney test. FIG. 14D is a graph showing serum ALT levels in vehicle or NTF3-treated mice. FIG. 14E is a graph showing serum albumin levels in vehicle or NTF3-treated mice. FIG. 14F is a diagram showing an experimental outline. FIG. 14G is a graph showing liver/body weight ratio 4 days after partial hepatectomy in control and NTF3-treated animals. FIG. 14H is a graph showing quantification of Ki67+ hepatocytes per high power field (HPF) in vehicle- or NTF3-treated mice. * p<0.05 by unpaired Mann-Whitney test. FIG. 14I is a graph showing serum ALT levels in vehicle or NTF3-treated mice. FIG. 14J is a graph showing serum albumin levels in vehicle or NTF3-treated mice. FIG. 14K is representative images of immunostaining for Ki67 of vehicle and NTF3-treated mice. 2 days and 4 days after partial hepatectomy. Scale bar indicates 50 mm.

FIGS. 15A-H shows that NTF3 administration increases number of proliferating hepatocytes 2 days and 4 days after partial hepatectomy. FIG. 15A is a diagram showing an experimental outline. FIG. 15B is a graph showing liver weight 2 days after partial hepatectomy in control and NTF3-treated animals. FIG. 15C is a graph showing body weight 2 days after partial hepatectomy in control and NTF3-treated animals. FIG. 15D is a graph showing serum ALP 2 days after partial hepatectomy in control and NTF3-treated animals. FIG. 15E is a diagram showing an experimental outline. FIG. 15F is a graph showing liver weight 4 days after partial hepatectomy in control and NTF3-treated animals. FIG. 15G is a graph showing body weight 4 days after partial hepatectomy in control and NTF3-treated animals. FIG. 15H is a graph showing serum ALP 4 days after partial hepatectomy in control and NTF3-treated animals. Mean+SD.

FIGS. 16A-E show that BDNF and NTF3 are expressed by hepatic stellate cells in mouse and human liver. FIG. 16A is representative immunofluorescence images for reelin and BDNF and reelin in adult mouse liver. FIG. 16B is representative images of immunoblotting for BDNF, NTF3, and β-actin in cell lysates from mouse and human HSC cell lines JS1, LX2, and TWNT4. FIG. 16C is a graph showing proliferation in primary mouse hepatocytes cultured in control media, with BDNF, BDNF and Ana-12, and Ana-12 alone. N=3 independent experiments. Mean+SD. FIG. 16D is representative immunofluorescence images of BDNF and reelin or NTF3 and reelin in normal adult human liver. FIG. 16E is a graph showing cell proliferation of primary mouse hepatocytes cultured in control media, NTF3, HGF, BDNF, and N-acetyl serotonin (NAS). N=3 experiments, Mean+SD. One-way ANOVA, post hoc Tukey's test.

FIGS. 17A-C show that BDNF is expressed by human HSCs, TRKB agonists LM22A-4 and 7,8-DHF do not induce proliferation in primary mouse hepatocytes. FIG. 17A is a graph showing single cell RNA-seq analysis of human liver from published dataset (Massalha et al., Mol Syst Biol. 2020; 16(12): e9682) and demonstrates BDNF expression in HSCs and cancer-associated fibroblasts only. FIG. 17B is a graph showing cell proliferation analysis of primary mouse hepatocytes incubated with TRKB agonist LM22A-4 at 100 mM, 200 mM, and 500 mM. FIG. 17C is a graph showing cell proliferation analysis of primary mouse hepatocytes incubated with reported TRKB agonist 7,8-DHF at 10 mM, 20 mM, and 50 mM. N=3 experiments each.

FIGS. 18A-I show human ductal organoids. FIG. 18A is representative images of primary human hepatocyte 1 day after plating on collagen coated plate (2D) and in Matrigel (3D). FIG. 18B is representative images of human ductal organoid formation 3 days and 9 days after isolation. FIG. 18C is representative images of human ductal organoids 3 days after passaging. FIG. 18D is a graph showing qRT-PCR of HDOs that had been cultured in organoid differentiation medium (ODM) vs. organoid growth medium (OGM). qRT-PCR for SOX9, KRT19, ALB, and HNF4A. FIG. 18E is representative images of EdU incorporation assays of HDOs that have been cultured in basal media, organoid differentiation media (ODM), and serum-free William's E media. FIG. 18F is representative images of HDO morphological changes after culturing in OGM, then ODM, and finally Williams E. medium. FIG. 18G is representative images of an immunoblot of hepatocytic differentiated HDOs that have been cultured in media containing Glutamine and or Torin2. Immunoblotting for P-S6RP^{Ser240/244}, S6RP, and GAPDH. FIG. 18H is representative images of immunostaining of mouse passaged primary human hepatocytes (mpPHH) with human species specific NuMA. FIG. 18I is a graph showing quantification of NuMA+ nuclei. Mean+SD.

FIGS. 19A-F show that BDNF and NTF3 induce proliferation in human hepatic organoids and mouse passaged primary human hepatocytes. FIG. 19A is representative images showing EdU incorporation in hepatocytic differentiated HDOs cultured with vehicle, BDNF, NTF3, or HGF. Inserts depict enlargements of marked areas. FIG. 19B is a graph showing quantification of EdU+ nuclei/Hoechst 33342+ nuclei. N=3 experiments. Mean+SD, Kruskal Wallis, and post hoc Dunn's test. FIG. 19C is representative images of EdU incorporation in hepatocytic differentiated HDOs cultured with vehicle, NTF3, or taletrectinib. FIG. 19D is a graph showing quantification of EdU+ nuclei/Hoechst 33342+ nuclei. N=3 experiments. Mean+SD, Kruskal Wallis, and post hoc Dunn's test. FIG. 19E is representative images showing EdU incorporation in mouse passaged primary human hepatocytes (mpPHH) cultured with vehicle, BDNF, NTF3, or HGF. FIG. 19F is a graph showing quantification of EdU+ nuclei/Hoechst 33342+ nuclei. N=3 experiments. Mean+SD, Kruskal Wallis, and post hoc Dunn's test. * p<0.05, ** p<0.01, *** p<0.005, **** p<0.0001.

FIGS. 20A-F show that NTF3 activates mTOR in human ductal organoids and mouse passaged primary human hepatocytes. FIG. 20A is representative images of immunoblotting of hepatocytic differentiated human ductal organoids (HDO) for P-S6RP (Ser235/236), P-S6RP ser240/244, S6RP, P-P70S6K (Thr389), P70S6K, and GAPDH. Coomassie staining of gel is shown at bottom. FIG. 20B is a graph showing densitometry of P-S6RP^{(Ser240/244)}/GAPDH. N=4, Kruskal-Wallis, and post hoc Dunn's test. FIG. 20C is a graph showing densitometry of immunoblots of HDOs for P-S6RP^{(Ser235/236)}/GAPDH. N=4, Kruskal-Wallis, and post hoc Dunn's test. FIG. 20D is representative images of immunoblotting of mouse passaged primary human hepatocytes (mpPHH) for P-S6RP^{(Ser235/236)}, S6RP, P-P70S6K Thr389, P70S6K, and GAPDH. N=3 experiments. FIG. 20F is representative images of immunoblotting of hepatocytic differentiated HDOs for that have been cultured with NTF3, Torin2, insulin for CCND1, PCNA, and GAPDH. Coomassie staining of gel is shown at bottom. FIG. 20G is representative images showing immunoblotting of mpPHH that have been cultured with NTF3, Torin2 for CCND1, PCNA, and GAPDH.

### DETAILED DESCRIPTION

Described herein are methods of stimulating liver cell proliferation in a subject in need thereof, that may comprise administering to the subject a tropomyosin kinase receptor (TRK) ligand in an amount effective to stimulate liver cell proliferation. Also described herein are methods of stimulating liver regeneration in a subject in need thereof, that may comprise administering to the subject a TRK ligand in an amount effective to stimulate liver regeneration. Finally, described herein are methods of stimulating proliferation in a liver cell, that may comprise contacting the liver cell with a TRK ligand in an amount effective to stimulate proliferation of the liver cell.

### 1. DEFINITIONS

Unless otherwise defined, all technical and scientific terms used herein have the same meaning as commonly understood by one of ordinary skill in the art. The meaning and scope of the terms should be clear. In case of conflict, the present document, including definitions, take precedent over any dictionary or extrinsic definition. Further, unless otherwise required by context, singular terms shall include pluralities and plural terms shall include the singular. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, in some embodiments within 5-fold, and in some embodiments within 2-fold, of a value.

The terms “active ingredient” or “active pharmaceutical ingredient” as used herein refer to a pharmaceutical agent, active ingredient, compound, substance, compositions, or mixtures thereof, that provide a pharmacological, often beneficial, effect.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Healthy or normal levels or ranges may be defined in accordance with standard practice. A control may be a subject or cell without a compound as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

The term “dose” as used herein denotes any form of the active ingredient or composition that contains an amount sufficient to produce a therapeutic effect with at least a single administration.

The term “dosage” as used herein refers to the administering of a specific amount, number, and frequency of doses over a specified period of time, typically 1 day.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB

Biochemical Nomenclature Commission. Amino acids include the side chain and polypeptide backbone portions.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

The term “cell” or “cells” as used herein refers to not only isolated cells, but cells within an organoid, cells in a tissue sample, cells in a subject, and anything containing the cell.

The terms “cell culture” or “culture” as used herein refer to the maintenance of cells in an artificial, e.g., an in vitro or ex vivo environments. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems, organoids or whole organisms, for which the terms “tissue culture,” “organ culture,” “organ system culture” or “organotypic culture” may be used interchangeably with the term “cell culture.”

The phrases “cell culture medium,” “culture medium,” “medium formulation,” or “medium” (plural “media” in each case) as used herein refer to a nutritive solution that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free, protein-free (one or both), may or may not require additional components like growth factors, additives, feeds, supplements, for efficient and robust cell performance.

The term “contacting” as used herein refers to the placing of cells to be cultivated into a culture vessel with the medium in which the cells are to be cultivated. The term “contacting” encompasses, for example, mixing cells with medium, perfusing cells with medium, pipetting medium onto cells in a culture vessel, and submerging cells in culture medium. “Contacting” or “in contact with each other” as used herein with includes the compounds described herein that are in physical contact with the cells and permits the components to interact or react with each other.

The term “culture vessel” as used herein refers to a receptacle for holding cells. The vessel may be glass, plastic, metal, or other material that can provide an aseptic environment for culturing, cultivating, holding, or storing cells.

The term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

A “feed” or “supplement” as used herein refers to a composition when added to cells in standard culture may be beneficial for its maintenance, or expansion, or growth, or viability, or affects its cell performance, or increases culture longevity or maintaining cells in a pseudo-stationary phase wherein product expression continues, or results in a significant increase in final product titer. A feed or supplement may be used interchangeably in this disclosure and refers to liquid formats of media components comprising one or more amino acids, sugars, vitamins, buffers, peptides, hydrolysates, fractions, growth factors, hormones, etc. required to rebalance or replenish or to modulate the growth or performance of a cell in culture, or a cell culture system. A feed or supplement may be distinguished from a cell culture medium in that it is added to a cell culture medium that can culture a cell. As would be understood by one of skill in the art, sometimes a feed/supplement may comprise those amino acids, sugars, vitamins, buffers, etc. required to rebalance or replenish or modulate the growth or performance of a cell in culture, or a cell culture system.

As used herein, the term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, carbohydrates, lipids, nucleic acids, hormones, vitamins, fatty acids, proteins, and the like. Other ingredients that promote or maintain cultivation of cells ex vivo can be selected by those of skill in the art, in accordance with the particular need.

“Liver cell proliferation” as used herein refers to the process by which liver cells, such as hepatocytes and cholangiocytes, increase in number through cell division. This process is crucial for liver regeneration after injury or disease, and for maintaining the overall liver mass and function.

“Liver regeneration” as used herein refers to the ability of the liver to repair and restore itself after injury or removal of a portion of its tissue. This process involves a complex series of events such as hepatocyte proliferation, transdifferentiation of hepatocytes and cholangiocytes, and upregulation or downregulation in growth factors, ultimately restoring the liver's mass and function.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A domain may be comprised of a series of the same type of motif.

As used herein “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. In some embodiments, “physiologic pH” is about pH 7.4±10%.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all. In another example, recombinant proteins are made using an engineered DNA sequence that contains genetic instructions to synthesize the protein of interest. Then, this DNA is delivered to a host cell where the cell's internal machinery is used to synthesize the recombinant protein.

As used herein, the terms “room temperature,” “RT,” or “ambient temperature” refer to typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ˜ 20° C., ˜22° C., ˜25° C., or ˜27° C., at standard atmospheric pressure.

“Sample” or “test sample” as used herein can mean any sample in which the presence and/or level of a molecule, such as a protein or expression marker, is to be detected or determined or any sample comprising a compound as detailed herein. Samples may include liquids, solutions, emulsions, or suspensions. Samples may include a medical sample. Samples may include any biological fluid or tissue, such as blood, whole blood, fractions of blood such as plasma and serum, liver tissue, muscle tissue, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, peripheral blood mononuclear cells, white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein a “solvent” is a liquid that dissolves or has dissolved another ingredient of the medium. Solvents may be used in preparing media, in preparing subgroups, supplements or other formulations, and in reconstituting media or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water are preferred because of their ease of preparation and handling.

“Subject,” “study participant,” “participant,” and “patient” as used herein interchangeably refers to any vertebrate, including, but not limited to, a mammal that wants or is in need of the herein described compounds, compositions, or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgus monkey, rhesus monkey, chimpanzee, gorilla, orangutan, and gibbon. The subject may be of any age or stage of development, such as, for example, an adult, an adolescent, or an infant. The subject may be male. The subject may be female. The subject may be undergoing other forms of treatment.

The term “a therapeutically effective amount” of a compound as described herein refers to an amount of the compound as described herein that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of a compound as described herein that, when administered to a subject, is effective to, at least partially alleviate, inhibit, prevent and/or ameliorate a condition or a disorder mediated by a liver disease or liver damage.

“Treatment” or “treating” when referring to protection of a subject from a disease or disorder, means suppressing, repressing, reversing, alleviating, ameliorating, or inhibiting the progress of disease or disorder, or completely eliminating a disease or disorder. A treatment may be either performed in an acute or chronic way. The term also refers to reducing the severity of a disease or a disorder or symptoms associated with such disease or disorder prior to affliction with the disease or disorder. Preventing the disease or disorder involves administering a composition or compound of the present disclosure to a subject prior to onset of the disease or disorder. Suppressing the disease or disorder involves administering a composition or compound of the present disclosure to a subject after induction of the disease or disorder but before its clinical appearance. Repressing or ameliorating the disease or disorder involves administering a composition or compound of the present disclosure to a subject after clinical appearance of the disease or disorder.

“Tropomyosin kinase receptor ligand” or “TRK ligand” or “Trk ligand” or “TRK agonist” or “Trk agonist” as used herein refers to a compound or molecule that is capable of inducing signaling through a Trk receptor such as TrkA, TrkB, and TrkC.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote a response in a cell. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. For example, amino acids having hydropathic indexes of ±2 may be substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. COMPOUNDS FOR STIMULATING LIVER CELL PROLIFERATION OR LIVER REGENERATION

Provided herein are methods of stimulating liver cell proliferation in a subject in need thereof. Also provided herein are methods of stimulating liver regeneration in a subject in need thereof. The methods may include administering to the subject a tropomyosin kinase receptor (TRK) ligand in an amount effective to stimulate liver cell proliferation. The method may include administering to the subject a TRK ligand in an amount effective to stimulate liver regeneration. Further provided herein are methods of stimulating proliferation of a liver cell in vitro or ex vivo. The method may include contacting the liver cell with a TRK ligand in an amount effective to stimulate proliferation of the liver cell. Further provided herein are cell culture media compositions. The cell culture media composition may comprise one or more TRK ligands.

Liver cells are broadly categorized into parenchymal and nonparenchymal types. The main parenchymal cells are hepatocytes. Nonparenchymal cells include cholangiocytes (bile duct cells), liver sinusoidal endothelial cells, hepatic stellate cells, Kupffer cells, and mesothelial cells. Hepatocytes are the primary functional cells of the liver, responsible for a wide range of metabolic processes, including protein synthesis, bile production, and detoxification. They make up about 70-85% of the liver's volume. Cholangiocytes line the bile ducts within the liver and are responsible for secreting bile. Liver sinusoidal endothelial cells line the sinusoids, which are the small blood vessels within the liver, and play a role in filtering blood. Hepatic stellate cells are in the space of Disse, a space between the sinusoids and hepatocytes, and are involved in collagen synthesis and storage of Vitamin A. Kupffer cells are phagocytic cells that reside within the sinusoids and remove debris and old cells from the blood. Mesothelial cells line the outer surface of the liver. The liver cell may be a hepatocyte, a cholangiocyte, or a combination thereof.

A TRK ligand may be a neurotrophin, a melatonin precursor, a neurotrophin mimetic, a flavone, or a combination thereof.

a. Neurotrophins

Neurotrophins are a family of proteins that are essential for the development and function of the vertebrate nervous system. Neurotrophins produce signals through two different types of cell surface receptors, Trk receptor tyrosine kinases and the p75 neurotrophin receptor (p75NTR).

Trk receptor tyrosine kinases are a family of three membrane-bound proteins (TrkA, TrkB, and TrkC) that are activated by neurotrophins. Trk receptors are transmembrane proteins consisting of an extracellular domain, a single transmembrane region, and an intracellular (or cytoplasmic) tyrosine kinase domain. The extracellular domain comprises leucine-rich motifs, cysteine clusters, and immunoglobulin (lg)-like domains, critical for neurotrophin binding. The intracellular domain contains the tyrosine kinase that initiates signaling cascades through, for example, phosphorylation of downstream proteins. Neurotrophins directly bind and dimerize Trk receptors, which results in activation of the tyrosine kinase present in the intracellular domain. The primary site at which Trk receptors interact with neurotrophins is the most proximal Ig-like domain of the receptor. Trk receptors can form homodimers or heterodimers when dimerizing.

p75NTR is a type I transmembrane protein, with a molecular weight of 75 kDa and consists of an extracellular domain, a transmembrane domain, and an intracellular domain. p75NTR is a member of the superfamily of tumor necrosis factor receptors that bind neurotrophins (and their respective pro-neurotrophins) and mediates neuronal growth, synapse plasticity, and apoptosis. p75NTR can interact with Trk receptors. p75NTR affects the binding affinity and specificity of Trk receptor activation by neurotrophins. p75NTR can increase neurotrophin binding affinity to Trk receptors and reduce ligand-induced receptor ubiquitination, and delay receptor internalization and degradation. For example, the presence of p75NTR increases the binding affinity of nerve growth factor (NGF) to TrkA.

Mammalian neurotrophins include four structurally related factors: NGF, brain-derived neurotrophic factor (BDNF), neurotrophin-3 (NT-3 or NTF3), and neurotrophin-4 (NT-4, also known as NT-5). These neurotrophins can form dimers such as NT-3/BDNF, NT-4/BDNF, and NGF/NGF. NGF is specific for TrkA. BDNF and NT-4 are specific for TrkB. NT-3 activates TrkC and is also able to activate TrkA and TrkB receptors, but with less efficiently. Isoforms of TrkA, TrkB, and TrkC as a result of differential mRNA splicing affect their extracellular domains and, therefore, their binding of NGF, BDNF, NT-3, and NT-4. The presence or absence of short amino acid sequences in the juxtamembrane domains of each receptor affect the ability of some neurotrophins to activate the receptor. For example, BDNF, NT-3, and NT-4 can activate a TrkB isoform containing a short amino acid sequence in the juxtamembrane domain, but only BDNF can activate a TrkB isoform lacking those amino acids. Similarly, an isoform of TrkA containing a short juxtamembrane sequence is activated by NGF and NT-3, whereas a TrkA isoform lacking those amino acids is activated by only NGF. In addition, NT-3 activation of TrkA and NT-3 and NT-4 activation of TrkB are negatively regulated by high levels of p75NTR.

The neurotrophin may be one or more of NGF, NTF3, NT-4, and BDNF. In some embodiments, the neurotrophin may be one or more of NTF3 and BDNF. The neurotrophin may be one or more of recombinant NTF3 and recombinant BDNF.

b. Melatonin Precursor

The melatonin production pathway includes four precursors: tryptophan, 5-hydroxytryptophan (5-HTP), serotonin, and N-acetyl serotonin (NAS, also known as normelatonin). Tryptophan is converted to 5-HTP by the enzyme tryptophan hydroxylase. Then, 5-HTP is converted to serotonin by the enzyme 5-HTP decarboxylase. Next, serotonin is converted to NAS by the enzyme arylalkylamine N-acetyltransferase (AANAT). Finally, NAS is converted to melatonin by the enzyme hydroxyindole-O-methyltransferase (HIOMT).

The melatonin precursor may be one or more of tryptophan, 5-HTP, serotonin, and NAS. In some embodiments, the melatonin precursor may be NAS.

NAS acts as an agonist at melatonin receptors (MT1, MT2, MT3), mimicking the effects of melatonin, and activates TrkB. NAS exhibits neurotrophic effects, including neuroprotection and promotion of neurogenesis. The molecular formula for NAS is C₁₂H₁₄N₂O₂ and it has a molecular weight of 218.256 g/mol. NAS has the following structural formula, Formula (I):

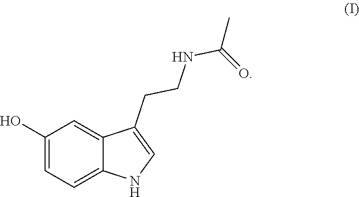 [SMILES: CC(=O)NCCc1c[nH]c2ccc(O)cc12]

c. Neurotrophin Mimetic

The neurotrophin mimetic may be LM22A-4, GSB-106, BNN-20, tricyclic dimeric peptide 6 (TDP6), LM11A-31, or a combination thereof.

LM22A-4 is a synthetic, selective small-molecule partial agonist of TrkB. LM22A-4 has an EC50 for TrkB activation of 200-500 pM, IC50 for inhibition of BDNF binding to TrkB of 47 nM, and an intrinsic activity (IA) of about 85%. The compound is known to produce neurogenic and neuroprotective effects in animals. The molecular formula for LM22A-4 is C₁₅H₂₁N₃O₆ and it has a molecular weight of 339.348 g/mol. LM22A-4 is described in U.S. Pat. No. 8,686,045, which is fully incorporated herein by reference. LM22A-4 has the following structural formula, Formula (II):

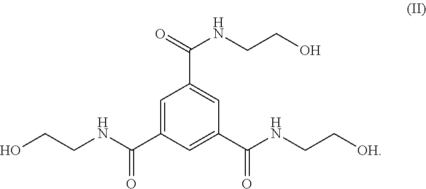 [SMILES: O=C(NCCO)c1cc(C(=O)NCCO)cc(C(=O)NCCO)c1]

GSB-106 (bis-[N-monosuccinyl-L-seryl-L-lysine] hexamethylenedia-mide) is a dimeric dipeptide that mimics the TrkB-interaction loop 4 of BDNF and has shown neurotrophic and neuroprotective effects. GSB-106 is described in U.S. Pat. No. 9,683,014, which is fully incorporated herein by reference.

BNN-20 is a derivative of dehydroepiandrosterone (DHEA), which binds to TrkB and has been shown to protect dopaminergic neurons in a mouse model of Parkinson's disease. The molecular formula for BNN-20 is C₂₀H₃₀O₂ and it has a molecular weight of 302.458 g/mol. BNN-20 is described in U.S. Pat. No. 9,777,037, which is fully incorporated herein by reference. BNN-20 has the following structural formula, Formula (III):

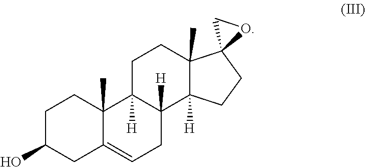

TDP6 is a tricyclic dimeric peptide that mimics BDNF and induces autophosphorylation of TrkB. TDP6 is described in U.S. Patent Application Publication No. 2019/0022101, which is fully incorporated herein by reference.

LM11A-31 is a small molecule ligand for p75NTR that has shown promising results in preclinical studies and is currently undergoing clinical trials for Alzheimer's disease. The molecular formula for LM11A-31 is C₁₂H₂₅N₃O₂ and it has a molecular weight of 243.351 g/mol. LM11A-31 is described in U.S. Patent Application Publication No. 2014/0100224, which is fully incorporated herein by reference. LM11A-31 has the following structural formula, Formula (IV):

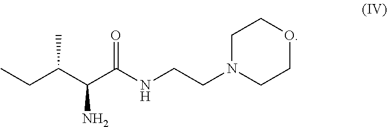 [SMILES: CC[C@H](C)[C@H](N)C(=O)NCCN1CCOCC1]

d. Flavone

Flavones are a type of flavonoid with a core structure consisting of two benzene rings (A and B) and a pyran ring (C) fused together. Specifically, the B ring is connected to the C ring at position 2, and the C ring contains a double bond between carbons 2 and 3, and a ketone group at position 4. The A ring typically has hydroxyl groups at positions 5 and 7, which can be free or esterified. Flavones have the following generic structural formula, Formula (V):

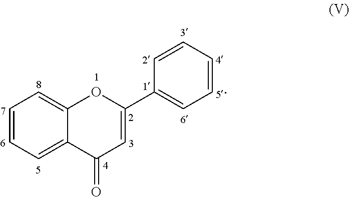 [SMILES: O=c1cc(-c2ccccc2)oc2ccccc12]

R³ may be —H. R⁵ may be —H, —OH, or —OCH₃. R⁶ may be —H, —OH, or —OCH₃. R⁷ may be —H, —OH, or —OCH₃. R⁸ may be —H, —OH, or —OCH₃. R² may be —H, —OH, or —OCH₃. R^{3′} may be —H, —OH, or —OCH₃. R^{4′} may be —H, —OH, or —OCH₃. R^{5′} may be —H, —OH, or —OCH₃. R⁶″ may be —H or —OCH₃.

The flavone may be one or more of 7,8-dihydroxyflavone (DHF, also known as tropoflavin), chrysin, quercetin, and apigenin.

Chrysin, also known as 5,7-dihydroxyflavone, is a flavone that activates both tropomyosin receptor kinase B (TrkB) and FGFR1 receptors while upregulating their ligands, promoting neurogenesis. It is found in honey, propolis, the passion flowers, Passiflora caerulea and Passiflora incarnata, and Oroxylum indicum. The molecular formula for chrysin is C₁₅H₁₀O₄ and it has a molecular weight of 254.241 g/mol. Chrysin has the following structural formula, Formula (VI):

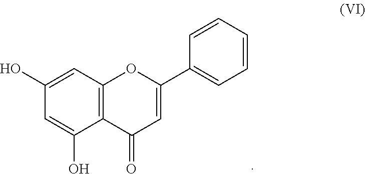 [SMILES: O=c1cc(-c2ccccc2)oc2cc(O)cc(O)c12]

Quercetin is a flavone that has been shown to have neuroprotective effects in SH-SY5Y cells, reduce Tau aggregation, and activate TrkB-mediated signaling pathways. It is found in many fruits, vegetables, leaves, seeds, and grains. For example, capers, red onions, and kale are common foods containing appreciable amounts of quercetin. The molecular formula for quercetin is C₁₅H₁₀O₇ and it has a molecular weight of 302.236 g/mol. Quercetin has the following structural formula, Formula (VII):

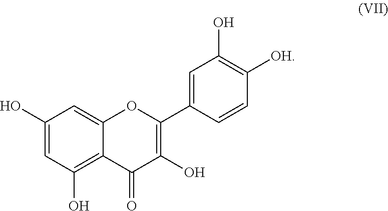 [SMILES: O=c1c(O)c(-c2ccc(O)c(O)c2)oc2cc(O)cc(O)c12]

Apigenin, also known as 4′,5,7-trihydroxyflavone, is a flavone that has been shown to have neuroprotective effects in SH-SY5Y cells, reduce Tau aggregation, and activate TrkB-mediated signaling pathways. Apigenin is found in many fruits and vegetables. For example, parsley, celery, celeriac, and chamomile tea are the most common sources of apigenin. The molecular formula for apigenin is C₁₅H₁₀O₅ and it has a molecular weight of 270.240 g/mol. Apigenin has the following structural formula, Formula (VIII):

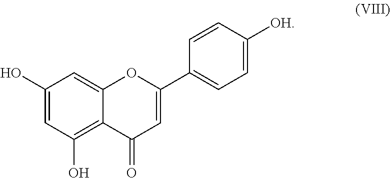 [SMILES: O=c1cc(-c2ccc(O)cc2)oc2cc(O)cc(O)c12]

7,8-DHF is a naturally occurring flavone found in Godmania aesculifolia, Tridax procumbens, and primula tree leaves. It has been found to act as a potent and selective small-molecule agonist of TrkB with a Kd of about 320 nM. 7,8-DHF is both orally bioavailable and able to penetrate the blood-brain barrier. The molecular formula for 7,8-DHF is C₁₅H₁₀O₄ and it has a molecular weight of 254.241 g/mol. 7,8-DHF has the following structural formula, Formula (IX):

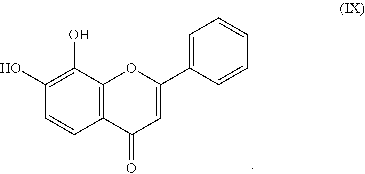 [SMILES: O=c1cc(-c2ccccc2)oc2c(O)c(O)ccc12]

### 3. COMPOSITIONS

Further provided herein are pharmaceutical compositions comprising the above-described compounds or pharmaceutically acceptable salts thereof or recombinants thereof. Also provided herein are culture media compositions comprising the above-described compounds or pharmaceutically acceptable salts thereof or recombinants thereof.

a. Pharmaceutical Compositions

The pharmaceutical composition may comprise a therapeutically effective amount of a TRK ligand. The pharmaceutical composition may comprise therapeutically effective amounts of one or more of a neurotrophin, a melatonin precursor, a neurotrophin mimetic, and a flavone. In some embodiments, the pharmaceutical composition may comprise therapeutically effective amounts of one or more of NGF, NTF3, NT-4, BDNF, LM22A-4, 7,8-DHF, and NAS. The pharmaceutical composition may further comprise a pharmaceutically acceptable excipient.

The pharmaceutical compositions may comprise about 10 ng/ml to about 100 ng/ml, about 20 ng/ml to about 100 ng/ml, about 30 ng/ml to about 100 ng/ml, about 40 ng/ml to about 100 ng/ml, about 50 ng/ml to about 100 ng/ml, about 60 ng/ml to about 100 ng/ml, about 70 ng/ml to about 100 ng/ml, about 80 ng/ml to about 100 ng/mL, about 90 ng/ml to about 100 ng/mL, about 10 ng/ml to about 90 ng/ml, about 10 ng/ml to about 80 ng/ml, about 10 ng/ml to about 70 ng/ml, about 10 ng/ml to about 60 ng/ml, about 10 ng/ml to about 50 ng/ml, about 10 ng/ml to about 40 ng/ml, about 10 ng/ml to about 30 ng/ml, or about 10 ng/ml to about 20 ng/ml of the TRK ligand. The pharmaceutical compositions may comprise at least 10 ng/ml, at least 20 ng/ml, at least 30 ng/ml, at least 40 ng/ml, at least 50 ng/ml, at least 60 ng/ml, at least 70 ng/ml, at least 80 ng/ml, or at least 90 ng/ml of the TRK ligand. The pharmaceutical compositions may comprise at most 100 ng/ml, at most 90 ng/ml, at most 80 ng/ml, at most 70 ng/ml, at most 60 ng/ml, at most 50 ng/ml, at most 40 ng/ml, at most 30 ng/ml, or at most 20 ng/ml of the TRK ligand.

The pharmaceutical composition may also comprise a growth factor other than a neurotrophin in a therapeutically effective amount such as insulin, fibroblast growth factor (FGF), FGF-10, noggin, growth hormone, vascular endothelial growth factor (VEGF), epidermal growth factor (EGF), platelet-derived growth factor (PDGF), nerve growth factor (NGF), insulin-like growth factor (IGF), transforming growth factor (TGF), hepatocyte growth factor (HGF), bone morphogenetic proteins (BMPs), erythropoietin (EPO), colony-stimulating factors (CSFs), hepatoma-derived growth factor (HDGF), or combinations thereof. The therapeutically effective amount of the growth factor other than a neurotrophin may be about 0 ng/ml to about 100 ng/ml, about 10 ng/ml to about 100 ng/ml, about 20 ng/ml to about 100 ng/mL, about 30 ng/ml to about 100 ng/ml, about 40 ng/ml to about 100 ng/ml, about 50 ng/ml to about 100 ng/ml, about 60 ng/ml to about 100 ng/ml, about 70 ng/ml to about 100 ng/ml, about 80 ng/ml to about 100 ng/mL, about 90 ng/ml to about 100 ng/ml, about 10 ng/ml to about 90 ng/ml, about 10 ng/ml to about 80 ng/mL, about 10 ng/ml to about 70 ng/ml, about 10 ng/ml to about 60 ng/ml, about 10 ng/ml to about 50 ng/ml, about 10 ng/ml to about 40 ng/ml, about 10 ng/ml to about 30 ng/ml, or about 10 ng/ml to about 20 ng/ml. The therapeutically effective amount of the growth factor other than a neurotrophin may be at least 10 ng/ml, at least 20 ng/ml, at least 30 ng/ml, at least 40 ng/ml, at least 50 ng/mL, at least 60 ng/ml, at least 70 ng/ml, at least 80 ng/ml, or at least 90 ng/ml. The therapeutically effective amount of the growth factor other than a neurotrophin may be at most 100 ng/ml, at most 90 ng/ml, at most 80 ng/ml, at most 70 ng/ml, at most 60 ng/ml, at most 50 ng/ml, at most 40 ng/ml, at most 30 ng/ml, or at most 20 ng/ml.

Compounds or pharmaceutically acceptable salts thereof or recombinants thereof as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is in some embodiments used. Generally, additives for isotonicity may include sodium chloride, dextrose, mannitol, sorbitol and lactose. In some cases, isotonic solutions such as phosphate buffered saline can be used. Stabilizers include gelatin and albumin. In some embodiments, a secondary treatment for liver disease, cardiogenic shock, ischemia reperfusion injury, preservation media of organs for transplantation, normothermic liver perfusion, hypothermic liver perfusion, acetaminophen intoxication, and/or drug intoxication, is added to the composition such as, but not limited to, epinephrine, dobutamine, norepinephrine, vitamin K, resmetirom, obeticholic acid, protone pump inhibitors, cholestyramine, anti-inflammatories, immunosuppressants, antibiotics, analgesics, vitamin supplements, mineral supplements, diuretics, beta-blockers, anti-itch therapies, and combinations thereof.

The composition may further comprise a pharmaceutically acceptable excipient. The pharmaceutically acceptable excipient may be functional molecules such as vehicles, adjuvants, carriers, or diluents. The term “pharmaceutically acceptable excipient,” may be a non-toxic, inert solid, semi-solid or liquid filler, diluent, encapsulating material or formulation auxiliary of any type. Pharmaceutically acceptable excipients include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. In some embodiments, the pharmaceutically acceptable excipient may be saline, water, albumin-supplemented ringer solution, methyl cellulose, polyethylene glycol 500 (PEG500), or a combination thereof.

In another aspect, the present disclosure provides a pharmaceutical composition comprising a compound as described herein, or a pharmaceutically acceptable salt thereof or recombinant thereof, and a pharmaceutically acceptable excipient. In a further embodiment, the composition comprises at least two pharmaceutically acceptable excipients, such as those described herein. For purposes of the present disclosure, unless designated otherwise, solvates and hydrates are generally considered compositions. Preferably, pharmaceutically acceptable excipients are sterile. The pharmaceutical composition can be formulated for particular routes of administration such as intravenous administration, oral administration, nasal administration, intraperitoneal administration, and parenteral administration, etc. In addition, the pharmaceutical compositions of the present disclosure can be made up in a solid form (including without limitation capsules, tablets, pills, granules, powders or suppositories), in a liquid form (including without limitation solutions, suspensions or emulsions), or aerosolized. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. In some embodiments, the pharmaceutical compositions are tablets or gelatin capsules comprising the active ingredient together with one or more of: diluents, e.g., lactose, dextrose, sucrose, mannitol, sorbitol, cellulose and/or glycine; lubricants, e.g., silica, talcum, stearic acid, its magnesium or calcium salt and/or polyethyleneglycol; binders, e.g., magnesium aluminum silicate, starch paste, gelatin, tragacanth, methylcellulose, sodium carboxymethylcellulose and/or polyvinylpyrrolidone; disintegrants, e.g., starches, agar, alginic acid or its sodium salt, or effervescent mixtures; and absorbents, colorants, flavors and sweeteners.

Certain injectable compositions are aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions may be prepared according to conventional mixing, granulating or coating methods, respectively, and contain about 0.1-100%, or about 0.1-75%, or contain about 1-50%, of the active ingredient.

In some embodiments, the composition may comprise a therapeutically effective amount of NTF3. In some embodiments, the composition may further comprise a pharmaceutically acceptable carrier.

In some embodiments, the composition may comprise a therapeutically effective amount of BDNF. In some embodiments, the composition may further comprise a pharmaceutically acceptable carrier.

In some embodiments, the composition may comprise a therapeutically effective amount of NAS. In some embodiments, the composition may further comprise a pharmaceutically acceptable carrier.

In some embodiments, the composition may comprise a therapeutically effective amount of LM22A-4. In some embodiments, the composition may further comprise a pharmaceutically acceptable carrier.

In some embodiments, the composition may comprise a therapeutically effective amount of 7,8-DHF. In some embodiments, the composition may further comprise a pharmaceutically acceptable carrier.

In some embodiments, the composition may comprise two or more of a therapeutically effective amount of NTF3, a therapeutically effective amount of BDNF, a therapeutically effective amount of NAS, a therapeutically effective amount of LM22A-4, and a therapeutically effective amount of 7,8-DHF. In some embodiments, the composition may further comprise a pharmaceutically acceptable carrier. The NTF3, the BDNF, the NAS, the LM22A-4, and/or the 7,8-DHF may be at any ratio.

b. Culture Media Compositions

A culture medium composition may comprise an effective amount of a TRK ligand. A cell culture medium composition may comprise a base cell culture medium comprising one or more amino acids, salts, buffers, trace minerals, lipids, nucleic acids, additives, and proteins; one or more TRK ligands described herein; one or more growth factors other than a neurotrophin described herein; and one or more additional additives. “Culture medium” and “cell culture medium” are used interchangeably herein.

The culture medium composition may comprise effective amounts of one or more of a neurotrophin, a melatonin precursor, a neurotrophin mimetic, and a flavone.

In some embodiments, the culture medium composition may comprise an effective amount of NTF3. In some embodiments, the culture medium composition may comprise an effective amount of BDNF. In some embodiments, the culture medium composition may comprise an effective amount of NAS. In some embodiments, the culture medium composition may comprise an effective amount of LM22A-4. In some embodiments, the culture medium composition may comprise an effective amount of 7,8-DHF.

In some embodiments, the culture medium composition may comprise two or more of an effective amount of NTF3, an effective amount of BDNF, an effective amount of NAS, an effective amount of LM22A-4, and an effective amount of 7,8-DHF. The NTF3, the BDNF, the NAS, the LM22A-4, and/or the 7,8-DHF may be at any ratio.

In some embodiments, the culture media composition may comprise effective amounts of one or more of NGF, NTF3, NT-4, BDNF, LM22A-4, 7,8-DHF, and NAS. The effective amount of the TRK ligand may be about 10 ng/mL to about 100 ng/mL, about 20 ng/ml to about 100 ng/ml, about 30 ng/ml to about 100 ng/ml, about 40 ng/ml to about 100 ng/ml, about 50 ng/ml to about 100 ng/ml, about 60 ng/ml to about 100 ng/ml, about 70 ng/ml to about 100 ng/ml, about 80 ng/ml to about 100 ng/ml, about 90 ng/mL to about 100 ng/ml, about 10 ng/ml to about 90 ng/ml, about 10 ng/ml to about 80 ng/ml, about 10 ng/ml to about 70 ng/ml, about 10 ng/ml to about 60 ng/ml, about 10 ng/ml to about 50 ng/ml, about 10 ng/ml to about 40 ng/mL, about 10 ng/mL to about 30 ng/ml, or about 10 ng/ml to about 20 ng/mL. The effective amount of the TRK ligand may be at least 10 ng/mL, at least 20 ng/ml, at least 30 ng/ml, at least 40 ng/mL, at least 50 ng/ml, at least 60 ng/ml, at least 70 ng/ml, at least 80 ng/ml, or at least 90 ng/ml. The effective amount of the TRK ligand may be at most 100 ng/ml, at most 90 ng/ml, at most 80 ng/ml, at most 70 ng/ml, at most 60 ng/ml, at most 50 ng/ml, at most 40 ng/ml, at most 30 ng/ml, or at most 20 ng/mL.

Compounds or salts thereof or recombinants thereof as detailed herein, or at least one component thereof, may be formulated into a culture medium in accordance with standard techniques well known to those skilled in the biological art. The culture media composition may further comprise mixtures of one or more nutrients and other substances that support the growth of cells in vitro or ex vivo. The culture medium or the base culture medium may be serum-reduced. In some embodiments, the culture medium or the base culture medium is serum free.

Examples of animal cell culture media include, but are not limited to, DMEM, AdvDMEM/F12+++, RSPO1-conditioned medium, Afamin-Wnt-3A serum free conditioned media, RPMI-1640, MCDB 131, MCDB 153, MDEM, IMDM, MEM, M199, McCoy's 5A, Williams' Media E, Leibovitz's L-15 Medium, F10 Nutrient Mixture, F12 Nutrient Mixture, and cell-specific serum-free media (SFM) such as those designed to support the culture of hepatocytes, cholangiocytes, organoids, liver slices, and the like. Media, media supplements, and media subgroups suitable for preparation are available commercially. Formulations for these media, media supplements and media subgroups, as well as many other commonly used animal cell culture media, media supplements and media subgroups are known in the art and available from commercial suppliers, e.g., Thermo Fisher Scientific Inc., Life Technologies, Gibco, Invitrogen, et al.

### i) Amino Acids and Nucleic Acids

The base culture medium may comprise one or more amino acids. The amino acids may be one or more of alanine, arginine, aspartic acid, glutamic acid, glycine, histidine, isoleucine, methionine, phenylalanine, proline, hydroxyproline, serine, threonine, tryptophan, valine, tyrosine, cysteine, and lysine. The one or more amino acids may be present in the base culture medium at about 20 wt. % to about 40 wt. %, about 25 wt. % to about 40 wt. %, about 30 wt. % to about 40 wt. %, about 35 wt. % to about 40 wt. %, about 20 wt. % to about 35 wt. %, about 20 wt. % to about 30 wt. %, or about 20 wt. % to about 25 wt. %.

The base culture medium may comprise one or more nucleic acids. The nucleic acids may be one or more of DNA (deoxyribonucleic acid) and RNA (ribonucleic acid). DNA and RNA may be present in the culture medium as a result of being released from cells. DNA and RNA may be added to the culture medium for DNA or RNA delivery and manipulation.

### ii) Salts

The base culture medium may comprise one or more salts. The salts may be one or more of salts of sodium, potassium, magnesium, calcium, ammonium, phosphate, carbonate, and sulfate. The one or more salts may be present in the base culture medium at about 2 wt. % to about 10 wt. %, about 4 wt. % to about 10 wt. %, about 6 wt. % to about 10 wt. %, about 8 wt. % to about 10 wt. %, about 2 wt. % to about 8 wt. %, about 2 wt. % to about 6 wt. %, or about 2 wt. % to about 4 wt. %.

iii) Buffers

The base culture medium may comprise one or more buffers. For example, buffers may include balanced salt solutions, which are isotonic for animal cells. The base culture media may comprise one or more buffer salts, such as sodium bicarbonate, at concentrations sufficient to provide optimal buffering capacity for the culture medium. The buffer may be acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, MOPSO, cholamine chloride, MOPS, BES, TES, HEPES, DIPSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof.

A pH-adjusting agent such as an acid (e.g., HCl) or a base (e.g., NaOH) may be added to the culture medium. The pH of the culture medium may be of between about 7.1 to about 7.5, more preferably between about 7.1 to about 7.4, and most preferably about 7.2 to about 7.4 or about 7.2 to about 7.5.

### iv) Trace Minerals

The base culture medium may comprise one or more trace minerals. The trace minerals may be one or more of iron, manganese, copper, iodine, zinc, cobalt, fluoride, chromium, molybdenum, selenium, nickel, silicon, and vanadium. The one or more trace minerals may be present in the base culture medium at about 0.01 wt. % to about 0.05 wt. %, about 0.02 wt. % to about 0.05 wt. %, about 0.03 wt. % to about 0.05 wt. %, about 0.04 wt. % to about 0.05 wt. %, about 0.01 wt. % to about 0.04 wt. %, about 0.01 wt. % to about 0.03 wt. %, or about 0.01 wt. % to about 0.02 wt. %.

### v) Lipids

The base culture medium may comprise one or more lipids. The lipids may be one or more of phospholipids, sphingolipids, fatty acids, Excyte, cholesterol, bovine cholesterol concentrate, fatty acids, and the like. The one or more lipids may be present in the base culture medium at about 0.1 wt. % to about 5.0 wt. %, about 0.5 wt. % to about 5.0 wt. %, about 1.0 wt. % to about 5.0 wt. %, about 2.0 wt. % to about 5.0 wt. %, about 3.0 wt. % to about 5.0 wt. %, about 4.0 wt. % to about 5.0 wt. %, about 0.1 wt. % to about 4.0 wt. %, about 0.1 wt. % to about 3.0 wt. %, about 0.1 wt. % to about 2.0 wt. %, about 0.1 wt. % to about 1.0 wt. %, or about 0.1 wt. % to about 0.5 wt. %.

### vi) Vitamins

The culture medium may further comprise vitamins such as retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof.

vii) Additives

The base culture medium may comprise one or more additives. The additives may be one or more of antibiotics (e.g., penicillin/streptomycin, Geneticin carbenicillin, cefotaxime, anti-PPLO, Fungizone, hygromycin, kanamycin, neomycin, nystatin, penicillin, streptomycin, and the like), antioxidants (e.g., N-acetyl-L-cysteine, vitamin C, glutathione, vitamin E, superoxide dismutase, catalase, carotenoids, and the like), pH indicators (e.g., phenol red, bromothymol blue, methyl red, and the like), surfactants (e.g., Tween 20, Tween 80, Pluronic F-68, SDS, and the like), amines (e.g., dopamine, epinephrine, norepinephrine, and the like), amino acid dipeptides (e.g., Glutamax (1×; L-alanyl-L-glutamine)), and supplements.

The base culture medium may be a nutritive medium that comprises a supplement. Media supplements may include, but are not limited to, extracts of bacterial, animal or plant cells, glands, tissues or organs, particularly bovine pituitary extract, bovine brain extract and chick embryo extract; and biological fluids (particularly animal sera, and most preferably bovine serum, particularly fetal bovine, newborn calf or normal calf serum, horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Such supplements may also include, but not limited to, B-27 supplement and Human [Leu15] Gastrin.

The one or more additives may be present in the base culture medium at about 0.1 wt. % to about 5.0 wt. %, about 0.5 wt. % to about 5.0 wt. %, about 1.0 wt. % to about 5.0 wt. %, about 2.0 wt. % to about 5.0 wt. %, about 3.0 wt. % to about 5.0 wt. %, about 4.0 wt. % to about 5.0 wt. %, about 0.1 wt. % to about 4.0 wt. %, about 0.1 wt. % to about 3.0 wt. %, about 0.1 wt. % to about 2.0 wt. %, about 0.1 wt. % to about 1.0 wt. %, or about 0.1 wt. % to about 0.5 wt. %.

viii) Proteins

The base culture medium and the culture medium may comprise one or more proteins. The proteins may be one or more of serum albumins, particularly bovine (e.g., fetal bovine serum (FBS)) or human serum albumins; serum replacement (e.g., KnockOut, Gibco); immunoglobulins and fragments or complexes thereof; aprotinin; hemoglobin; haemin or haematin; enzymes (such as trypsin, collagenases, pancreatinin, or dispase); lipoproteins; fetuin; ferritin; R-spondin 1 (RSPO1); complex of the protein Wnt3a and the serum glycoprotein Afamin; and the like. The proteins may be natural or recombinant. The one or more proteins may be present in the base culture medium at about 1 wt. % to about 10 wt. %, about 2 wt. % to about 10 wt. %, about 4 wt. % to about 10 wt. %, about 6 wt. % to about 10 wt. %, about 8 wt. % to about 10 wt. %, about 1 wt. % to about 8 wt. %, about 1 wt. % to about 6 wt. %, about 1 wt. % to about 4 wt. %, or about 1 wt. % to about 2 wt. %.

### ix) Growth Factors

The culture medium may also comprise a growth factor other than a neurotrophin in an effective amount such as insulin, fibroblast growth factor (FGF), FGF-10, noggin, growth hormone, vascular endothelial growth factor (VEGF), epidermal growth factor (EGF), platelet-derived growth factor (PDGF), nerve growth factor (NGF), insulin-like growth factor (IGF), transforming growth factor (TGF), hepatocyte growth factor (HGF), bone morphogenetic proteins (BMPs), erythropoietin (EPO), colony-stimulating factors (CSFs), hepatoma-derived growth factor (HDGF), or combinations thereof. The effective amount of the growth factor other than a neurotrophin may be about 0 ng/ml to about 100 ng/ml, about 10 ng/ml to about 100 ng/ml, about 20 ng/ml to about 100 ng/ml, about 30 ng/ml to about 100 ng/mL, about 40 ng/mL to about 100 ng/mL, about 50 ng/ml to about 100 ng/ml, about 60 ng/ml to about 100 ng/ml, about 70 ng/mL to about 100 ng/ml, about 80 ng/ml to about 100 ng/ml, about 90 ng/mL to about 100 ng/ml, about 10 ng/ml to about 90 ng/ml, about 10 ng/ml to about 80 ng/ml, about 10 ng/ml to about 70 ng/mL, about 10 ng/ml to about 60 ng/ml, about 10 ng/ml to about 50 ng/ml, about 10 ng/ml to about 40 ng/mL, about 10 ng/ml to about 30 ng/ml, or about 10 ng/ml to about 20 ng/ml. The effective amount of the growth factor other than a neurotrophin may be at least 10 ng/ml, at least 20 ng/ml, at least 30 ng/ml, at least 40 ng/ml, at least 50 ng/ml, at least 60 ng/ml, at least 70 ng/ml, at least 80 ng/ml, or at least 90 ng/ml. The effective amount of the growth factor other than a neurotrophin may be at most 100 ng/mL, at most 90 ng/ml, at most 80 ng/ml, at most 70 ng/ml, at most 60 ng/ml, at most 50 ng/ml, at most 40 ng/mL, at most 30 ng/mL, or at most 20 ng/ml.

### x) Additional Additives

The culture medium may also include one or more additional additives, such as interleukins, colony-stimulating factors, interferons, attachment factors, extracellular matrix components (e.g., collagens, laminins, proteoglycans, glycosaminoglycans, fibronectin, vitronectin, and the like); extracts of animal tissues, glands or organs; viral inhibitors (e.g., protease inhibitors, nucleoside analogues, and the like); inhibitors (e.g., A 83-01, DAPT); natural compounds (e.g., forskolin and the like); cytokines (e.g., IL-6, oncostatin, and the like); and hormones (e.g., prolactin, cortisol, steroid hormones such as corticosteroids, estrogens, androgens (e.g., testosterone), and the like).

An exemplary base medium, such as DMEM/F12, composition may comprise: about 1% to about 10% R-spondin 1 (RSPO1); about 0.5× to about 2× of a supplement (e.g., 1×B-27 supplement); about 0.5 to about 2 mM of an antioxidant (e.g., 1 mM N-acetyl-L-cysteine); about 1 nM to about 20 nM Human [Leu15] Gastrin; about 1% to about 1% of an antibiotic (e.g., Penicillin/Streptomycin 1%); about 1% of a buffer (e.g., HEPES); about 0% to about 1% of an amino acid or amino acid dipeptide (e.g., Glutamax (1×; L-alanyl-L-glutamine).

An exemplary expansion medium may comprise the base medium plus: about 0% to about 30% Afamin-Wnt-3A serum free conditioned media; about 0% to about 10% of R-spondin 1 (RSPO1); about 0 ng/ml to about 100 ng/ml of one or more growth factors other than a neurotrophin; about 0 μM to about 10 μM of an inhibitor (e.g., A 83-01); about 0 M to about 50 μM of Forskolin; about 0 ng/ml to about 500 ng/ml of a cytokine (e.g., IL-6, oncostatin); about 10 to about 100 ng/ml of one or more TRK ligands.

An exemplary differentiation medium for zone 1 organoids may comprise the base medium plus: about 0 ng/ml to about 100 ng/ml of one or more growth factors other than a neurotrophin; about 0 μM to about 50 μM of an inhibitor (e.g., A 83-01, DAPT); about 0 μM to about 100 μM of Forskolin; about 0 ng/ml to about 1 mg/ml of a hormone (e.g., prolactin, cortisol, growth hormone); about 10 to about 100 ng/ml of one or more TRK ligands.

An exemplary differentiation medium for zone 3 organoids may comprise the differentiation medium for zone 1 organoids plus: about 0% to about 50% Afamin-Wnt-3A serum free conditioned media; and about 0% to about 20% of R-spondin 1 (RSPO1).

### 4. ADMINISTRATION

The compounds or pharmaceutically acceptable salts thereof or recombinants thereof as detailed herein, or at least one component thereof, may be administered or delivered to a subject or a cell of the subject. Compounds or pharmaceutically acceptable salts thereof or recombinants thereof as detailed herein, or at least one component thereof, may be administered or delivered to a subject or a cell of the subject in a therapeutically effective amount. Such compositions can be administered in dosages and by techniques well known to those skilled in the medical arts taking into consideration such factors as the age, sex, weight, and condition of the particular subject, and the route of administration. The presently disclosed compounds or pharmaceutically acceptable salts thereof or recombinants thereof, or compositions comprising the same, may be administered to a subject by different routes including intravenously, orally, parenterally, sublingually, transdermally, rectally, transmucosally, topically, intranasal, intravaginal, via inhalation, via buccal administration, intraperitoneally, intraarterial, intraperitoneal, subcutaneous, intradermally, epidermally, intramuscular, intranasal, intrathecal, intracranial, and intraarticular, or combinations thereof. In some embodiments, the composition as described herein may be administered intravenously, subcutaneously, or intramuscularly.

In some embodiments, a composition described herein may be administered more than once. In some embodiments, a composition described herein may be administered to a subject more than once per day. In some embodiments, a composition described herein may be administered after the liver disease or liver damage.

In some embodiments, a composition described herein may be administered with a secondary treatment for a liver disease or liver damage. The secondary treatment for liver disease or liver damage may be epinephrine, dobutamine, norepinephrine, vitamin K, resmetirom, obeticholic acid, protone pump inhibitors, cholestyramine, anti-inflammator immunosuppressants, antibiotics, analgesics, vitamin supplements, mineral supplements, diuretics, beta-blockers, anti-itch therapies, or combinations thereof. For example, a TRK ligand described herein may be administered as a component for hypothermic liver perfusions. In another example, a TRK ligand described herein may be administered as a component for normothermic liver perfusions.

Upon delivery of the presently disclosed compounds or pharmaceutical compositions as detailed herein, or at least one component thereof, liver cell proliferation is stimulated, liver regeneration is stimulated, or a combination thereof.

Any of the delivery methods and/or routes of administration detailed herein can be utilized with a myriad of cell types, for example, those cell types currently under investigation for liver diseases or liver damage, including, but not limited to, primary hepatocytes, HepG2 cells, AML12 cells, THLE-2 cells, primary cholangiocytes, MMNK-1 cells, H69 cells, hepatocarcinoma cell lines (such as HepaRG cells), mesenchymal stem cells (MSCs), hepatic stellate cells (HSCs), Kupffer cells, liver sinusoidal endothelial cells (LSECs), stem cells such as those derived from induced pluripotent stem cells (iPSCs), cells of liver organoids, cells of ductal organoids, cells of hepatic organoids, cells of liver-on-a-chip, or cells of precision liver slices. Cells can be modified in vitro, in vivo, or ex vivo.

### 5. METHODS

a. Methods of Stimulating Liver Cell Proliferation In Vivo

Provided herein are methods of stimulating liver cell proliferation in a subject. The methods may include administering to the subject a TRK ligand in an amount effective to stimulate liver cell proliferation. The TRK ligand may be in a pharmaceutical composition as described herein. The liver cell may be a hepatocyte, a cholangiocyte, or a combination thereof. The subject may be a human, a monkey, a pig, a mouse, a dog, a guinea pig, or a rat.

The TRK ligand may be a neurotrophin, a melatonin precursor, a neurotrophin mimetic, a flavone, or a combination thereof as described herein. The effective amount of the TRK ligand may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the TRK ligand may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the TRK ligand may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg.

The TRK ligand may be administered daily for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered daily for at least 1 day, at least 2 days, at least 3 days, at least 4 days, at least 5 days, at least 6 days, at least 7 days, at least 8 days, at least 9 days, at least 10 days, at least 11 days, at least 12 days, or at least 13 days. The TRK ligand may be administered daily for at most 2 days, at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered every other day for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered every other day for at least 3 days, at least 4 days, at least 5 days, at least 6 days, at least 7 days, at least 8 days, at least 9 days, at least 10 days, at least 11 days, at least 12 days, or at least 13 days. The TRK ligand may be administered every other day for at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered to the subject in combination with an effective amount of a growth factor other than a neurotrophin as described herein. The effective amount of the growth factor other than a neurotrophin may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the growth factor other than a neurotrophin may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the growth factor other than a neurotrophin may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg. The TRK ligand and the growth factor other than a neurotrophin may be administered concomitantly, such as simultaneously or successively.

The administration of the TRK ligand may increase liver cell proliferation at least 2 days following administration as compared to liver cell proliferation before administration of the TRK ligand. The administration of the TRK ligand may increase liver cell proliferation at least 2 days following administration as compared to a control subject.

The TRK ligand may be administered as described herein, for example, intravenously. The use of a composition as described herein may be an emergency treatment when the patient is hospitalized, for example, due to acute liver failure, when a subject receives other intravenous treatments. Therefore, the TRK ligand may be administered with another intravenous treatment. The TRK ligand and the other intravenous treatment may be administered concomitantly, such as simultaneously or successively.

The TRK ligand may be administered following liver damage. The liver damage may be acute. The liver damage may be chronic. The liver damage may be a result of hepatectomy, partial liver resection, post liver transplantation, cirrhosis of the liver, hepatic malignancies, exposure to alcohol, hepatotoxic drugs, infectious agents, side effects of gene therapy, exposure to anti-tuberculosis agents, exposure to chemotherapeutic agents, acetaminophen overdoses, mushroom poisoning, infarction, latrogenic injury (e.g., from transarterial chemoembolization (TACE), chemoembolisation), cardiogenic shock, other kind of shock with decrease in blood pressure and resulting hypoperfusion of the liver, ischemia-reperfusion injury, or combinations thereof.

b. Methods of Stimulating Liver Regeneration

Provided herein are methods of stimulating liver regeneration in a subject. The methods may include administering to the subject a TRK ligand as described herein in an amount effective to stimulate liver regeneration. The TRK ligand may be in a pharmaceutical composition as described herein. The subject may be a human, a monkey, a pig, a mouse, a guinea pig, a dog, or a rat.

The TRK ligand may be a neurotrophin, a melatonin precursor, a neurotrophin mimetic, a flavone, or a combination thereof as described herein. The effective amount of the TRK ligand may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the TRK ligand may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the TRK ligand may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg.

The TRK ligand may be administered daily for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered daily for at least 1 day, at least 2 days, at least 3 days, at least 4 days, at least 5 days, at least 6 days, at least 7 days, at least 8 days, at least 9 days, at least 10 days, at least 11 days, at least 12 days, or at least 13 days. The TRK ligand may be administered daily for at most 2 days, at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered every other day for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered every other day for at least 3 days, at least 4 days, at least 5 days, at least 6 days, at least 7 days, at least 8 days, at least 9 days, at least 10 days, at least 11 days, at least 12 days, or at least 13 days. The TRK ligand may be administered every other day for at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered to the subject in combination with an effective amount of a growth factor other than a neurotrophin as described herein. The effective amount of the growth factor other than a neurotrophin may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the growth factor other than a neurotrophin may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the growth factor other than a neurotrophin may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg. The TRK ligand and the growth factor other than a neurotrophin may be administered concomitantly, such as simultaneously or successively.

The administration of the TRK ligand may increase liver cell proliferation at least 2 days following administration as compared to liver cell proliferation before administration of the TRK ligand. The administration of the TRK ligand may increase liver cell proliferation at least 2 days following administration as compared to a control subject. The administration of the TRK ligand may increase liver weight at least 3 days following administration as compared to liver weight before administration of the TRK ligand. The administration of the TRK ligand may increase liver weight at least 3 days following administration as compared to liver weight of a control subject.

The TRK ligand may be administered as described herein, for example, intravenously. The use of a composition as described herein may be an emergency treatment when the patient is hospitalized, for example, due to acute liver failure, when a subject receives other intravenous treatments. Therefore, the TRK ligand may be administered with another intravenous treatment. The TRK ligand and the other intravenous treatment may be administered concomitantly, such as simultaneously or successively.

The TRK ligand may be administered following liver damage. The liver damage may be acute. The liver damage may be chronic. The liver damage may be a result of hepatectomy, partial liver resection, post liver transplantation, cirrhosis of the liver, hepatic malignancies, exposure to alcohol, hepatotoxic drugs, infectious agents, side effects of gene therapy, exposure to anti-tuberculosis agents, exposure to chemotherapeutic agents, acetaminophen overdoses, mushroom poisoning, infarction, latrogenic injury (e.g., from transarterial chemoembolization (TACE), chemoembolisation), cardiogenic shock, other kind of shock with decrease in blood pressure and resulting hypoperfusion of the liver, ischemia-reperfusion injury, or combinations thereof.

c. Methods of Treating Liver Damage

Provided herein are methods of treating liver damage in subject. The methods may include administering to the subject a TRK ligand as described herein in an amount effective to stimulate liver cell proliferation and/or liver regeneration. The TRK ligand may be in a pharmaceutical composition as described herein. The subject may be a human, a monkey, a pig, a mouse, a guinea pig, dog, or a rat.

The liver damage may be acute. The liver damage may be chronic. The liver damage may be a result of hepatectomy, partial liver resection, post liver transplantation, cirrhosis of the liver, hepatic malignancies, exposure to alcohol, hepatotoxic drugs, infectious agents, side effects of gene therapy, exposure to anti-tuberculosis agents, exposure to chemotherapeutic agents, acetaminophen overdoses, mushroom poisoning, infarction, latrogenic injury (e.g., from transarterial chemoembolization (TACE), chemoembolisation), cardiogenic shock, other kind of shock with decrease in blood pressure and resulting hypoperfusion of the liver, ischemia-reperfusion injury, or combinations thereof. The TRK ligand may be administered following liver damage.

The TRK ligand may be a neurotrophin, a melatonin precursor, a neurotrophin mimetic, a flavone, or a combination thereof as described herein. The effective amount of the TRK ligand may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the TRK ligand may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the TRK ligand may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg.

The TRK ligand may be administered daily for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered daily for at least 1 day, at least 2 days, at least 3 days, at least 4 days, at least 5 days, at least 6 days, at least 7 days, at least 8 days, at least 9 days, at least 10 days, at least 11 days, at least 12 days, or at least 13 days. The TRK ligand may be administered daily for at most 2 days, at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered every other day for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The TRK ligand may be administered every other day for at least 3 days, at least 4 days, at least 5 days, at least 6 days, at least 7 days, at least 8 days, at least 9 days, at least 10 days, at least 11 days, at least 12 days, or at least 13 days. The TRK ligand may be administered every other day for at most 3 days, at most 4 days, at most 5 days, at most 6 days, at most 7 days, at most 8 days, at most 9 days, at most 10 days, at most 11 days, at most 12 days, at most 13 days, or at most 14 days.

The TRK ligand may be administered to the subject in combination with an effective amount of a growth factor other than a neurotrophin as described herein. The effective amount of the growth factor other than a neurotrophin may be about 10 ng/kg to about 100 μg/kg, about 50 ng/kg to about 100 μg/kg, about 100 ng/kg to about 100 μg/kg, about 150 ng/kg to about 100 μg/kg, about 200 ng/kg to about 100 μg/kg, about 250 ng/kg to about 100 μg/kg, about 300 ng/kg to about 100 μg/kg, about 350 ng/kg to about 100 μg/kg, about 400 ng/kg to about 100 μg/kg, about 450 ng/kg to about 100 μg/kg, about 500 ng/kg to about 100 μg/kg, about 550 ng/kg to about 100 μg/kg, about 600 ng/kg to about 100 μg/kg, about 650 ng/kg to about 100 μg/kg, about 700 ng/kg to about 100 μg/kg, about 750 ng/kg to about 100 μg/kg, about 800 ng/kg to about 100 μg/kg, about 850 ng/kg to about 100 μg/kg, about 900 ng/kg to about 100 μg/kg, about 950 ng/kg to about 100 μg/kg, about 1 μg/kg to about 100 μg/kg, about 20 μg/kg to about 100 μg/kg, about 30 μg/kg to about 100 μg/kg, about 40 μg/kg to about 100 μg/kg, about 50 μg/kg to about 100 μg/kg, about 60 μg/kg to about 100 μg/kg, about 70 μg/kg to about 100 μg/kg, about 80 μg/kg to about 100 μg/kg, about 90 μg/kg to about 100 μg/kg, about 10 ng/kg to about 90 μg/kg, about 10 ng/kg to about 80 μg/kg, about 10 ng/kg to about 70 μg/kg, about 10 ng/kg to about 60 μg/kg, about 10 ng/kg to about 50 μg/kg, about 10 ng/kg to about 40 μg/kg, about 10 ng/kg to about 30 μg/kg, about 10 ng/kg to about 20 μg/kg, about 10 ng/kg to about 10 μg/kg, about 10 ng/kg to about 1 μg/kg, about 10 ng/kg to about 950 ng/kg, about 10 ng/kg to about 900 ng/kg, about 10 ng/kg to about 850 ng/kg, about 10 ng/kg to about 800 ng/kg, about 10 ng/kg to about 750 ng/kg, about 10 ng/kg to about 700 ng/kg, about 10 ng/kg to about 650 ng/kg, about 10 ng/kg to about 600 ng/kg, about 10 ng/kg to about 550 ng/kg, about 10 ng/kg to about 500 ng/kg, about 10 ng/kg to about 450 ng/kg, about 10 ng/kg to about 400 ng/kg, about 10 ng/kg to about 350 ng/kg, about 10 ng/kg to about 300 ng/kg, about 10 ng/kg to about 250 ng/kg, about 10 ng/kg to about 200 ng/kg, about 10 ng/kg to about 150 ng/kg, about 10 ng/kg to about 100 ng/kg, or about 10 ng/kg to about 50 ng/kg.

The effective amount of the growth factor other than a neurotrophin may be at least 10 ng/kg at least 50 ng/kg at least 100 ng/kg at least 150 ng/kg at least 200 ng/kg at least 250 ng/kg at least 300 ng/kg at least 350 ng/kg at least 400 ng/kg at least 450 ng/kg at least 500 ng/kg at least 550 ng/kg at least 600 ng/kg at least 650 ng/kg at least 700 ng/kg at least 750 ng/kg at least 800 ng/kg at least 850 ng/kg at least 900 ng/kg at least 950 ng/kg at least 1 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, or at least 90 μg/kg. The effective amount of the growth factor other than a neurotrophin may be at most 50 ng/kg at most 100 ng/kg at most 150 ng/kg at most 200 ng/kg at most 250 ng/kg at most 300 ng/kg at most 350 ng/kg at most 400 ng/kg at most 450 ng/kg at most 500 ng/kg at most 550 ng/kg at most 600 ng/kg at most 650 ng/kg at most 700 ng/kg at most 750 ng/kg at most 800 ng/kg at most 850 ng/kg at most 900 ng/kg at most 950 ng/kg at most 1 μg/kg, at most 10 μg/kg, at most 20 μg/kg, at most 30 μg/kg, at most 40 μg/kg, at most 50 μg/kg, at most 60 μg/kg, at most 70 μg/kg, at most 80 μg/kg, at most 90 μg/kg, or at most 100 μg/kg. The TRK ligand and the growth factor other than a neurotrophin may be administered concomitantly, such as simultaneously or successively.

The administration of the TRK ligand may increase liver cell proliferation at least 2 days following administration as compared to liver cell proliferation before administration of the TRK ligand. The administration of the TRK ligand may increase liver cell proliferation at least 2 days following administration as compared to a control subject. The administration of the TRK ligand may increase liver weight at least 3 days following administration as compared to liver weight before administration of the TRK ligand. The administration of the TRK ligand may increase liver weight at least 3 days following administration as compared to liver weight of a control subject.

The TRK ligand may be administered as described herein, for example, intravenously. The use of a composition as described herein may be an emergency treatment when the patient is hospitalized, for example, due to acute liver failure, when a subject receives other intravenous treatments. Therefore, the TRK ligand may be administered with another intravenous treatment. The TRK ligand and the other intravenous treatment may be administered concomitantly, such as simultaneously or successively.

d. Methods of Stimulating Proliferation of a Liver Cell Ex Vivo or In Vitro

Provided herein are methods of stimulating proliferation of a liver cell. The methods may include contacting a liver cell with a TRK ligand as described herein in an amount effective to stimulate proliferation of the liver cell. The method may induce liver organoid differentiation. The method may induce proliferation of liver cells in a liver slice ex vivo. The method may induce proliferation of liver cells in vitro. The liver cell may be a hepatocyte, a cholangiocyte, or a combination thereof. The TRK ligand may be in a culture medium composition as described herein.

The TRK ligand may be a neurotrophin, a melatonin precursor, a neurotrophin mimetic, a flavone, or a combination thereof as described herein. The effective amount of the TRK ligand may be about 10 ng/ml to about 100 ng/ml, about 20 ng/mL to about 100 ng/ml, about 30 ng/ml to about 100 ng/mL, about 40 ng/ml to about 100 ng/ml, about 50 ng/mL to about 100 ng/ml, about 60 ng/ml to about 100 ng/ml, about 70 ng/ml to about 100 ng/ml, about 80 ng/ml to about 100 ng/ml, about 90 ng/ml to about 100 ng/ml, about 10 ng/ml to about 90 ng/ml, about 10 ng/ml to about 80 ng/mL, about 10 ng/ml to about 70 ng/ml, about 10 ng/ml to about 60 ng/ml, about 10 ng/ml to about 50 ng/ml, about 10 ng/ml to about 40 ng/ml, about 10 ng/ml to about 30 ng/ml, or about 10 ng/ml to about 20 ng/ml. The effective amount of the TRK ligand may be at least 10 ng/ml, at least 20 ng/ml, at least 30 ng/ml, at least 40 ng/ml, at least 50 ng/ml, at least 60 ng/ml, at least 70 ng/ml, at least 80 ng/ml, or at least 90 ng/ml. The effective amount of the TRK ligand may be at most 100 ng/ml, at most 90 ng/ml, at most 80 ng/mL, at most 70 ng/ml, at most 60 ng/ml, at most 50 ng/ml, at most 40 ng/ml, at most 30 ng/ml, or at most 20 ng/mL.

The liver cell may be contacted with the TRK ligand daily for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. For example, the TRK ligand may be added to a culture medium comprising the liver cells every day for 14 days. The liver cell may be contacted with the TRK ligand daily for at least 1 day, at least 2 days, at least 3 days, at least 4 days, at least 5 days, at least 6 days, at least 7 days, at least 8 days, at least 9 days, at least 10 days, at least 11 days, at least 12 days, or at least 13 days. The liver cell may be contacted with the TRK ligand daily for at most 2 days, at most 3 days, at most 4 days, at most 5 days, at most 6 days, at most 7 days, at most 8 days, at most 9 days, at most 10 days, at most 11 days, at most 12 days, at most 13 days, or at most 14 days. For example, the TRK ligand may be added to a culture medium comprising the liver cells on days 0, 1, 2, 3, 4, 5, 6, 7, 8, 9, 10, 11, 12, 13, and/or 14 of culture.

The liver cell may be contacted with the TRK ligand every other day for about 1 day to about 14 days, about 2 days to about 14 days, about 4 days to about 14 days, about 6 days to about 14 days, about 8 days to about 14 days, about 10 days to about 14 days, about 12 days to about 14 days, about 1 day to about 12 days, about 1 day to about 10 days, about 1 day to about 8 days, about 1 day to about 6 days, about 1 day to about 4 days, or about 1 day to about 2 days. The liver cell may be contacted with the TRK ligand every other day for at least 3 days, at least 4 days, at least 5 days, at least 6 days, at least 7 days, at least 8 days, at least 9 days, at least 10 days, at least 11 days, at least 12 days, or at least 13 days. The liver cell may be contacted with the TRK ligand every other day for at most 3 days, at most 4 days, at most 5 days, at most 6 days, at most 7 days, at most 8 days, at most 9 days, at most 10 days, at most 11 days, at most 12 days, at most 13 days, or at most 14 days. For example, the TRK ligand may be added to a culture medium comprising the liver cells on days 0, 2, 4, 6, 8, 10, 12, and/or 14 of culture.

The liver cell may be contacted by the TRK ligand in combination with an effective amount of a growth factor other than a neurotrophin as described herein. The growth factor other than a neurotrophin may be comprised by the culture medium as described herein. The effective amount of the growth factor other than a neurotrophin may be about 10 ng/ml to about 100 ng/ml, about 20 ng/ml to about 100 ng/ml, about 30 ng/ml to about 100 ng/ml, about 40 ng/ml to about 100 ng/ml, about 50 ng/ml to about 100 ng/ml, about 60 ng/ml to about 100 ng/ml, about 70 ng/mL to about 100 ng/ml, about 80 ng/ml to about 100 ng/ml, about 90 ng/ml to about 100 ng/ml, about 10 ng/ml to about 90 ng/ml, about 10 ng/ml to about 80 ng/ml, about 10 ng/ml to about 70 ng/mL, about 10 ng/ml to about 60 ng/ml, about 10 ng/ml to about 50 ng/ml, about 10 ng/mL to about 40 ng/ml, about 10 ng/ml to about 30 ng/ml, or about 10 ng/ml to about 20 ng/ml. The effective amount of the growth factor other than a neurotrophin may be at least 10 ng/ml, at least 20 ng/ml, at least 30 ng/ml, at least 40 ng/mL, at least 50 ng/ml, at least 60 ng/ml, at least 70 ng/ml, at least 80 ng/ml, or at least 90 ng/ml. The effective amount of the growth factor other than a neurotrophin may be at most 100 ng/ml, at most 90 ng/ml, at most 80 ng/ml, at most 70 ng/ml, at most 60 ng/ml, at most 50 ng/ml, at most 40 ng/ml, at most 30 ng/ml, or at most 20 ng/ml.

Contacting the liver cell with the TRK ligand may increase liver cell proliferation at least 2 days following contact as compared to liver cell proliferation before contacting the liver cell with the TRK ligand. Contacting the liver cell with the TRK ligand may increase liver cell proliferation at least 2 days following contact as compared to a control cell.

### 6. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods for Examples 2-7

Mice. Jedi mice were obtained from Jackson Laboratories (Bar Harbor, ME; Jax 028062) and are homozygous for CD45.1 and H-2K^{d} haplotype. Pdgfrb-BAC-eGFP cryopreserved sperm was obtained from Mutant Mouse Resource and Research Centers (MMRRC, Bethesda, Maryland; 031796-UCD), and mice were rederived in C57Bl/6, sub strain B10.D2 females (Jax 000463) that express CD45.2 and H-2K^{d} haplotype. Rederived Pdgfrb-GFP mice were backcrossed into B10.D2 and were maintained heterozygous for GFP, homozygous for CD45.2 and homozygous for H-2K^{d} haplotype. All experiments were performed when mice were 6 weeks to 10 months old and both male and females were used. All animal procedures were performed according to protocols approved by the Institutional Animal Care and Use Committees at Icahn School of Medicine at Mount Sinai and the Institutional Animal Care and Use Committees at Vanderbilt University Medical Center.

Genotyping. Jedi mice were genotyped by PCR genotyping using the following primers: TCRα-For: GAGGAGCCAGCAGAAGGT (SEQ ID NO: 1); TCRα-Rev: TCCCACCCTACACTCACTACA (SEQ ID NO: 2); TCRβ-For: TCAAGTCGCTTCCAACCTCAA (SEQ ID NO: 3); TCRβ-Rev: TGTCACAGTGAGCCGGGTG (SEQ ID NO: 4). Pdgfrb-GFP mice were genotyped by PCR using the following primers: GFP-For: GTGGAAGCAGAGAGGAGAGCATTTG (SEQ ID NO: 5), GFP-Rev: GGTCGGGGTAGCGGCTGAA (SEQ ID NO: 6). H-2K^{d} haplotype, CD45.1, CD45.2 status were determined by flow analysis of PBMCs using the following antibodies purchased from ebiosciences (Thermo Fisher Scientific, Waltham, MA): CD45.1-eFluor-450 (48-0453-80), CD45.2-PE (12-0454-82), H-2K^{d}-APC (17-5957-80).

T cell isolation and adoptive transfer. CD8+ T cells were isolated from spleen and lymph nodes (brachial, inguinal, axillar, submandibular, superficial cervical and mesenteric) of Jedi and controls. Spleens and lymph nodes were collected in ice cold T cell media (RPMI [Gibco® 11875-093] supplemented by 10% FBS, 2 mM L-glutamine [Thermo Fisher Scientific, Waltham, MA; 25030-081], 1% MEM nonessential amino acids [Thermo Fisher Scientific, Waltham, MA; 11140050] and 1 mM sodium pyruvate [Thermo Fisher Scientific, Waltham, MA; 11360-070]). Spleen and lymph nodes were mechanically homogenized and filtered through a 70 μm cell strainer (Falcon®). The cell suspension was centrifuged for 10 min at 4° C., 300 g. Red blood cell (RBC) lysis was performed with RBC buffer (Thermo Fisher Scientific, Waltham, MA; A10492). Cells were then washed with T cell media, filtered through a 70 μm cell strainer and centrifuged for 10 min at 4° C., 300 g. The cell pellet was then resuspended in flow buffer (PBS, pH 7.4, 2 mM EDTA, 0.05% BSA) and CD8+ T cells enriched using the mouse CD8+ T cell isolation kit (STEMCELL technologies, Vancouver, Canada; 19853A) according to the manufacturer's instructions. Enriched CD8+ T cells were centrifuged for 10 min at 4° C., 300 g, and resuspended in PBS. Mice with less than 25 g body weight were injected with up to 12 million Jedi T cells and mice greater than 25 g body weight were injected by tail vein injection with 18 million Jedi T cells. Control mice were injected with either T cells from H-2K^{d} mice or with vehicle only. Mice were not injected with lentiviral GFP particles as previously described (Agudo et al., Nat Biotechnol. 2015; 33(12): 1287-1292; Agudo et al., 2018; 48(2): 271-285.e5) to avoid lentiviral GFP-expression of other cells such as hepatocytes.

Partial hepatectomy. All surgeries were performed per the National Institutes of Health (NIH) recommendations contained in the Guide for the Care and Use of Laboratory. Mice were anesthetized with isoflurane. An incision was made inferior to the xiphoid process. Evisceration of the median and left lateral lobes allowed suture ligation and ˜70% hepatectomy. Skin and abdominal muscles were closed in a two-step procedure and postoperative analgesia administered. Mice were sacrificed 72 hours after partial hepatectomy, and tissues collected for processing.

Treatment with recombinant NTF3. Four months old mice were injected via tail vein for 4 consecutive days with recombinant NTF3 (STEMCELL technology, Vancouver, Canada; cat. 78074, lots 1000070683, 1000077434) diluted in sterile 0.9% saline (0.9% NaCl) or vehicle only. Mice were weighed daily and recombinant NTF3 given at 20 ng/g body weight or 100 ng/g body weight, respectively. Lyophilized NTF3 was solubilized in 0.1% BSA/PBS (bovine serum albumin, Sigma-Aldrich, St. Louis, MO; A3059) and stored at −80° C. until use. As activity and protein concentration declined significantly at approximately 3 months, only aliquots stored for 3 months or less were used for experiments and freeze-thaw cycles minimized.

Mouse blood chemistry analysis. Mouse serum or EDTA-plasma were stored at −80° C. after collection and analyzed on a VetScan® vs2 (Abaxis, Union City, CA) using mammalian liver profile analysis discs (Abaxis, Union City, CA; 500-0040) according to the manufacturer's instructions.

RNA extraction and qRT-PCR. Bulk liver total RNA was isolated using the RNeasy® Kit (Qiagen®) following the manufacturer's instructions. Five μg of total RNA was transcribed to cDNA using the RNA-to-cDNA ecoDry® Premix (Takara Bio, San Jose, CA). IQ SYBR Green Supermix (Bio-Rad Laboratories, Hercules, CA) was used to quantitative on a LightCycler® 480 System (Roche, Basel, Switzerland) or CFX96 Real-time System (Bio-Rad Laboratories, Hercules, CA). Primer sequences used were Desmin: For-GCCACCATGAGCCAGGCCTACT (SEQ ID NO: 7), Rev-TGCTCGAGGGAAC-ACGGGAGA (SEQ ID NO: 8), Pdgfrb: For-ACTACATCTCCAAAGGCAGCACCT (SEQ ID NO: 9), Rev-TGTAGAACTGGTCGTTCATGGGCA (SEQ ID NO: 10), Ntrk2 (encoding for TrkB): For-GTCTGGAGGGTGCTATGCTA (SEQ ID NO: 11), Rev-CAGGGGCAGAAACTCCAGAA (SEQ ID NO: 12), GAPDH: For-CAATGACCCCTTCATTGACC (SEQ ID NO: 13), Rev-GATCTCGCTCCTGGAAGATG (SEQ ID NO: 14).

RNA-sequencing. Bulk total RNA was isolated using RNeasy® Kit (Qiagen®) following the manufacturer's instructions, and 1 μg were used for polyA selection and library preparation using the Illumina® TruSeq™ protocol. Samples were sequenced 50 bp paired-end on a NovaSeq™ 6000 by the Genomics Core Facility at The Rockefeller University. Raw FASTQ files were aligned to the mouse mm¹⁰ genome using the STAR aligner (v. 2.7.1a). The STAR command used was:

STAR --runThreadN 10 \\
--genomeDir mm10 \\
--readFilesIn INPUT_R1_001.fastq.gz INPUT_L001_R2_001.fastq.gz
\\
--clip3pAdapterSeq GATCGGAAGAGC \\
--outFilterMismatchNmax 2 \\
--outFileNamePrefix OUTFILE \\
--readFilesCommand gunzip -c \\
--outSAMunmapped Within \\
--outStd BAM_Unsorted \\
--outSAMtype BAM Unsorted \\
--outStd BAM_Unsorted

The resulting BAM file was sorted using samtools (v. 1.15.1). Reads were summarized using featureCounts (v1.6.4) against the Gencode mouse vM6 release disregarding multi-mapping and overlapping reads. The command used was:
featureCounts -a INPUT.gtf -T 4 -s 2 -o OUTFILE.txt
LIST_OF_BAMFILES

Differential and gene set analysis was done in R session (development version, 2022 Jan. 7 r81454) using the quasi-likelihood framework in edgeR (v. 3.37.0) and the mroast function on the mouse version of the MSigDB gene sets based on release v5.2 provided by the Bioinformatics group at the Walter+Eliza Hall Institute for Medical Research (bioinf.wehi.edu.au/software/MSigDB/). For other packages used to generate figures see section Olink® below. The Gene Expression Omnibus accession number for the transcriptome profiles reported here is GSE211370.

Olink®. Fresh frozen liver tissue samples were homogenized using a TissueLyser® LT (Qiagen, Hilden, Germany) in RIPA buffer (Sigma-Aldrich, St. Louis, MO; R0278). Protein concentration was determined by Bio-Rad Protein Assay (Bio-Rad Laboratories, Hercules, CA), lysate protein concentration adjusted to 0.5 μg/μl protein liver lysate and submitted to the Human Immune Monitoring Center (HIMC), a core facility at Icahn School of Medicine at Mount Sinai.

Olink® data were generated using the Olink® mouse exploratory panel (v.3801), and the data was exported in NPX format from the Olink® NPX Manager (v. 2.0.0.173) to Microscoft® Excel® xlsx files. These files were imported into an R session (development version, 2022 Jan. 7 r81454) using the Olink® Analyze package (v. 3.0.0). Groups were compared using a one-way ANOVA with Tukey's post-hoc test as implemented in the Olink® Analyze package and using the default adjusted P value of 0.05. Other R packages used to generate figures and plots were the packages NMF (v. 0.23.0), ggplot2 (v. 3.3.5), and cowplot (v. 1.1.1).

Immunohistochemistry. For immunohistochemistry, formalin-fixed paraffin embedded sections were cut at 4 μm thickness and slides incubated at 65° C. for 30 min. Slides were deparaffinized in Histo-Clear® (National Diagnostics, Charlotte, NC; HS2001GLL) and rehydrated by an ethanol gradient. Heat antigen retrieval was performed in 10 mM sodium citrate (pH 6.0) in a pressure cooker (Cuisinart®, CPC-600C), 20 min at high-pressure, natural pressure release for 30 min, and cooled at room temperature (i.e., about 20° C. to about 22° C.) for 30 min. Endogenous peroxidase quenching was performed with 1.5% hydrogen peroxide (Sigma-Aldrich, St. Louis, MO; H1009) for 20 min, and slides washed twice in phosphate buffered saline (PBS) twice. Blocking was performed for 30 min with Protein Block, Serum-free (Agilent, Santa Clara, CA; X090930-2). The primary antibody (TABLE 1) was diluted in antibody diluent (Agilent, Santa Clara, CA; S080983-2) and applied to the slides in a wet chamber overnight at 4° C. After 2 washes in PBS, slides were incubated with the appropriate secondary antibody (TABLE 1) for 30 min followed by two washes in PBS. The antigen was revealed with DAB (Vector Laboratories, Newark, CA; SK-4105) until the appropriate staining on a control slide was obtained. The same reveal time was then used on all experimental slides. Slides were stained with hematoxylin (Sigma-Aldrich, St. Louis, MO; GHS332) for 30 sec, washed gently in lukewarm tap water for 5 min, and washed twice in distilled water, followed by two washes in phosphate buffered saline (PBS, pH 7.4). Slides were washed in increasing ethanol/water gradients, followed by three washes in Histo-Clear®. Whole-slide scans for subsequent analyses and publishing were obtained using an VS200 Research Slide Scanner (Olympus, Tokyo, Japan) and a SCN400 (Leica, Wetzlar, Germany). Whole-slide images were processed and annotated in QuPath 0.3.2. and 0.4.2. Ki67-positive hepatocyte nuclei were counted over ten random high-power fields (ROIs of 0.27 mm²). Hepatocytes and cleaved caspase 3-positive hepatocyte nuclei were counted over 5 random high-power fields and mean % of cleaved caspase 3-positive hepatocytes per mouse calculated.

TABLE 1
List of primary and secondary antibodies for immunohistochemistry,
immunofluorescence, and multiplex immunohistochemistry.

| | | | | | Research |
|---|---|---|---|---|---|
| | | | | | Resource |
| | | | Application | | Identifiers |
| Name | Cat# | Vendor | (IHC/IF) | Dilution | (#RRID) |
| cleaved caspase 3 | 9661 | Cell | IHC | 1:3000 | AB_2341188 |
| | | Signaling | | | |
| | | Technology, | | | |
| | | Danvers, MA | | | |
| CCND1 | ab16663 | Abcam, | IHC | 1:800 | AB_443423 |
| | | Cambridge, | | | |
| | | UK | | | |
| CD31/Pecam1 | 550274 | BD | IHC | 1:100 | AB_393571 |
| | | Pharmingen | | | |
| desmin (rabbit host) | ab15200 | Abcam, | IHC | 1:8000 | AB_301744 |
| | | Cambridge, | | | |
| | | UK | | | |
| desmin (goat host) | AF3844 | R&D | IF | 1:25 | AB_2092419 |
| | | Systems, | | | |
| | | Minneapolis, | | | |
| | | MN | | | |
| E-cadherin | 3195S | Cell | IHC | 1:3000 | AB_2291471 |
| | | Signaling | | | |
| | | Technology, | | | |
| | | Danvers, MA | | | |
| F4/80 | 14-4801-82 | eBioscience, | IHC | 1:100 | AB_467558 |
| | | San Diego, | | | |
| | | CA | | | |
| GFAP | NB300-141 | Novus | IHC | 1:1000 | AB_10001722 |
| | | Biologicals, | IF | 1:50 | |
| | | Centennial, | | | |
| | | CO | | | |
| GFP | ab183735 | Abcam, | IHC | 1:1000 | AB_2924655 |
| | | Cambridge, | IF | 1:200 | |
| | | UK | | | |
| glutamine synthetase | ab49873 | Abcam, | IHC | 1:20,000 | AB_880241 |
| | | Cambridge, | | | |
| | | UK | | | |
| granzyme B | ab4059 | Abcam, | IHC | 1:5000 | AB_304251 |
| | | Cambridge, | | | |
| | | UK | | | |
| HNF4a | ab181604 | Abcam, | IHC | 1:400 | AB_2890918 |
| | | Cambridge, | | | |
| | | UK | | | |
| Ki67 | M7249 | Dako, | IHC | 1:50 | AB_2250503 |
| | | Glostrup, | | | |
| | | Denmark | | | |
| | ab15580 | Abcam, | IF | 1:5000 | AB_443209 |
| | | Cambridge, | | | |
| | | UK | | | |
| NTF3 | PA5-14861 | Invitrogen, | IHC | 1:500 | AB_2154265 |
| | | Waltham, MA | IF | 1:50 | |
| TRKA (NTRK1) | AF1056SP | R&D | IF | 1:100 | AB_2283049 |
| | | Systems, | | | |
| | | Minneapolis, | | | |
| | | MN | | | |
| TRKB (NTRK2) | AF1494 | R&D | IF | 1:100 | AB_2155264 |
| | | Systems, | | | |
| | | Minneapolis, | | | |
| | | MN | | | |
| reelin | AF3820 | R&D | IF | 1:50 | AB_2253745 |
| | | Systems, | | | |
| | | Minneapolis, | | | |
| | | MN | | | |
| Phalloidin-AF488 | A12379 | Invitrogen, | IF | 1:40 | AB_2759222 |
| | | Waltham, MA | | | |
| Anti-Mouse IgG, | MP-7402 | Vector | IHC | 1:2000 | AB_2336528 |
| Secondary antibody, | | Laboratories, | | | |
| Peroxidase | | Newark, CA | | | |
| Goat Anti-Rat IgG, | MP-7404 | Vector | IHC | 1:2000 | AB_2336531 |
| Secondary antibody, | | Laboratories, | | | |
| Peroxidase | | Newark, CA | | | |
| Goat anti-Rabbit IgG, | MP-7451 | Vector | IHC | 1:2000 | AB_2631198 |
| Secondary antibody, | | Laboratories, | | | |
| Peroxidase | | Newark, CA | | | |
| Donkey Anti-Goat | A-21447 | Invitrogen, | IF | 1:2000 | AB_141844 |
| Secondary Antibody, | | Waltham, MA | | | |
| Cy3 | | | | | |
| Goat anti-Rabbit | A-21245 | Invitrogen, | IF | 1:2000 | AB_141775 |
| Secondary Antibody, | | Waltham, MA | | | |
| Alexa Fluor 647 | | | | | |

Immunofluorescence. Primary mouse hepatic stellate cells that had been seeded on glass coverslips were washed in PBS, incubated for 10 min in cold (−20° C.) 100% acetone then rehydrated for 5 min in PBS (pH 7.4). Coverslips were incubated with respective primary antibodies (TABLE 1; dilution buffer PBS) in a wet chamber for 1 hour at room temperature. Coverslips were then washed 5 times for 5 min each in PBS and incubated with respective secondary antibodies (TABLE 2) for 1 h at room temperature. Coverslips were washed 5 times in PBS for 5 min each and mounted with ProLong™ Gold antifade reagent with DAPI (Invitrogen™, P36931).

TABLE 2
List of primary for western blotting.

| | | | | Research |
|---|---|---|---|---|
| | | | | Resource |
| | | | | Identifiers |
| Name | Cat# | Vendor | Dilution | (#RRID) |
| b-actin | ab6276 | Abcam, | 1:10,000 | AB_2223210 |
| | | Cambridge, | | |
| | | UK | | |
| CCND1 | ab16663 | Abcam, | 1:750 | AB_443423 |
| | | Cambridge, | | |
| | | UK | | |
| desmin | ab15200 | Abcam, | 1:1000 | AB_301744 |
| | | Cambridge, | | |
| | | UK | | |
| IGFBP2 | ab188200 | Abcam, | 1:1000 | N/A |
| | | Cambridge, | | |
| | | UK | | |
| tubulin | 3873 | Cell Signaling | 1:10,000 | AB_1904178 |
| | | Technology, | | |
| | | Danvers, MA | | |
| GAPDH | CB1001 | Calbiochem, | 1:5,000 | AB_2107426 |
| | | San Diego, | | |
| | | CA | | |
| NTF3 | PA1-18385 | Invitrogen, | 1:1000 | AB_1077209 |
| | | Waltham, MA | | |
| PCNA | 13110 | Cell Signaling | 1:1000 | AB_2636979 |
| | | Technology, | | |
| | | Danvers, MA | | |
| Pan-TRK | 92991S | Cell Signaling | 1:1000 | AB_2800196 |
| | | Technology, | | |
| | | Danvers, MA | | |
| Phospho-TRK (P-TRKA | 4621S | Cell Signaling | 1:1000 | AB_916186 |
| (Tyr674/675)/ | | Technology, | | |
| TrkB (Tyr706/707) | | Danvers, MA | | |

For immunofluorescence, 4 μm cryosections from fresh frozen liver samples embedded in Optimal Cutting Temperature (OCT) compound (Sakura, Tokyo, Japan) were cut and dried for a minimum of 1 h at room temperature and stored at −80° C. until use. Frozen sections were thawed and rehydrated in PBS for 5 min at room temperature, fixed in 100% acetone for 10 min at −20° C. and washed in PBS. The primary antibody (TABLE 1) was diluted in antibody diluent (Agilent, Santa Clara, CA; S080983-2) and incubated for 60 min at room temperature in a wet chamber. Slides were then washed 3×5 min in PBS. The secondary antibody (TABLE 1) was diluted in antibody diluent (Agilent, Santa Clara, CA; S080983-2 or PBS), and slides incubated in a wet chamber for 30-60 min. Slides were washed three times in PBS, followed by two washes in distilled H₂O. The slides were then mounted with a DAPI-containing medium (ProLong™ Antifade, Thermo Fisher Scientific, Waltham, MA; P36931). Images were taken with either a Nikon® A1R confocal microscope (Nikon, Tokyo, Japan), an Axio Imager® M2, or a Axio Imager® 7 (Zeiss, Oberkochen, Germany) microscope. For phalloidin staining, Alexa Fluor™ 488 Phalloidin (Invitrogen™, A12379) was added to the diluent containing the secondary antibody (40× dilution).

Multiplex immunohistochemistry. For multiplex immunohistochemistry, formalin-fixed paraffin-embedded (FFPE) sections were incubated at 65° C. for 30 min and cooled for 15 min at room temperature for 2 cycles to strongly fix the tissue to the slide. After deparaffinization and rehydration, heat antigen retrieval was performed with 10 mM sodium citrate (pH 6). The slides were then stained with hematoxylin to avoid staining after the signal reveal which would unbind the chromogen. Endogenous peroxidase quenching was performed with 1.5% hydrogen peroxide (Sigma-Aldrich, St. Louis, MO; H1009) for 20 min, and slides washed twice in phosphate buffered saline (PBS). Blocking was performed for 30 min with Protein Block, Serum-free (Agilent, Santa Clara, CA; X090930-2). The CCND1 primary antibody (TABLE 1) was diluted in antibody diluent (Agilent, Santa Clara, CA; S080983-2) and incubated on the slides for 60 min. Slides were washed in PBS and incubated with secondary antibody for 30 min. Slides were washed in PBS and slides incubated with 3-amino-9-ethylcarbazole (AEC, Agilent, Santa Clara, CA; K346111-2) until the appropriate staining on a control slide was obtained. The same reveal time was used on all experimental slides. The slides were washed in PBS, and cover slipped with a PBS mounting medium immediately prior to imaging. Slides were scanned with the VS200 research slide scanner (Olympus, Tokyo, Japan). After imaging, the cover glass was carefully removed in a PBS wash, and the slides were de-stained in a gradient of dH₂O, 70% ethanol, 95% ethanol, 70% ethanol, dH₂O, and conserved in PBS at 4° C. Slides were incubated in 10 mM sodium citrate (pH 6.0) in a pressure cooker (Cuisinart®, CPC-600C), for 20 min at high-pressure, natural pressure release for 30 min, and cooled at room temperature for 30 min. The hematoxylin staining was repeated, and the steps were repeated from the blocking step but with anti-HNF4a antibody (TABLE 1). Images were registered in QuPath 0.3.2 with the image combiner extension 0.2.3 using rigid transform based on image intensity at 5-pixel resolution. Ten high-power fields (ROIs measuring 0.27 mm²) evenly and blindly distributed across tissue sections per sample were assessed.

Cell lines. Human HepG2 (ATCC HB-8065) were maintained in MEM media with 10% FBS and the mouse hepatocyte cell line AML12 (ATCC, Manassas, VA; CRL-2254) in DMEM/F12 media supplemented with dexamethasone (40 ng/ml) and Insulin-Transferrin-Selenium (Gibco, Waltham, MA; 41400045), containing 10% FBS and 1% penicillin and streptomycin. JS1 (RRID: CVCL_C7PL), LX2 (RRID: CVCL_5792, MilliporeSigma, Burlington, MA; SCC064) and TWNT4 (RRID: CVCL_W296) cells were cultured in DMEM supplemented with 10% FBS. 293T cells (ATCC, Manassas, VA; CRL-3216) were cultured in DMEM supplemented with 10% FBS.

Primary hepatic stellate cell isolation. Pdgfrb-GFP-positive mice were anesthetized and cannulated via the portal vein. Livers were perfused with pre-warmed (37° C.) buffers: about 25 ml of liver perfusion medium (Gibco, Waltham, MA; 17707-038), followed by about 30 ml 0.2% Pronase™/HBSS (Roche, Basel, Switzerland; cat. 11459643001, Gibco, Waltham, MA; 24020-117). Livers were then perfused by 25 ml 0.05% Pronase™/0.05% Collagenase B (Roche, Basel, Switzerland; 11088831001) diluted in HBSS (Gibco, Waltham, MA; 24020-117). Livers were then resected and placed in ice cold 5 ml hepatocyte wash medium (Gibco, Waltham, MA; 17704024), the liver capsule gently teased apart with tweezers and cells released. Cells were taken up in hepatocyte wash medium (Gibco, Waltham, MA; 17704-024) and filtered through a 100 μm mesh. Cells were then centrifuged for 2 min at 100 g, 4° C. The supernatant containing non-parenchymal cells was filtered through 70 μm mesh and centrifuged for 5 min at 580 g, 4° C. The cell pellet was resuspended in 25 ml ice cold hepatocyte wash medium (Gibco, Waltham, MA), filtered through a 70 μm mesh, and centrifuged for 5 min at 580 g, 4° C. The resulting cell pellet was then resuspended in 2.5 ml 50% Percoll®/PBS (Percoll®, GE HealthCare, Chicago, IL; GE17-0891-01), transferred into a 15 ml reaction tube and overlayed with 5 ml of 35% Percoll®/PBS, followed by 1 ml sterile 1% BSA/PBS. Cells were centrifuged at 1380 g for 30 min at 4° C. The cell layer on top of the 35% Percoll®/PBS gradient was collected, resuspended in 25 ml hepatocyte wash buffer, filtered through 70 μm mesh and centrifuged for 5 min at 580 g, 4° C. The cell pellet was then resuspended in DMEM/10% FBS and seeded on coverslips for further analyses.

Primary hepatocyte isolation. Pdgfrb-GFP-negative or H-2Kd mice older than 4 months were anesthetized and cannulated via the portal vein. Livers were perfused with about 25 ml of pre-warmed Liver Perfusion Medium (Gibco, Waltham, MA; 17707-038) followed by about 50 ml Liver Digestion Medium (Gibco, Waltham, MA; 17703-034). Liver capsule was gently teased apart with tweezers and hepatocytes released. Cells were taken up in Hepatocyte Wash Buffer (Gibco, Waltham, MA; 17704-024) and filtered through 100 μm mesh. Cells were then centrifuged for 2 min at 100 g, 4° C. and cell pellet resuspended in ice cold 10 ml DMEM (Gibco, Waltham, MA; 11885-084)/5% FBS. 10 ml of ice cold 100% Percoll®/PBS (GE HealthCare, Chicago, IL; GE17-0891-01) was added for gradient purification and cells centrifuged for 10 min at 200 g, 4° C. Cell pellet was resuspended with 20 ml DMEM/5% FBS and centrifuged for 2 min at 100 g, 4° C. 1-2×10³ hepatocytes were plated on collagen-coated 96-well plates in DMEM/5% FBS/Pen/Strep. Rat tail collagen I (Gibco, Waltham, MA; A1048301) was used for coating. Cell culture media was changed 3 hours after plating with either serum-free William's E media (Gibco, Waltham, MA) containing Pen/Strep (Gibco, Waltham, MA) or DMEM/5% FBS/Pen/Strep. NTF3 (STEMCELL technologies, Vancouver, Canada) was added 1 day after plating at a final concentration of 20 ng/ml and primary hepatocytes cultured for 3 days with NTF3 until analysis.

Ntrk2 knockdown in primary hepatocytes. Lentivirus containing short hairpin RNAs (shRNAs) were expressed in a lentiviral vector (pLKO.1 puro). Lentivirus with shNon-target (shNT, Addgene, Watertown, MA; #109012) and mouse shNTRK2 [MilliporeSigma, Burlington, MA; TRCN0000023703, target sequence: CATTCCAAGTTTGGCATGAAA (shNtrk2_1; SEQ ID NO: 15) and TRCN0000361390, target sequence: CAGCAACCTGCGGCACATAAA (shNtrk2_2; SEQ ID NO: 16)] were generated in 293 T cells (ATCC CRL-3216). Primary mouse hepatocytes (2×10³ cells per well) were incubated with lentivirus for 2 days. For cell proliferation experiments, media was and changed to serum-free William E media without L-glutamine containing NTF3 (20 ng/ml) or vehicle only. Ntrk2 knock down (encoding for TRKB) was verified by qRT-PCR.

Cell proliferation assays. Primary mouse hepatocytes, HepG2 or AML 12 cells were grown in opaque-walled 96-well plates. NTF3 (final concentration 20 ng/ml) (STEMCELL Technologies, Vancouver, Canada; cat. 78074) and/or Loxo-195 (final concentration 5 nM) (Selleckchem, Houston, TX; S8636) were added into complete growth media without L-glutamine. Primary mouse hepatocytes were also grown in serum-free William's E media without supplements. Cells were analyzed by CellTiter-Glo® (Promega G7570, Madison, WI) which detects the number of viable cells on day 0 to day 5 by on detection systems GloMax® (Promega) or Bioteck Synergy H1 (Agilent, Santa Clara, CA). Mouse recombinant HGF (Abcam, Cambridge, UK; ab281797) was added to cell culture media at a final concentration 5 ng/ml. TRKB inhibitor ANA-12 (Selleckchem, Houston, TX; S7745) was diluted in DMSO (10 mM stock solution) and added to cell culture media at a final concentration of 10 nM.

Western blotting. Fifty micrograms of snap frozen liver were homogenized with a PowerLyzer® (Qiagen, Hilden, Germany) in RIPA buffer containing proteinase inhibitors (Roche, Basel, Switzerland; complete Mini proteinase inhibitor, 2 tab/10 ml buffer) and 1×HALT phosphatase inhibitors (Thermo Scientific, Waltham, MA; cat. 78428). Lysates were sonicated and then centrifuged for 10 min at 18,800×g. Protein concentration of supernatants was determined by Direct Detect Spectrophotometer (MilliporeSigma, Burlington, MA).

Human HepG2 and mouse AML12 cells were grown in 6-well plates. Cells were treated with NTF3 (20 ng/ml) purchased from STEMCELL technologies, Vancouver, Canada (cat. 78074), R&D Systems, Minneapolis, MN (267-N3), Gibco, Waltham, MA (PHC7036) and/or Loxo-195 (5-10 nM) (Selleckchem, Houston, TX; S8636) in complete growth media without L-glutamine for 2 hours. Lyophilized NTF3 was solubilized with sterile filtered PBS supplemented with 0.1% BSA, aliquoted and stored at −80° C. until use. Only aliquots with <3 months shelf life were used for experiments as activity and protein concentration declined significantly after 3 months. HepG2 and AML12 cells were collected on ice in PBS, centrifuged for 10 sec at and cell pellet homogenized in RIPA buffer containing inhibitors (Roche, Basel, Switzerland; complete Mini proteinase inhibitor, 1×HALT phosphatase inhibitors, Thermo Scientific, Waltham, MA; cat. 78428). Cell lysates were sonicated and centrifuged for 10 min at 18,800×g. Protein concentration of supernatants were determined by Direct Detect Spectrophotometer (MilliporeSigma, Burlington, MA).

For immunoblot analysis, 30-50 μg of protein diluted in LSD sample buffer (Invitrogen, Waltham, MA) were analyzed per SDS-PAGE (NuPAGE™, Invitrogen, Waltham, MA) using MOPS buffer or MES buffer (Invitrogen, Waltham, MA). Gels were stained with Coomassie (Bio-Safe Coomassie G-250 Stain, Bio-Rad, Hercules, CA) and transferred onto PVDF membranes using semi-dry blotting (Trans-Blot SD, Bio-Rad, Hercules, CA). Antibodies and dilutions used are indicated in TABLE 2. Images were visualized on an Amersham™ Imager 680 (Cytiva, Marlborough, MA). Semi-quantitative band densitometry was performed using ImageJ software (version 1.53).

Statistical Analysis. For analysis of the Olink® and RNA-seq data see the corresponding sections. Other data analysis was performed using GraphPad Prism (version 9.4.1). Two groups were compared by unpaired two-tailed Mann-Whitney test. Data of three or more groups were assessed by Kruskal-Wallis test with post hoc Dunn's test. Three or more groups were analyzed by two-way ANOVA with post hoc test Sidak, testing for vehicle only treated cells vs. NTF3-treated cells.

### Example 2

### Jedi T Cells Deplete eGFP-Expressing HSCs In Vivo

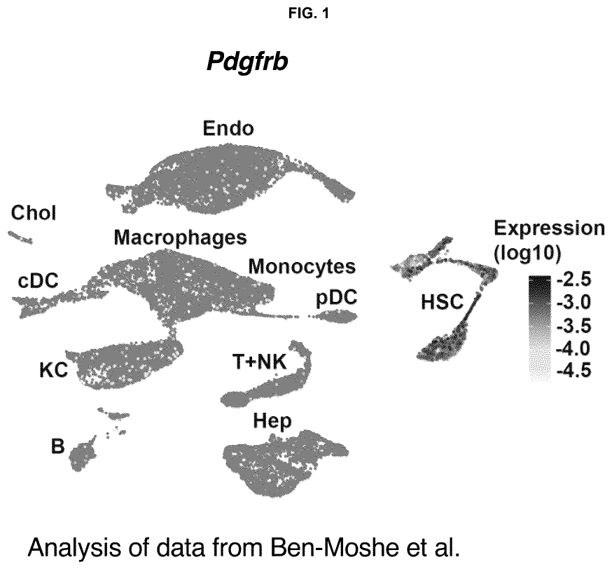

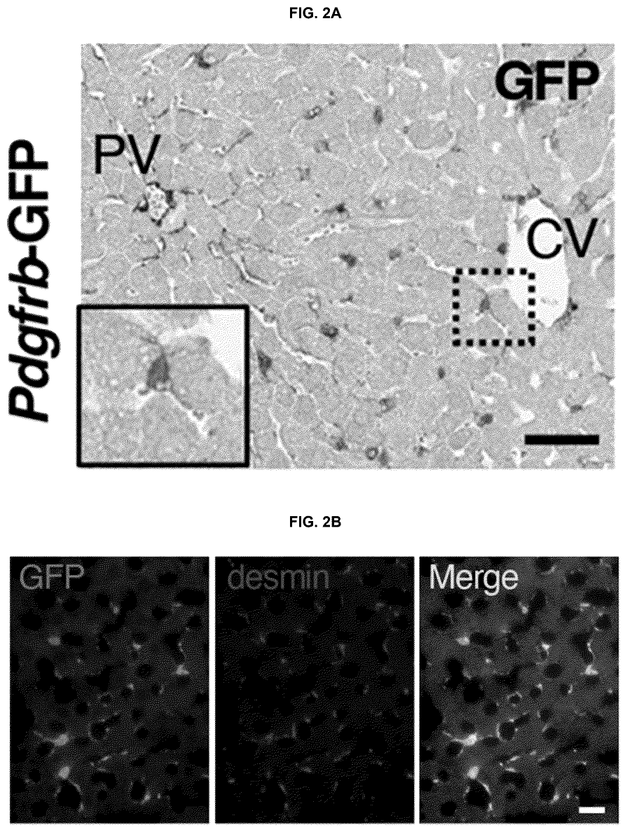

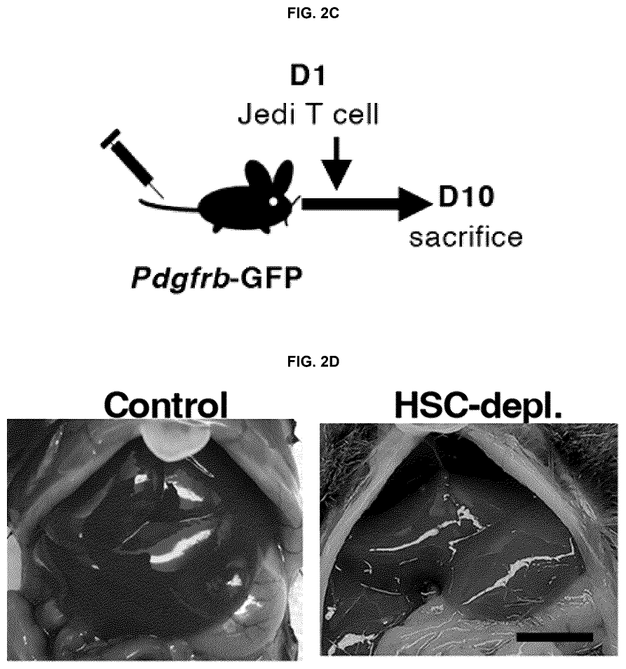

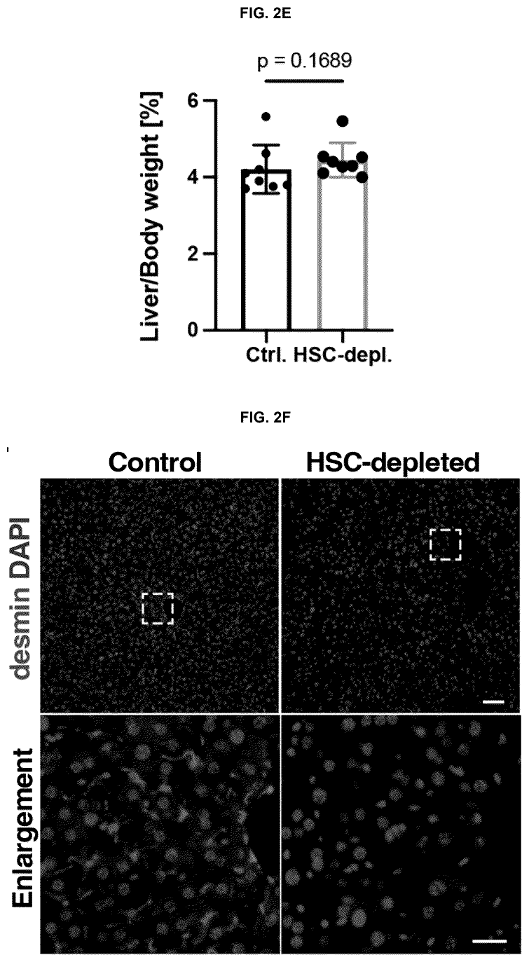

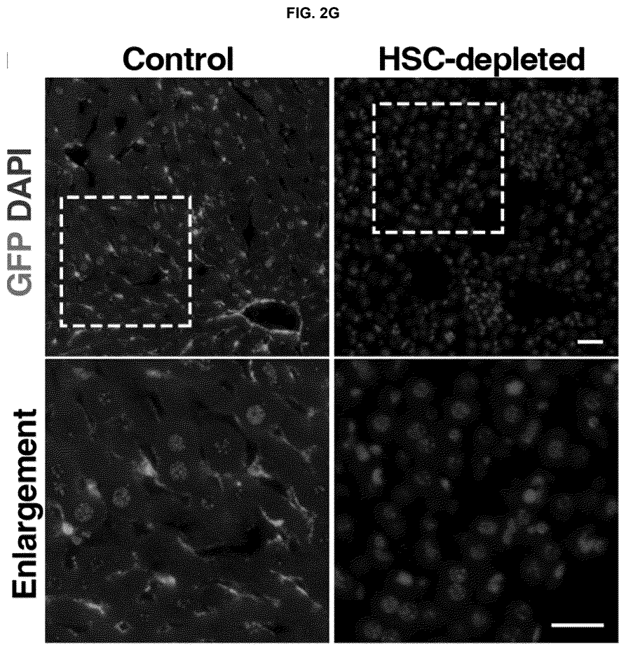

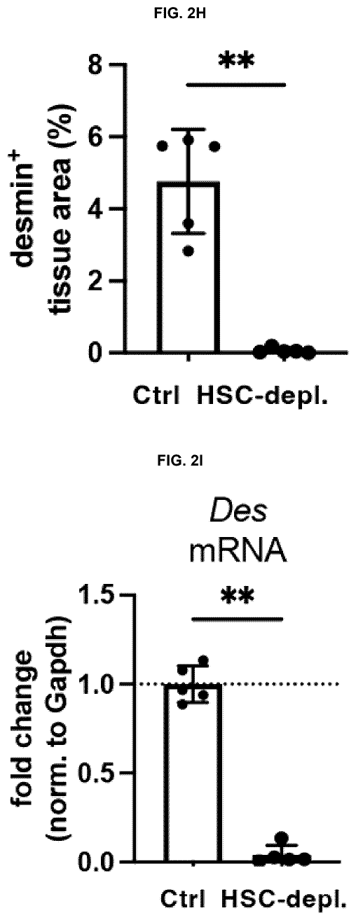

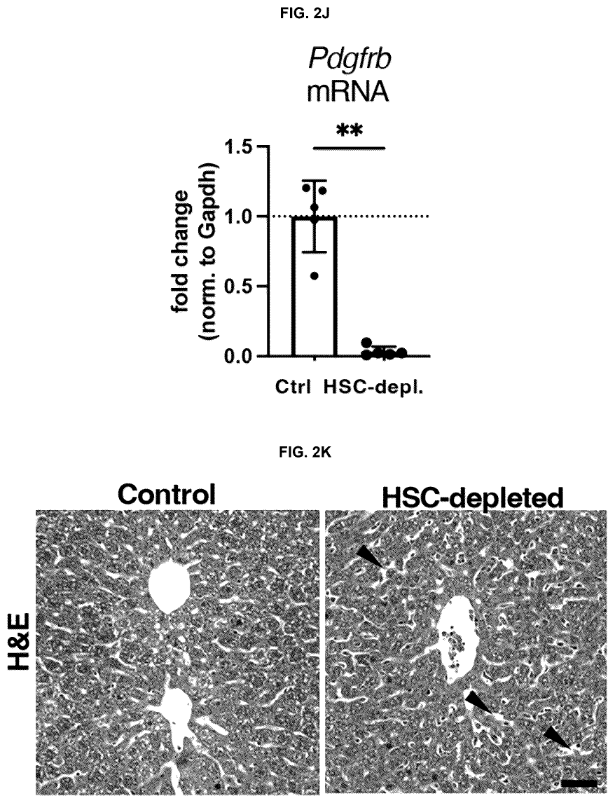

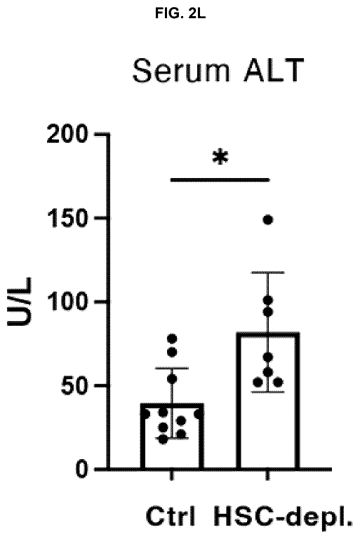

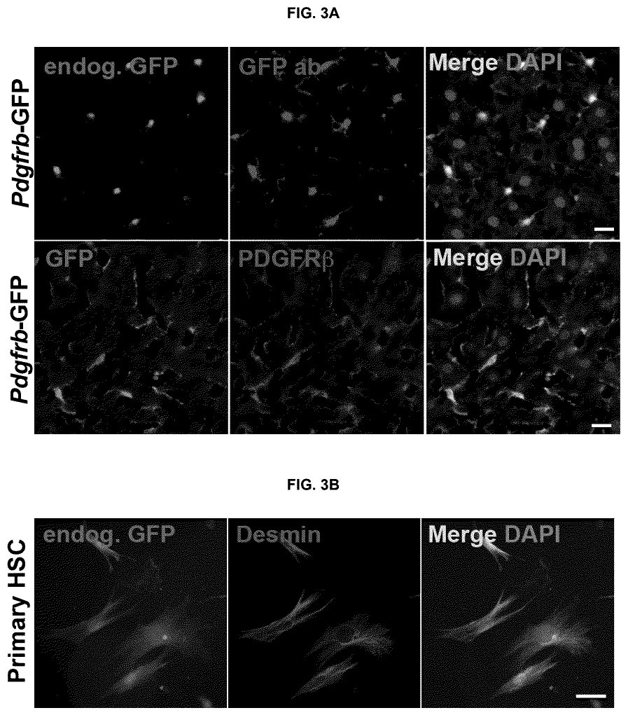

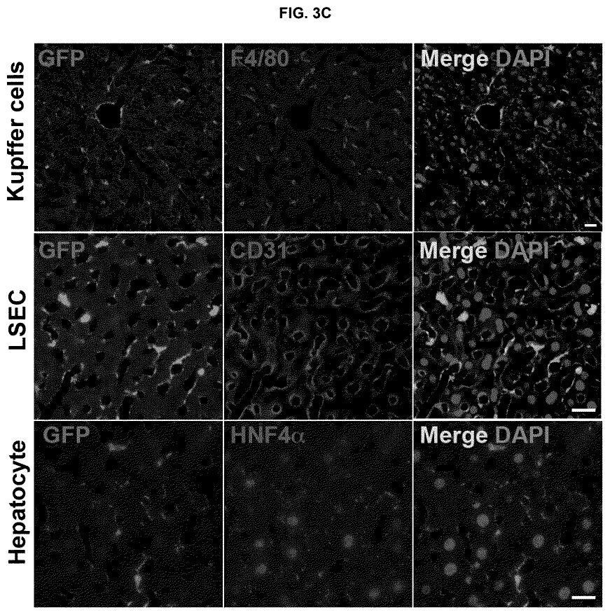

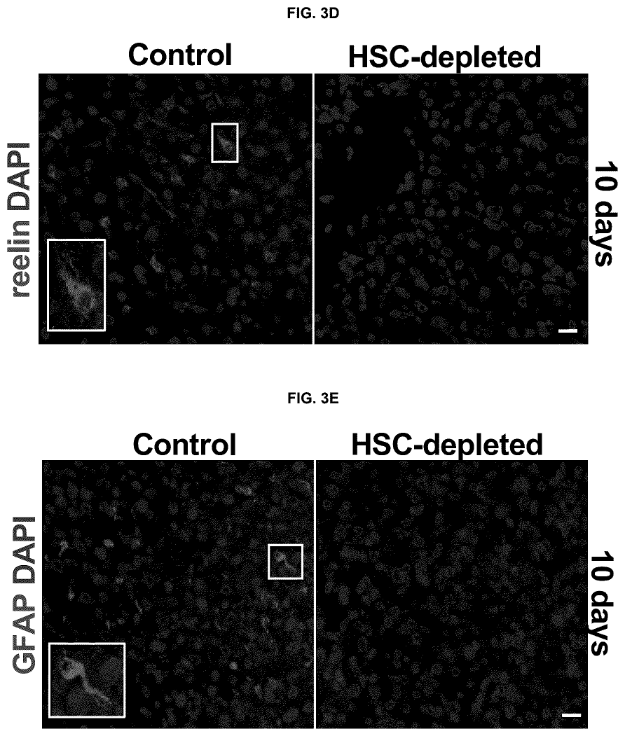

Agudo and colleagues engineered mice to make T cells that specifically target cells displaying enhanced green fluorescent protein (eGFP). These just eGFP death-inducing (Jedi) mice produce T cells bearing a T cell receptor (TCR) specific for the immunodominant epitope of eGFP presented by MHC class I (MHC-I). Adoptive transfer of Jedi T cells into an eGFP reporter mouse with the same MHC-I haplotype H-2K^{d} efficiently ablates eGFP+ cells within days. Single-cell RNA sequencing (scRNA-seq) analyses from human and mouse livers have revealed the heterogeneity and spatial zonation of HSCs. Because analysis of published scRNA-seq datasets showed high expression and specificity of platelet-derived growth factor receptor B (Pdgfrb) in HSCs in both injured and healthy livers (FIG. 1), Pdgfrb-BAC-eGFP transgenic mice (henceforth called Pdgfrb-GFP) were used, which express eGFP in HSCs. Immunostaining for GFP in livers of Pdgfrb-GFP mice showed staining only in small non-parenchymal cells (FIG. 2A and FIG. 3A), and coimmunofluorescence analysis for GFP and desmin, one of the most reliable markers for HSCs in mouse liver, confirmed HSC-specific GFP expression (FIG. 2B). Primary HSCs isolated from Pdgfrb-GFP mice also coexpressed desmin and GFP (FIG. 3B). Coexpression of GFP was not observed with markers of Kupffer cells (F4/80), liver sinusoidal endothelial cells (CD31/PECAM1), or hepatocytes (HNF4a) (FIG. 3C), indicating that, in the liver, GFP was expressed only in HSCs.

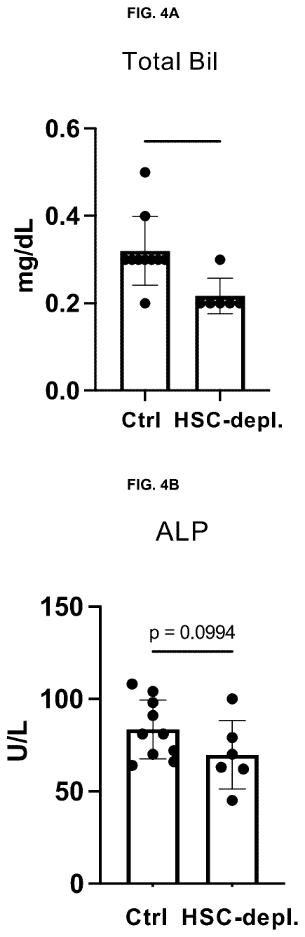

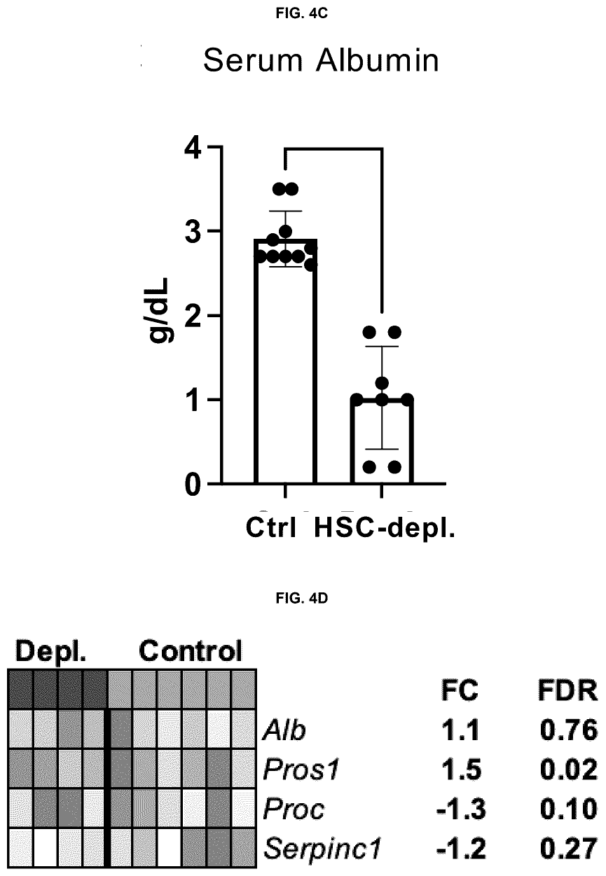

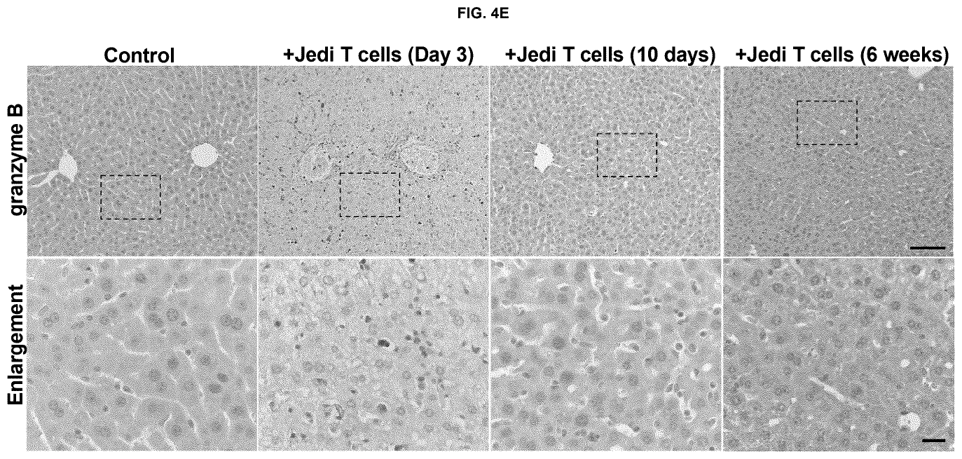

Adoptive transfer of Jedi T cells isolated from Jedi mice, but not control T cells from H-2K^{d} control mice, almost completely ablated HSCs within 10 days in Pdgfrb-GFP mice (FIGS. 2C-J). Livers appeared macroscopically normal, and HSC-depleted mice displayed a similar liver-to-body weight ratio as control mice at this time point (FIGS. 2D-E). No cells positive for GFP or the HSC markers desmin (DES), reelin, or glial fibrillary acidic protein (GFAP), could be detected by immunofluorescence, indicating efficient ablation of HSCs (FIGS. 2F-G and FIGS. 3D-E). Quantification of the desmin+ area by tissue morphometry revealed significantly fewer HSCs in HSC-ablated livers (FIG. 2H), a result that was confirmed by measuring Des and Pdgfrb gene expression in whole liver RNA by qRT-PCR (FIGS. 2I-J). By hematoxylin and eosin (H&E) staining, sinusoids were slightly distended (FIG. 2K). Serum alanine aminotransferase (ALT) was increased (FIG. 2L), an indication of discrete hepatocyte injury, likely resulting from a transient, reversible increase in ALT due to T cell-induced cytokine release, similar to what is seen after chimeric antigen receptor (CAR) T cell administration in patients. Serum measurements of total bilirubin and alkaline phosphatase (ALP) (FIGS. 4A-B) were comparable in control and HSC-depleted mice. Serum albumin concentration was decreased in HSC-depleted mice (FIG. 4C), but gene expression analysis for albumin and coagulation factors synthesized by the liver were similar in control and HSC-depleted mice (FIGS. 4C-D).

Although Jedi T cells were readily detectable in the liver by staining for granzyme B, a CD8+ T cell marker, at 3 days after adoptive transfer, no Jedi T cells were detected at 10 days or up to 6 weeks after adoptive transfer (FIG. 4E). Similarly, no Jedi TCRa or TCRb mRNA expression was detected by whole liver RNA-seq at 10 days. These observations agree with earlier reports in which Jedi T cells become undetectable 2-3 weeks after adoptive transfer. In summary, these results demonstrate efficient ablation of HSCs with no detectable Jedi T cells following days after adoptive transfer in liver indicating that subsequent liver changes are due to ablation of HSCs rather than indirect effects of persistent Jedi T cells.

### Example 3

### Sustained HSC Depletion Causes Loss of Liver Mass and Reduces the Number of Midlobular Hepatocytes

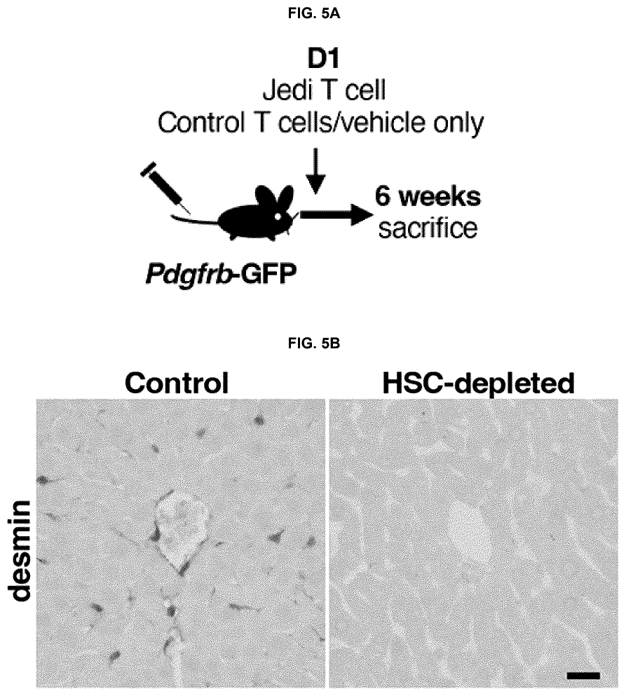

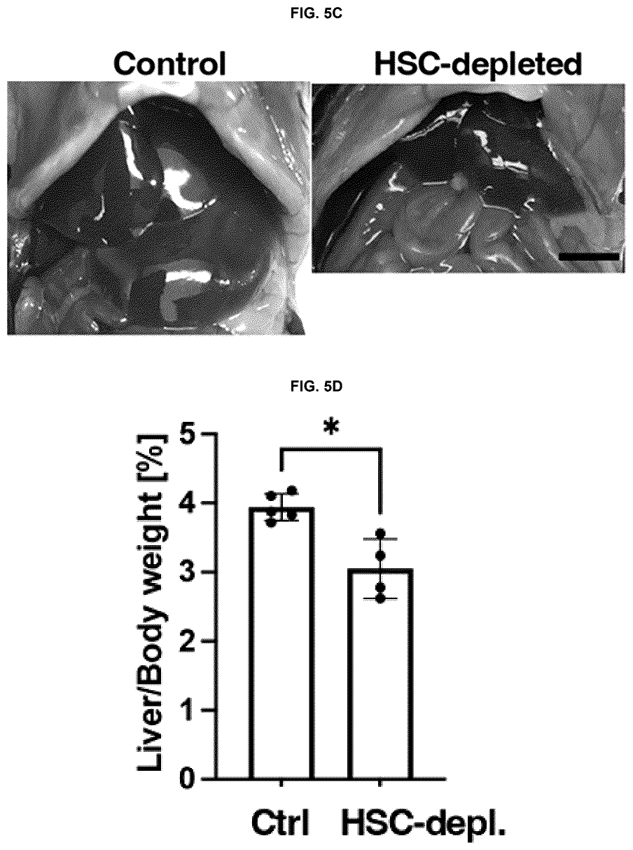

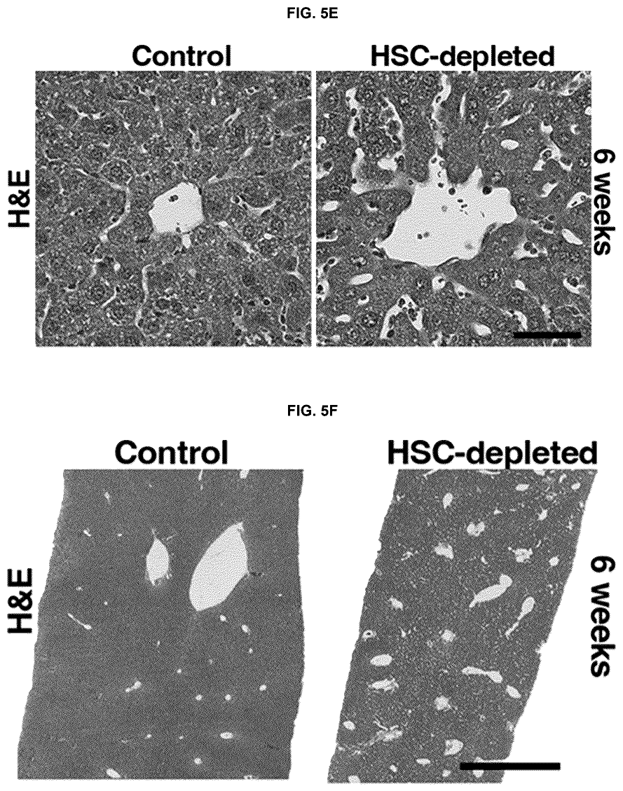

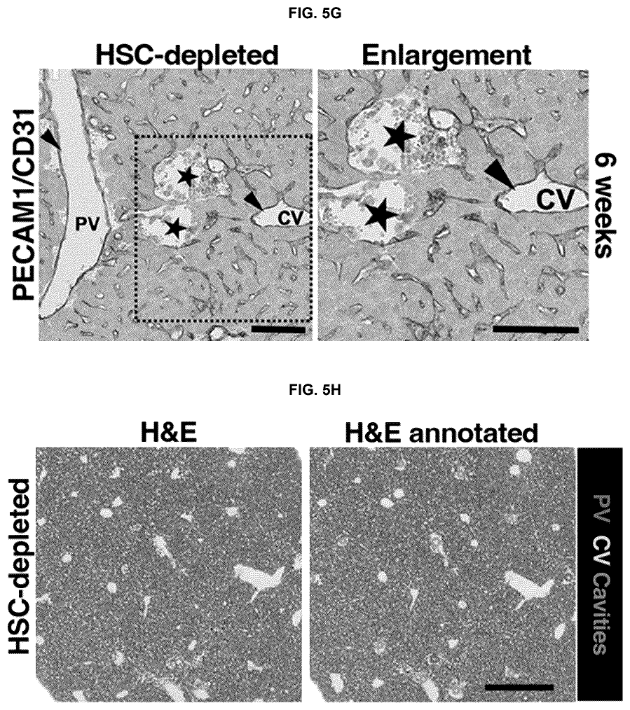

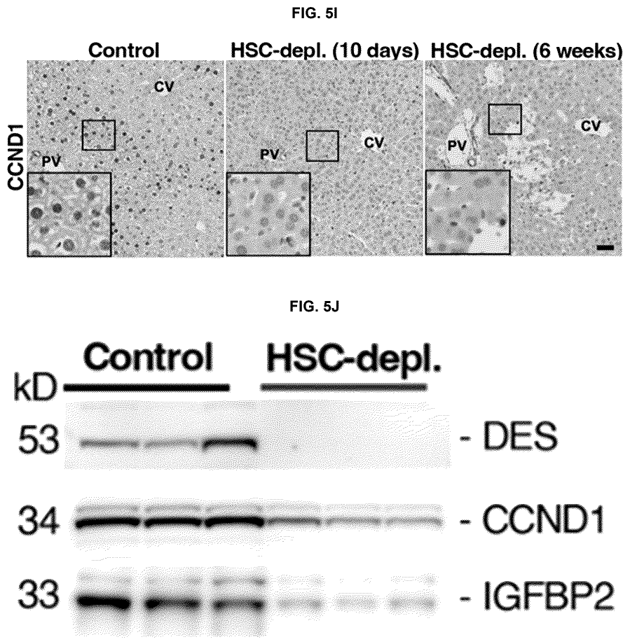

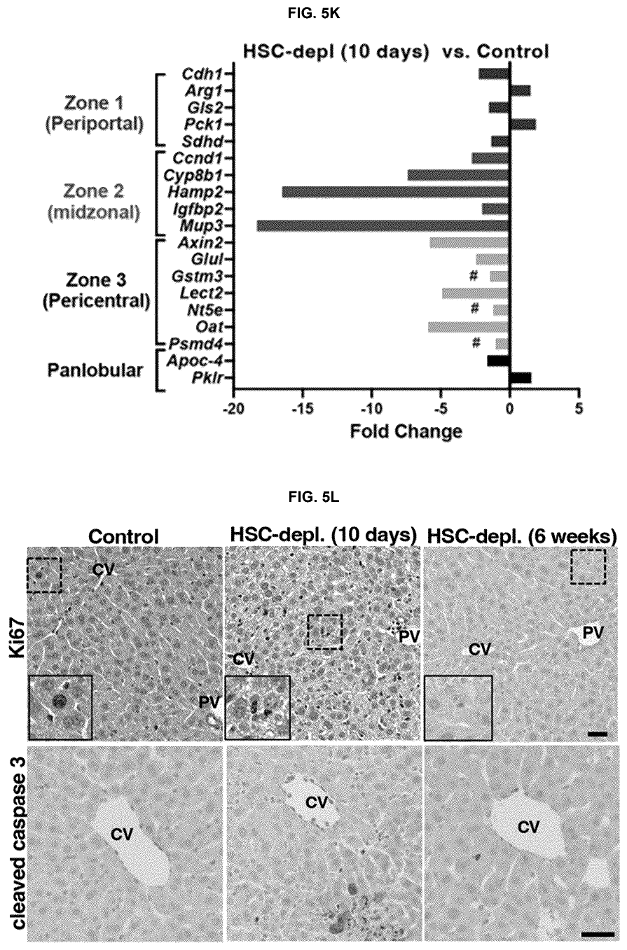

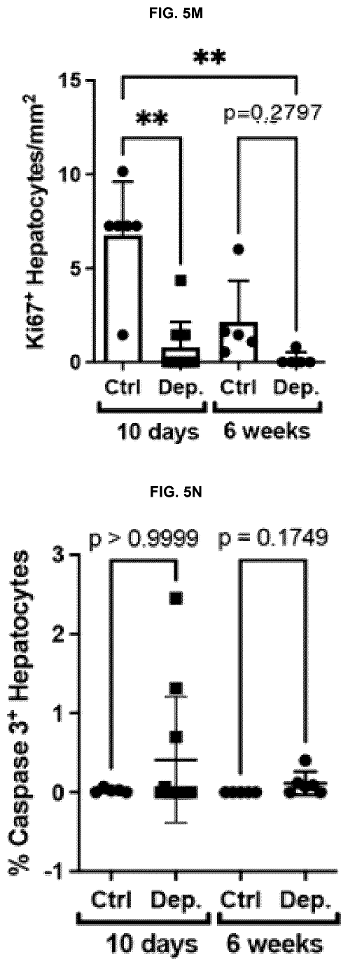

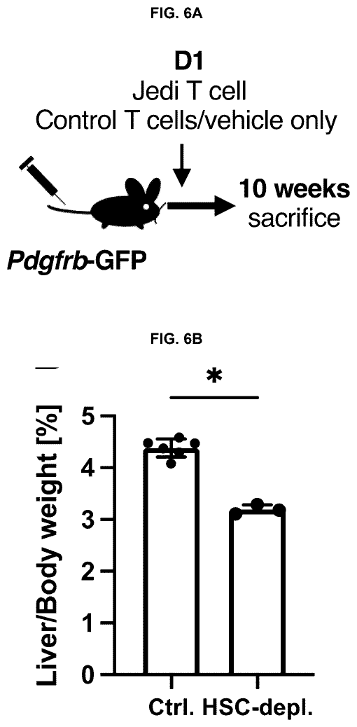

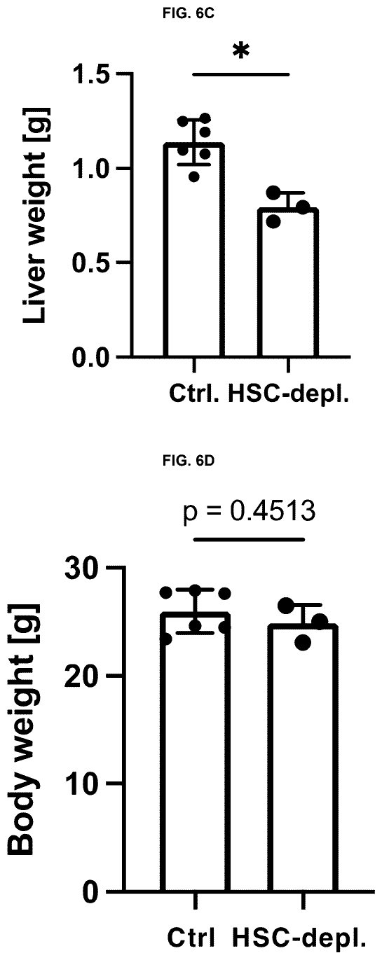

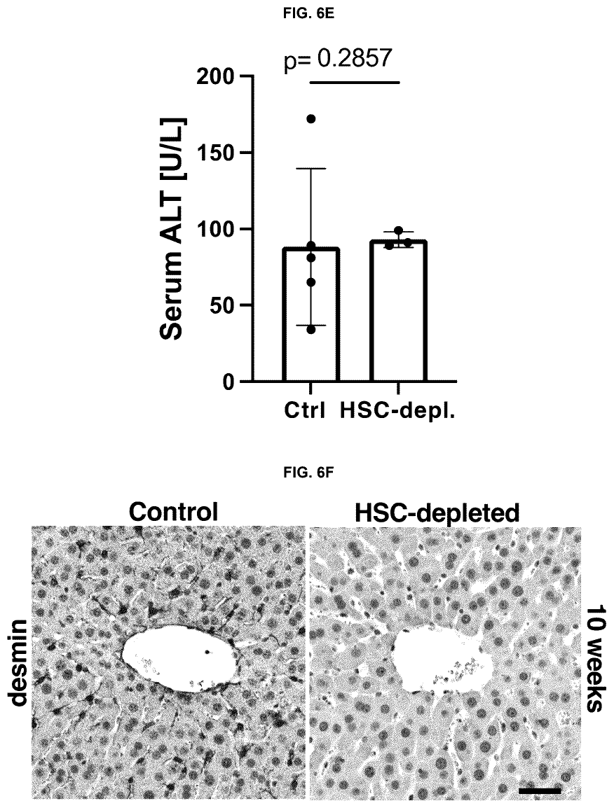

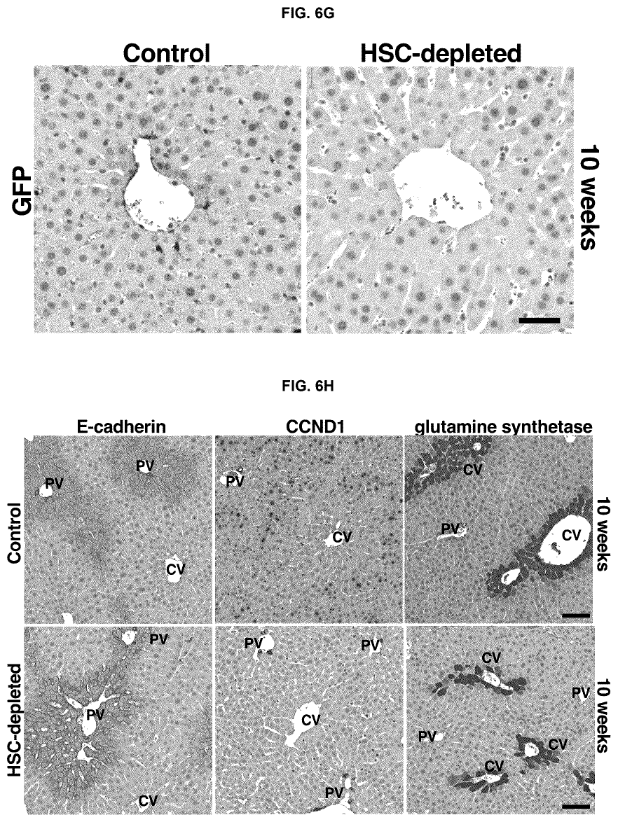

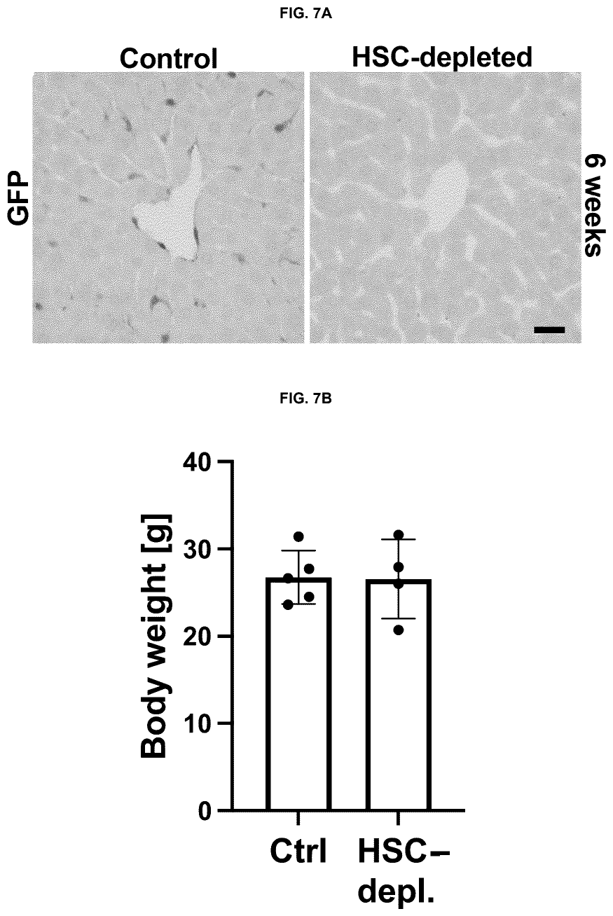

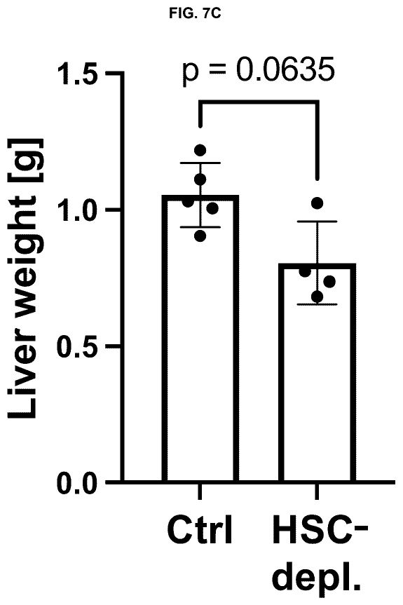

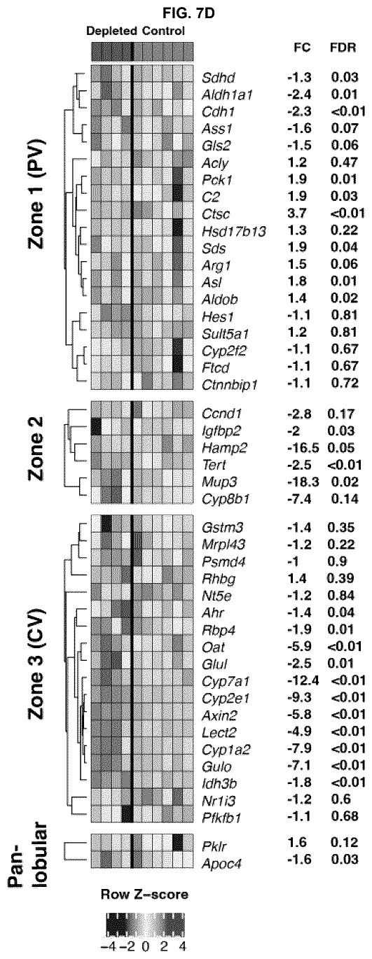

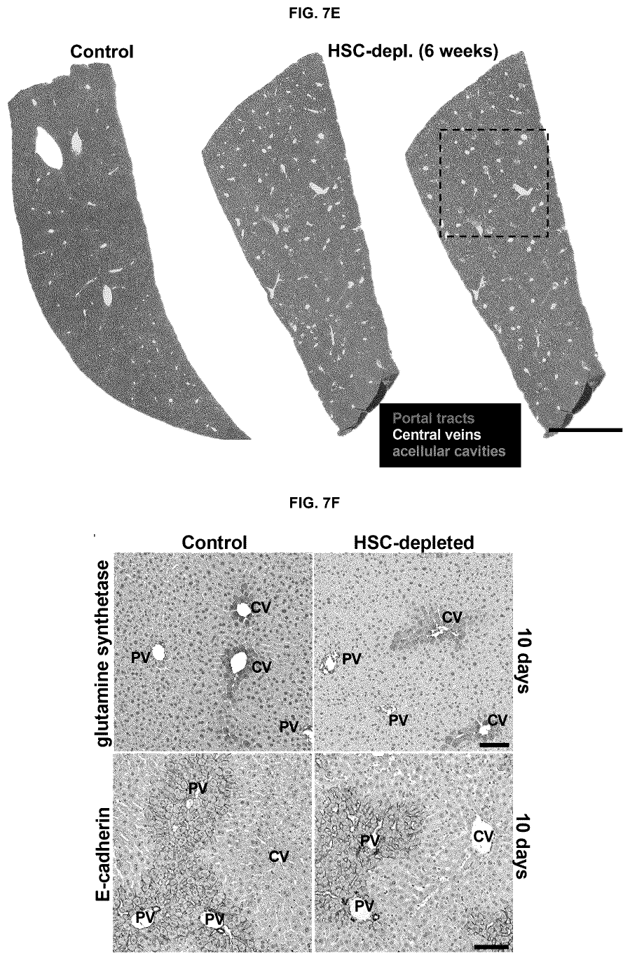

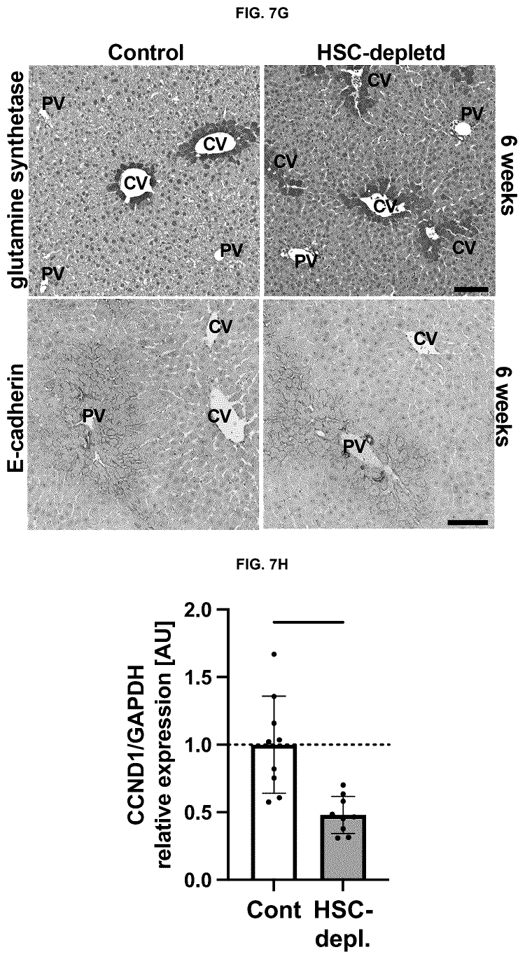

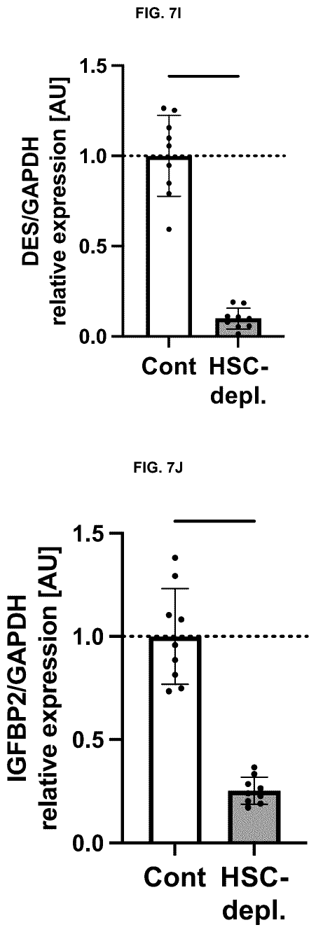

Mice were sacrificed 6 weeks and 10 weeks after HSC ablation (FIG. 5A and FIG. 6A) and no evidence of desmin+ or GFP+ HSCs was found (FIG. 5B; FIGS. 6F-G; FIG. 7A), indicating that HSCs were completely depleted for up to 10 weeks. Macroscopic inspection showed smaller livers in HSC-depleted mice (FIG. 5C), which was supported by measurements of body and liver weights (FIG. 5D; FIGS. 6B-D; FIGS. 7B-C). Serum ALT amounts were similar in control and HSC-depleted mice 10 weeks after depletion (FIG. 6E). Although body weights were similar among both HSC-depleted and control (vehicle-only injection) groups immediately after HSC depletion (10 days) (FIG. 2E), a significant decrease was observed in the liver-to-body weight ratio of up to ˜45% after sustained HSC depletion (6 weeks and 10 weeks after HSC-depletion). Mean liver/body weight ratios were 3.94% vs. 3.05% and 4.38% vs. 3.19% at 6 weeks and 10 weeks after HSC depletion, respectively (FIG. 5D; FIG. 6B). Liver-to-body weight ratios decreased by ˜22.5% at 6 weeks after HSC depletion and by ˜27.1% at 10 weeks, suggesting a progressive liver mass loss with sustained HSC depletion. H&E staining showed distended sinusoids and an increase in acellular areas reminiscent of large hepatic vessels (FIGS. 5E-F; FIG. 7E). However, based on immunostaining with the endothelial cell marker CD31/PECAM1, these structures were not lined by endothelial cells (FIG. 5G) and thus acellular cavities. These cavities (annotated with blue circles) were consistently localized between central veins (yellow circles) and portal veins (green circles) within the midlobular zone 2 of the hepatic lobule (FIG. 5H; FIG. 7E).

Several studies have established the role of midlobular zone 2 hepatocytes in maintaining liver homeostasis and liver mass in healthy livers. Central to this process is CCND1, which is constitutively expressed in most midlobular hepatocytes and critical for their proliferation. Thus, liver zonation and expression of CCND1 in HSC-depleted livers and control livers was investigated. As expected, control livers showed strong nuclear staining in midlobular zone 2 hepatocytes. By contrast, there was a substantial decrease in nuclear hepatocyte CCND1 staining 10 days after HSC ablation, and staining remained low for up to 10 weeks after HSC ablation (FIG. 5I; FIG. 6H). The rapid decrease in CCND1 was confirmed by western blotting of whole liver lysates for CCND1, as well as for another zone 2 hepatocyte marker, insulin growth factor-binding protein 2 (IGFBP2) (FIG. 5J; FIGS. 7H-J). Marked reduction in genes characteristic for zone 2 hepatocytes was also apparent by liver transcriptome analysis assessed at 10 days after HSC ablation. Based on whole liver RNA-seq analysis, there was a greater (up to −18.3-fold) and more consistent decrease in marker genes for zone 2 hepatocytes (Hamp2, Cyp8b1, Mup3, Ccnd1 and Igfbp2) than in genes characteristic for zone 3 (Axin2, Lect2, Oat, Nt5e, GluI), or zone 1 (Gls2, Arg1, Cdh1) hepatocytes or in panlobular genes (Apoc4, Pklr) (FIG. 5K, FIG. 7D). Whereas the zone 3 genes Axin2 and GluI were significantly decreased (−5.8-fold and −2.5-fold, respectively, FDR<0.01), other zone 3 hepatocyte genes (Gstm3, Nt5e and Psmd4) were not differentially expressed (FIG. 5D; FIG. 7D). Gene expression analysis for signaling and metabolic pathways in HSC-depleted livers compared to control livers in KEGG, BioCarta, and Reactome signatures (FDR<0.1) showed decreases in Wnt-β-catenin signaling, hedgehog signaling, and transforming growth factor β (TGF-β) signaling, and decreases in insulin receptor signaling, amino acid metabolism, and cytochrome P450 metabolism, some of which are to be expected, given the metabolic function of midlobular hepatocytes and HSC-derived growth factor signaling pathways (TABLES 3-5). Even though gene expression analyses suggested that metabolic zonation was affected by HSC depletion, we observed stable expression of both glutamine synthetase (GS), a pericentral marker and Wnt-β-catenin target, and E-cadherin, a periportal marker, at 10 days and 6 weeks after HSC depletion (FIGS. 7F-G). At 10 weeks after HSC depletion, E-cadherin staining was preserved but the number of GS-positive hepatocytes was decreased compared to controls (FIG. 6H). Because staining for GS and E-cadherin or similar markers has been commonly used as an indicator of liver zonation, these findings suggest that zone 1 and zone 3 hepatocytes were preserved in HSC-depleted livers despite decreased Wnt-β-catenin signaling based on gene expression analysis. The data herein suggest that assessment of these markers alone might not be sufficient to characterize liver zonation and that more detailed analyses are required.

TABLE 3
Gene expression analysis for signaling and metabolic pathways in HSC-depleted livers compared to control livers in Reactome signatures.

| Pathway | NGenes | PropDown | PropUp | Direction | PValue | FDR | PValue.Mixed | FDR.Mixed |
|---|---|---|---|---|---|---|---|---|
| REACTOME_ACTIVATED_AMPK_{—} | 21 | 0.48 | 0.29 | Down | 0.0005 | 0.0009 | 0.0005 | 0.0005 |
| STIMULATES_FATTY_ACID_{—} | | | | | | | | |
| OXIDATION_IN_MUSCLE | | | | | | | | |
| REACTOME_ALPHA_LINOLENIC_{—} | 15 | 0.73 | 0.07 | Down | 0.0005 | 0.0009 | 0.0005 | 0.0005 |
| ACID_ALA_METABOLISM | | | | | | | | |
| REACTOME_BILE_ACID_AND_BILE_{—} | 39 | 0.59 | 0.15 | Down | 0.0005 | 0.0009 | 0.0005 | 0.0005 |
| SALT_METABOLISM | | | | | | | | |
| REACTOME_BRANCHED_CHAIN_{—} | 19 | 0.84 | 0.05 | Down | 0.0005 | 0.0009 | 0.0005 | 0.0005 |
| AMINO_ACID_CATABOLISM | | | | | | | | |
| REACTOME_CITRIC_ACID_CYCLE_{—} | 20 | 0.80 | 0.00 | Down | 0.0005 | 0.0009 | 0.0005 | 0.0005 |
| TCA_CYCLE | | | | | | | | |
| REACTOME_ENERGY_DEPENDENT_{—} | 20 | 0.50 | 0.20 | Down | 0.0005 | 0.0009 | 0.0005 | 0.0005 |
| REGULATION_OF_MTOR_BY_{—} | | | | | | | | |
| LKB1_AMPK | | | | | | | | |
| REACTOME_FATTY_ACID_{—} | 199 | 0.37 | 0.20 | Down | 0.0005 | 0.0009 | 0.0005 | 0.0005 |
| TRIACYLGLYCEROL_AND_{—} | | | | | | | | |
| KETONE_BODY_METABOLISM | | | | | | | | |
| REACTOME_MITOCHONDRIAL_{—} | 17 | 0.88 | 0.00 | Down | 0.0005 | 0.0009 | 0.0005 | 0.0005 |
| FATTY_ACID_BETA_OXIDATION | | | | | | | | |
| REACTOME_PEROXISOMAL_LIPID_{—} | 22 | 0.55 | 0.18 | Down | 0.0005 | 0.0009 | 0.0010 | 0.0010 |
| METABOLISM | | | | | | | | |
| REACTOME_PKB_MEDIATED_{—} | 35 | 0.46 | 0.20 | Down | 0.0005 | 0.0009 | 0.0005 | 0.0005 |
| EVENTS | | | | | | | | |
| REACTOME_PPARA_ACTIVATES_{—} | 132 | 0.31 | 0.20 | Down | 0.0005 | 0.0009 | 0.0010 | 0.0010 |
| GENE_EXPRESSION | | | | | | | | |
| REACTOME_PYRUVATE_{—} | 39 | 0.62 | 0.10 | Down | 0.0005 | 0.0009 | 0.0005 | 0.0005 |
| METABOLISM_AND_CITRIC_{—} | | | | | | | | |
| ACID_TCA_CYCLE | | | | | | | | |
| REACTOME_REGULATION_OF_{—} | 17 | 0.47 | 0.24 | Down | 0.0005 | 0.0009 | 0.0005 | 0.0005 |
| AMPK_ACTIVITY_VIA_LKB1 | | | | | | | | |
| REACTOME_SYNTHESIS_OF_BILE_{—} | 27 | 0.67 | 0.15 | Down | 0.0005 | 0.0009 | 0.0005 | 0.0005 |
| ACIDS_AND_BILE_SALTS | | | | | | | | |
| REACTOME_SYNTHESIS_OF_BILE_{—} | 23 | 0.70 | 0.09 | Down | 0.0005 | 0.0009 | 0.0005 | 0.0005 |
| ACIDS_AND_BILE_SALTS_VIA_{—} | | | | | | | | |
| 7ALPHA_HYDROXYCHOLESTEROL | | | | | | | | |
| REACTOME_TIGHT_JUNCTION_{—} | 18 | 0.61 | 0.28 | Down | 0.0005 | 0.0009 | 0.0005 | 0.0005 |
| INTERACTIONS | | | | | | | | |
| REACTOME_BIOLOGICAL_{—} | 161 | 0.39 | 0.19 | Down | 0.0010 | 0.0020 | 0.0010 | 0.0010 |
| OXIDATIONS | | | | | | | | |
| REACTOME_CELL_CELL_JUNCTION_{—} | 33 | 0.52 | 0.21 | Down | 0.0010 | 0.0020 | 0.0005 | 0.0005 |
| ORGANIZATION | | | | | | | | |
| REACTOME_GLUCURONIDATION | 17 | 0.71 | 0.12 | Down | 0.0010 | 0.0020 | 0.0015 | 0.0015 |
| REACTOME_SYNTHESIS_OF_BILE_{—} | 15 | 0.80 | 0.07 | Down | 0.0010 | 0.0020 | 0.0005 | 0.0005 |
| ACIDS_AND_BILE_SALTS_VIA_24_{—} | | | | | | | | |
| HYDROXYCHOLESTEROL | | | | | | | | |
| REACTOME_FATTY_ACYL_COA_{—} | 15 | 0.47 | 0.13 | Down | 0.0015 | 0.0031 | 0.0015 | 0.0015 |
| BIOSYNTHESIS | | | | | | | | |
| REACTOME_METABOLISM_OF_{—} | 52 | 0.50 | 0.10 | Down | 0.0015 | 0.0031 | 0.0005 | 0.0005 |
| VITAMINS_AND_COFACTORS | | | | | | | | |
| REACTOME_DEADENYLATION_OF_{—} | 23 | 0.30 | 0.04 | Down | 0.0020 | 0.0040 | 0.0020 | 0.0020 |
| MRNA | | | | | | | | |
| REACTOME_TRIGLYCERIDE_{—} | 36 | 0.39 | 0.25 | Down | 0.0020 | 0.0040 | 0.0005 | 0.0005 |
| BIOSYNTHESIS | | | | | | | | |
| REACTOME_MITOCHONDRIAL_{—} | 27 | 0.48 | 0.07 | Down | 0.0025 | 0.0049 | 0.0005 | 0.0005 |
| TRNA_AMINOACYLATION | | | | | | | | |
| REACTOME_NUCLEAR_RECEPTOR_{—} | 38 | 0.39 | 0.18 | Down | 0.0025 | 0.0049 | 0.0005 | 0.0005 |
| TRANSCRIPTION_PATHWAY | | | | | | | | |
| REACTOME_PHASE_II_{—} | 74 | 0.46 | 0.16 | Down | 0.0025 | 0.0049 | 0.0015 | 0.0015 |
| CONJUGATION | | | | | | | | |
| REACTOME_CYTOCHROME_P450_{—} | 70 | 0.36 | 0.19 | Down | 0.0035 | 0.0067 | 0.0010 | 0.0010 |
| ARRANGED_BY_SUBSTRATE_{—} | | | | | | | | |
| TYPE | | | | | | | | |
| REACTOME_P75NTR_RECRUITS_{—} | 15 | 0.40 | 0.13 | Down | 0.0035 | 0.0067 | 0.0005 | 0.0005 |
| SIGNALLING_COMPLEXES | | | | | | | | |
| REACTOME_TETRAHYDROBIOPTERIN_{—} | 15 | 0.27 | 0.27 | Down | 0.0035 | 0.0067 | 0.0005 | 0.0005 |
| BH4_SYNTHESIS_RECYCLING_{—} | | | | | | | | |
| SALVAGE_AND_REGULATION | | | | | | | | |
| REACTOME_TCA_CYCLE_AND_{—} | 125 | 0.74 | 0.04 | Down | 0.0045 | 0.0083 | 0.0020 | 0.0020 |
| RESPIRATORY_ELECTRON_{—} | | | | | | | | |
| TRANSPORT | | | | | | | | |
| REACTOME_MITOCHONDRIAL_{—} | 64 | 0.53 | 0.08 | Down | 0.0065 | 0.0116 | 0.0010 | 0.0010 |
| PROTEIN_IMPORT | | | | | | | | |
| REACTOME_NOTCH1_{—} | 46 | 0.30 | 0.20 | Down | 0.0065 | 0.0116 | 0.0005 | 0.0005 |
| INTRACELLULAR_DOMAIN_{—} | | | | | | | | |
| REGULATES_TRANSCRIPTION | | | | | | | | |
| REACTOME_DOWNREGULATION_{—} | 26 | 0.31 | 0.08 | Down | 0.0070 | 0.0123 | 0.0005 | 0.0005 |
| OF_SMAD2_3_SMAD4_{—} | | | | | | | | |
| TRANSCRIPTIONAL_ACTIVITY | | | | | | | | |
| REACTOME_RESPIRATORY_{—} | 90 | 0.80 | 0.01 | Down | 0.0080 | 0.0139 | 0.0050 | 0.0050 |
| ELECTRON_TRANSPORT_ATP_{—} | | | | | | | | |
| SYNTHESIS_BY_CHEMIOSMOTIC_{—} | | | | | | | | |
| COUPLING_AND_HEAT_PRODUCTION_{—} | | | | | | | | |
| BY_UNCOUPLING_PROTEINS | | | | | | | | |
| REACTOME_FORMATION_OF_ATP_{—} | 15 | 0.87 | 0.00 | Down | 0.0085 | 0.0146 | 0.0085 | 0.0086 |
| BY_CHEMIOSMOTIC_COUPLING | | | | | | | | |
| REACTOME_RESPIRATORY_{—} | 74 | 0.80 | 0.00 | Down | 0.0090 | 0.0154 | 0.0090 | 0.0091 |
| ELECTRON_TRANSPORT | | | | | | | | |
| REACTOME_METABOLISM_OF_{—} | 177 | 0.41 | 0.16 | Down | 0.0100 | 0.0168 | 0.0005 | 0.0005 |
| AMINO_ACIDS_AND_DERIVATIVES | | | | | | | | |
| REACTOME_RNA_POL_I_{—} | 25 | 0.40 | 0.08 | Down | 0.0100 | 0.0168 | 0.0025 | 0.0025 |
| TRANSCRIPTION_INITIATION | | | | | | | | |
| REACTOME_XENOBIOTICS | 39 | 0.31 | 0.08 | Down | 0.0105 | 0.0175 | 0.0060 | 0.0060 |
| REACTOME_ABC_FAMILY_{—} | 32 | 0.50 | 0.19 | Down | 0.0110 | 0.0183 | 0.0005 | 0.0005 |
| PROTEINS_MEDIATED_TRANSPORT | | | | | | | | |
| REACTOME_PHASE1_{—} | 89 | 0.33 | 0.20 | Down | 0.0120 | 0.0197 | 0.0005 | 0.0005 |
| FUNCTIONALIZATION_OF_{—} | | | | | | | | |
| COMPOUNDS | | | | | | | | |
| REACTOME_RNA_POL_III_{—} | 32 | 0.19 | 0.06 | Down | 0.0135 | 0.0219 | 0.0335 | 0.0339 |
| TRANSCRIPTION | | | | | | | | |
| REACTOME_SIGNALING_BY_BMP | 21 | 0.43 | 0.05 | Down | 0.0135 | 0.0219 | 0.0080 | 0.0081 |
| REACTOME_CTNNB1_{—} | 16 | 0.19 | 0.06 | Down | 0.0145 | 0.0233 | 0.0015 | 0.0015 |
| PHOSPHORYLATION_CASCADE | | | | | | | | |
| REACTOME_RNA_POL_I_RNA_{—} | 76 | 0.29 | 0.09 | Down | 0.0155 | 0.0248 | 0.0005 | 0.0005 |
| POL_III_AND_MITOCHONDRIAL_{—} | | | | | | | | |
| TRANSCRIPTION | | | | | | | | |
| REACTOME_ASSOCIATION_OF_{—} | 32 | 0.31 | 0.09 | Down | 0.0170 | 0.0270 | 0.0005 | 0.0005 |
| TRIC_CCT_WITH_TARGET_PROTEINS_{—} | | | | | | | | |
| DURING_BIOSYNTHESIS | | | | | | | | |
| REACTOME_RECYCLING_OF_BILE_{—} | 18 | 0.39 | 0.17 | Down | 0.0170 | 0.0270 | 0.0005 | 0.0005 |
| ACIDS_AND_SALTS | | | | | | | | |
| REACTOME_CHYLOMICRON_{—} | 18 | 0.28 | 0.06 | Down | 0.0205 | 0.0316 | 0.0030 | 0.0030 |
| MEDIATED_LIPID_TRANSPORT | | | | | | | | |
| REACTOME_RNA_POL_III_{—} | 22 | 0.23 | 0.05 | Down | 0.0225 | 0.0344 | 0.0375 | 0.0379 |
| TRANSCRIPTION_INITIATION_{—} | | | | | | | | |
| FROM_TYPE_2_PROMOTER | | | | | | | | |
| REACTOME_COLLAGEN_{—} | 38 | 0.39 | 0.21 | Down | 0.0235 | 0.0357 | 0.0010 | 0.0010 |
| FORMATION | | | | | | | | |
| REACTOME_INSULIN_RECEPTOR_{—} | 83 | 0.37 | 0.24 | Down | 0.0265 | 0.0397 | 0.0005 | 0.0005 |
| SIGNALLING_CASCADE | | | | | | | | |
| REACTOME_ABCA_TRANSPORTERS_{—} | 18 | 0.50 | 0.17 | Down | 0.0300 | 0.0443 | 0.0005 | 0.0005 |
| IN_LIPID_HOMEOSTASIS | | | | | | | | |
| REACTOME_TRANSPORT_OF_{—} | 28 | 0.32 | 0.07 | Down | 0.0315 | 0.0462 | 0.0010 | 0.0010 |
| RIBONUCLEOPROTEINS_INTO_{—} | | | | | | | | |
| THE_HOST_NUCLEUS | | | | | | | | |
| REACTOME_METABOLISM_OF_{—} | 25 | 0.36 | 0.32 | Down | 0.0330 | 0.0482 | 0.0005 | 0.0005 |
| STEROID_HORMONES_AND_{—} | | | | | | | | |
| VITAMINS_A_AND_D | | | | | | | | |
| REACTOME_REGULATORY_RNA_{—} | 26 | 0.23 | 0.19 | Down | 0.0350 | 0.0508 | 0.0685 | 0.0691 |
| PATHWAYS | | | | | | | | |
| REACTOME_RNA_POL_III_{—} | 19 | 0.32 | 0.05 | Down | 0.0400 | 0.0574 | 0.0055 | 0.0055 |
| TRANSCRIPTION_TERMINATION | | | | | | | | |
| REACTOME_BIOSYNTHESIS_OF_{—} | 28 | 0.36 | 0.07 | Down | 0.0445 | 0.0632 | 0.0035 | 0.0035 |
| THE_N_GLYCAN_PRECURSOR_{—} | | | | | | | | |
| DOLICHOL_LIPID_LINKED_{—} | | | | | | | | |
| OLIGOSACCHARIDE_LLO_AND_{—} | | | | | | | | |
| TRANSFER_TO_A NASCENT_{—} | | | | | | | | |
| PROTEIN | | | | | | | | |
| REACTOME_RNA_POL_I_{—} | 20 | 0.30 | 0.10 | Down | 0.0465 | 0.0656 | 0.0110 | 0.0111 |
| TRANSCRIPTION_TERMINATION | | | | | | | | |
| REACTOME_ENOS_ACTIVATION_{—} | 23 | 0.22 | 0.26 | Down | 0.0470 | 0.0661 | 0.0005 | 0.0005 |
| AND_REGULATION | | | | | | | | |
| REACTOME_ADHERENS_{—} | 15 | 0.40 | 0.13 | Down | 0.0520 | 0.0727 | 0.0005 | 0.0005 |
| JUNCTIONS_INTERACTIONS | | | | | | | | |
| REACTOME_TRNA_{—} | 43 | 0.40 | 0.07 | Down | 0.0605 | 0.0833 | 0.0020 | 0.0020 |
| AMINOACYLATION | | | | | | | | |
| REACTOME_PI3K_CASCADE | 63 | 0.40 | 0.24 | Down | 0.0620 | 0.0853 | 0.0005 | 0.0005 |
| REACTOME_CIRCADIAN_{—} | 27 | 0.37 | 0.11 | Down | 0.0635 | 0.0872 | 0.0005 | 0.0005 |
| REPRESSION_OF_EXPRESSION_{—} | | | | | | | | |
| BY_REV_ERBA | | | | | | | | |
| REACTOME_RNA_POL_III_CHAIN_{—} | 17 | 0.29 | 0.06 | Down | 0.0645 | 0.0885 | 0.0330 | 0.0334 |
| ELONGATION | | | | | | | | |
| REACTOME_GLUTATHIONE_{—} | 23 | 0.43 | 0.13 | Down | 0.0675 | 0.0920 | 0.0035 | 0.0035 |
| CONJUGATION | | | | | | | | |
| REACTOME_REGULATION_OF_{—} | 22 | 0.18 | 0.05 | Down | 0.0675 | 0.0920 | 0.0315 | 0.0319 |
| HYPOXIA_INDUCIBLE_FACTOR_{—} | | | | | | | | |
| HIF_BY_OXYGEN | | | | | | | | |
| REACTOME_RNA_POL_I_{—} | 43 | 0.35 | 0.12 | Down | 0.0700 | 0.0949 | 0.0005 | 0.0005 |
| TRANSCRIPTION | | | | | | | | |
| REACTOME_ACTIVATION_OF_{—} | 60 | 0.23 | 0.12 | Down | 0.0705 | 0.0955 | 0.0020 | 0.0020 |
| CHAPERONE_GENES_BY_XBP1S | | | | | | | | |
| REACTOME_RORA_ACTIVATES_{—} | 29 | 0.38 | 0.14 | Down | 0.0735 | 0.0988 | 0.0005 | 0.0005 |
| CIRCADIAN_EXPRESSION | | | | | | | | |
| REACTOME_ACTIVATED_TAK1_{—} | 17 | 0.12 | 0.41 | Up | 0.0005 | 0.0009 | 0.0005 | 0.0005 |
| MEDIATES_P38_MAPK_ACTIVATION | | | | | | | | |
| REACTOME_ACTIVATED_TLR4_{—} | 109 | 0.13 | 0.52 | Up | 0.0005 | 0.0009 | 0.0005 | 0.0005 |
| SIGNALLING | | | | | | | | |
| REACTOME_ACTIVATION_OF_IRF3_{—} | 22 | 0.18 | 0.59 | Up | 0.0005 | 0.0009 | 0.0005 | 0.0005 |
| IRF7_MEDIATED_BY_TBK1_IKK_{—} | | | | | | | | |
| EPSILON | | | | | | | | |
| REACTOME_ACTIVATION_OF_{—} | 33 | 0.21 | 0.36 | Up | 0.0005 | 0.0009 | 0.0005 | 0.0005 |
| NMDA_RECEPTOR_UPON_GLUTAMATE | | | | | | | | |
| BINDING_AND_POSTSYNAPTIC_{—} | | | | | | | | |
| EVENTS | | | | | | | | |
| REACTOME_ADAPTIVE_IMMUNE_{—} | 536 | 0.16 | 0.44 | Up | 0.0005 | 0.0009 | 0.0005 | 0.0005 |
| SYSTEM | | | | | | | | |
| REACTOME_ANTIGEN_ACTIVATES_{—} | 42 | 0.17 | 0.57 | Up | 0.0005 | 0.0009 | 0.0005 | 0.0005 |
| B_CELL_RECEPTOR_LEADING_{—} | | | | | | | | |
| TO_GENERATION_OF_SECOND_{—} | | | | | | | | |
| MESSENGERS | | | | | | | | |
| REACTOME_ANTIGEN_PRESENTATION_{—} | 42 | 0.10 | 0.55 | Up | 0.0005 | 0.0009 | 0.0005 | 0.0005 |
| FOLDING_ASSEMBLY_AND_{—} | | | | | | | | |
| PEPTIDE_LOADING_OF_CLASS_I_{—} | | | | | | | | |
| MHC | | | | | | | | |
| REACTOME_ANTIGEN_PROCESSING_{—} | 95 | 0.12 | 0.40 | Up | 0.0005 | 0.0009 | 0.0005 | 0.0005 |
| CROSS_PRESENTATION | | | | | | | | |
| REACTOME_APOPTOSIS | 164 | 0.20 | 0.32 | Up | 0.0005 | 0.0009 | 0.0005 | 0.0005 |
| REACTOME_BASIGIN_{—} | 17 | 0.00 | 0.59 | Up | 0.0005 | 0.0009 | 0.0005 | 0.0005 |
| INTERACTIONS | | | | | | | | |
| REACTOME_CA_DEPENDENT_{—} | 27 | 0.22 | 0.48 | Up | 0.0005 | 0.0009 | 0.0005 | 0.0005 |
| EVENTS | | | | | | | | |
| REACTOME_CD28_CO_{—} | 38 | 0.24 | 0.63 | Up | 0.0005 | 0.0009 | 0.0005 | 0.0005 |
| STIMULATION | | | | | | | | |
| REACTOME_CD28_DEPENDENT_{—} | 28 | 0.29 | 0.57 | Up | 0.0005 | 0.0009 | 0.0005 | 0.0005 |
| PI3K_AKT_SIGNALING | | | | | | | | |
| REACTOME_CD28_DEPENDENT_{—} | 19 | 0.11 | 0.84 | Up | 0.0005 | 0.0009 | 0.0005 | 0.0005 |
| VAV1_PATHWAY | | | | | | | | |
| REACTOME_CELL_SURFACE_{—} | 100 | 0.19 | 0.60 | Up | 0.0005 | 0.0009 | 0.0005 | 0.0005 |
| INTERACTIONS_AT_THE_{—} | | | | | | | | |
| VASCULAR_WALL | | | | | | | | |
| REACTOME_CHEMOKINE_{—} | 25 | 0.20 | 0.72 | Up | 0.0005 | 0.0009 | 0.0005 | 0.0005 |
| RECEPTORS_BIND_CHEMOKINES | | | | | | | | |
| REACTOME_CLASS_A1_{—} | 103 | 0.22 | 0.61 | Up | 0.0005 | 0.0009 | 0.0005 | 0.0005 |
| RHODOPSIN_LIKE_RECEPTORS | | | | | | | | |
| REACTOME_COSTIMULATION_BY_{—} | 65 | 0.17 | 0.63 | Up | 0.0005 | 0.0009 | 0.0005 | 0.0005 |
| THE_CD28_FAMILY | | | | | | | | |
| REACTOME_CTLA4_INHIBITORY_{—} | 22 | 0.18 | 0.50 | Up | 0.0005 | 0.0009 | 0.0005 | 0.0005 |
| SIGNALING | | | | | | | | |
| REACTOME_CYTOKINE_{—} | 294 | 0.15 | 0.57 | Up | 0.0005 | 0.0009 | 0.0005 | 0.0005 |
| SIGNALING_IN_IMMUNE_SYSTEM | | | | | | | | |
| REACTOME_DAG_AND_IP3_{—} | 31 | 0.23 | 0.48 | Up | 0.0005 | 0.0009 | 0.0005 | 0.0005 |
| SIGNALING | | | | | | | | |
| REACTOME_DOWNSTREAM_TCR_{—} | 39 | 0.15 | 0.64 | Up | 0.0005 | 0.0009 | 0.0005 | 0.0005 |
| SIGNALING | | | | | | | | |
| REACTOME_ENDOSOMAL_{—} | 21 | 0.05 | 0.90 | Up | 0.0005 | 0.0009 | 0.0005 | 0.0005 |
| VACUOLAR_PATHWAY | | | | | | | | |
| REACTOME_FACTORS_INVOLVED_{—} | 104 | 0.13 | 0.40 | Up | 0.0005 | 0.0009 | 0.0005 | 0.0005 |
| IN_MEGAKARYOCYTE_DEVELOPMENT_{—} | | | | | | | | |
| AND_PLATELET_PRODUCTION | | | | | | | | |
| REACTOME_G_ALPHA_I_{—} | 84 | 0.23 | 0.64 | Up | 0.0005 | 0.0009 | 0.0005 | 0.0005 |
| SIGNALLING_EVENTS | | | | | | | | |
| REACTOME_G_ALPHA_Q_{—} | 90 | 0.21 | 0.52 | Up | 0.0005 | 0.0009 | 0.0005 | 0.0005 |
| SIGNALLING_EVENTS | | | | | | | | |
| REACTOME_GASTRIN_CREB_{—} | 119 | 0.22 | 0.50 | Up | 0.0005 | 0.0009 | 0.0005 | 0.0005 |
| SIGNALLING_PATHWAY_VIA_{—} | | | | | | | | |
| PKC_AND_MAPK | | | | | | | | |
| REACTOME_GENERATION_OF_{—} | 29 | 0.03 | 0.90 | Up | 0.0005 | 0.0009 | 0.0005 | 0.0005 |
| SECOND_MESSENGER_MOLECULES | | | | | | | | |
| REACTOME_GLUTAMATE_{—} | 16 | 0.25 | 0.44 | Up | 0.0005 | 0.0009 | 0.0005 | 0.0005 |
| NEUROTRANSMITTER_RELEASE_{—} | | | | | | | | |
| CYCLE | | | | | | | | |
| REACTOME_GLYCOSPHINGOLIPID_{—} | 31 | 0.06 | 0.55 | Up | 0.0005 | 0.0009 | 0.0005 | 0.0005 |
| METABOLISM | | | | | | | | |
| REACTOME_GOLGI_ASSOCIATED_{—} | 68 | 0.12 | 0.31 | Up | 0.0005 | 0.0009 | 0.0005 | 0.0005 |
| VESICLE_BIOGENESIS | | | | | | | | |
| REACTOME_GPCR_DOWNSTREAM_{—} | 250 | 0.25 | 0.51 | Up | 0.0005 | 0.0009 | 0.0005 | 0.0005 |
| SIGNALING | | | | | | | | |
| REACTOME_GPVI_MEDIATED_{—} | 51 | 0.27 | 0.55 | Up | 0.0005 | 0.0009 | 0.0005 | 0.0005 |
| ACTIVATION_CASCADE | | | | | | | | |
| REACTOME_GROWTH_HORMONE_{—} | 25 | 0.16 | 0.56 | Up | 0.0005 | 0.0009 | 0.0005 | 0.0005 |
| RECEPTOR_SIGNALING | | | | | | | | |
| REACTOME_HEMOSTASIS | 431 | 0.19 | 0.48 | Up | 0.0005 | 0.0009 | 0.0005 | 0.0005 |
| REACTOME_IKK_COMPLEX_{—} | 17 | 0.29 | 0.59 | Up | 0.0005 | 0.0009 | 0.0005 | 0.0005 |
| RECRUITMENT_MEDIATED_BY_RIP1 | | | | | | | | |
| REACTOME_IL_2_SIGNALING | 48 | 0.15 | 0.56 | Up | 0.0005 | 0.0009 | 0.0005 | 0.0005 |
| REACTOME_IL_3_5_AND_GM_CSF_{—} | 55 | 0.15 | 0.60 | Up | 0.0005 | 0.0009 | 0.0005 | 0.0005 |
| SIGNALING | | | | | | | | |
| REACTOME_IL_RECEPTOR_SHC_{—} | 29 | 0.17 | 0.62 | Up | 0.0005 | 0.0009 | 0.0005 | 0.0005 |
| SIGNALING | | | | | | | | |
| REACTOME_IL1_SIGNALING | 44 | 0.20 | 0.39 | Up | 0.0005 | 0.0009 | 0.0005 | 0.0005 |
| REACTOME_IMMUNE_SYSTEM | 892 | 0.14 | 0.49 | Up | 0.0005 | 0.0009 | 0.0005 | 0.0005 |
| REACTOME_IMMUNOREGULATORY_{—} | 70 | 0.07 | 0.84 | Up | 0.0005 | 0.0009 | 0.0005 | 0.0005 |
| INTERACTIONS_BETWEEN_A_{—} | | | | | | | | |
| LYMPHOID_AND_A_NON_{—} | | | | | | | | |
| LYMPHOID_CELL | | | | | | | | |
| REACTOME_INFLAMMASOMES | 19 | 0.00 | 0.74 | Up | 0.0005 | 0.0009 | 0.0005 | 0.0005 |
| REACTOME_INITIAL_TRIGGERING_{—} | 15 | 0.13 | 0.53 | Up | 0.0005 | 0.0009 | 0.0005 | 0.0005 |
| OF_COMPLEMENT | | | | | | | | |
| REACTOME_INNATE_IMMUNE_{—} | 249 | 0.10 | 0.52 | Up | 0.0005 | 0.0009 | 0.0005 | 0.0005 |
| SYSTEM | | | | | | | | |
| REACTOME_INTEGRATION_OF_{—} | 104 | 0.20 | 0.39 | Up | 0.0005 | 0.0009 | 0.0005 | 0.0005 |
| ENERGY_METABOLISM | | | | | | | | |
| REACTOME_INTERFERON_ALPHA_{—} | 78 | 0.03 | 0.87 | Up | 0.0005 | 0.0009 | 0.0005 | 0.0005 |
| BETA_SIGNALING | | | | | | | | |
| REACTOME_INTERFERON_GAMMA_{—} | 83 | 0.05 | 0.88 | Up | 0.0005 | 0.0009 | 0.0005 | 0.0005 |
| SIGNALING | | | | | | | | |
| REACTOME_INTERFERON_{—} | 177 | 0.12 | 0.62 | Up | 0.0005 | 0.0009 | 0.0005 | 0.0005 |
| SIGNALING | | | | | | | | |
| REACTOME_INTRINSIC_PATHWAY_{—} | 39 | 0.13 | 0.54 | Up | 0.0005 | 0.0009 | 0.0005 | 0.0005 |
| FOR_APOPTOSIS | | | | | | | | |
| REACTOME_ION_TRANSPORT_BY_{—} | 23 | 0.09 | 0.61 | Up | 0.0005 | 0.0009 | 0.0005 | 0.0005 |
| P_TYPE_ATPASES | | | | | | | | |
| REACTOME_LATENT_INFECTION_{—} | 29 | 0.03 | 0.48 | Up | 0.0005 | 0.0009 | 0.0005 | 0.0005 |
| OF_HOMO_{—}SAPIENS_WITH_{—} | | | | | | | | |
| MYCOBACTERIUM_TUBERCULOSIS | | | | | | | | |
| REACTOME_LYSOSOME_VESICLE_{—} | 32 | 0.06 | 0.44 | Up | 0.0005 | 0.0009 | 0.0005 | 0.0005 |
| BIOGENESIS | | | | | | | | |
| REACTOME_MAPK_TARGETS_{—} | 34 | 0.12 | 0.47 | Up | 0.0005 | 0.0009 | 0.0005 | 0.0005 |
| NUCLEAR_EVENTS_MEDIATED_BY_{—} | | | | | | | | |
| MAP_KINASES | | | | | | | | |
| REACTOME_METAL_ION_SLC_{—} | 19 | 0.21 | 0.53 | Up | 0.0005 | 0.0009 | 0.0005 | 0.0005 |
| TRANSPORTERS | | | | | | | | |
| REACTOME_MHC_CLASS_II_ANTIGEN_{—} | 96 | 0.08 | 0.46 | Up | 0.0005 | 0.0009 | 0.0005 | 0.0005 |
| PRESENTATION | | | | | | | | |
| REACTOME_MYD88_MAL_CASCADE_{—} | 98 | 0.14 | 0.49 | Up | 0.0005 | 0.0009 | 0.0005 | 0.0005 |
| INITIATED_ON_PLASMA_MEMBRANE | | | | | | | | |
| REACTOME_NEF_MEDIATED_{—} | 28 | 0.04 | 0.89 | Up | 0.0005 | 0.0009 | 0.0005 | 0.0005 |
| DOWNREGULATION_OF_MHC_CLASS_I_{—} | | | | | | | | |
| COMPLEX_CELL_SURFACE_EXPRESSION | | | | | | | | |
| REACTOME_NEF_MEDIATES_DOWN_{—} | 42 | 0.02 | 0.81 | Up | 0.0005 | 0.0009 | 0.0005 | 0.0005 |
| MODULATION_OF_CELL_SURFACE_{—} | | | | | | | | |
| RECEPTORS_BY_RECRUITING_{—} | | | | | | | | |
| THEM_TO_CLATHRIN_ADAPTERS | | | | | | | | |
| REACTOME_NFKB_AND_MAP_KINASES_{—} | 85 | 0.13 | 0.47 | Up | 0.0005 | 0.0009 | 0.0005 | 0.0005 |
| ACTIVATION_MEDIATED_BY_{—} | | | | | | | | |
| TLR4_SIGNALING_REPERTOIRE | | | | | | | | |
| REACTOME_NGF_SIGNALLING_VIA_{—} | 147 | 0.23 | 0.39 | Up | 0.0005 | 0.0009 | 0.0005 | 0.0005 |
| TRKA_FROM_THE_PLASMA_{—} | | | | | | | | |
| MEMBRANE | | | | | | | | |
| REACTOME_NOD1_2_SIGNALING_{—} | 36 | 0.08 | 0.56 | Up | 0.0005 | 0.0009 | 0.0005 | 0.0005 |
| PATHWAY | | | | | | | | |
| REACTOME_NUCLEOTIDE_BINDING_{—} | 54 | 0.06 | 0.61 | Up | 0.0005 | 0.0009 | 0.0005 | 0.0005 |
| DOMAIN_LEUCINE_RICH_REPEAT_{—} | | | | | | | | |
| CONTAINING_RECEPTOR_NLR_{—} | | | | | | | | |
| SIGNALING_PATHWAYS | | | | | | | | |
| REACTOME_O_LINKED_{—} | 27 | 0.15 | 0.59 | Up | 0.0005 | 0.0009 | 0.0005 | 0.0005 |
| GLYCOSYLATION_OF_MUCINS | | | | | | | | |
| REACTOME_PD1_SIGNALING | 17 | 0.00 | 0.88 | Up | 0.0005 | 0.0009 | 0.0005 | 0.0005 |
| REACTOME_PECAM1_{—} | 16 | 0.19 | 0.69 | Up | 0.0005 | 0.0009 | 0.0005 | 0.0005 |
| INTERACTIONS | | | | | | | | |
| REACTOME_PEPTIDE_LIGAND_{—} | 59 | 0.27 | 0.63 | Up | 0.0005 | 0.0009 | 0.0005 | 0.0005 |
| BINDING_RECEPTORS | | | | | | | | |
| REACTOME_PHOSPHOLIPID_{—} | 171 | 0.23 | 0.39 | Up | 0.0005 | 0.0009 | 0.0005 | 0.0005 |
| METABOLISM | | | | | | | | |
| REACTOME_PHOSPHORYLATION_{—} | 15 | 0.00 | 0.93 | Up | 0.0005 | 0.0009 | 0.0005 | 0.0005 |
| OF_CD3_AND_TCR_ZETA_CHAINS | | | | | | | | |
| REACTOME_PLATELET_ACTIVATION_{—} | 227 | 0.21 | 0.49 | Up | 0.0005 | 0.0009 | 0.0005 | 0.0005 |
| SIGNALING_AND_AGGREGATION | | | | | | | | |
| REACTOME_PLATELET_{—} | 22 | 0.14 | 0.41 | Up | 0.0005 | 0.0009 | 0.0005 | 0.0005 |
| SENSITIZATION_BY_LDL | | | | | | | | |
| REACTOME_POST_NMDA_RECEPTOR_{—} | 32 | 0.19 | 0.38 | Up | 0.0005 | 0.0009 | 0.0005 | 0.0005 |
| ACTIVATION_EVENTS | | | | | | | | |
| REACTOME_POTASSIUM_CHANNELS | 31 | 0.26 | 0.52 | Up | 0.0005 | 0.0009 | 0.0005 | 0.0005 |
| REACTOME_PURINE_METABOLISM | 32 | 0.22 | 0.38 | Up | 0.0005 | 0.0009 | 0.0005 | 0.0005 |
| REACTOME_RAP1_SIGNALLING | 18 | 0.22 | 0.44 | Up | 0.0005 | 0.0009 | 0.0005 | 0.0005 |
| REACTOME_REGULATION_OF_IFNA_{—} | 16 | 0.00 | 0.81 | Up | 0.0005 | 0.0009 | 0.0005 | 0.0005 |
| SIGNALING | | | | | | | | |
| REACTOME_REGULATION_OF_IFNG_{—} | 18 | 0.11 | 0.72 | Up | 0.0005 | 0.0009 | 0.0005 | 0.0005 |
| SIGNALING | | | | | | | | |
| REACTOME_REGULATION_OF_{—} | 72 | 0.19 | 0.43 | Up | 0.0005 | 0.0009 | 0.0005 | 0.0005 |
| INSULIN_SECRETION | | | | | | | | |
| REACTOME_REGULATION_OF_KIT_{—} | 22 | 0.14 | 0.77 | Up | 0.0005 | 0.0009 | 0.0005 | 0.0005 |
| SIGNALING | | | | | | | | |
| REACTOME_REGULATION_OF_{—} | 24 | 0.17 | 0.54 | Up | 0.0005 | 0.0009 | 0.0005 | 0.0005 |
| SIGNALING_BY_CBL | | | | | | | | |
| REACTOME_RESPONSE_TO_{—} | 107 | 0.18 | 0.50 | Up | 0.0005 | 0.0009 | 0.0005 | 0.0005 |
| ELEVATED_PLATELET_CYTOSOLIC_CA2_{—} | | | | | | | | |
| REACTOME_RIG_I_MDA5_MEDIATED_{—} | 65 | 0.09 | 0.48 | Up | 0.0005 | 0.0009 | 0.0005 | 0.0005 |
| INDUCTION_OF_IFN_ALPHA_BETA_{—} | | | | | | | | |
| PATHWAYS | | | | | | | | |
| REACTOME_RIP_MEDIATED_NFKB_{—} | 28 | 0.18 | 0.68 | Up | 0.0005 | 0.0009 | 0.0005 | 0.0005 |
| ACTIVATION_VIA_DAI | | | | | | | | |
| REACTOME_SIGNAL_REGULATORY_{—} | 15 | 0.07 | 0.80 | Up | 0.0005 | 0.0009 | 0.0005 | 0.0005 |
| PROTEIN_SIRP_FAMILY_{—} | | | | | | | | |
| INTERACTIONS | | | | | | | | |
| REACTOME_SIGNALING_BY_FGFR1_{—} | 31 | 0.26 | 0.42 | Up | 0.0005 | 0.0009 | 0.0005 | 0.0005 |
| FUSION_MUTANTS | | | | | | | | |
| REACTOME_SIGNALING_BY_FGFR1_{—} | 37 | 0.27 | 0.38 | Up | 0.0005 | 0.0009 | 0.0005 | 0.0005 |
| MUTANTS | | | | | | | | |
| REACTOME_SIGNALING_BY_GPCR | 327 | 0.25 | 0.46 | Up | 0.0005 | 0.0009 | 0.0005 | 0.0005 |
| REACTOME_SIGNALING_BY_ILS | 121 | 0.17 | 0.53 | Up | 0.0005 | 0.0009 | 0.0005 | 0.0005 |
| REACTOME_SIGNALING_BY_ROBO_{—} | 26 | 0.12 | 0.69 | Up | 0.0005 | 0.0009 | 0.0005 | 0.0005 |
| RECEPTOR | | | | | | | | |
| REACTOME_SIGNALING_BY_SCF_{—} | 100 | 0.24 | 0.48 | Up | 0.0005 | 0.0009 | 0.0005 | 0.0005 |
| KIT | | | | | | | | |
| REACTOME_SIGNALLING_TO_ERKS | 39 | 0.26 | 0.44 | Up | 0.0005 | 0.0009 | 0.0005 | 0.0005 |
| REACTOME_SLC_MEDIATED_{—} | 158 | 0.26 | 0.37 | Up | 0.0005 | 0.0009 | 0.0005 | 0.0005 |
| TRANSMEMBRANE_TRANSPORT | | | | | | | | |
| REACTOME_TAK1_ACTIVATES_NF | 25 | 0.12 | 0.52 | Up | 0.0005 | 0.0009 | 0.0005 | 0.0005 |
| KB_BY_PHOSPHORYLATION_AND_{—} | | | | | | | | |
| ACTIVATION_OF_IKKS_COMPLEX | | | | | | | | |
| REACTOME_TCR_SIGNALING | 59 | 0.12 | 0.69 | Up | 0.0005 | 0.0009 | 0.0005 | 0.0005 |
| REACTOME_THE_NLRP3_{—} | 15 | 0.00 | 0.73 | Up | 0.0005 | 0.0009 | 0.0005 | 0.0005 |
| INFLAMMASOME | | | | | | | | |
| REACTOME_THE_ROLE_OF_NEF_IN_{—} | 58 | 0.05 | 0.76 | Up | 0.0005 | 0.0009 | 0.0005 | 0.0005 |
| HIV1_REPLICATION_AND_{—} | | | | | | | | |
| DISEASE_PATHOGENESIS | | | | | | | | |
| REACTOME_TOLL_RECEPTOR_{—} | 142 | 0.12 | 0.50 | Up | 0.0005 | 0.0009 | 0.0005 | 0.0005 |
| CASCADES | | | | | | | | |
| REACTOME_TRAF6_MEDIATED_{—} | 107 | 0.14 | 0.48 | Up | 0.0005 | 0.0009 | 0.0005 | 0.0005 |
| INDUCTION_OF_NFKB_AND_MAP_{—} | | | | | | | | |
| KINASES_UPON_TLR7_8_OR_9_{—} | | | | | | | | |
| ACTIVATION | | | | | | | | |
| REACTOME_TRAF6_MEDIATED_{—} | 22 | 0.23 | 0.41 | Up | 0.0005 | 0.0009 | 0.0005 | 0.0005 |
| INDUCTION_OF_TAK1_COMPLEX | | | | | | | | |
| REACTOME_TRAF6_MEDIATED_{—} | 17 | 0.06 | 0.53 | Up | 0.0005 | 0.0009 | 0.0005 | 0.0005 |
| IRF7_ACTIVATION | | | | | | | | |
| REACTOME_TRAF6_MEDIATED_{—} | 34 | 0.12 | 0.59 | Up | 0.0005 | 0.0009 | 0.0005 | 0.0005 |
| IRF7_ACTIVATION_IN_TLR7_8_OR_9_{—} | | | | | | | | |
| SIGNALING | | | | | | | | |
| REACTOME_TRAF6_MEDIATED_{—} | 23 | 0.13 | 0.61 | Up | 0.0005 | 0.0009 | 0.0005 | 0.0005 |
| NFKB_ACTIVATION | | | | | | | | |
| REACTOME_TRAFFICKING_AND_{—} | 36 | 0.11 | 0.56 | Up | 0.0005 | 0.0009 | 0.0005 | 0.0005 |
| PROCESSING_OF_ENDOSOMAL_TLR | | | | | | | | |
| REACTOME_TRANS_GOLGI_NETWORK_{—} | 75 | 0.11 | 0.33 | Up | 0.0005 | 0.0009 | 0.0005 | 0.0005 |
| VESICLE_BUDDING | | | | | | | | |
| REACTOME_TRANSCRIPTIONAL_{—} | 79 | 0.16 | 0.32 | Up | 0.0005 | 0.0009 | 0.0005 | 0.0005 |
| REGULATION_OF_WHITE_ADIPOCYTE_{—} | | | | | | | | |
| DIFFERENTIATION | | | | | | | | |
| REACTOME_TRANSMEMBRANE_{—} | 278 | 0.26 | 0.36 | Up | 0.0005 | 0.0009 | 0.0005 | 0.0005 |
| TRANSPORT_OF_SMALL_MOLECULES | | | | | | | | |
| REACTOME_TRANSPORT_OF_{—} | 53 | 0.25 | 0.40 | Up | 0.0005 | 0.0009 | 0.0005 | 0.0005 |
| GLUCOSE_AND_OTHER_SUGARS_BILE_{—} | | | | | | | | |
| SALTS_AND_ORGANIC_ACIDS_METAL_{—} | | | | | | | | |
| IONS_AND_AMINE_COMPOUNDS | | | | | | | | |
| REACTOME_TRIF_MEDIATED_TLR3_{—} | 88 | 0.13 | 0.48 | Up | 0.0005 | 0.0009 | 0.0005 | 0.0005 |
| SIGNALING | | | | | | | | |
| REACTOME_ACTIVATION_OF_BH3_{—} | 21 | 0.19 | 0.57 | Up | 0.0010 | 0.0020 | 0.0005 | 0.0005 |
| ONLY_PROTEINS | | | | | | | | |
| REACTOME_ACTIVATION_OF_KAINATE_{—} | 22 | 0.23 | 0.41 | Up | 0.0010 | 0.0020 | 0.0005 | 0.0005 |
| RECEPTORS_UPON_GLUTAMATE_{—} | | | | | | | | |
| BINDING | | | | | | | | |
| REACTOME_CELL_EXTRACELLULAR_{—} | 17 | 0.12 | 0.47 | Up | 0.0010 | 0.0020 | 0.0005 | 0.0005 |
| MATRIX_INTERACTIONS | | | | | | | | |
| REACTOME_CREB_PHOSPHORYLATION_{—} | 24 | 0.13 | 0.38 | Up | 0.0010 | 0.0020 | 0.0005 | 0.0005 |
| THROUGH_THE_ACTIVATION_{—} | | | | | | | | |
| OF_RAS | | | | | | | | |
| REACTOME_DEGRADATION_OF_THE_{—} | 17 | 0.41 | 0.35 | Up | 0.0010 | 0.0020 | 0.0005 | 0.0005 |
| EXTRACELLULAR_MATRIX | | | | | | | | |
| REACTOME_DESTABILIZATION_OF_{—} | 20 | 0.15 | 0.45 | Up | 0.0010 | 0.0020 | 0.0010 | 0.0010 |
| MRNA_BY_BRF1 | | | | | | | | |
| REACTOME_EARLY_PHASE_OF_HIV_{—} | 16 | 0.13 | 0.50 | Up | 0.0010 | 0.0020 | 0.0005 | 0.0005 |
| LIFE_CYCLE | | | | | | | | |
| REACTOME_ER_PHAGOSOME_{—} | 76 | 0.11 | 0.36 | Up | 0.0010 | 0.0020 | 0.0005 | 0.0005 |
| PATHWAY | | | | | | | | |
| REACTOME_G_ALPHA_Z_SIGNALLING_{—} | 28 | 0.29 | 0.50 | Up | 0.0010 | 0.0020 | 0.0005 | 0.0005 |
| EVENTS | | | | | | | | |
| REACTOME_G_PROTEIN_BETA_{—} | 23 | 0.13 | 0.57 | Up | 0.0010 | 0.0020 | 0.0010 | 0.0010 |
| GAMMA_SIGNALLING | | | | | | | | |
| REACTOME_GLYCOLYSIS | 26 | 0.08 | 0.46 | Up | 0.0010 | 0.0020 | 0.0005 | 0.0005 |
| REACTOME_GPCR_LIGAND_BINDING | 143 | 0.29 | 0.52 | Up | 0.0010 | 0.0020 | 0.0005 | 0.0005 |
| REACTOME_HOST_INTERACTIONS_{—} | 158 | 0.13 | 0.37 | Up | 0.0010 | 0.0020 | 0.0005 | 0.0005 |
| OF_HIV_FACTORS | | | | | | | | |
| REACTOME_INHIBITION_OF_INSULIN_{—} | 17 | 0.24 | 0.47 | Up | 0.0010 | 0.0020 | 0.0005 | 0.0005 |
| SECRETION_BY_ADRENALINE_{—} | | | | | | | | |
| NORADRENALINE | | | | | | | | |
| REACTOME_INTRINSIC_PATHWAY | 17 | 0.29 | 0.47 | Up | 0.0010 | 0.0020 | 0.0010 | 0.0010 |
| REACTOME_ION_CHANNEL_{—} | 26 | 0.12 | 0.54 | Up | 0.0010 | 0.0020 | 0.0005 | 0.0005 |
| TRANSPORT | | | | | | | | |
| REACTOME_KINESINS | 25 | 0.00 | 0.56 | Up | 0.0010 | 0.0020 | 0.0015 | 0.0015 |
| REACTOME_METABOLISM_OF_{—} | 201 | 0.20 | 0.37 | Up | 0.0010 | 0.0020 | 0.0005 | 0.0005 |
| CARBOHYDRATES | | | | | | | | |
| REACTOME_NEGATIVE_REGULATORS_{—} | 35 | 0.11 | 0.34 | Up | 0.0010 | 0.0020 | 0.0005 | 0.0005 |
| OF_RIG_I_MDA5_SIGNALING | | | | | | | | |
| REACTOME_NUCLEAR_EVENTS_{—} | 29 | 0.07 | 0.48 | Up | 0.0010 | 0.0020 | 0.0005 | 0.0005 |
| KINASE_AND_TRANSCRIPTION_{—} | | | | | | | | |
| FACTOR_ACTIVATION | | | | | | | | |
| REACTOME_OPIOID_SIGNALLING | 67 | 0.22 | 0.37 | Up | 0.0010 | 0.0020 | 0.0005 | 0.0005 |
| REACTOME_PLC_BETA_MEDIATED_{—} | 37 | 0.24 | 0.49 | Up | 0.0010 | 0.0020 | 0.0005 | 0.0005 |
| EVENTS | | | | | | | | |
| REACTOME_RECYCLING_PATHWAY_{—} | 28 | 0.14 | 0.39 | Up | 0.0010 | 0.0020 | 0.0005 | 0.0005 |
| OF_L1 | | | | | | | | |
| REACTOME_SIGNALING_BY_FGFR_{—} | 44 | 0.32 | 0.36 | Up | 0.0010 | 0.0020 | 0.0005 | 0.0005 |
| MUTANTS | | | | | | | | |
| REACTOME_SIGNALING_BY_THE_{—} | 143 | 0.21 | 0.36 | Up | 0.0010 | 0.0020 | 0.0005 | 0.0005 |
| B_CELL_RECEPTOR_BCR | | | | | | | | |
| REACTOME_SYNTHESIS_OF_PIPS_{—} | 34 | 0.24 | 0.38 | Up | 0.0010 | 0.0020 | 0.0005 | 0.0005 |
| AT_THE_PLASMA_MEMBRANE | | | | | | | | |
| REACTOME_ANTIVIRAL_MECHANISM_{—} | 79 | 0.22 | 0.30 | Up | 0.0015 | 0.0031 | 0.0010 | 0.0010 |
| BY_IFN_STIMULATED_GENES | | | | | | | | |
| REACTOME_CLASS_I_MHC_MEDIATED_{—} | 255 | 0.18 | 0.29 | Up | 0.0015 | 0.0031 | 0.0005 | 0.0005 |
| ANTIGEN_PROCESSING_{—} | | | | | | | | |
| PRESENTATION | | | | | | | | |
| REACTOME_EFFECTS_OF_PIP2_{—} | 18 | 0.22 | 0.50 | Up | 0.0015 | 0.0031 | 0.0005 | 0.0005 |
| HYDROLYSIS | | | | | | | | |
| REACTOME_G_BETA_GAMMA_{—} | 19 | 0.11 | 0.58 | Up | 0.0015 | 0.0031 | 0.0005 | 0.0005 |
| SIGNALLING_THROUGH_PI3KGAMMA | | | | | | | | |
| REACTOME_GLUCOSE_TRANSPORT | 36 | 0.22 | 0.25 | Up | 0.0015 | 0.0031 | 0.0005 | 0.0005 |
| REACTOME_HYALURONAN_METABOLISM | 17 | 0.12 | 0.71 | Up | 0.0015 | 0.0031 | 0.0005 | 0.0005 |
| REACTOME_IRON_UPTAKE_AND_{—} | 36 | 0.14 | 0.39 | Up | 0.0015 | 0.0031 | 0.0005 | 0.0005 |
| TRANSPORT | | | | | | | | |
| REACTOME_MAP_KINASE_ACTIVATION_{—} | 53 | 0.11 | 0.38 | Up | 0.0015 | 0.0031 | 0.0005 | 0.0005 |
| IN_TLR_CASCADE | | | | | | | | |
| REACTOME_NEURONAL_SYSTEM | 152 | 0.28 | 0.36 | Up | 0.0015 | 0.0031 | 0.0005 | 0.0005 |
| REACTOME_P75_NTR_RECEPTOR_{—} | 94 | 0.21 | 0.40 | Up | 0.0015 | 0.0031 | 0.0005 | 0.0005 |
| MEDIATED_SIGNALLING | | | | | | | | |
| REACTOME_SIGNAL_AMPLIFICATION | 25 | 0.20 | 0.48 | Up | 0.0015 | 0.0031 | 0.0010 | 0.0010 |
| REACTOME_SIGNALING_BY_{—} | 106 | 0.25 | 0.41 | Up | 0.0015 | 0.0031 | 0.0005 | 0.0005 |
| RHO_GTPASES | | | | | | | | |
| REACTOME_AMYLOIDS | 33 | 0.15 | 0.55 | Up | 0.0020 | 0.0040 | 0.0010 | 0.0010 |
| REACTOME_CELL_CELL_{—} | 105 | 0.30 | 0.41 | Up | 0.0020 | 0.0040 | 0.0005 | 0.0005 |
| COMMUNICATION | | | | | | | | |
| REACTOME_CHONDROITIN_SULFATE_{—} | 15 | 0.13 | 0.53 | Up | 0.0020 | 0.0040 | 0.0005 | 0.0005 |
| BIOSYNTHESIS | | | | | | | | |
| REACTOME_CHONDROITIN_SULFATE_{—} | 38 | 0.08 | 0.45 | Up | 0.0020 | 0.0040 | 0.0010 | 0.0010 |
| DERMATAN_SULFATE_METABOLISM | | | | | | | | |
| REACTOME_G1_S_SPECIFIC_{—} | 18 | 0.06 | 0.56 | Up | 0.0020 | 0.0040 | 0.0005 | 0.0005 |
| TRANSCRIPTION | | | | | | | | |
| REACTOME_GABA_B_RECEPTOR_{—} | 23 | 0.35 | 0.48 | Up | 0.0020 | 0.0040 | 0.0005 | 0.0005 |
| ACTIVATION | | | | | | | | |
| REACTOME_INSULIN_SYNTHESIS_{—} | 27 | 0.11 | 0.33 | Up | 0.0020 | 0.0040 | 0.0005 | 0.0005 |
| AND_PROCESSING | | | | | | | | |
| REACTOME_NEUROTRANSMITTER_{—} | 83 | 0.25 | 0.36 | Up | 0.0020 | 0.0040 | 0.0010 | 0.0010 |
| RECEPTOR_BINDING_AND_{—} | | | | | | | | |
| DOWNSTREAM_TRANSMISSION_IN_{—} | | | | | | | | |
| THE_POSTSYNAPTIC_CELL | | | | | | | | |
| REACTOME_PRE_NOTCH_{—} | 21 | 0.24 | 0.38 | Up | 0.0020 | 0.0040 | 0.0005 | 0.0005 |
| PROCESSING_IN_GOLGI | | | | | | | | |
| REACTOME_REGULATION_OF_{—} | 27 | 0.26 | 0.33 | Up | 0.0020 | 0.0040 | 0.0005 | 0.0005 |
| WATER_BALANCE_BY_RENAL_{—} | | | | | | | | |
| AQUAPORINS | | | | | | | | |
| REACTOME_SIGNALLING_BY_NGF | 236 | 0.22 | 0.40 | Up | 0.0020 | 0.0040 | 0.0005 | 0.0005 |
| REACTOME_SIGNALLING_TO_RAS | 30 | 0.27 | 0.47 | Up | 0.0020 | 0.0040 | 0.0005 | 0.0005 |
| REACTOME_SPHINGOLIPID_{—} | 61 | 0.15 | 0.43 | Up | 0.0020 | 0.0040 | 0.0005 | 0.0005 |
| METABOLISM | | | | | | | | |
| REACTOME_TRAFFICKING_OF_{—} | 20 | 0.20 | 0.35 | Up | 0.0020 | 0.0040 | 0.0005 | 0.0005 |
| AMPA_RECEPTORS | | | | | | | | |
| REACTOME_ADP_SIGNALLING_{—} | 19 | 0.16 | 0.47 | Up | 0.0025 | 0.0049 | 0.0010 | 0.0010 |
| THROUGH_P2RY1 | | | | | | | | |
| REACTOME_INWARDLY_RECTIFYING_{—} | 19 | 0.26 | 0.53 | Up | 0.0025 | 0.0049 | 0.0005 | 0.0005 |
| K_CHANNELS | | | | | | | | |
| REACTOME_PI_METABOLISM | 48 | 0.23 | 0.33 | Up | 0.0025 | 0.0049 | 0.0005 | 0.0005 |
| REACTOME_REGULATION_OF_{—} | 32 | 0.22 | 0.38 | Up | 0.0025 | 0.0049 | 0.0005 | 0.0005 |
| INSULIN_SECRETION_BY_GLUCAGON_{—} | | | | | | | | |
| LIKE_PEPTIDE1 | | | | | | | | |
| REACTOME_TRANSPORT_OF_{—} | 54 | 0.24 | 0.48 | Up | 0.0025 | 0.0049 | 0.0005 | 0.0005 |
| INORGANIC_CATIONS_ANIONS_AND_{—} | | | | | | | | |
| AMINO_ACIDS_OLIGOPEPTIDES | | | | | | | | |
| REACTOME_APOPTOTIC_EXECUTION_{—} | 60 | 0.23 | 0.37 | Up | 0.0030 | 0.0058 | 0.0005 | 0.0005 |
| PHASE | | | | | | | | |
| REACTOME_DEVELOPMENTAL_{—} | 341 | 0.23 | 0.36 | Up | 0.0030 | 0.0058 | 0.0005 | 0.0005 |
| BIOLOGY | | | | | | | | |
| REACTOME_DOWNSTREAM_SIGNAL_{—} | 105 | 0.30 | 0.36 | Up | 0.0030 | 0.0058 | 0.0005 | 0.0005 |
| TRANSDUCTION | | | | | | | | |
| REACTOME_ERK_MAPK_TARGETS | 25 | 0.08 | 0.48 | Up | 0.0030 | 0.0058 | 0.0005 | 0.0005 |
| REACTOME_FORMATION_OF_FIBRIN_{—} | 30 | 0.27 | 0.43 | Up | 0.0030 | 0.0058 | 0.0005 | 0.0005 |
| CLOT_CLOTTING_CASCADE | | | | | | | | |
| REACTOME_INTEGRIN_ALPHAIIB_{—} | 38 | 0.21 | 0.47 | Up | 0.0030 | 0.0058 | 0.0005 | 0.0005 |
| BETA3_SIGNALING | | | | | | | | |
| REACTOME_LOSS_OF_NLP_FROM_{—} | 75 | 0.15 | 0.35 | Up | 0.0030 | 0.0058 | 0.0010 | 0.0010 |
| MITOTIC_CENTROSOMES | | | | | | | | |
| REACTOME_SIGNALING_BY_FGFR_{—} | 130 | 0.29 | 0.33 | Up | 0.0030 | 0.0058 | 0.0005 | 0.0005 |
| IN_DISEASE | | | | | | | | |
| REACTOME_HS_GAG_DEGRADATION | 17 | 0.06 | 0.47 | Up | 0.0035 | 0.0067 | 0.0005 | 0.0005 |
| REACTOME_MITOTIC_G2_G2_M_{—} | 92 | 0.14 | 0.36 | Up | 0.0035 | 0.0067 | 0.0005 | 0.0005 |
| PHASES | | | | | | | | |
| REACTOME_PHOSPHOLIPASE_C_{—} | 38 | 0.26 | 0.42 | Up | 0.0035 | 0.0067 | 0.0005 | 0.0005 |
| MEDIATED_CASCADE | | | | | | | | |
| REACTOME_SEMAPHORIN_{—} | 72 | 0.22 | 0.44 | Up | 0.0035 | 0.0067 | 0.0005 | 0.0005 |
| INTERACTIONS | | | | | | | | |
| REACTOME_THROMBIN_SIGNALLING_{—} | 23 | 0.22 | 0.39 | Up | 0.0035 | 0.0067 | 0.0005 | 0.0005 |
| THROUGH_PROTEINASE_ACTIVATED_{—} | | | | | | | | |
| RECEPTORS_PARS | | | | | | | | |
| REACTOME_ACTIVATED_NOTCH1_{—} | 24 | 0.21 | 0.38 | Up | 0.0040 | 0.0075 | 0.0010 | 0.0010 |
| TRANSMITS_SIGNAL_TO_THE_{—} | | | | | | | | |
| NUCLEUS | | | | | | | | |
| REACTOME_BOTULINUM_{—} | 15 | 0.20 | 0.47 | Up | 0.0040 | 0.0075 | 0.0005 | 0.0005 |
| NEUROTOXICITY | | | | | | | | |
| REACTOME_HIV_INFECTION | 232 | 0.17 | 0.32 | Up | 0.0040 | 0.0075 | 0.0005 | 0.0005 |
| REACTOME_TIE2_SIGNALING | 21 | 0.33 | 0.43 | Up | 0.0040 | 0.0075 | 0.0005 | 0.0005 |
| REACTOME_GLYCEROPHOSPHOLIPID_{—} | 63 | 0.33 | 0.38 | Up | 0.0045 | 0.0083 | 0.0005 | 0.0005 |
| BIOSYNTHESIS | | | | | | | | |
| REACTOME_THROMBOXANE_{—} | 16 | 0.19 | 0.38 | Up | 0.0045 | 0.0083 | 0.0005 | 0.0005 |
| SIGNALLING_THROUGH_TP_{—} | | | | | | | | |
| RECEPTOR | | | | | | | | |
| REACTOME_INTEGRIN_CELL_{—} | 86 | 0.29 | 0.47 | Up | 0.0050 | 0.0092 | 0.0005 | 0.0005 |
| SURFACE_INTERACTIONS | | | | | | | | |
| REACTOME_PLATELET_{—} | 41 | 0.22 | 0.44 | Up | 0.0050 | 0.0092 | 0.0005 | 0.0005 |
| AGGREGATION_PLUG_FORMATION | | | | | | | | |
| REACTOME_SEMA3A_PAK_{—} | 25 | 0.20 | 0.52 | Up | 0.0050 | 0.0092 | 0.0005 | 0.0005 |
| DEPENDENT_AXON_REPULSION | | | | | | | | |
| REACTOME_TRANSMISSION_{—} | 125 | 0.29 | 0.32 | Up | 0.0050 | 0.0092 | 0.0005 | 0.0005 |
| ACROSS_CHEMICAL_SYNAPSES | | | | | | | | |
| REACTOME_CELL_DEATH_{—} | 69 | 0.23 | 0.33 | Up | 0.0055 | 0.0100 | 0.0005 | 0.0005 |
| SIGNALLING_VIA_NRAGE_NRIF_{—} | | | | | | | | |
| AND_NADE | | | | | | | | |
| REACTOME_RECRUITMENT_OF_{—} | 82 | 0.15 | 0.34 | Up | 0.0055 | 0.0100 | 0.0005 | 0.0005 |
| MITOTIC_CENTROSOME_PROTEINS_{—} | | | | | | | | |
| AND_COMPLEXES | | | | | | | | |
| REACTOME_G_ALPHA1213_{—} | 75 | 0.23 | 0.37 | Up | 0.0060 | 0.0108 | 0.0005 | 0.0005 |
| SIGNALLING_EVENTS | | | | | | | | |
| REACTOME_GRB2_SOS_PROVIDES_{—} | 21 | 0.24 | 0.48 | Up | 0.0065 | 0.0116 | 0.0005 | 0.0005 |
| LINKAGE_TO_MAPK_SIGNALING_{—} | | | | | | | | |
| FOR_INTERGRINS | | | | | | | | |
| REACTOME_APOPTOTIC_CLEAVAGE_{—} | 48 | 0.27 | 0.40 | Up | 0.0070 | 0.0123 | 0.0005 | 0.0005 |
| OF_CELLULAR_PROTEINS | | | | | | | | |
| REACTOME_G1_PHASE | 38 | 0.18 | 0.39 | Up | 0.0070 | 0.0123 | 0.0005 | 0.0005 |
| REACTOME_P130CAS_LINKAGE_{—} | 20 | 0.20 | 0.45 | Up | 0.0075 | 0.0131 | 0.0005 | 0.0005 |
| TO_MAPK_SIGNALING_FOR_{—} | | | | | | | | |
| INTEGRINS | | | | | | | | |
| REACTOME_PLATELET_{—} | 55 | 0.22 | 0.40 | Up | 0.0075 | 0.0131 | 0.0005 | 0.0005 |
| HOMEOSTASIS | | | | | | | | |
| REACTOME_NETRIN1_SIGNALING | 39 | 0.28 | 0.44 | Up | 0.0080 | 0.0139 | 0.0005 | 0.0005 |
| REACTOME_JNK_C_JUN_KINASES_{—} | 15 | 0.20 | 0.20 | Up | 0.0085 | 0.0146 | 0.0005 | 0.0005 |
| PHOSPHORYLATION_AND_{—} | | | | | | | | |
| ACTIVATION_MEDIATED_BY_{—} | | | | | | | | |
| ACTIVATED_HUMAN_TAK1 | | | | | | | | |
| REACTOME_AXON_GUIDANCE | 214 | 0.26 | 0.40 | Up | 0.0090 | 0.0154 | 0.0005 | 0.0005 |
| REACTOME_GABA_RECEPTOR_{—} | 26 | 0.35 | 0.42 | Up | 0.0090 | 0.0154 | 0.0005 | 0.0005 |
| ACTIVATION | | | | | | | | |
| REACTOME_SEMA4D_IN_{—} | 38 | 0.26 | 0.42 | Up | 0.0090 | 0.0154 | 0.0005 | 0.0005 |
| SEMAPHORIN_SIGNALING | | | | | | | | |
| REACTOME_COMPLEMENT_{—} | 31 | 0.16 | 0.39 | Up | 0.0095 | 0.0161 | 0.0005 | 0.0005 |
| CASCADE | | | | | | | | |
| REACTOME_GLUCAGON_SIGNALING_{—} | 21 | 0.33 | 0.29 | Up | 0.0095 | 0.0161 | 0.0005 | 0.0005 |
| IN_METABOLIC_REGULATION | | | | | | | | |
| REACTOME_GLYCOSAMINOGLYCAN_{—} | 83 | 0.14 | 0.47 | Up | 0.0100 | 0.0168 | 0.0005 | 0.0005 |
| METABOLISM | | | | | | | | |
| REACTOME_INSULIN_RECEPTOR_{—} | 19 | 0.05 | 0.32 | Up | 0.0100 | 0.0168 | 0.0105 | 0.0106 |
| RECYCLING | | | | | | | | |
| REACTOME_METABOLISM_OF_{—} | 71 | 0.23 | 0.31 | Up | 0.0100 | 0.0168 | 0.0005 | 0.0005 |
| NUCLEOTIDES | | | | | | | | |
| REACTOME_E2F_MEDIATED_{—} | 35 | 0.09 | 0.46 | Up | 0.0105 | 0.0175 | 0.0010 | 0.0010 |
| REGULATION_OF_DNA_REPLICATION | | | | | | | | |
| REACTOME_MITOTIC_PROMETAPHASE | 92 | 0.11 | 0.40 | Up | 0.0125 | 0.0204 | 0.0010 | 0.0010 |
| REACTOME_DESTABILIZATION_OF_{—} | 19 | 0.21 | 0.37 | Up | 0.0130 | 0.0211 | 0.0005 | 0.0005 |
| MRNA_BY_TRISTETRAPROLIN_TTP | | | | | | | | |
| REACTOME_AQUAPORIN_MEDIATED_{—} | 29 | 0.28 | 0.31 | Up | 0.0145 | 0.0233 | 0.0005 | 0.0005 |
| TRANSPORT | | | | | | | | |
| REACTOME_FANCONI_ANEMIA_{—} | 19 | 0.16 | 0.32 | Up | 0.0145 | 0.0233 | 0.0005 | 0.0005 |
| PATHWAY | | | | | | | | |
| REACTOME_REGULATION_OF_{—} | 15 | 0.07 | 0.53 | Up | 0.0145 | 0.0233 | 0.0005 | 0.0005 |
| COMPLEMENT_CASCADE | | | | | | | | |
| REACTOME_TRANSPORT_TO_THE_{—} | 32 | 0.06 | 0.31 | Up | 0.0155 | 0.0248 | 0.0015 | 0.0015 |
| GOLGI_AND_SUBSEQUENT_{—} | | | | | | | | |
| MODIFICATION | | | | | | | | |
| REACTOME_NEUROTRANSMITTER_{—} | 33 | 0.30 | 0.27 | Up | 0.0160 | 0.0256 | 0.0005 | 0.0005 |
| RELEASE_CYCLE | | | | | | | | |
| REACTOME_SYNTHESIS_AND_{—} | 17 | 0.12 | 0.41 | Up | 0.0170 | 0.0270 | 0.0005 | 0.0005 |
| INTERCONVERSION_OF_NUCLEOTIDE_{—} | | | | | | | | |
| DI_AND_TRIPHOSPHATES | | | | | | | | |
| REACTOME_TRANSFERRIN_{—} | 24 | 0.08 | 0.33 | Up | 0.0175 | 0.0276 | 0.0010 | 0.0010 |
| ENDOCYTOSIS_AND_RECYCLING | | | | | | | | |
| REACTOME_DCC_MEDIATED_{—} | 20 | 0.25 | 0.45 | Up | 0.0180 | 0.0282 | 0.0005 | 0.0005 |
| ATTRACTIVE_SIGNALING | | | | | | | | |
| REACTOME_NRAGE_SIGNALS_{—} | 50 | 0.30 | 0.30 | Up | 0.0185 | 0.0289 | 0.0005 | 0.0005 |
| DEATH_THROUGH_JNK | | | | | | | | |
| REACTOME_SEMA4D_INDUCED_{—} | 33 | 0.24 | 0.39 | Up | 0.0185 | 0.0289 | 0.0005 | 0.0005 |
| CELL_MIGRATION_AND_GROWTH_{—} | | | | | | | | |
| CONE_COLLAPSE | | | | | | | | |
| REACTOME_AMINO_ACID_AND_{—} | 30 | 0.33 | 0.47 | Up | 0.0190 | 0.0295 | 0.0005 | 0.0005 |
| OLIGOPEPTIDE_SLC_TRANSPORTERS | | | | | | | | |
| REACTOME_ERKS_ARE_INACTIVATED | 15 | 0.13 | 0.33 | Up | 0.0190 | 0.0295 | 0.0005 | 0.0005 |
| REACTOME_POST_TRANSLATIONAL_{—} | 154 | 0.18 | 0.22 | Up | 0.0220 | 0.0338 | 0.0005 | 0.0005 |
| PROTEIN_MODIFICATION | | | | | | | | |
| REACTOME_SIGNALING_BY_EGFR_{—} | 125 | 0.29 | 0.32 | Up | 0.0220 | 0.0338 | 0.0005 | 0.0005 |
| IN_CANCER | | | | | | | | |
| REACTOME_CELL_CYCLE | 392 | 0.16 | 0.32 | Up | 0.0235 | 0.0357 | 0.0005 | 0.0005 |
| REACTOME_SIGNALING_BY_ERBB2 | 110 | 0.30 | 0.33 | Up | 0.0245 | 0.0371 | 0.0005 | 0.0005 |
| REACTOME_G0_AND_EARLY_G1 | 25 | 0.12 | 0.40 | Up | 0.0250 | 0.0377 | 0.0005 | 0.0005 |
| REACTOME_SYNTHESIS_OF_PA | 20 | 0.35 | 0.45 | Up | 0.0250 | 0.0377 | 0.0005 | 0.0005 |
| REACTOME_DNA_REPLICATION | 194 | 0.11 | 0.39 | Up | 0.0255 | 0.0384 | 0.0005 | 0.0005 |
| REACTOME_NEPHRIN_INTERACTIONS | 25 | 0.24 | 0.44 | Up | 0.0255 | 0.0384 | 0.0005 | 0.0005 |
| REACTOME_HOMOLOGOUS_{—} | 19 | 0.11 | 0.32 | Up | 0.0260 | 0.0391 | 0.0020 | 0.0020 |
| RECOMBINATION_REPAIR_OF_{—} | | | | | | | | |
| REPLICATION_INDEPENDENT_DOUBLE_{—} | | | | | | | | |
| STRAND_BREAKS | | | | | | | | |
| REACTOME_DOWNSTREAM_SIGNALING_{—} | 92 | 0.32 | 0.34 | Up | 0.0265 | 0.0397 | 0.0005 | 0.0005 |
| OF_ACTIVATED_FGFR | | | | | | | | |
| REACTOME_SEMA3A_PLEXIN_REPULSION_{—} | 25 | 0.24 | 0.44 | Up | 0.0265 | 0.0397 | 0.0005 | 0.0005 |
| SIGNALING_BY_INHIBITING_{—} | | | | | | | | |
| INTEGRIN_ADHESION | | | | | | | | |
| REACTOME_CELL_CYCLE_MITOTIC | 335 | 0.14 | 0.35 | Up | 0.0270 | 0.0404 | 0.0005 | 0.0005 |
| REACTOME_CRMPS_IN_SEMA3A_{—} | 18 | 0.33 | 0.50 | Up | 0.0285 | 0.0423 | 0.0005 | 0.0005 |
| SIGNALING | | | | | | | | |
| REACTOME_DOUBLE_STRAND_BREAK_{—} | 25 | 0.16 | 0.28 | Up | 0.0290 | 0.0430 | 0.0015 | 0.0015 |
| REPAIR | | | | | | | | |
| REACTOME_PRE_NOTCH_{—} | 47 | 0.19 | 0.36 | Up | 0.0305 | 0.0449 | 0.0005 | 0.0005 |
| EXPRESSION_AND_PROCESSING | | | | | | | | |
| REACTOME_SIGNALING_BY_FGFR | 106 | 0.30 | 0.32 | Up | 0.0310 | 0.0455 | 0.0005 | 0.0005 |
| REACTOME_MITOTIC_M_M_G1_PHASES | 175 | 0.11 | 0.37 | Up | 0.0315 | 0.0462 | 0.0005 | 0.0005 |
| REACTOME_G1_S_TRANSITION | 109 | 0.10 | 0.36 | Up | 0.0335 | 0.0488 | 0.0005 | 0.0005 |
| REACTOME_ACTIVATION_OF_THE_{—} | 27 | 0.11 | 0.48 | Up | 0.0350 | 0.0508 | 0.0040 | 0.0040 |
| PRE_REPLICATIVE_COMPLEX | | | | | | | | |
| REACTOME_SIGNALING_BY_PDGF | 131 | 0.33 | 0.33 | Up | 0.0370 | 0.0534 | 0.0005 | 0.0005 |
| REACTOME_MITOTIC_G1_G1_S_PHASES | 136 | 0.13 | 0.35 | Up | 0.0380 | 0.0547 | 0.0005 | 0.0005 |
| REACTOME_KERATAN_SULFATE_{—} | 22 | 0.23 | 0.50 | Up | 0.0415 | 0.0593 | 0.0005 | 0.0005 |
| KERATIN_METABOLISM | | | | | | | | |
| REACTOME_GLUCOSE_METABOLISM | 61 | 0.25 | 0.33 | Up | 0.0430 | 0.0612 | 0.0005 | 0.0005 |
| REACTOME_PLATELET_ADHESION_{—} | 21 | 0.33 | 0.43 | Up | 0.0455 | 0.0644 | 0.0005 | 0.0005 |
| TO_EXPOSED_COLLAGEN | | | | | | | | |
| REACTOME_AMINO_ACID_{—} | 21 | 0.33 | 0.43 | Up | 0.0465 | 0.0656 | 0.0005 | 0.0005 |
| TRANSPORT_ACROSS_THE_PLASMA_{—} | | | | | | | | |
| MEMBRANE | | | | | | | | |
| REACTOME_TRANSPORT_OF_VITAMINS_{—} | 22 | 0.41 | 0.23 | Up | 0.0480 | 0.0675 | 0.0005 | 0.0005 |
| NUCLEOSIDES_AND_RELATED_{—} | | | | | | | | |
| MOLECULES | | | | | | | | |
| REACTOME_G2_M_CHECKPOINTS | 38 | 0.13 | 0.39 | Up | 0.0520 | 0.0727 | 0.0010 | 0.0010 |
| REACTOME_DNA_STRAND_ELONGATION | 30 | 0.13 | 0.50 | Up | 0.0525 | 0.0734 | 0.0020 | 0.0020 |
| REACTOME_REGULATION_OF_MRNA_{—} | 97 | 0.16 | 0.30 | Up | 0.0670 | 0.0914 | 0.0005 | 0.0005 |
| STABILITY_BY_PROTEINS_THAT_{—} | | | | | | | | |
| BIND_AU_RICH_ELEMENTS | | | | | | | | |
| REACTOME_TGF_BETA_RECEPTOR_{—} | 24 | 0.29 | 0.33 | Up | 0.0680 | 0.0926 | 0.0005 | 0.0005 |
| SIGNALING_IN_EMT_EPITHELIAL_{—} | | | | | | | | |
| TO_MESENCHYMAL_TRANSITION | | | | | | | | |
| REACTOME_MUSCLE_CONTRACTION | 38 | 0.29 | 0.34 | Up | 0.0700 | 0.0949 | 0.0005 | 0.0005 |
| REACTOME_MEMBRANE_TRAFFICKING | 135 | 0.17 | 0.24 | Up | 0.0710 | 0.0961 | 0.0005 | 0.0005 |
| REACTOME_ACTIVATION_OF_ATR_{—} | 33 | 0.15 | 0.39 | Up | 0.0715 | 0.0966 | 0.0010 | 0.0010 |
| IN_RESPONSE_TO_REPLICATION_{—} | | | | | | | | |
| STRESS | | | | | | | | |
| REACTOME_G_ALPHA_S_SIGNALLING_{—} | 54 | 0.35 | 0.41 | Up | 0.0740 | 0.0995 | 0.0005 | 0.0005 |
| EVENTS | | | | | | | | |

TABLE 4
Gene expression analysis for signaling and metabolic pathways in HSC-depleted livers compared to control livers in Biocarta signatures.

| Pathway | NGenes | PropDown | PropUp | Direction | PValue | FDR | PValue.Mixed | FDR.Mixed |
|---|---|---|---|---|---|---|---|---|
| BIOCARTA_ACE2_PATHWAY | 15 | 0.47 | 0.33 | Down | 0.1310 | 0.1663 | 0.0010 | 0.0010 |
| BIOCARTA_ALK_PATHWAY | 38 | 0.39 | 0.13 | Down | 0.0130 | 0.0211 | 0.0010 | 0.0010 |
| BIOCARTA_AMI_PATHWAY | 24 | 0.33 | 0.25 | Down | 0.3560 | 0.4085 | 0.0005 | 0.0005 |
| BIOCARTA_EGFR_SMRTE_PATHWAY | 17 | 0.35 | 0.24 | Down | 0.1310 | 0.1663 | 0.0005 | 0.0005 |
| BIOCARTA_EIF_PATHWAY | 21 | 0.29 | 0.14 | Down | 0.1065 | 0.1381 | 0.0010 | 0.0010 |
| BIOCARTA_EIF2_PATHWAY | 15 | 0.27 | 0.07 | Down | 0.0110 | 0.0183 | 0.0595 | 0.0600 |
| BIOCARTA_EIF4_PATHWAY | 34 | 0.32 | 0.26 | Down | 0.3305 | 0.3828 | 0.0005 | 0.0005 |
| BIOCARTA_INTRINSIC_PATHWAY | 30 | 0.37 | 0.33 | Down | 0.6330 | 0.6769 | 0.0005 | 0.0005 |
| BIOCARTA_MTOR_PATHWAY | 31 | 0.32 | 0.16 | Down | 0.1220 | 0.1563 | 0.0005 | 0.0005 |
| BIOCARTA_NUCLEARRS_PATHWAY | 21 | 0.43 | 0.19 | Down | 0.0005 | 0.0009 | 0.0005 | 0.0005 |
| BIOCARTA_P35ALZHEIMERS_PATHWAY | 15 | 0.20 | 0.27 | Down | 0.2425 | 0.2900 | 0.0005 | 0.0005 |
| BIOCARTA_PITX2_PATHWAY | 20 | 0.15 | 0.20 | Down | 0.9145 | 0.9252 | 0.0005 | 0.0005 |
| BIOCARTA_PLATELETAPP_PATHWAY | 20 | 0.40 | 0.35 | Down | 0.7395 | 0.7726 | 0.0005 | 0.0005 |
| BIOCARTA_PROTEASOME_PATHWAY | 32 | 0.09 | 0.03 | Down | 0.6430 | 0.6853 | 0.2410 | 0.2419 |
| BIOCARTA_PS1_PATHWAY | 25 | 0.24 | 0.20 | Down | 0.0995 | 0.1300 | 0.0010 | 0.0010 |
| BIOCARTA_PTEN_PATHWAY | 22 | 0.45 | 0.27 | Down | 0.7590 | 0.7906 | 0.0005 | 0.0005 |
| BIOCARTA_RARRXR_PATHWAY | 18 | 0.22 | 0.17 | Down | 0.1270 | 0.1618 | 0.0015 | 0.0015 |
| BIOCARTA_SPRY_PATHWAY | 26 | 0.38 | 0.19 | Down | 0.0065 | 0.0116 | 0.0005 | 0.0005 |
| BIOCARTA_TGFB_PATHWAY | 22 | 0.32 | 0.18 | Down | 0.7130 | 0.7479 | 0.0005 | 0.0005 |
| BIOCARTA_VDR_PATHWAY | 16 | 0.25 | 0.13 | Down | 0.1380 | 0.1744 | 0.0010 | 0.0010 |
| BIOCARTA_VITCB_PATHWAY | 17 | 0.47 | 0.29 | Down | 0.4260 | 0.4775 | 0.0005 | 0.0005 |
| BIOCARTA_WNT_PATHWAY | 40 | 0.28 | 0.20 | Down | 0.0675 | 0.0920 | 0.0005 | 0.0005 |
| BIOCARTA_41BB_PATHWAY | 20 | 0.20 | 0.60 | Up | 0.0005 | 0.0009 | 0.0005 | 0.0005 |
| BIOCARTA_ACH_PATHWAY | 16 | 0.31 | 0.50 | Up | 0.0055 | 0.0100 | 0.0005 | 0.0005 |
| BIOCARTA_ACTINY_PATHWAY | 24 | 0.08 | 0.58 | Up | 0.0005 | 0.0009 | 0.0005 | 0.0005 |
| BIOCARTA_AGPCR_PATHWAY | 15 | 0.13 | 0.53 | Up | 0.0005 | 0.0009 | 0.0010 | 0.0010 |
| BIOCARTA_AGR_PATHWAY | 40 | 0.25 | 0.35 | Up | 0.0910 | 0.1202 | 0.0005 | 0.0005 |
| BIOCARTA_AKAP95_PATHWAY | 18 | 0.06 | 0.22 | Up | 0.0230 | 0.0351 | 0.0115 | 0.0116 |
| BIOCARTA_AKAPCENTROSOME_PATHWAY | 23 | 0.09 | 0.22 | Up | 0.2315 | 0.2786 | 0.0005 | 0.0005 |
| BIOCARTA_AKT_PATHWAY | 30 | 0.23 | 0.40 | Up | 0.0035 | 0.0067 | 0.0005 | 0.0005 |
| BIOCARTA_ARAP_PATHWAY | 21 | 0.05 | 0.29 | Up | 0.0050 | 0.0092 | 0.0005 | 0.0005 |
| BIOCARTA_ARENRF2_PATHWAY | 15 | 0.13 | 0.47 | Up | 0.0005 | 0.0009 | 0.0005 | 0.0005 |
| BIOCARTA_ARF_PATHWAY | 22 | 0.14 | 0.55 | Up | 0.0005 | 0.0009 | 0.0005 | 0.0005 |
| BIOCARTA_AT1R_PATHWAY | 47 | 0.26 | 0.34 | Up | 0.0235 | 0.0357 | 0.0005 | 0.0005 |
| BIOCARTA_ATM_PATHWAY | 27 | 0.15 | 0.56 | Up | 0.0005 | 0.0009 | 0.0005 | 0.0005 |
| BIOCARTA_ATRBRCA_PATHWAY | 21 | 0.19 | 0.24 | Up | 0.2900 | 0.3407 | 0.0005 | 0.0005 |
| BIOCARTA_BAD_PATHWAY | 34 | 0.15 | 0.53 | Up | 0.0005 | 0.0009 | 0.0005 | 0.0005 |
| BIOCARTA_BARR_MAPK_PATHWAY | 20 | 0.25 | 0.35 | Up | 0.0205 | 0.0316 | 0.0005 | 0.0005 |
| BIOCARTA_BARRESTIN_PATHWAY | 17 | 0.24 | 0.41 | Up | 0.0080 | 0.0139 | 0.0005 | 0.0005 |
| BIOCARTA_BARRESTIN_SRC_PATHWAY | 28 | 0.25 | 0.46 | Up | 0.0010 | 0.0020 | 0.0005 | 0.0005 |
| BIOCARTA_BCELLSURVIVAL_PATHWAY | 24 | 0.17 | 0.54 | Up | 0.0040 | 0.0075 | 0.0010 | 0.0010 |
| BIOCARTA_BCR_PATHWAY | 49 | 0.18 | 0.51 | Up | 0.0005 | 0.0009 | 0.0005 | 0.0005 |
| BIOCARTA_BIOPEPTIDES_PATHWAY | 58 | 0.22 | 0.48 | Up | 0.0010 | 0.0020 | 0.0005 | 0.0005 |
| BIOCARTA_CALCINEURIN_PATHWAY | 20 | 0.15 | 0.45 | Up | 0.0010 | 0.0020 | 0.0010 | 0.0010 |
| BIOCARTA_CARDIACEGF_PATHWAY | 28 | 0.25 | 0.57 | Up | 0.0010 | 0.0020 | 0.0005 | 0.0005 |
| BIOCARTA_CARM_ER_PATHWAY | 34 | 0.21 | 0.29 | Up | 0.0315 | 0.0462 | 0.0005 | 0.0005 |
| BIOCARTA_CASPASE_PATHWAY | 28 | 0.04 | 0.64 | Up | 0.0005 | 0.0009 | 0.0005 | 0.0005 |
| BIOCARTA_CBL_PATHWAY | 18 | 0.39 | 0.50 | Up | 0.1585 | 0.1983 | 0.0005 | 0.0005 |
| BIOCARTA_CCR3_PATHWAY | 37 | 0.16 | 0.46 | Up | 0.0005 | 0.0009 | 0.0005 | 0.0005 |
| BIOCARTA_CCR5_PATHWAY | 21 | 0.19 | 0.57 | Up | 0.0005 | 0.0009 | 0.0005 | 0.0005 |
| BIOCARTA_CD40_PATHWAY | 18 | 0.11 | 0.67 | Up | 0.0005 | 0.0009 | 0.0005 | 0.0005 |
| BIOCARTA_CDC42RAC_PATHWAY | 24 | 0.13 | 0.63 | Up | 0.0010 | 0.0020 | 0.0005 | 0.0005 |
| BIOCARTA_CDMAC_PATHWAY | 27 | 0.15 | 0.56 | Up | 0.0005 | 0.0009 | 0.0005 | 0.0005 |
| BIOCARTA_CELLCYCLE_PATHWAY | 25 | 0.16 | 0.56 | Up | 0.0025 | 0.0049 | 0.0005 | 0.0005 |
| BIOCARTA_CERAMIDE_PATHWAY | 29 | 0.17 | 0.31 | Up | 0.0030 | 0.0058 | 0.0005 | 0.0005 |
| BIOCARTA_CHEMICAL_PATHWAY | 28 | 0.11 | 0.54 | Up | 0.0005 | 0.0009 | 0.0005 | 0.0005 |
| BIOCARTA_CHREBP2_PATHWAY | 39 | 0.21 | 0.28 | Up | 0.4035 | 0.4556 | 0.0005 | 0.0005 |
| BIOCARTA_CK1_PATHWAY | 21 | 0.19 | 0.19 | Up | 0.3685 | 0.4204 | 0.0005 | 0.0005 |
| BIOCARTA_COMP_PATHWAY | 19 | 0.16 | 0.47 | Up | 0.0075 | 0.0131 | 0.0005 | 0.0005 |
| BIOCARTA_CREB_PATHWAY | 37 | 0.24 | 0.49 | Up | 0.0005 | 0.0009 | 0.0005 | 0.0005 |
| BIOCARTA_CSK_PATHWAY | 26 | 0.12 | 0.58 | Up | 0.0005 | 0.0009 | 0.0005 | 0.0005 |
| BIOCARTA_CTCF_PATHWAY | 29 | 0.21 | 0.41 | Up | 0.0010 | 0.0020 | 0.0005 | 0.0005 |
| BIOCARTA_CTL_PATHWAY | 31 | 0.03 | 0.94 | Up | 0.0005 | 0.0009 | 0.0005 | 0.0005 |
| BIOCARTA_CTLA4_PATHWAY | 24 | 0.04 | 0.83 | Up | 0.0005 | 0.0009 | 0.0005 | 0.0005 |
| BIOCARTA_CXCR4_PATHWAY | 41 | 0.22 | 0.46 | Up | 0.0005 | 0.0009 | 0.0005 | 0.0005 |
| BIOCARTA_D4GDI_PATHWAY | 15 | 0.07 | 0.60 | Up | 0.0005 | 0.0009 | 0.0005 | 0.0005 |
| BIOCARTA_DC_PATHWAY | 37 | 0.11 | 0.68 | Up | 0.0005 | 0.0009 | 0.0005 | 0.0005 |
| BIOCARTA_DEATH_PATHWAY | 41 | 0.10 | 0.59 | Up | 0.0005 | 0.0009 | 0.0005 | 0.0005 |
| BIOCARTA_ECM_PATHWAY | 39 | 0.23 | 0.38 | Up | 0.0030 | 0.0058 | 0.0005 | 0.0005 |
| BIOCARTA_EDG1_PATHWAY | 42 | 0.29 | 0.48 | Up | 0.0015 | 0.0031 | 0.0005 | 0.0005 |
| BIOCARTA_EGF_PATHWAY | 47 | 0.26 | 0.43 | Up | 0.0010 | 0.0020 | 0.0005 | 0.0005 |
| BIOCARTA_EPHA4_PATHWAY | 18 | 0.22 | 0.50 | Up | 0.0010 | 0.0020 | 0.0005 | 0.0005 |
| BIOCARTA_EPO_PATHWAY | 31 | 0.29 | 0.35 | Up | 0.0010 | 0.0020 | 0.0005 | 0.0005 |
| BIOCARTA_EPONFKB_PATHWAY | 15 | 0.00 | 0.60 | Up | 0.0010 | 0.0020 | 0.0015 | 0.0015 |
| BIOCARTA_ERK_PATHWAY | 46 | 0.28 | 0.33 | Up | 0.3120 | 0.3638 | 0.0010 | 0.0010 |
| BIOCARTA_ERK5_PATHWAY | 26 | 0.19 | 0.50 | Up | 0.0005 | 0.0009 | 0.0005 | 0.0005 |
| BIOCARTA_ETS_PATHWAY | 26 | 0.15 | 0.54 | Up | 0.0005 | 0.0009 | 0.0005 | 0.0005 |
| BIOCARTA_FAS_PATHWAY | 30 | 0.13 | 0.43 | Up | 0.0005 | 0.0009 | 0.0005 | 0.0005 |
| BIOCARTA_FCER1_PATHWAY | 53 | 0.19 | 0.51 | Up | 0.0005 | 0.0009 | 0.0005 | 0.0005 |
| BIOCARTA_FMLP_PATHWAY | 51 | 0.12 | 0.55 | Up | 0.0005 | 0.0009 | 0.0005 | 0.0005 |
| BIOCARTA_G1_PATHWAY | 31 | 0.16 | 0.48 | Up | 0.0015 | 0.0031 | 0.0005 | 0.0005 |
| BIOCARTA_G2_PATHWAY | 31 | 0.06 | 0.45 | Up | 0.0005 | 0.0009 | 0.0005 | 0.0005 |
| BIOCARTA_GCR_PATHWAY | 28 | 0.14 | 0.54 | Up | 0.0005 | 0.0009 | 0.0005 | 0.0005 |
| BIOCARTA_GH_PATHWAY | 43 | 0.23 | 0.44 | Up | 0.0015 | 0.0031 | 0.0005 | 0.0005 |
| BIOCARTA_GLEEVEC_PATHWAY | 41 | 0.22 | 0.46 | Up | 0.0005 | 0.0009 | 0.0005 | 0.0005 |
| BIOCARTA_GLYCOLYSIS_PATHWAY | 16 | 0.06 | 0.63 | Up | 0.0010 | 0.0020 | 0.0005 | 0.0005 |
| BIOCARTA_GPCR_PATHWAY | 45 | 0.22 | 0.40 | Up | 0.0005 | 0.0009 | 0.0005 | 0.0005 |
| BIOCARTA_GRANULOCYTES_PATHWAY | 16 | 0.06 | 0.81 | Up | 0.0005 | 0.0009 | 0.0005 | 0.0005 |
| BIOCARTA_GSK3_PATHWAY | 46 | 0.22 | 0.41 | Up | 0.0010 | 0.0020 | 0.0005 | 0.0005 |
| BIOCARTA_HCMV_PATHWAY | 25 | 0.12 | 0.52 | Up | 0.0005 | 0.0009 | 0.0005 | 0.0005 |
| BIOCARTA_HDAC_PATHWAY | 35 | 0.14 | 0.46 | Up | 0.0020 | 0.0040 | 0.0005 | 0.0005 |
| BIOCARTA_HER2_PATHWAY | 32 | 0.25 | 0.38 | Up | 0.0045 | 0.0083 | 0.0005 | 0.0005 |
| BIOCARTA_HIF_PATHWAY | 18 | 0.17 | 0.17 | Up | 0.3780 | 0.4298 | 0.0035 | 0.0035 |
| BIOCARTA_HIVNEF_PATHWAY | 71 | 0.07 | 0.54 | Up | 0.0005 | 0.0009 | 0.0005 | 0.0005 |
| BIOCARTA_HSP27_PATHWAY | 16 | 0.00 | 0.69 | Up | 0.0010 | 0.0020 | 0.0005 | 0.0005 |
| BIOCARTA_IGF1_PATHWAY | 35 | 0.29 | 0.34 | Up | 0.0070 | 0.0123 | 0.0005 | 0.0005 |
| BIOCARTA_IGF1MTOR_PATHWAY | 30 | 0.23 | 0.20 | Up | 0.8225 | 0.8448 | 0.0005 | 0.0005 |
| BIOCARTA_IGF1R_PATHWAY | 36 | 0.28 | 0.33 | Up | 0.1830 | 0.2256 | 0.0005 | 0.0005 |
| BIOCARTA_IL10_PATHWAY | 17 | 0.00 | 0.76 | Up | 0.0005 | 0.0009 | 0.0005 | 0.0005 |
| BIOCARTA_IL12_PATHWAY | 24 | 0.04 | 0.79 | Up | 0.0005 | 0.0009 | 0.0005 | 0.0005 |
| BIOCARTA_IL1R_PATHWAY | 34 | 0.15 | 0.56 | Up | 0.0010 | 0.0020 | 0.0010 | 0.0010 |
| BIOCARTA_IL2_PATHWAY | 34 | 0.24 | 0.47 | Up | 0.0005 | 0.0009 | 0.0005 | 0.0005 |
| BIOCARTA_IL2RB_PATHWAY | 53 | 0.15 | 0.49 | Up | 0.0005 | 0.0009 | 0.0005 | 0.0005 |
| BIOCARTA_IL3_PATHWAY | 26 | 0.19 | 0.46 | Up | 0.0005 | 0.0009 | 0.0005 | 0.0005 |
| BIOCARTA_IL4_PATHWAY | 16 | 0.13 | 0.50 | Up | 0.0010 | 0.0020 | 0.0005 | 0.0005 |
| BIOCARTA_IL6_PATHWAY | 32 | 0.22 | 0.53 | Up | 0.0015 | 0.0031 | 0.0005 | 0.0005 |
| BIOCARTA_IL7_PATHWAY | 25 | 0.08 | 0.72 | Up | 0.0005 | 0.0009 | 0.0005 | 0.0005 |
| BIOCARTA_INSULIN_PATHWAY | 35 | 0.29 | 0.37 | Up | 0.0010 | 0.0020 | 0.0005 | 0.0005 |
| BIOCARTA_INTEGRIN_PATHWAY | 57 | 0.21 | 0.40 | Up | 0.0005 | 0.0009 | 0.0005 | 0.0005 |
| BIOCARTA_KERATINOCYTE_PATHWAY | 56 | 0.14 | 0.50 | Up | 0.0005 | 0.0009 | 0.0010 | 0.0010 |
| BIOCARTA_LAIR_PATHWAY | 19 | 0.05 | 0.79 | Up | 0.0005 | 0.0009 | 0.0005 | 0.0005 |
| BIOCARTA_LONGEVITY_PATHWAY | 21 | 0.38 | 0.38 | Up | 0.7245 | 0.7590 | 0.0005 | 0.0005 |
| BIOCARTA_MAL_PATHWAY | 34 | 0.18 | 0.41 | Up | 0.0005 | 0.0009 | 0.0005 | 0.0005 |
| BIOCARTA_MAPK_PATHWAY | 94 | 0.16 | 0.43 | Up | 0.0005 | 0.0009 | 0.0005 | 0.0005 |
| BIOCARTA_MCALPAIN_PATHWAY | 33 | 0.27 | 0.36 | Up | 0.0140 | 0.0226 | 0.0005 | 0.0005 |
| BIOCARTA_MCM_PATHWAY | 17 | 0.06 | 0.71 | Up | 0.0015 | 0.0031 | 0.0010 | 0.0010 |
| BIOCARTA_MEF2D_PATHWAY | 21 | 0.10 | 0.43 | Up | 0.0005 | 0.0009 | 0.0005 | 0.0005 |
| BIOCARTA_MET_PATHWAY | 55 | 0.22 | 0.44 | Up | 0.0010 | 0.0020 | 0.0005 | 0.0005 |
| BIOCARTA_MITOCHONDRIA_PATHWAY | 25 | 0.12 | 0.48 | Up | 0.0005 | 0.0009 | 0.0005 | 0.0005 |
| BIOCARTA_MPR_PATHWAY | 41 | 0.20 | 0.54 | Up | 0.0025 | 0.0049 | 0.0005 | 0.0005 |
| BIOCARTA_MTA3_PATHWAY | 23 | 0.17 | 0.43 | Up | 0.0720 | 0.0972 | 0.0005 | 0.0005 |
| BIOCARTA_MYOSIN_PATHWAY | 40 | 0.20 | 0.38 | Up | 0.0865 | 0.1148 | 0.0005 | 0.0005 |
| BIOCARTA_NDKDYNAMIN_PATHWAY | 18 | 0.17 | 0.28 | Up | 0.1795 | 0.2219 | 0.0020 | 0.0020 |
| BIOCARTA_NFAT_PATHWAY | 64 | 0.23 | 0.33 | Up | 0.0060 | 0.0108 | 0.0005 | 0.0005 |
| BIOCARTA_NFKB_PATHWAY | 32 | 0.19 | 0.59 | Up | 0.0005 | 0.0009 | 0.0005 | 0.0005 |
| BIOCARTA_NGF_PATHWAY | 30 | 0.40 | 0.33 | Up | 0.0275 | 0.0410 | 0.0005 | 0.0005 |
| BIOCARTA_NKCELLS_PATHWAY | 44 | 0.09 | 0.73 | Up | 0.0005 | 0.0009 | 0.0005 | 0.0005 |
| BIOCARTA_NKT_PATHWAY | 16 | 0.00 | 1.00 | Up | 0.0005 | 0.0009 | 0.0005 | 0.0005 |
| BIOCARTA_NO1_PATHWAY | 30 | 0.17 | 0.40 | Up | 0.1235 | 0.1580 | 0.0005 | 0.0005 |
| BIOCARTA_NO2IL12_PATHWAY | 17 | 0.00 | 0.94 | Up | 0.0005 | 0.0009 | 0.0005 | 0.0005 |
| BIOCARTA_NOS1_PATHWAY | 16 | 0.25 | 0.38 | Up | 0.0100 | 0.0168 | 0.0005 | 0.0005 |
| BIOCARTA_NTHI_PATHWAY | 35 | 0.23 | 0.51 | Up | 0.0005 | 0.0009 | 0.0005 | 0.0005 |
| BIOCARTA_P27_PATHWAY | 15 | 0.07 | 0.53 | Up | 0.0715 | 0.0966 | 0.0135 | 0.0137 |
| BIOCARTA_P38MAPK_PATHWAY | 51 | 0.06 | 0.47 | Up | 0.0005 | 0.0009 | 0.0005 | 0.0005 |
| BIOCARTA_P53_PATHWAY | 19 | 0.11 | 0.58 | Up | 0.0005 | 0.0009 | 0.0005 | 0.0005 |
| BIOCARTA_P53HYPOXIA_PATHWAY | 37 | 0.16 | 0.27 | Up | 0.2155 | 0.2613 | 0.0005 | 0.0005 |
| BIOCARTA_PAR1_PATHWAY | 50 | 0.20 | 0.44 | Up | 0.0025 | 0.0049 | 0.0005 | 0.0005 |
| BIOCARTA_PDGF_PATHWAY | 47 | 0.26 | 0.45 | Up | 0.0005 | 0.0009 | 0.0005 | 0.0005 |
| BIOCARTA_PGC1A_PATHWAY | 24 | 0.17 | 0.38 | Up | 0.0005 | 0.0009 | 0.0005 | 0.0005 |
| BIOCARTA_PML_PATHWAY | 20 | 0.10 | 0.50 | Up | 0.0005 | 0.0009 | 0.0005 | 0.0005 |
| BIOCARTA_PPARA_PATHWAY | 70 | 0.23 | 0.31 | Up | 0.0020 | 0.0040 | 0.0005 | 0.0005 |
| BIOCARTA_PTDINS_PATHWAY | 39 | 0.23 | 0.41 | Up | 0.0005 | 0.0009 | 0.0005 | 0.0005 |
| BIOCARTA_PYK2_PATHWAY | 41 | 0.24 | 0.39 | Up | 0.0010 | 0.0020 | 0.0005 | 0.0005 |
| BIOCARTA_RAB_PATHWAY | 22 | 0.18 | 0.36 | Up | 0.0025 | 0.0049 | 0.0005 | 0.0005 |
| BIOCARTA_RAC1_PATHWAY | 31 | 0.29 | 0.52 | Up | 0.0005 | 0.0009 | 0.0005 | 0.0005 |
| BIOCARTA_RACCYCD_PATHWAY | 43 | 0.21 | 0.53 | Up | 0.0005 | 0.0009 | 0.0005 | 0.0005 |
| BIOCARTA_RANKL_PATHWAY | 15 | 0.07 | 0.67 | Up | 0.0005 | 0.0009 | 0.0005 | 0.0005 |
| BIOCARTA_RAS_PATHWAY | 42 | 0.19 | 0.48 | Up | 0.0010 | 0.0020 | 0.0005 | 0.0005 |
| BIOCARTA_RB_PATHWAY | 15 | 0.13 | 0.47 | Up | 0.0075 | 0.0131 | 0.0005 | 0.0005 |
| BIOCARTA_RELA_PATHWAY | 19 | 0.16 | 0.47 | Up | 0.0010 | 0.0020 | 0.0005 | 0.0005 |
| BIOCARTA_RHO_PATHWAY | 43 | 0.19 | 0.56 | Up | 0.0005 | 0.0009 | 0.0005 | 0.0005 |
| BIOCARTA_SALMONELLA_PATHWAY | 17 | 0.06 | 0.65 | Up | 0.0015 | 0.0031 | 0.0005 | 0.0005 |
| BIOCARTA_SODD_PATHWAY | 15 | 0.07 | 0.73 | Up | 0.0005 | 0.0009 | 0.0005 | 0.0005 |
| BIOCARTA_SPPA_PATHWAY | 33 | 0.18 | 0.42 | Up | 0.0010 | 0.0020 | 0.0005 | 0.0005 |
| BIOCARTA_STATHMIN_PATHWAY | 17 | 0.06 | 0.65 | Up | 0.0005 | 0.0009 | 0.0005 | 0.0005 |
| BIOCARTA_STRESS_PATHWAY | 32 | 0.13 | 0.56 | Up | 0.0005 | 0.0009 | 0.0005 | 0.0005 |
| BIOCARTA_TALL1_PATHWAY | 19 | 0.11 | 0.68 | Up | 0.0005 | 0.0009 | 0.0005 | 0.0005 |
| BIOCARTA_TCR_PATHWAY | 66 | 0.17 | 0.58 | Up | 0.0005 | 0.0009 | 0.0005 | 0.0005 |
| BIOCARTA_TCRA_PATHWAY | 17 | 0.06 | 0.88 | Up | 0.0005 | 0.0009 | 0.0005 | 0.0005 |
| BIOCARTA_TEL_PATHWAY | 32 | 0.25 | 0.38 | Up | 0.0535 | 0.0746 | 0.0005 | 0.0005 |
| BIOCARTA_TFF_PATHWAY | 33 | 0.27 | 0.33 | Up | 0.0240 | 0.0364 | 0.0005 | 0.0005 |
| BIOCARTA_TID_PATHWAY | 27 | 0.11 | 0.52 | Up | 0.0005 | 0.0009 | 0.0010 | 0.0010 |
| BIOCARTA_TNFR1_PATHWAY | 32 | 0.13 | 0.47 | Up | 0.0005 | 0.0009 | 0.0005 | 0.0005 |
| BIOCARTA_TNFR2_PATHWAY | 21 | 0.10 | 0.76 | Up | 0.0005 | 0.0009 | 0.0005 | 0.0005 |
| BIOCARTA_TOB1_PATHWAY | 16 | 0.13 | 0.56 | Up | 0.0005 | 0.0009 | 0.0005 | 0.0005 |
| BIOCARTA_TOLL_PATHWAY | 61 | 0.16 | 0.52 | Up | 0.0005 | 0.0009 | 0.0005 | 0.0005 |
| BIOCARTA_TPO_PATHWAY | 37 | 0.24 | 0.46 | Up | 0.0010 | 0.0020 | 0.0010 | 0.0010 |
| BIOCARTA_TRKA_PATHWAY | 19 | 0.37 | 0.53 | Up | 0.0020 | 0.0040 | 0.0005 | 0.0005 |
| BIOCARTA_UCALPAIN_PATHWAY | 29 | 0.14 | 0.52 | Up | 0.0005 | 0.0009 | 0.0005 | 0.0005 |
| BIOCARTA_VEGF_PATHWAY | 39 | 0.18 | 0.36 | Up | 0.0005 | 0.0009 | 0.0025 | 0.0025 |
| BIOCARTA_VIP_PATHWAY | 29 | 0.17 | 0.45 | Up | 0.0005 | 0.0009 | 0.0005 | 0.0005 |

TABLE 5
Gene expression analysis for signaling and metabolic pathways in HSC-depleted livers compared to control livers in KEGG signatures.

| Pathway | NGenes | PropDown | PropUp | Direction |
|---|---|---|---|---|
| KEGG_ALANINE_ASPARTATE_AND_GLUTAMATE_METABOLISM | 29 | 0.55 | 0.14 | Down |
| KEGG_ARGININE_AND_PROLINE_METABOLISM | 49 | 0.51 | 0.20 | Down |
| KEGG_ASCORBATE_AND_ALDARATE_METABOLISM | 21 | 0.67 | 0.10 | Down |
| KEGG_BASAL_CELL_CARCINOMA | 34 | 0.44 | 0.09 | Down |
| KEGG_BETA_ALANINE_METABOLISM | 21 | 0.62 | 0.05 | Down |
| KEGG_BIOSYNTHESIS_OF_UNSATURATED_FATTY_ACIDS | 27 | 0.52 | 0.07 | Down |
| KEGG_BUTANOATE_METABOLISM | 38 | 0.68 | 0.16 | Down |
| KEGG_DRUG_METABOLISM_CYTOCHROME_P450 | 90 | 0.43 | 0.11 | Down |
| KEGG_FATTY_ACID_METABOLISM | 47 | 0.72 | 0.02 | Down |
| KEGG_LYSINE_DEGRADATION | 44 | 0.48 | 0.09 | Down |
| KEGG_METABOLISM_OF_XENOBIOTICS_BY_CYTOCHROME_P450 | 82 | 0.51 | 0.10 | Down |
| KEGG_PROPANOATE_METABOLISM | 32 | 0.63 | 0.09 | Down |
| KEGG_RETINOL_METABOLISM | 77 | 0.45 | 0.10 | Down |
| KEGG_VALINE_LEUCINE_AND_ISOLEUCINE_DEGRADATION | 50 | 0.78 | 0.08 | Down |
| KEGG_CITRATE_CYCLE_TCA_CYCLE | 29 | 0.62 | 0.07 | Down |
| KEGG_GLYOXYLATE_AND_DICARBOXYLATE_METABOLISM | 16 | 0.63 | 0.13 | Down |
| KEGG_TRYPTOPHAN_METABOLISM | 37 | 0.57 | 0.08 | Down |
| KEGG_TYROSINE_METABOLISM | 36 | 0.50 | 0.11 | Down |
| KEGG_STEROID_HORMONE_BIOSYNTHESIS | 60 | 0.52 | 0.18 | Down |
| KEGG_ARACHIDONIC_ACID_METABOLISM | 61 | 0.43 | 0.21 | Down |
| KEGG_PEROXISOME | 81 | 0.59 | 0.12 | Down |
| KEGG_PRIMARY_BILE_ACID_BIOSYNTHESIS | 22 | 0.68 | 0.18 | Down |
| KEGG_PENTOSE_AND_GLUCURONATE_INTERCONVERSIONS | 26 | 0.62 | 0.27 | Down |
| KEGG_PHENYLALANINE_METABOLISM | 19 | 0.47 | 0.11 | Down |
| KEGG_PPAR_SIGNALING_PATHWAY | 65 | 0.46 | 0.20 | Down |
| KEGG_REGULATION_OF_AUTOPHAGY | 22 | 0.27 | 0.14 | Down |
| KEGG_HEDGEHOG_SIGNALING_PATHWAY | 35 | 0.40 | 0.14 | Down |
| KEGG_DRUG_METABOLISM_OTHER_ENZYMES | 61 | 0.41 | 0.20 | Down |
| KEGG_AMINOACYL_TRNA_BIOSYNTHESIS | 41 | 0.46 | 0.05 | Down |
| KEGG_LINOLEIC_ACID_METABOLISM | 37 | 0.41 | 0.22 | Down |
| KEGG_GLYCINE_SERINE_AND_THREONINE_METABOLISM | 31 | 0.48 | 0.23 | Down |
| KEGG_PANTOTHENATE_AND_COA_BIOSYNTHESIS | 18 | 0.33 | 0.17 | Down |
| KEGG_NITROGEN_METABOLISM | 19 | 0.42 | 0.21 | Down |
| KEGG_HISTIDINE_METABOLISM | 27 | 0.37 | 0.15 | Down |
| KEGG_ALZHEIMERS_DISEASE | 151 | 0.54 | 0.19 | Down |
| KEGG_RIBOFLAVIN_METABOLISM | 17 | 0.41 | 0.24 | Down |
| KEGG_PARKINSONS_DISEASE | 114 | 0.63 | 0.11 | Down |
| KEGG_PYRUVATE_METABOLISM | 43 | 0.37 | 0.28 | Down |
| KEGG_PORPHYRIN_AND_CHLOROPHYLL_METABOLISM | 36 | 0.53 | 0.22 | Down |
| KEGG_OXIDATIVE_PHOSPHORYLATION | 116 | 0.66 | 0.08 | Down |
| KEGG_SELENOAMINO_ACID_METABOLISM | 26 | 0.31 | 0.12 | Down |
| KEGG_HUNTINGTONS_DISEASE | 174 | 0.49 | 0.16 | Down |
| KEGG_ACUTE_MYELOID_LEUKEMIA | 68 | 0.22 | 0.46 | Up |
| KEGG_ALLOGRAFT_REJECTION | 34 | 0.03 | 0.97 | Up |
| KEGG_ANTIGEN_PROCESSING_AND_PRESENTATION | 68 | 0.07 | 0.68 | Up |
| KEGG_APOPTOSIS | 90 | 0.17 | 0.52 | Up |
| KEGG_AUTOIMMUNE_THYROID_DISEASE | 33 | 0.03 | 0.97 | Up |
| KEGG_B_CELL_RECEPTOR_SIGNALING_PATHWAY | 94 | 0.17 | 0.59 | Up |
| KEGG_CELL_ADHESION_MOLECULES_CAMS | 106 | 0.19 | 0.69 | Up |
| KEGG_CHEMOKINE_SIGNALING_PATHWAY | 165 | 0.18 | 0.56 | Up |
| KEGG_CHRONIC_MYELOID_LEUKEMIA | 78 | 0.21 | 0.46 | Up |
| KEGG_COLORECTAL_CANCER | 70 | 0.21 | 0.34 | Up |
| KEGG_CYTOKINE_CYTOKINE_RECEPTOR_INTERACTION | 141 | 0.18 | 0.66 | Up |
| KEGG_CYTOSOLIC_DNA_SENSING_PATHWAY | 41 | 0.20 | 0.54 | Up |
| KEGG_ENDOCYTOSIS | 187 | 0.18 | 0.43 | Up |
| KEGG_EPITHELIAL_CELL_SIGNALING_IN_HELICOBACTER_PYLORI_INFECTION | 75 | 0.17 | 0.49 | Up |
| KEGG_ETHER_LIPID_METABOLISM | 23 | 0.26 | 0.61 | Up |
| KEGG_FC_EPSILON_RI_SIGNALING_PATHWAY | 68 | 0.21 | 0.50 | Up |
| KEGG_FC_GAMMA_R_MEDIATED_PHAGOCYTOSIS | 95 | 0.13 | 0.57 | Up |
| KEGG_FRUCTOSE_AND_MANNOSE_METABOLISM | 37 | 0.22 | 0.43 | Up |
| KEGG_GLYCOSAMINOGLYCAN_BIOSYNTHESIS_CHONDROITIN_SULFATE | 20 | 0.05 | 0.55 | Up |
| KEGG_GLYCOSAMINOGLYCAN_DEGRADATION | 19 | 0.05 | 0.53 | Up |
| KEGG_GRAFT_VERSUS_HOST_DISEASE | 37 | 0.03 | 0.97 | Up |
| KEGG_HEMATOPOIETIC_CELL_LINEAGE | 67 | 0.07 | 0.81 | Up |
| KEGG_INTESTINAL_IMMUNE_NETWORK_FOR_IGA_PRODUCTION | 33 | 0.09 | 0.79 | Up |
| KEGG_JAK_STAT_SIGNALING_PATHWAY | 94 | 0.21 | 0.56 | Up |
| KEGG_LEISHMANIA_INFECTION | 77 | 0.06 | 0.78 | Up |
| KEGG_LEUKOCYTE_TRANSENDOTHELIAL_MIGRATION | 100 | 0.19 | 0.59 | Up |
| KEGG_NATURAL_KILLER_CELL_MEDIATED_CYTOTOXICITY | 110 | 0.14 | 0.71 | Up |
| KEGG_NOD_LIKE_RECEPTOR_SIGNALING_PATHWAY | 56 | 0.14 | 0.57 | Up |
| KEGG_OLFACTORY_TRANSDUCTION | 20 | 0.20 | 0.55 | Up |
| KEGG_P53_SIGNALING_PATHWAY | 68 | 0.16 | 0.47 | Up |
| KEGG_PANCREATIC_CANCER | 79 | 0.27 | 0.43 | Up |
| KEGG_PATHOGENIC_ESCHERICHIA_COLI_INFECTION | 81 | 0.12 | 0.59 | Up |
| KEGG_PENTOSE_PHOSPHATE_PATHWAY | 25 | 0.20 | 0.44 | Up |
| KEGG_PHOSPHATIDYLINOSITOL_SIGNALING_SYSTEM | 64 | 0.20 | 0.42 | Up |
| KEGG_PRIMARY_IMMUNODEFICIENCY | 34 | 0.03 | 0.74 | Up |
| KEGG_REGULATION_OF_ACTIN_CYTOSKELETON | 172 | 0.19 | 0.46 | Up |
| KEGG_RIG_I_LIKE_RECEPTOR_SIGNALING_PATHWAY | 51 | 0.14 | 0.49 | Up |
| KEGG_SYSTEMIC_LUPUS_ERYTHEMATOSUS | 55 | 0.09 | 0.62 | Up |
| KEGG_T_CELL_RECEPTOR_SIGNALING_PATHWAY | 118 | 0.19 | 0.60 | Up |
| KEGG_TOLL_LIKE_RECEPTOR_SIGNALING_PATHWAY | 104 | 0.12 | 0.57 | Up |
| KEGG_TYPE_I_DIABETES_MELLITUS | 37 | 0.05 | 0.92 | Up |
| KEGG_VASOPRESSIN_REGULATED_WATER_REABSORPTION | 43 | 0.21 | 0.37 | Up |
| KEGG_VIRAL_MYOCARDITIS | 71 | 0.11 | 0.72 | Up |
| KEGG_ERBB_SIGNALING_PATHWAY | 75 | 0.23 | 0.41 | Up |
| KEGG_GALACTOSE_METABOLISM | 32 | 0.16 | 0.47 | Up |
| KEGG_LYSOSOME | 118 | 0.11 | 0.47 | Up |
| KEGG_NEUROTROPHIN_SIGNALING_PATHWAY | 126 | 0.19 | 0.46 | Up |
| KEGG_O_GLYCAN_BIOSYNTHESIS | 17 | 0.06 | 0.65 | Up |
| KEGG_PRION_DISEASES | 43 | 0.26 | 0.40 | Up |
| KEGG_SPHINGOLIPID_METABOLISM | 37 | 0.11 | 0.46 | Up |
| KEGG_TYPE_II_DIABETES_MELLITUS | 43 | 0.26 | 0.47 | Up |
| KEGG_VEGF_SIGNALING_PATHWAY | 64 | 0.22 | 0.45 | Up |
| KEGG_ADIPOCYTOKINE_SIGNALING_PATHWAY | 68 | 0.25 | 0.51 | Up |
| KEGG_AMINO_SUGAR_AND_NUCLEOTIDE_SUGAR_METABOLISM | 44 | 0.20 | 0.36 | Up |
| KEGG_GLIOMA | 60 | 0.30 | 0.38 | Up |
| KEGG_INOSITOL_PHOSPHATE_METABOLISM | 49 | 0.22 | 0.37 | Up |
| KEGG_MAPK_SIGNALING_PATHWAY | 202 | 0.20 | 0.43 | Up |
| KEGG_PROXIMAL_TUBULE_BICARBONATE_RECLAMATION | 19 | 0.26 | 0.47 | Up |
| KEGG_RENAL_CELL_CARCINOMA | 67 | 0.28 | 0.34 | Up |
| KEGG_AMYOTROPHIC_LATERAL_SCLEROSIS_ALS | 45 | 0.24 | 0.38 | Up |
| KEGG_GLYCEROPHOSPHOLIPID_METABOLISM | 56 | 0.27 | 0.39 | Up |
| KEGG_PATHWAYS_IN_CANCER | 305 | 0.28 | 0.39 | Up |
| KEGG_GLYCOLYSIS_GLUCONEOGENESIS | 51 | 0.27 | 0.39 | Up |
| KEGG_OTHER_GLYCAN_DEGRADATION | 16 | 0.00 | 0.63 | Up |
| KEGG_CELL_CYCLE | 119 | 0.15 | 0.46 | Up |
| KEGG_GNRH_SIGNALING_PATHWAY | 80 | 0.29 | 0.40 | Up |
| KEGG_NICOTINATE_AND_NICOTINAMIDE_METABOLISM | 24 | 0.38 | 0.38 | Up |
| KEGG_NON_SMALL_CELL_LUNG_CANCER | 57 | 0.32 | 0.37 | Up |
| KEGG_PROSTATE_CANCER | 96 | 0.23 | 0.31 | Up |
| KEGG_DILATED_CARDIOMYOPATHY | 55 | 0.22 | 0.51 | Up |
| KEGG_CALCIUM_SIGNALING_PATHWAY | 93 | 0.29 | 0.37 | Up |
| KEGG_OOCYTE_MEIOSIS | 95 | 0.20 | 0.35 | Up |
| KEGG_LONG_TERM_POTENTIATION | 59 | 0.20 | 0.36 | Up |
| KEGG_VIBRIO_CHOLERAE_INFECTION | 49 | 0.12 | 0.33 | Up |
| KEGG_HYPERTROPHIC_CARDIOMYOPATHY_HCM | 53 | 0.17 | 0.51 | Up |
| KEGG_PROGESTERONE_MEDIATED_OOCYTE_MATURATION | 74 | 0.23 | 0.38 | Up |
| KEGG_SMALL_CELL_LUNG_CANCER | 102 | 0.29 | 0.46 | Up |
| KEGG_SNARE_INTERACTIONS_IN_VESICULAR_TRANSPORT | 32 | 0.19 | 0.31 | Up |
| KEGG_COMPLEMENT_AND_COAGULATION_CASCADES | 75 | 0.19 | 0.40 | Up |
| KEGG_ARRHYTHMOGENIC_RIGHT_VENTRICULAR_CARDIOMYOPATHY_ARVC | 48 | 0.21 | 0.40 | Up |
| KEGG_ALDOSTERONE_REGULATED_SODIUM_REABSORPTION | 34 | 0.26 | 0.41 | Up |
| KEGG_NEUROACTIVE_LIGAND_RECEPTOR_INTERACTION | 84 | 0.37 | 0.39 | Up |
| KEGG_ADHERENS_JUNCTION | 90 | 0.26 | 0.28 | Up |
| KEGG_TASTE_TRANSDUCTION | 27 | 0.30 | 0.41 | Up |
| KEGG_FOCAL_ADHESION | 188 | 0.28 | 0.38 | Up |
| KEGG_MELANOMA | 59 | 0.31 | 0.36 | Up |
| KEGG_MELANOGENESIS | 75 | 0.28 | 0.31 | Up |
| KEGG_SULFUR_METABOLISM | 16 | 0.13 | 0.44 | Up |
| KEGG_INSULIN_SIGNALING_PATHWAY | 130 | 0.27 | 0.33 | Up |
| KEGG_DNA_REPLICATION | 35 | 0.09 | 0.51 | Up |
| KEGG_GAP_JUNCTION | 76 | 0.33 | 0.37 | Up |
| KEGG_PURINE_METABOLISM | 136 | 0.26 | 0.29 | Up |
| KEGG_AXON_GUIDANCE | 105 | 0.30 | 0.41 | Up |
| KEGG_BASE_EXCISION_REPAIR | 31 | 0.19 | 0.42 | Up |

| Pathway | PValue | FDR | PValue.Mixed | FDR.Mixed |
|---|---|---|---|---|
| KEGG_ALANINE_ASPARTATE_AND_GLUTAMATE_METABOLISM | 0.0005 | 0.0009 | 0.0005 | 0.0005 |
| KEGG_ARGININE_AND_PROLINE_METABOLISM | 0.0005 | 0.0009 | 0.0005 | 0.0005 |
| KEGG_ASCORBATE_AND_ALDARATE_METABOLISM | 0.0005 | 0.0009 | 0.0005 | 0.0005 |
| KEGG_BASAL_CELL_CARCINOMA | 0.0005 | 0.0009 | 0.0005 | 0.0005 |
| KEGG_BETA_ALANINE_METABOLISM | 0.0005 | 0.0009 | 0.0005 | 0.0005 |
| KEGG_BIOSYNTHESIS_OF_UNSATURATED_FATTY_ACIDS | 0.0005 | 0.0009 | 0.0005 | 0.0005 |
| KEGG_BUTANOATE_METABOLISM | 0.0005 | 0.0009 | 0.0005 | 0.0005 |
| KEGG_DRUG_METABOLISM_CYTOCHROME_P450 | 0.0005 | 0.0009 | 0.0005 | 0.0005 |
| KEGG_FATTY_ACID_METABOLISM | 0.0005 | 0.0009 | 0.0005 | 0.0005 |
| KEGG_LYSINE_DEGRADATION | 0.0005 | 0.0009 | 0.0005 | 0.0005 |
| KEGG_METABOLISM_OF_XENOBIOTICS_BY_CYTOCHROME_P450 | 0.0005 | 0.0009 | 0.0005 | 0.0005 |
| KEGG_PROPANOATE_METABOLISM | 0.0005 | 0.0009 | 0.0005 | 0.0005 |
| KEGG_RETINOL_METABOLISM | 0.0005 | 0.0009 | 0.0005 | 0.0005 |
| KEGG_VALINE_LEUCINE_AND_ISOLEUCINE_DEGRADATION | 0.0005 | 0.0009 | 0.0005 | 0.0005 |
| KEGG_CITRATE_CYCLE_TCA_CYCLE | 0.0010 | 0.0020 | 0.0005 | 0.0005 |
| KEGG_GLYOXYLATE_AND_DICARBOXYLATE_METABOLISM | 0.0010 | 0.0020 | 0.0005 | 0.0005 |
| KEGG_TRYPTOPHAN_METABOLISM | 0.0010 | 0.0020 | 0.0005 | 0.0005 |
| KEGG_TYROSINE_METABOLISM | 0.0010 | 0.0020 | 0.0005 | 0.0005 |
| KEGG_STEROID_HORMONE_BIOSYNTHESIS | 0.0010 | 0.0020 | 0.0010 | 0.0010 |
| KEGG_ARACHIDONIC_ACID_METABOLISM | 0.0015 | 0.0031 | 0.0005 | 0.0005 |
| KEGG_PEROXISOME | 0.0015 | 0.0031 | 0.0010 | 0.0010 |
| KEGG_PRIMARY_BILE_ACID_BIOSYNTHESIS | 0.0020 | 0.0040 | 0.0010 | 0.0010 |
| KEGG_PENTOSE_AND_GLUCURONATE_INTERCONVERSIONS | 0.0030 | 0.0058 | 0.0005 | 0.0005 |
| KEGG_PHENYLALANINE_METABOLISM | 0.0035 | 0.0067 | 0.0005 | 0.0005 |
| KEGG_PPAR_SIGNALING_PATHWAY | 0.0035 | 0.0067 | 0.0005 | 0.0005 |
| KEGG_REGULATION_OF_AUTOPHAGY | 0.0055 | 0.0100 | 0.0015 | 0.0015 |
| KEGG_HEDGEHOG_SIGNALING_PATHWAY | 0.0070 | 0.0123 | 0.0005 | 0.0005 |
| KEGG_DRUG_METABOLISM_OTHER_ENZYMES | 0.0105 | 0.0175 | 0.0005 | 0.0005 |
| KEGG_AMINOACYL_TRNA_BIOSYNTHESIS | 0.0130 | 0.0211 | 0.0005 | 0.0005 |
| KEGG_LINOLEIC_ACID_METABOLISM | 0.0145 | 0.0233 | 0.0005 | 0.0005 |
| KEGG_GLYCINE_SERINE_AND_THREONINE_METABOLISM | 0.0175 | 0.0276 | 0.0005 | 0.0005 |
| KEGG_PANTOTHENATE_AND_COA_BIOSYNTHESIS | 0.0250 | 0.0377 | 0.0015 | 0.0015 |
| KEGG_NITROGEN_METABOLISM | 0.0255 | 0.0384 | 0.0005 | 0.0005 |
| KEGG_HISTIDINE_METABOLISM | 0.0295 | 0.0437 | 0.0005 | 0.0005 |
| KEGG_ALZHEIMERS_DISEASE | 0.0300 | 0.0443 | 0.0020 | 0.0020 |
| KEGG_RIBOFLAVIN_METABOLISM | 0.0305 | 0.0449 | 0.0005 | 0.0005 |
| KEGG_PARKINSONS_DISEASE | 0.0335 | 0.0488 | 0.0055 | 0.0055 |
| KEGG_PYRUVATE_METABOLISM | 0.0355 | 0.0514 | 0.0005 | 0.0005 |
| KEGG_PORPHYRIN_AND_CHLOROPHYLL_METABOLISM | 0.0385 | 0.0554 | 0.0005 | 0.0005 |
| KEGG_OXIDATIVE_PHOSPHORYLATION | 0.0390 | 0.0561 | 0.0055 | 0.0055 |
| KEGG_SELENOAMINO_ACID_METABOLISM | 0.0595 | 0.0820 | 0.0005 | 0.0005 |
| KEGG_HUNTINGTONS_DISEASE | 0.0710 | 0.0961 | 0.0030 | 0.0030 |
| KEGG_ACUTE_MYELOID_LEUKEMIA | 0.0005 | 0.0009 | 0.0005 | 0.0005 |
| KEGG_ALLOGRAFT_REJECTION | 0.0005 | 0.0009 | 0.0005 | 0.0005 |
| KEGG_ANTIGEN_PROCESSING_AND_PRESENTATION | 0.0005 | 0.0009 | 0.0005 | 0.0005 |
| KEGG_APOPTOSIS | 0.0005 | 0.0009 | 0.0005 | 0.0005 |
| KEGG_AUTOIMMUNE_THYROID_DISEASE | 0.0005 | 0.0009 | 0.0005 | 0.0005 |
| KEGG_B_CELL_RECEPTOR_SIGNALING_PATHWAY | 0.0005 | 0.0009 | 0.0005 | 0.0005 |
| KEGG_CELL_ADHESION_MOLECULES_CAMS | 0.0005 | 0.0009 | 0.0005 | 0.0005 |
| KEGG_CHEMOKINE_SIGNALING_PATHWAY | 0.0005 | 0.0009 | 0.0005 | 0.0005 |
| KEGG_CHRONIC_MYELOID_LEUKEMIA | 0.0005 | 0.0009 | 0.0005 | 0.0005 |
| KEGG_COLORECTAL_CANCER | 0.0005 | 0.0009 | 0.0005 | 0.0005 |
| KEGG_CYTOKINE_CYTOKINE_RECEPTOR_INTERACTION | 0.0005 | 0.0009 | 0.0005 | 0.0005 |
| KEGG_CYTOSOLIC_DNA_SENSING_PATHWAY | 0.0005 | 0.0009 | 0.0005 | 0.0005 |
| KEGG_ENDOCYTOSIS | 0.0005 | 0.0009 | 0.0005 | 0.0005 |
| KEGG_EPITHELIAL_CELL_SIGNALING_IN_HELICOBACTER_PYLORI_INFECTION | 0.0005 | 0.0009 | 0.0005 | 0.0005 |
| KEGG_ETHER_LIPID_METABOLISM | 0.0005 | 0.0009 | 0.0005 | 0.0005 |
| KEGG_FC_EPSILON_RI_SIGNALING_PATHWAY | 0.0005 | 0.0009 | 0.0005 | 0.0005 |
| KEGG_FC_GAMMA_R_MEDIATED_PHAGOCYTOSIS | 0.0005 | 0.0009 | 0.0005 | 0.0005 |
| KEGG_FRUCTOSE_AND_MANNOSE_METABOLISM | 0.0005 | 0.0009 | 0.0005 | 0.0005 |
| KEGG_GLYCOSAMINOGLYCAN_BIOSYNTHESIS_CHONDROITIN_SULFATE | 0.0005 | 0.0009 | 0.0005 | 0.0005 |
| KEGG_GLYCOSAMINOGLYCAN_DEGRADATION | 0.0005 | 0.0009 | 0.0010 | 0.0010 |
| KEGG_GRAFT_VERSUS_HOST_DISEASE | 0.0005 | 0.0009 | 0.0005 | 0.0005 |
| KEGG_HEMATOPOIETIC_CELL_LINEAGE | 0.0005 | 0.0009 | 0.0005 | 0.0005 |
| KEGG_INTESTINAL_IMMUNE_NETWORK_FOR_IGA_PRODUCTION | 0.0005 | 0.0009 | 0.0005 | 0.0005 |
| KEGG_JAK_STAT_SIGNALING_PATHWAY | 0.0005 | 0.0009 | 0.0005 | 0.0005 |
| KEGG_LEISHMANIA_INFECTION | 0.0005 | 0.0009 | 0.0005 | 0.0005 |
| KEGG_LEUKOCYTE_TRANSENDOTHELIAL_MIGRATION | 0.0005 | 0.0009 | 0.0005 | 0.0005 |
| KEGG_NATURAL_KILLER_CELL_MEDIATED_CYTOTOXICITY | 0.0005 | 0.0009 | 0.0005 | 0.0005 |
| KEGG_NOD_LIKE_RECEPTOR_SIGNALING_PATHWAY | 0.0005 | 0.0009 | 0.0005 | 0.0005 |
| KEGG_OLFACTORY_TRANSDUCTION | 0.0005 | 0.0009 | 0.0005 | 0.0005 |
| KEGG_P53_SIGNALING_PATHWAY | 0.0005 | 0.0009 | 0.0005 | 0.0005 |
| KEGG_PANCREATIC_CANCER | 0.0005 | 0.0009 | 0.0005 | 0.0005 |
| KEGG_PATHOGENIC_ESCHERICHIA_COLI_INFECTION | 0.0005 | 0.0009 | 0.0005 | 0.0005 |
| KEGG_PENTOSE_PHOSPHATE_PATHWAY | 0.0005 | 0.0009 | 0.0005 | 0.0005 |
| KEGG_PHOSPHATIDYLINOSITOL_SIGNALING_SYSTEM | 0.0005 | 0.0009 | 0.0005 | 0.0005 |
| KEGG_PRIMARY_IMMUNODEFICIENCY | 0.0005 | 0.0009 | 0.0005 | 0.0005 |
| KEGG_REGULATION_OF_ACTIN_CYTOSKELETON | 0.0005 | 0.0009 | 0.0005 | 0.0005 |
| KEGG_RIG_I_LIKE_RECEPTOR_SIGNALING_PATHWAY | 0.0005 | 0.0009 | 0.0005 | 0.0005 |
| KEGG_SYSTEMIC_LUPUS_ERYTHEMATOSUS | 0.0005 | 0.0009 | 0.0005 | 0.0005 |
| KEGG_T_CELL_RECEPTOR_SIGNALING_PATHWAY | 0.0005 | 0.0009 | 0.0005 | 0.0005 |
| KEGG_TOLL_LIKE_RECEPTOR_SIGNALING_PATHWAY | 0.0005 | 0.0009 | 0.0005 | 0.0005 |
| KEGG_TYPE_I_DIABETES_MELLITUS | 0.0005 | 0.0009 | 0.0005 | 0.0005 |
| KEGG_VASOPRESSIN_REGULATED_WATER_REABSORPTION | 0.0005 | 0.0009 | 0.0005 | 0.0005 |
| KEGG_VIRAL_MYOCARDITIS | 0.0005 | 0.0009 | 0.0005 | 0.0005 |
| KEGG_ERBB_SIGNALING_PATHWAY | 0.0010 | 0.0020 | 0.0005 | 0.0005 |
| KEGG_GALACTOSE_METABOLISM | 0.0010 | 0.0020 | 0.0005 | 0.0005 |
| KEGG_LYSOSOME | 0.0010 | 0.0020 | 0.0005 | 0.0005 |
| KEGG_NEUROTROPHIN_SIGNALING_PATHWAY | 0.0010 | 0.0020 | 0.0005 | 0.0005 |
| KEGG_O_GLYCAN_BIOSYNTHESIS | 0.0010 | 0.0020 | 0.0005 | 0.0005 |
| KEGG_PRION_DISEASES | 0.0010 | 0.0020 | 0.0005 | 0.0005 |
| KEGG_SPHINGOLIPID_METABOLISM | 0.0010 | 0.0020 | 0.0005 | 0.0005 |
| KEGG_TYPE_II_DIABETES_MELLITUS | 0.0010 | 0.0020 | 0.0005 | 0.0005 |
| KEGG_VEGF_SIGNALING_PATHWAY | 0.0010 | 0.0020 | 0.0005 | 0.0005 |
| KEGG_ADIPOCYTOKINE_SIGNALING_PATHWAY | 0.0015 | 0.0031 | 0.0005 | 0.0005 |
| KEGG_AMINO_SUGAR_AND_NUCLEOTIDE_SUGAR_METABOLISM | 0.0015 | 0.0031 | 0.0005 | 0.0005 |
| KEGG_GLIOMA | 0.0015 | 0.0031 | 0.0005 | 0.0005 |
| KEGG_INOSITOL_PHOSPHATE_METABOLISM | 0.0015 | 0.0031 | 0.0005 | 0.0005 |
| KEGG_MAPK_SIGNALING_PATHWAY | 0.0015 | 0.0031 | 0.0005 | 0.0005 |
| KEGG_PROXIMAL_TUBULE_BICARBONATE_RECLAMATION | 0.0015 | 0.0031 | 0.0005 | 0.0005 |
| KEGG_RENAL_CELL_CARCINOMA | 0.0015 | 0.0031 | 0.0010 | 0.0010 |
| KEGG_AMYOTROPHIC_LATERAL_SCLEROSIS_ALS | 0.0020 | 0.0040 | 0.0005 | 0.0005 |
| KEGG_GLYCEROPHOSPHOLIPID_METABOLISM | 0.0020 | 0.0040 | 0.0005 | 0.0005 |
| KEGG_PATHWAYS_IN_CANCER | 0.0020 | 0.0040 | 0.0005 | 0.0005 |
| KEGG_GLYCOLYSIS_GLUCONEOGENESIS | 0.0025 | 0.0049 | 0.0005 | 0.0005 |
| KEGG_OTHER_GLYCAN_DEGRADATION | 0.0025 | 0.0049 | 0.0020 | 0.0020 |
| KEGG_CELL_CYCLE | 0.0030 | 0.0058 | 0.0005 | 0.0005 |
| KEGG_GNRH_SIGNALING_PATHWAY | 0.0030 | 0.0058 | 0.0005 | 0.0005 |
| KEGG_NICOTINATE_AND_NICOTINAMIDE_METABOLISM | 0.0030 | 0.0058 | 0.0005 | 0.0005 |
| KEGG_NON_SMALL_CELL_LUNG_CANCER | 0.0030 | 0.0058 | 0.0005 | 0.0005 |
| KEGG_PROSTATE_CANCER | 0.0030 | 0.0058 | 0.0005 | 0.0005 |
| KEGG_DILATED_CARDIOMYOPATHY | 0.0035 | 0.0067 | 0.0005 | 0.0005 |
| KEGG_CALCIUM_SIGNALING_PATHWAY | 0.0040 | 0.0075 | 0.0005 | 0.0005 |
| KEGG_OOCYTE_MEIOSIS | 0.0040 | 0.0075 | 0.0005 | 0.0005 |
| KEGG_LONG_TERM_POTENTIATION | 0.0045 | 0.0083 | 0.0005 | 0.0005 |
| KEGG_VIBRIO_CHOLERAE_INFECTION | 0.0045 | 0.0083 | 0.0005 | 0.0005 |
| KEGG_HYPERTROPHIC_CARDIOMYOPATHY_HCM | 0.0050 | 0.0092 | 0.0005 | 0.0005 |
| KEGG_PROGESTERONE_MEDIATED_OOCYTE_MATURATION | 0.0050 | 0.0092 | 0.0005 | 0.0005 |
| KEGG_SMALL_CELL_LUNG_CANCER | 0.0055 | 0.0100 | 0.0005 | 0.0005 |
| KEGG_SNARE_INTERACTIONS_IN_VESICULAR_TRANSPORT | 0.0060 | 0.0108 | 0.0005 | 0.0005 |
| KEGG_COMPLEMENT_AND_COAGULATION_CASCADES | 0.0075 | 0.0131 | 0.0010 | 0.0010 |
| KEGG_ARRHYTHMOGENIC_RIGHT_VENTRICULAR_CARDIOMYOPATHY_ARVC | 0.0085 | 0.0146 | 0.0005 | 0.0005 |
| KEGG_ALDOSTERONE_REGULATED_SODIUM_REABSORPTION | 0.0110 | 0.0183 | 0.0005 | 0.0005 |
| KEGG_NEUROACTIVE_LIGAND_RECEPTOR_INTERACTION | 0.0110 | 0.0183 | 0.0005 | 0.0005 |
| KEGG_ADHERENS_JUNCTION | 0.0135 | 0.0219 | 0.0005 | 0.0005 |
| KEGG_TASTE_TRANSDUCTION | 0.0140 | 0.0226 | 0.0005 | 0.0005 |
| KEGG_FOCAL_ADHESION | 0.0145 | 0.0233 | 0.0005 | 0.0005 |
| KEGG_MELANOMA | 0.0160 | 0.0256 | 0.0005 | 0.0005 |
| KEGG_MELANOGENESIS | 0.0190 | 0.0295 | 0.0005 | 0.0005 |
| KEGG_SULFUR_METABOLISM | 0.0355 | 0.0514 | 0.0045 | 0.0045 |
| KEGG_INSULIN_SIGNALING_PATHWAY | 0.0435 | 0.0619 | 0.0005 | 0.0005 |
| KEGG_DNA_REPLICATION | 0.0445 | 0.0632 | 0.0015 | 0.0015 |
| KEGG_GAP_JUNCTION | 0.0460 | 0.0650 | 0.0005 | 0.0005 |
| KEGG_PURINE_METABOLISM | 0.0560 | 0.0776 | 0.0005 | 0.0005 |
| KEGG_AXON_GUIDANCE | 0.0585 | 0.0808 | 0.0005 | 0.0005 |
| KEGG_BASE_EXCISION_REPAIR | 0.0725 | 0.0977 | 0.0005 | 0.0005 |

The liver's size is tightly controlled, and loss in liver mass—such as by partial liver resection—elicits a robust regenerative response. Thus, the loss in liver mass and decrease in midlobular CCND1 expression led us to investigate whether the regenerative response was impaired. Indeed, staining for the proliferation marker Ki67 showed a lack of proliferating hepatocytes at both 10 days and 6 weeks after HSC depletion (FIGS. 5L-M). Immunostaining for cleaved caspase 3, an apoptosis marker, showed positive hepatocyte staining in some mice though the increase was not significant and observed only in some HSC-depleted mice (n=3 out of 11 animals) (FIGS. 5L and N). In summary, the results herein suggest that HSCs are critical for maintaining midlobular hepatocyte CCND1 expression and liver mass.

### Example 4

### HSC-Derived NTF-3 Induces CCND1 and is a Hepatocyte Mitogen

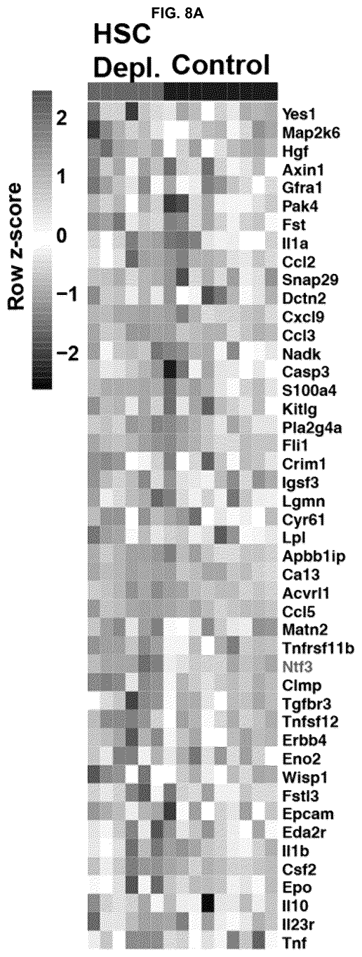

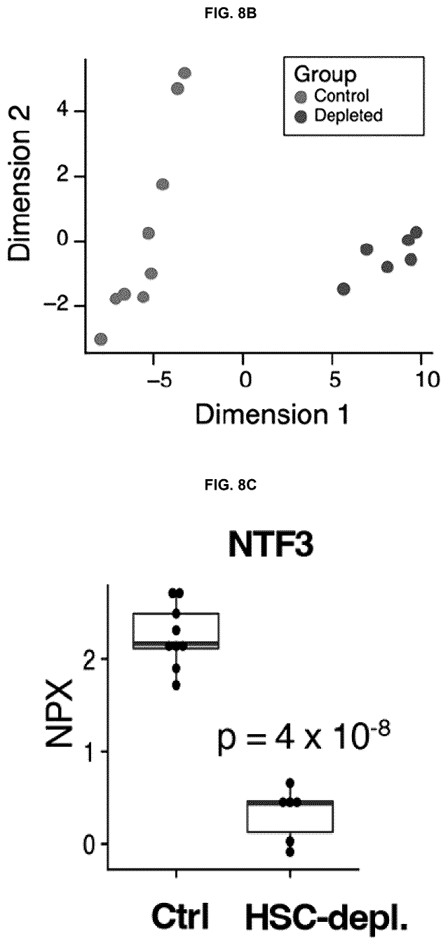

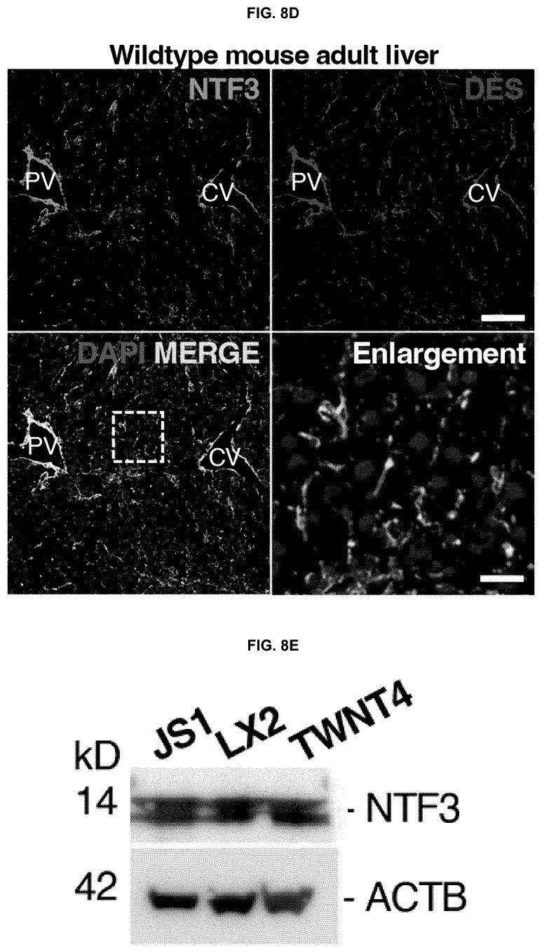

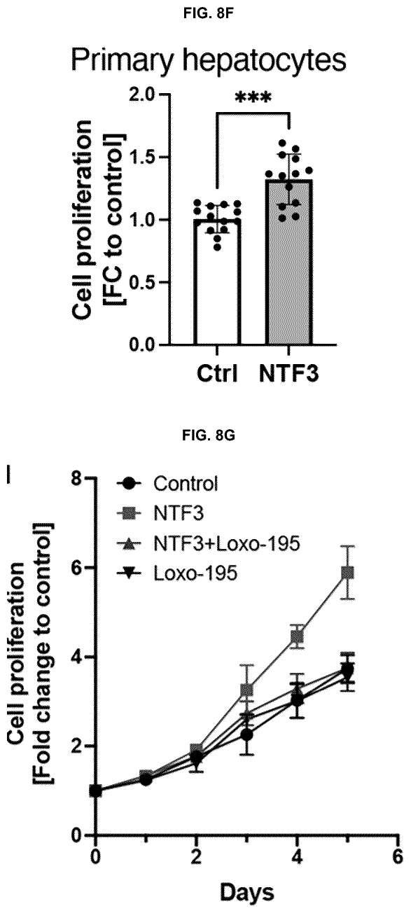

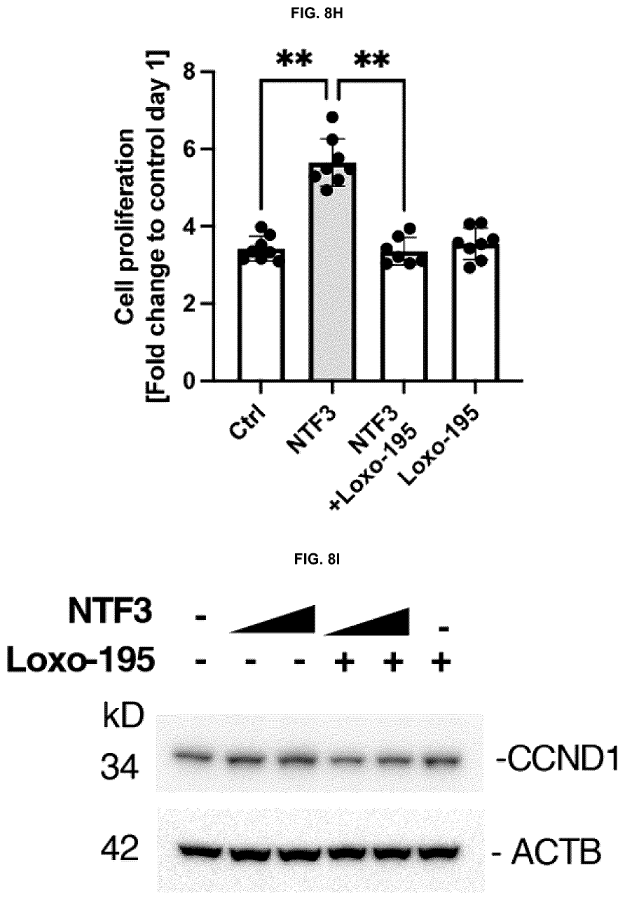

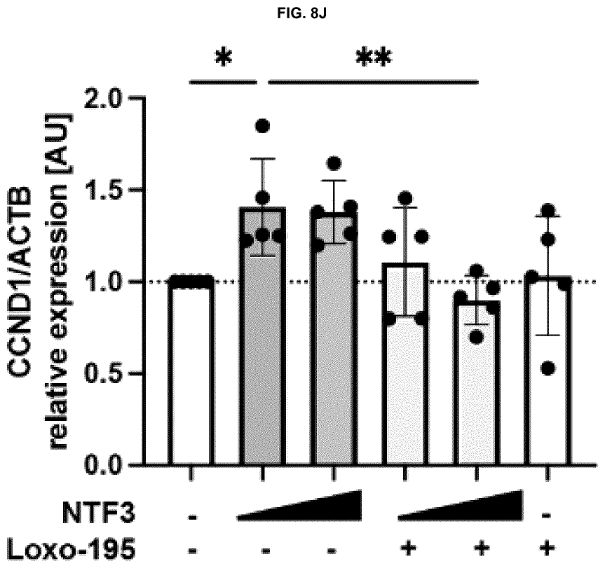

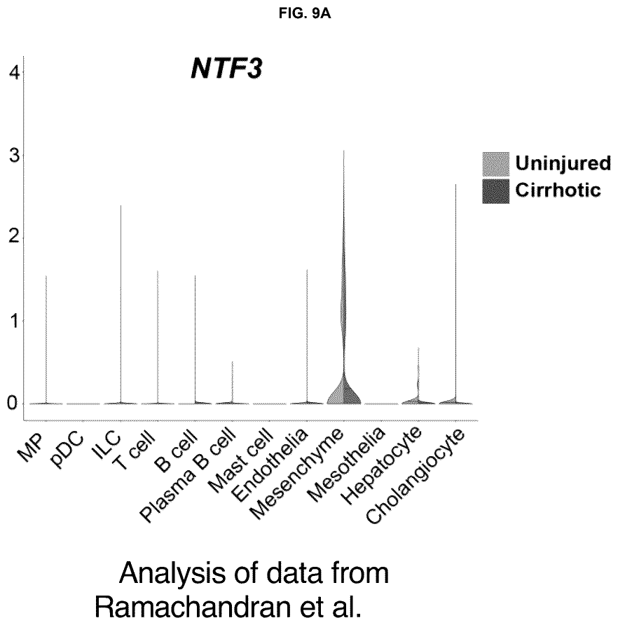

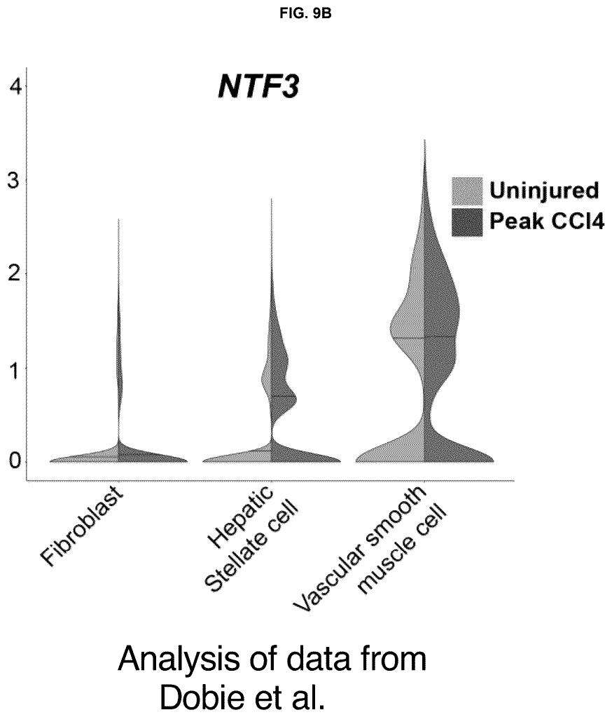

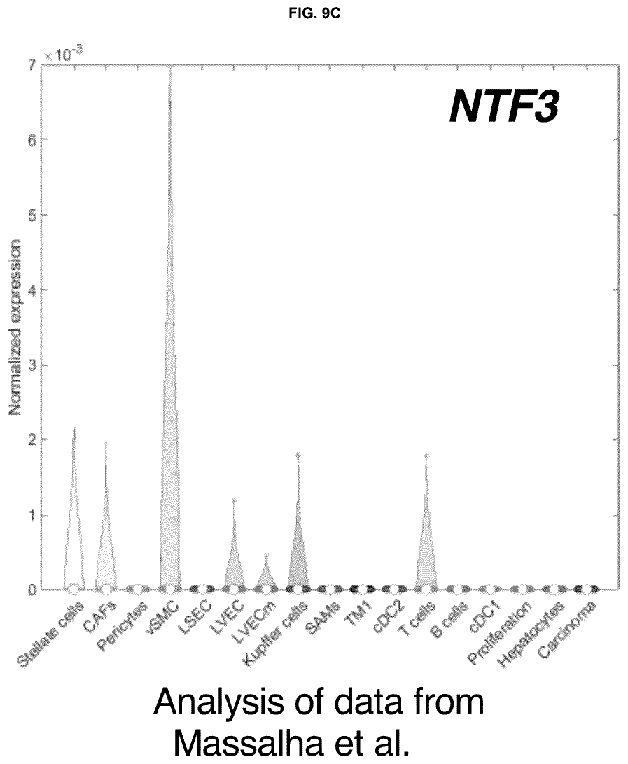

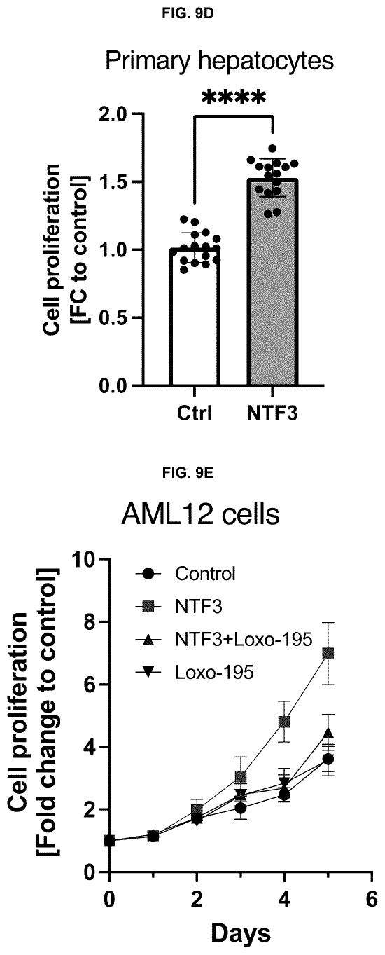

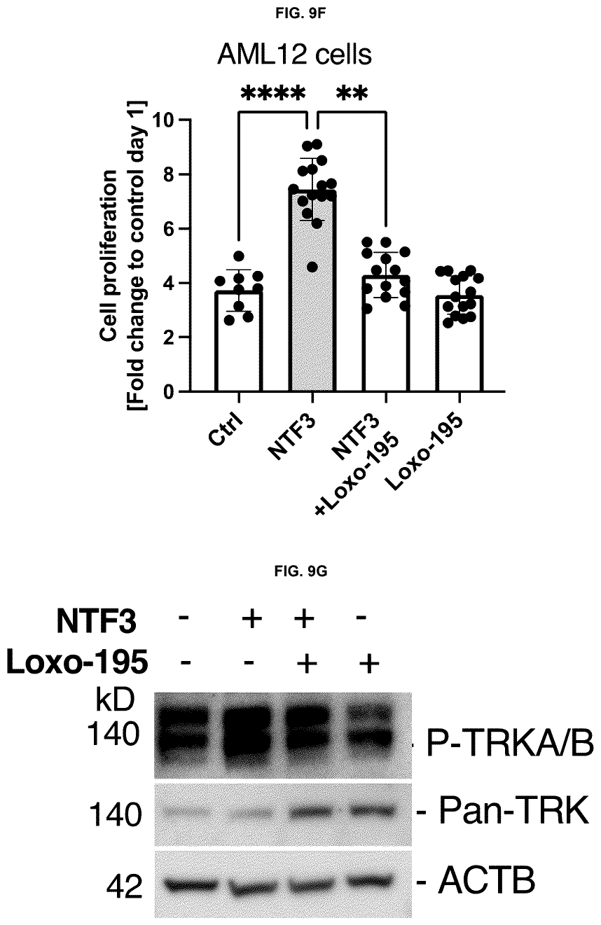

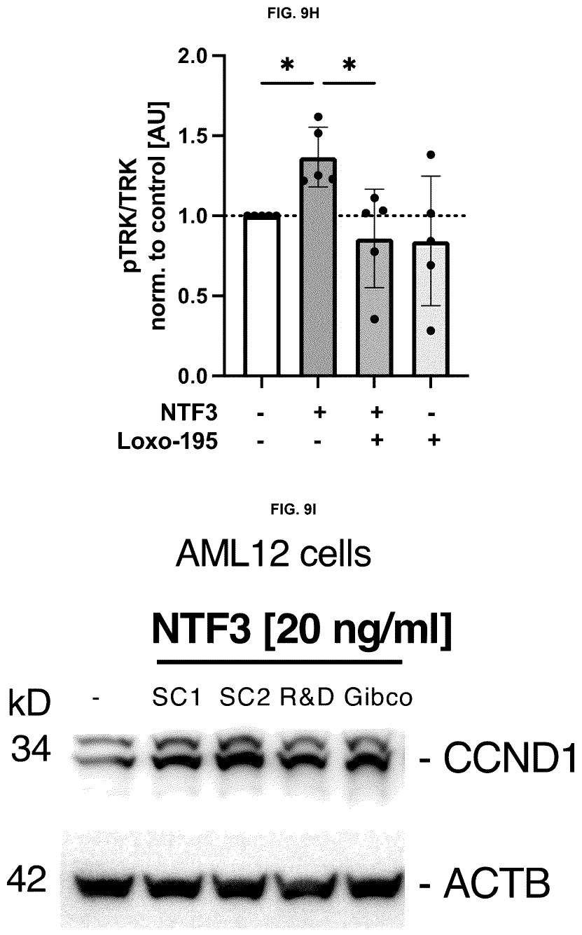

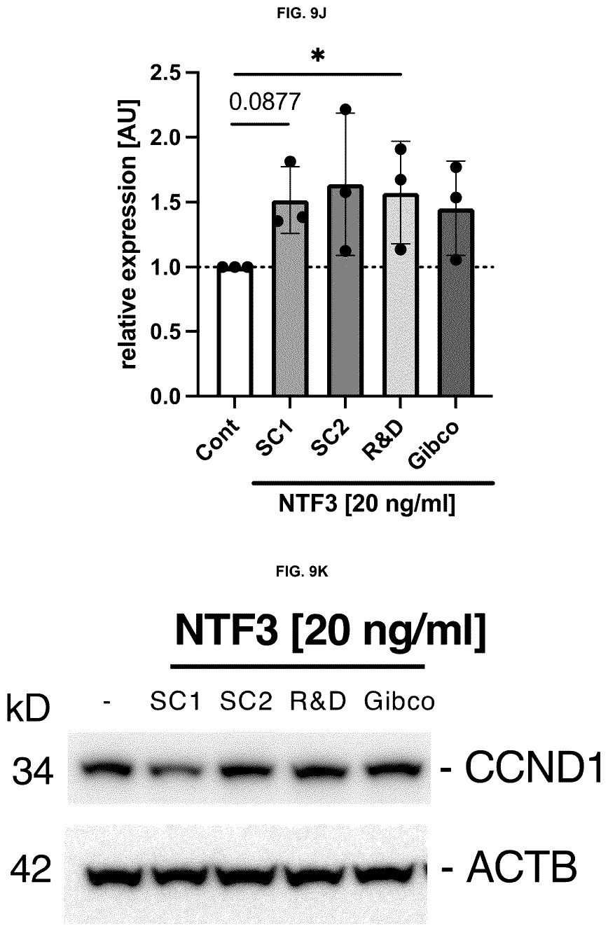

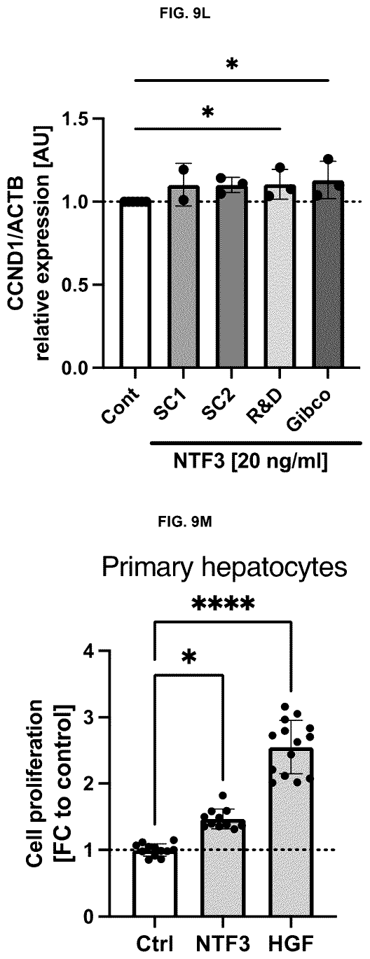

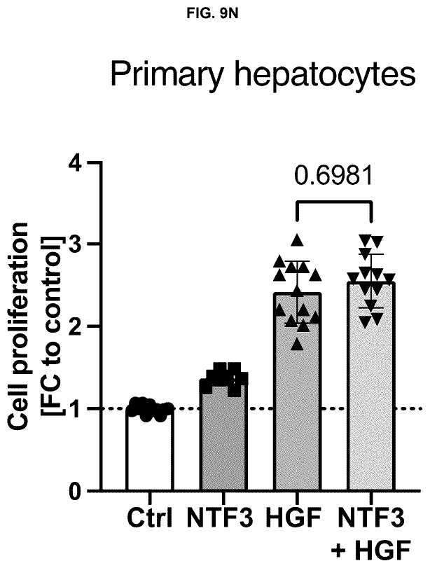

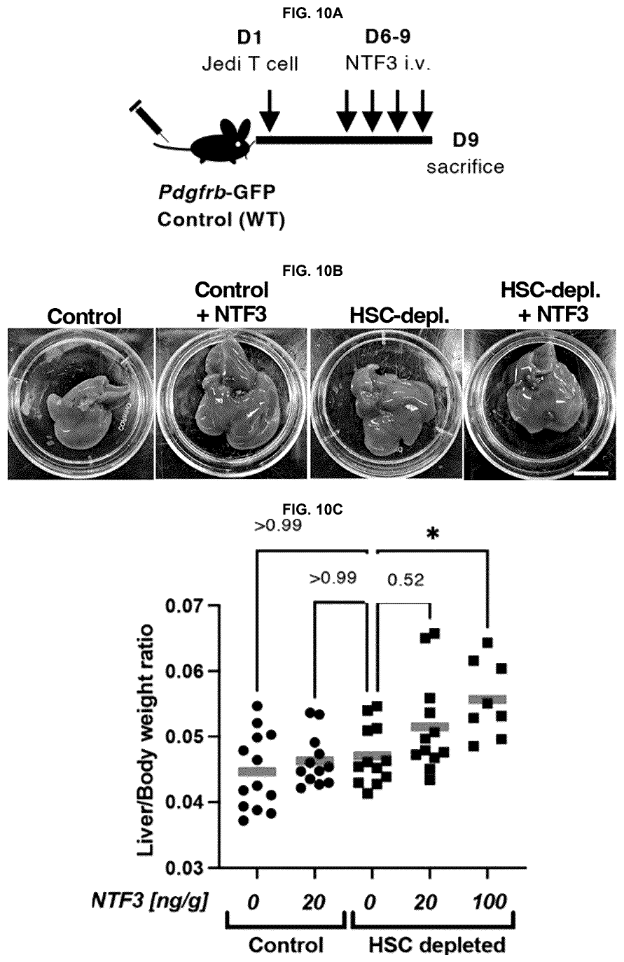

To investigate proteomic changes due to HSC ablation, whole liver protein lysates were assessed from control and HSC-depleted livers using the Olink® mouse exploratory assay, which employs proximity extension assay technology for protein biomarker analysis of 92 proteins involved in diverse cellular processes (Assarsson et al., PLOS One. 2014; 9(4): e95192). The Olink® analysis separated control and HSC-depleted livers harvested 10 days after depletion into two clearly distinct groups (FIGS. 8A-B; TABLE 6). In total, robust differences were found in 46 proteins between depleted and control livers, with amounts of 30 proteins higher and 16 proteins lower in HSC-depleted than in control livers (adjusted p-value <0.05). HGF was among the proteins expressed in lower amounts, which was expected, given that it is produced and released by both HSCs and liver sinusoidal endothelial cells. Among the reduced proteins with a less well-established role in HSCs was neurotrophin-3 (NTF3) (FIG. 8C), a member of the nerve growth factor family. HSCs are known to express genes encoding both neuronal and glial proteins such as GFAP and nerve growth factor receptor [NGFR, also known as p75NTR and tumor necrosis factor receptor superfamily member 16 (TNFR 16)]. Although it has been long known that HSCs express NTF3, its paracrine function in the liver remains largely unexplored save for a report identifying NTF3 as an autocrine signaling pathway in HSCs that contributes to fibrosis. It was confirmed herein that NTF3 is expressed in HSCs by analyses of three published mouse and human liver scRNA-seq datasets (Massalha et al., Mol Syst Biol. 2020; 16(12): e9682; Dobie et al., Cell Rep. 2019; 29(7): 1832-1847.e8; Ramachandran et al., Nature. 2019; 575(7783): 512-518), which showed highly specific NTF3 expression in mesenchymal cells and HSCs (FIGS. 9A-C). Furthermore, immunofluorescence analysis for NTF3 and desmin in mouse liver demonstrated pan-lobular expression in HSCs (FIG. 8D). Western blotting of mouse and human HSC cell lines JS1, LX2 and TWNT4 also showed robust NTF3 expression (FIG. 8E).

TABLE 6
Olink ® protein biomarker analysis.

| | Olink ® | Olink ® | Olink ® | Olink ® | Olink ® | Olink ® | Olink ® | Olink ® | Olink ® |
|---|---|---|---|---|---|---|---|---|---|
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Clmp | Matn2 | Cpe | Inc Ctrl 1 | Gcg | Gdnf | Yes1 | Il17a | Foxo1 |
| Uniprot ID | Q8R373 | O08746 | Q00493 | — | P55095 | P48540 | Q04736 | Q62386 | Q9R1E0 |
| OlinkID | OID05027 | OID05028 | OID05029 | — | OID05031 | OID05032 | OID05033 | OID05034 | OID05035 |
| IPC1 | 7.09118 | 7.98429 | 7.56907 | 0.00000 | 9.15390 | 9.08043 | 8.91961 | 9.07242 | 7.90291 |
| IPC2 | 7.28680 | 8.09992 | 7.57167 | −0.00868 | 9.32683 | 9.21355 | 9.06345 | 9.16131 | 7.93581 |
| IPC3 | 7.33297 | 8.12776 | 7.55192 | 0.15567 | 9.23423 | 9.22638 | 8.95082 | 9.06000 | 7.85159 |
| NC1 | −0.31712 | −0.20068 | 0.38250 | 0.00759 | −1.37901 | −0.51777 | −1.26486 | −1.46303 | 0.40856 |
| NC2 | 0.30427 | −0.29631 | 1.03676 | 0.04130 | −0.81548 | −0.03895 | −0.83191 | −0.64892 | 0.61220 |
| NC3 | 0.36896 | 0.05158 | 0.78099 | 0.08560 | −0.62944 | 0.34874 | −0.65161 | −0.59166 | 0.69428 |
| Sample 1 | 3.32449 | 1.93019 | 1.53691 | −0.20290 | −0.59522 | −0.08651 | 7.46179 | −0.84772 | 6.09602 |
| Sample 7 | 0.54233 | 1.00899 | 1.82399 | 0.14286 | −0.12184 | 0.29857 | 7.11453 | −0.80326 | 6.05081 |
| Sample 3 | 2.60146 | 3.11508 | 1.24545 | −0.01913 | −1.34057 | 0.34043 | 7.20179 | −0.98341 | 5.74141 |
| Sample 11 | 0.62045 | 1.43168 | 1.56210 | 0.04244 | −0.20956 | 0.15849 | 6.93773 | −0.90252 | 6.52662 |
| Sample 5 | 2.90187 | 2.49485 | 1.50394 | 0.02127 | −0.31489 | 0.10166 | 7.34380 | −0.79156 | 6.00866 |
| Sample 8 | 0.47427 | 1.14239 | 1.57899 | 0.04678 | −0.56634 | 0.21068 | 7.11620 | −0.91245 | 6.57148 |
| Sample 9 | 0.56628 | 1.14719 | 1.07427 | 0.38097 | −1.37530 | 0.26038 | 7.09706 | −0.53686 | 6.45840 |
| Sample 12 | 3.17179 | 2.19813 | 1.55122 | 0.02474 | −0.78984 | 0.40875 | 7.28351 | −1.09450 | 5.78161 |
| Sample 13 | 2.91629 | 2.13270 | 1.86634 | −0.04855 | −0.69789 | −0.36756 | 7.43469 | −0.64616 | 6.01458 |
| Sample 14 | 2.97815 | 2.15935 | 1.86866 | 3.04475 | −0.56581 | 0.65218 | 7.40923 | −0.04460 | 6.23646 |
| Sample 15 | 2.29466 | 1.79640 | 1.49198 | 2.91802 | −0.51381 | 0.13048 | 7.14164 | −0.50237 | 5.69585 |
| Sample 16 | 1.62397 | 1.67908 | 1.58950 | 1.99454 | −0.51814 | 0.08712 | 6.80547 | −0.70717 | 5.43659 |
| Sample 17 | 0.65364 | 0.94234 | 1.65350 | 0.30467 | −0.87850 | −0.29361 | 7.13125 | −0.38171 | 6.07079 |
| Sample 18 | 3.43461 | 3.05114 | 1.78317 | −0.19086 | −0.16158 | 0.49115 | 7.42179 | −0.48478 | 6.09371 |
| Sample 19 | 3.24431 | 3.00801 | 1.51072 | 0.48144 | −0.16896 | 0.43230 | 7.43514 | −0.54727 | 6.15841 |
| LOD | 0.54436 | 0.60895 | 1.18304 | 0.04483 | −0.18498 | 0.65569 | −0.40151 | −0.48089 | 0.94603 |
| Missing | 10% | 6% | 31% | 0% | 88% | 94% | 5% | 83% | 5% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Tnfrsf11b | Tgfb1 | Pla2g4a | Il6 | Prdx5 | Tgfa | Ccl5 | Epo | Axin1 |
| Uniprot ID | O08712 | P04202 | P47713 | P08505 | P99029 | P48030 | P30882 | P07321 | O35625 |
| OlinkID | OID05036 | OID05037 | OID05038 | OID05039 | OID05040 | OID05041 | OID05042 | OID05043 | OID05044 |
| IPC1 | 7.16965 | 7.52270 | 6.11236 | 7.73224 | 10.22338 | 7.39070 | 8.53904 | 8.18362 | 7.97146 |
| IPC2 | 7.26609 | 7.63194 | 6.21424 | 7.85347 | 10.35189 | 7.44347 | 8.72332 | 8.36252 | 8.05808 |
| IPC3 | 7.25558 | 7.68712 | 6.21413 | 7.83025 | 10.30913 | 7.39362 | 8.64215 | 8.29215 | 8.06575 |
| NC1 | 0.36989 | 0.32670 | 0.33713 | −0.34804 | −1.01736 | −0.42869 | −1.94312 | 0.07243 | 0.08581 |
| NC2 | 0.68613 | 0.39781 | 1.08092 | 0.15435 | −0.69251 | −0.13967 | −1.09736 | 0.24385 | 0.48111 |
| NC3 | 0.70391 | 0.58471 | 1.13931 | 0.27867 | −0.45101 | −0.09062 | −0.87922 | 0.15646 | 0.40486 |
| Sample 1 | 1.82413 | 1.85345 | 3.22619 | −0.08250 | 7.88165 | 4.15896 | 0.27716 | 0.28554 | 6.29064 |
| Sample 7 | 1.06823 | 2.16575 | 5.50460 | 0.71797 | 7.93543 | 5.21137 | 5.90760 | 0.68063 | 7.24444 |
| Sample 3 | 1.69484 | 1.54675 | 3.75418 | 0.17305 | 7.24416 | 3.58394 | 0.51221 | 0.13614 | 5.77083 |
| Sample 11 | 1.04844 | 1.89644 | 5.61292 | 0.78085 | 8.02576 | 4.64855 | 7.19465 | 0.42485 | 7.46853 |
| Sample 5 | 2.47230 | 1.44390 | 4.15626 | 0.33537 | 7.22678 | 4.10769 | 1.18400 | 0.17331 | 6.27882 |
| Sample 8 | 1.10957 | 1.62193 | 5.70049 | 0.36347 | 7.96668 | 3.91423 | 5.41328 | 0.51217 | 6.97646 |
| Sample 9 | 0.93932 | 2.49410 | 6.47054 | 1.40410 | 8.05526 | 4.44743 | 6.89117 | 0.55902 | 7.26601 |
| Sample 12 | 2.87880 | 1.99631 | 3.24569 | 2.10595 | 7.71517 | 4.67586 | 0.71098 | 0.36738 | 6.66497 |
| Sample 13 | 2.21817 | 1.78943 | 3.71248 | 1.89161 | 7.40774 | 4.32086 | 0.57317 | 0.32896 | 6.40050 |
| Sample 14 | 2.31430 | 2.38501 | 3.46601 | 0.11840 | 8.00904 | 5.34913 | −0.03420 | 0.12298 | 6.32194 |
| Sample 15 | 1.82369 | 1.15370 | 2.85050 | 0.33489 | 7.72553 | 4.10518 | −0.25302 | 0.17532 | 5.76897 |
| Sample 16 | 1.36014 | 2.37046 | 6.48653 | 1.28757 | 8.08763 | 6.53130 | 6.73299 | 1.59764 | 6.67150 |
| Sample 17 | 1.09998 | 2.08777 | 6.97332 | 1.15054 | 7.57496 | 3.45220 | 7.07946 | 1.44162 | 7.02280 |
| Sample 18 | 2.30948 | 2.01866 | 4.15709 | 0.34300 | 7.19029 | 4.42289 | 0.62599 | 0.38930 | 6.21150 |
| Sample 19 | 2.28902 | 1.70575 | 4.10358 | 0.69247 | 7.90009 | 4.74398 | 0.96720 | 0.14988 | 6.54178 |
| LOD | 0.95284 | 0.76968 | 1.16824 | 0.41070 | −0.17004 | 0.29418 | −0.89683 | 0.52060 | 0.85580 |
| Missing | 10% | 22% | 5% | 53% | 5% | 5% | 4% | 78% | 5% |
| Data freq. | | | | | | | | | |

| | | Olink ® | Olink ® | Olink ® | Olink ® | Olink ® | Olink ® | Olink ® | Olink ® |
|---|---|---|---|---|---|---|---|---|---|
| | | MOUSE | MOUSE | MOUSE | MOUSE | MOUSE | MOUSE | MOUSE | MOUSE |
| | | EXPLOR- | EXPLOR- | EXPLOR- | EXPLOR- | EXPLOR- | EXPLOR- | EXPLOR- | EXPLOR- |
| | | ATORY | ATORY | ATORY | ATORY | ATORY | ATORY | ATORY | ATORY |
| Panel | 0.00000 | (v.3801) | (v.3801) | (v.3801) | (v.3801) | (v.3801) | (v.3801) | (v.3801) | (v.3801) |
| Assay | 0.00000 | Fst | Rgma | Nadk | Tnni3 | Notch3 | Snap29 | Cntn1 | Clstn2 |
| Uniprot ID | 0.00000 | P47931 | Q6PCX7 | P58058 | P48787 | Q61982 | Q9ERBO | P12960 | Q9ER65 |
| OlinkID | 0.00000 | OID05046 | OID05047 | OID05048 | OID05049 | OID05050 | OID05051 | OID05052 | OID05053 |
| IPC1 | 0.00000 | 8.44001 | 8.37212 | 8.62653 | 10.21503 | 9.69694 | 8.58020 | 6.95195 | 8.39352 |
| IPC2 | 0.00000 | 8.52622 | 8.47897 | 8.75096 | 10.32734 | 9.79332 | 8.57767 | 7.06296 | 8.49799 |
| IPC3 | 0.00000 | 8.43941 | 8.41112 | 8.56141 | 10.18714 | 9.61998 | 8.33265 | 6.99934 | 8.40722 |
| NC1 | 0.00000 | −0.23797 | −0.45882 | −0.24942 | −0.33439 | −0.75356 | 0.19994 | −0.71360 | −0.37180 |
| NC2 | 0.00000 | 0.45087 | −0.54331 | 0.01063 | −0.16934 | −0.77872 | 0.64269 | −0.64973 | 0.01802 |
| NC3 | 0.00000 | 0.47914 | −0.10996 | 0.28670 | −0.20275 | −0.74551 | 0.91793 | −0.40786 | 0.17818 |
| Sample 1 | 0.00000 | 6.55388 | 0.01670 | 10.14805 | 1.42241 | −1.03536 | 8.01030 | −0.00265 | 0.33451 |
| Sample 7 | 0.00000 | 8.27333 | 0.05580 | 9.46183 | 0.37651 | −0.22949 | 8.50812 | −0.07757 | 0.22378 |
| Sample 3 | 0.00000 | 6.72757 | 0.03711 | 10.04509 | 0.48799 | −0.63170 | 8.18081 | −0.13113 | −0.41578 |
| Sample 11 | 0.00000 | 8.07455 | 0.00848 | 9.31882 | 0.57880 | −0.52121 | 8.45064 | −0.00872 | 0.20076 |
| Sample 5 | 0.00000 | 6.89791 | 0.20894 | 9.72586 | 0.47881 | −0.62939 | 8.24429 | −0.17917 | 0.00186 |
| Sample 8 | 0.00000 | 7.93382 | 0.23775 | 9.70952 | 0.41948 | −0.27608 | 8.44687 | −0.05710 | 0.21379 |
| Sample 9 | 0.00000 | 7.62356 | −0.03761 | 9.46029 | 1.00444 | −0.81772 | 8.47861 | 0.03073 | 0.01331 |
| Sample 12 | 0.00000 | 7.09376 | −0.08729 | 9.27697 | 0.72827 | −0.45655 | 8.13643 | −0.12011 | 0.60350 |
| Sample 13 | 0.00000 | 6.95043 | −0.35722 | 9.66318 | 1.33252 | −0.77937 | 8.35002 | −0.02493 | 0.61742 |
| Sample 14 | 0.00000 | 6.99637 | −0.16723 | 10.01294 | 0.56372 | −0.59912 | 8.40783 | 0.28267 | 0.44353 |
| Sample 15 | | 6.48955 | 0.17520 | 10.21952 | 1.01382 | −0.45106 | 8.12791 | −0.12615 | −0.00157 |
| Sample 16 | 0.00000 | 7.39548 | 0.14393 | 9.43941 | 0.57850 | −0.32198 | 8.40143 | −0.18801 | 0.30369 |
| Sample 17 | 0% | 7.52464 | −0.33689 | 9.20507 | 1.11775 | −0.38736 | 8.50163 | −0.08694 | −0.01887 |
| Sample 18 | | 6.89009 | 0.10257 | 9.61414 | 0.06373 | −0.14531 | 8.39462 | −0.00121 | 0.69710 |
| Sample 19 | | 7.16370 | 0.03651 | 9.78007 | 0.56858 | −0.65687 | 8.13298 | −0.09318 | 0.73644 |
| LOD | — | 0.63165 | 0.04380 | 0.44723 | 0.30008 | −0.10324 | 1.01978 | −0.25333 | 0.54014 |

| | | | | | | | | |
|---|---|---|---|---|---|---|---|---|
| Missing Data freq. | 5% | 47% | 5% | 18% | 95% | 5% | 27% | 70% |

| | Olink ® | Olink ® | Olink ® | Olink ® | Olink ® | Olink ® | Olink ® | Olink ® | Olink ® |
|---|---|---|---|---|---|---|---|---|---|
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | S100a4 | Ca13 | Mia | Cant1 | Kitlg | Gfra1 | Ppp1r2 | Adam23 | Itgb6 |
| Uniprot ID | P07091 | Q9D6N1 | Q61865 | Q8VCF1 | P20826 | P97785 | Q9DCL8 | Q9R1V7 | Q9ZOT9 |
| OlinkID | OID05054 | OID05055 | OID05056 | OID05057 | OID05058 | OID05059 | OID05060 | OID05061 | OID05062 |
| IPC1 | 9.58548 | 10.66843 | 6.98712 | 9.17833 | 7.51348 | 8.46520 | 10.73851 | 7.56053 | 10.79590 |
| IPC2 | 9.65707 | 10.77059 | 7.06122 | 9.20497 | 7.62854 | 8.57338 | 10.82051 | 7.64999 | 10.83163 |
| IPC3 | 9.59977 | 10.65191 | 7.10165 | 8.97436 | 7.60327 | 8.47833 | 10.72420 | 7.48317 | 10.52077 |
| NC1 | −0.89930 | −0.69233 | −0.12343 | −1.24513 | −0.36881 | −0.26646 | −1.30317 | −1.97880 | 0.95116 |
| NC2 | −0.18594 | −0.15788 | 0.21234 | −0.86491 | −0.19386 | −0.64676 | −0.65019 | −1.68479 | 0.75152 |
| NC3 | −0.46962 | −0.41362 | 0.54001 | −0.52918 | 0.27194 | −0.12385 | −0.39878 | −1.45052 | 1.47140 |
| Sample 1 | 3.81499 | 2.23285 | 0.29607 | −0.24916 | 4.42810 | 6.07137 | 4.42812 | −1.70810 | 1.73198 |
| Sample 7 | 6.57311 | 4.45628 | 0.43771 | 0.17156 | 5.43020 | 6.69202 | 4.72334 | −1.69498 | 1.57345 |
| Sample 3 | 3.78098 | 1.84881 | 0.26588 | −0.30446 | 3.48312 | 5.24427 | 4.55909 | −1.50700 | 0.89611 |
| Sample 11 | 6.31786 | 4.79123 | 0.44345 | 0.14223 | 5.78048 | 7.41862 | 4.63170 | −1.09500 | 1.57652 |
| Sample 5 | 4.49023 | 1.86606 | 0.51787 | −0.15083 | 4.13377 | 5.38733 | 4.65062 | −1.08652 | 1.14872 |
| Sample 8 | 6.50586 | 4.32947 | 0.43791 | −0.21547 | 5.33453 | 7.03321 | 4.80465 | −1.16718 | 1.41537 |
| Sample 9 | 6.70154 | 4.79026 | 0.52936 | 0.04140 | 5.56941 | 7.23251 | 4.86999 | −1.06231 | 1.51810 |
| Sample 12 | 4.70836 | 2.27628 | 0.40138 | −0.21585 | 5.15758 | 5.68819 | 4.72194 | −1.54359 | 1.61152 |
| Sample 13 | 4.24934 | 2.07020 | 0.32683 | −0.13531 | 4.39076 | 5.86771 | 4.70200 | −1.33169 | 1.82601 |
| Sample 14 | 3.48781 | 2.05132 | 0.43989 | 0.26726 | 3.94730 | 6.31536 | 4.98500 | −1.36619 | 1.42739 |
| Sample 15 | 2.82032 | 1.63524 | 0.15233 | −0.32273 | 3.54424 | 5.52271 | 4.53663 | −1.57456 | 1.05435 |
| Sample 16 | 6.52533 | 4.89437 | 0.44444 | 0.06621 | 4.90921 | 5.89195 | 4.37283 | −1.36928 | 1.77798 |
| Sample 17 | 6.58141 | 4.94319 | 0.52705 | 0.18376 | 5.12460 | 6.06473 | 4.96264 | −1.14801 | 1.70376 |
| Sample 18 | 3.95149 | 2.34378 | 0.61721 | −0.33804 | 4.11788 | 6.15918 | 4.76310 | −1.18521 | 1.42745 |
| Sample 19 | 4.41543 | 2.52345 | 0.73903 | 0.06344 | 4.68019 | 5.87201 | 4.59938 | −1.32062 | 1.20032 |
| LOD | 0.24800 | 0.32988 | 0.58034 | −0.29244 | 0.19114 | 0.48043 | 0.12797 | −1.33607 | 1.91274 |
| Missing | 5% | 5% | 77% | 36% | 5% | 5% | 5% | 38% | 77% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Cyr61 | DIk1 | Ahr | Ccl2 | Eno2 | Plin1 | Wfikkn2 | Flrt2 | Qdpr |
| Uniprot ID | P18406 | Q09163 | P30561 | P10148 | P17183 | Q8CGN5 | Q7TQN3 | Q8BLUO | Q8BVI4 |
| OlinkID | OID05063 | OID05064 | OID05065 | OID05066 | OID05067 | OID05068 | OID05069 | OID05070 | OID05071 |
| IPC1 | 7.06619 | 7.06299 | 8.74209 | 7.20998 | 7.49283 | 9.68273 | 6.33351 | 6.78288 | 8.72799 |
| IPC2 | 7.11627 | 7.18283 | 8.81266 | 7.25445 | 7.59629 | 9.78545 | 6.40557 | 6.80768 | 8.81210 |
| IPC3 | 7.14451 | 7.19947 | 8.60834 | 7.11176 | 7.60322 | 9.69685 | 6.21796 | 6.77689 | 8.77714 |
| NC1 | 0.44876 | 0.24105 | −0.21737 | 0.30871 | 0.90788 | −0.26542 | −0.44098 | 0.32017 | −0.69087 |
| NC2 | 0.78920 | 0.47923 | 0.32627 | 0.56855 | 1.06103 | −0.61024 | −0.39385 | 1.34453 | −0.32044 |
| NC3 | 0.84336 | 0.77993 | 0.75621 | 0.49820 | 1.23564 | 0.60544 | −0.07747 | 1.57157 | 0.19372 |
| Sample 1 | 4.54836 | 0.67615 | 7.50402 | 5.57795 | 1.49137 | 0.35846 | 0.02529 | 2.65901 | 7.16141 |
| Sample 7 | 1.77265 | 0.36938 | 8.25395 | 8.43752 | 0.95629 | 0.32228 | 0.00982 | 2.11840 | 7.22346 |
| Sample 3 | 3.09167 | 0.72447 | 7.70335 | 6.45098 | 1.35497 | 0.17421 | −0.09891 | 1.83686 | 7.18738 |
| Sample 11 | 2.19731 | 0.13778 | 8.33780 | 8.55326 | 1.12138 | 0.08172 | −0.11584 | 1.84262 | 7.09414 |
| Sample 5 | 3.45810 | 0.61292 | 7.76482 | 6.54573 | 1.55506 | 0.28889 | −0.07460 | 2.26903 | 7.15885 |
| Sample 8 | 1.83196 | 0.68890 | 7.84852 | 7.76070 | 1.33048 | 0.45584 | 0.18753 | 2.20748 | 7.25835 |
| Sample 9 | 1.79064 | 0.69847 | 8.64016 | 9.99382 | 1.20102 | −0.03351 | 0.20566 | 1.77101 | 7.26602 |
| Sample 12 | 2.83658 | 0.80204 | 8.69669 | 8.55747 | 1.15433 | 0.08196 | −0.06359 | 2.32841 | 7.33025 |
| Sample 13 | 2.77018 | 0.72811 | 8.54056 | 8.25473 | 1.03547 | 0.18802 | −0.04178 | 2.46422 | 7.28804 |
| Sample 14 | 5.19639 | 0.63502 | 8.05870 | 6.44949 | 1.67574 | 0.14343 | 0.23950 | 2.60529 | 7.25572 |
| Sample 15 | 4.34033 | 0.67911 | 7.39566 | 5.89524 | 1.33542 | 0.20606 | −0.19580 | 2.16711 | 7.06260 |
| Sample 16 | 3.00052 | 0.66623 | 8.18387 | 11.21860 | 0.97608 | 0.44198 | −0.09578 | 2.18989 | 6.94633 |
| Sample 17 | 2.19583 | 0.61707 | 8.02791 | 9.72244 | 1.07066 | 0.67561 | 0.13739 | 2.52616 | 7.21322 |
| Sample 18 | 3.01797 | 0.73244 | 8.65094 | 6.69126 | 1.39452 | 0.30948 | 0.12435 | 2.63552 | 7.25254 |
| Sample 19 | 4.00216 | 0.79735 | 8.29572 | 7.01789 | 1.24966 | 0.20013 | 0.09183 | 2.42633 | 7.23362 |
| LOD | 1.06690 | 0.98484 | 0.91349 | 0.88685 | 1.50882 | 0.87105 | 0.08369 | 1.44420 | 0.46537 |
| Missing | 5% | 83% | 5% | 5% | 69% | 91% | 60% | 4% | 5% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Inc Ctrl 2 | Det Ctrl | Fas | Erbb4 | Riox2 | Plxna4 | Epcam | Ccl3 | Crim1 |
| Uniprot ID | — | — | P25446 | Q61527 | Q8CD15 | Q80UG2 | Q99JW5 | P10855 | Q9JLLO |
| OlinkID | — | — | OID05074 | OID05075 | OID05076 | OID05077 | OID05078 | OID05079 | OID05080 |
| IPC1 | −0.02471 | 0.00932 | 6.05338 | 9.00771 | 10.40755 | 7.32203 | 9.50789 | 6.97043 | 10.13986 |
| IPC2 | −0.06066 | −0.08964 | 6.13932 | 8.94549 | 10.41027 | 7.38746 | 9.64136 | 7.01101 | 10.22559 |
| IPC3 | −0.08255 | −0.24767 | 6.02770 | 8.68147 | 10.24336 | 7.40221 | 9.57118 | 6.85885 | 10.14583 |
| NC1 | −0.05053 | −0.03561 | −0.82720 | −0.09834 | −0.59189 | −1.52216 | −0.31519 | 0.59124 | −0.77189 |
| NC2 | −0.26270 | −0.14182 | −0.77799 | 0.14720 | −1.53039 | −0.92131 | −0.77141 | 0.75700 | −0.15702 |
| NC3 | −0.04329 | 0.00813 | −0.32421 | 0.25823 | −0.95720 | −0.38778 | 0.09594 | 0.77980 | 0.09149 |
| Sample 1 | 0.38736 | −0.01817 | 8.08971 | 1.93094 | 6.68614 | −0.22195 | 1.54746 | 5.06274 | 2.93976 |
| Sample 7 | −0.24639 | 0.16140 | 8.23023 | 1.13024 | 6.57421 | −0.26787 | 2.08875 | 9.59998 | 3.46100 |
| Sample 3 | −0.23344 | 0.12000 | 8.22386 | 1.56448 | 6.69079 | −0.37608 | 1.62676 | 4.38664 | 2.37945 |
| Sample 11 | −0.40636 | 0.16333 | 7.94870 | 1.27714 | 6.21289 | −0.66770 | 2.37418 | 10.84552 | 3.48380 |
| Sample 5 | −0.35386 | 0.03730 | 8.15635 | 1.51796 | 6.78915 | −0.24167 | 1.36888 | 4.43770 | 2.73009 |
| Sample 8 | −0.25956 | 0.13373 | 8.21947 | 1.21645 | 6.59636 | −0.29271 | 2.21768 | 9.46790 | 3.52954 |
| Sample 9 | −0.17171 | 0.33127 | 8.13953 | 1.17366 | 6.52248 | −0.05338 | 2.15588 | 11.53389 | 3.01138 |
| Sample 12 | −0.46474 | 0.02158 | 8.19713 | 1.94444 | 6.02028 | −0.26824 | 1.47113 | 5.55541 | 2.88297 |
| Sample 13 | −0.33055 | 0.15884 | 8.28872 | 2.15065 | 6.55488 | −0.13407 | 1.10912 | 5.00809 | 2.69086 |
| Sample 14 | −0.24755 | 0.14837 | 8.22998 | 2.07612 | 6.50159 | 0.09694 | 1.10640 | 4.85219 | 3.13077 |
| Sample 15 | −0.40201 | 0.00354 | 8.00681 | 1.58707 | 6.12515 | −0.19300 | 0.62652 | 3.91704 | 2.46633 |
| Sample 16 | −0.48118 | 0.10267 | 8.10875 | 0.79462 | 6.77147 | −0.45151 | 1.73627 | 11.57893 | 2.96842 |
| Sample 17 | −0.25169 | 0.26245 | 8.16612 | 0.98619 | 6.97340 | −0.10424 | 2.24136 | 11.12489 | 3.19295 |
| Sample 18 | −0.24668 | 0.17667 | 8.36431 | 1.95380 | 7.04047 | 0.16374 | 1.63103 | 4.53560 | 2.71024 |
| Sample 19 | −0.33486 | 0.13160 | 8.15128 | 1.99375 | 6.92785 | 0.05976 | 1.30641 | 4.77806 | 2.83589 |
| LOD | −0.11884 | −0.05643 | −0.05902 | 0.57415 | −0.09343 | −0.39414 | 0.24732 | 1.01493 | 0.50858 |
| Missing | 0% | 0% | 5% | 5% | 5% | 32% | 5% | 5% | 5% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Vsig2 | Hgf | Sez612 | Il1a | Il23r | Dll1 | Ddah1 | Il10 | Tnfrsf12a |
| Uniprot ID | Q9Z109 | Q08048 | Q4V9Z5 | P01582 | Q8K4B4 | Q61483 | Q9CWS0 | P18893 | Q9CR75 |
| OlinkID | OID05081 | OID05082 | OID05083 | OID05084 | OID05085 | OID05086 | OID05087 | OID05088 | OID05089 |
| IPC1 | 9.25086 | 6.13933 | 5.02236 | 11.04316 | 7.71683 | 6.71124 | 6.40764 | 7.75775 | 8.53896 |
| IPC2 | 9.29580 | 6.23464 | 5.12652 | 11.05052 | 7.78498 | 6.71099 | 6.46274 | 7.83152 | 8.57177 |
| IPC3 | 9.16488 | 6.11351 | 5.05418 | 10.75977 | 7.67898 | 6.59545 | 6.50315 | 7.76742 | 8.53589 |
| NC1 | −1.36291 | 0.64797 | −1.32315 | 0.47768 | −1.60583 | −1.69566 | −1.26928 | −1.31852 | −0.83091 |
| NC2 | −1.47859 | 0.74273 | 1.23843 | 0.39579 | −1.06375 | 1.80867 | −0.49077 | −0.67244 | −0.45223 |
| NC3 | −1.36906 | 0.70543 | −1.10019 | 0.57991 | −0.52407 | −1.71050 | −0.69507 | −0.48144 | −0.39976 |
| Sample 1 | −0.54540 | 7.41757 | −0.54294 | 5.37536 | −0.91137 | −1.18954 | 6.30567 | −0.26762 | −0.72198 |
| Sample 7 | −1.10033 | 6.21963 | −0.40224 | 7.87345 | −0.72283 | −1.03360 | 7.34802 | −0.21962 | −0.67484 |
| Sample 3 | −0.76715 | 6.98490 | −0.67610 | 6.76682 | −0.84610 | −1.10708 | 6.94736 | −1.46550 | −0.52589 |
| Sample 11 | −0.75079 | 6.00894 | −0.74856 | 7.88917 | −0.22166 | −0.79888 | 6.47895 | 0.30015 | −0.64443 |
| Sample 5 | −1.59575 | 7.26989 | −0.74033 | 6.80255 | −0.60605 | −1.04863 | 6.63298 | −0.52100 | −0.72534 |
| Sample 8 | −0.73802 | 5.79858 | −0.69010 | 7.23114 | −0.71795 | −0.61613 | 6.83657 | −0.12833 | −0.35746 |
| Sample 9 | −0.77627 | 6.42109 | −0.52064 | 8.55662 | −0.38832 | −1.28168 | 6.30136 | 0.13363 | −0.30725 |
| Sample 12 | −1.36082 | 7.68482 | −0.51941 | 7.47314 | −0.67305 | −0.90554 | 7.73170 | −0.24216 | −0.74363 |
| Sample 13 | −0.96194 | 7.76483 | −0.62857 | 7.07746 | −0.58299 | −1.07639 | 7.07055 | −0.68607 | −0.44396 |
| Sample 14 | −0.80307 | 7.92925 | −0.66210 | 5.59095 | −0.68400 | −1.27996 | 7.28682 | −0.48606 | −0.45656 |
| Sample 15 | −0.73086 | 6.95000 | −0.76743 | 5.49904 | −0.83243 | −1.49442 | 6.23593 | −0.71822 | −0.55789 |
| Sample 16 | −0.34284 | 6.31077 | −0.57742 | 9.27233 | −0.46173 | −1.30403 | 5.10228 | −0.10874 | −0.51593 |
| Sample 17 | −1.21585 | 6.22259 | −0.41261 | 8.74077 | −0.38259 | −0.89933 | 5.28984 | −0.33431 | −0.31011 |
| Sample 18 | −0.72638 | 7.90357 | −0.36774 | 7.10599 | −0.81069 | −0.81471 | 6.61812 | −0.30115 | −0.60641 |
| Sample 19 | −1.50599 | 7.61629 | −0.60691 | 6.48721 | −0.77137 | −1.25521 | 6.54290 | −0.65102 | −0.60384 |
| LOD | −0.73989 | 0.97847 | −0.92975 | 1.33569 | −0.45622 | −1.37248 | −0.34019 | −0.34862 | −0.16634 |
| Missing | 74% | 5% | 10% | 5% | 83% | 29% | 5% | 57% | 90% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Acvrl1 | Lgmn | Csf2 | Cxcl9 | Map2k6 | Itgb1bp2 | Il17f | Il1b | Casp3 |
| Uniprot ID | Q61288 | O89017 | P01587 | P18340 | P70236 | Q9R000 | Q7TNI7 | P10749 | P70677 |
| OlinkID | OID05090 | OID05091 | OID05092 | OID05093 | OID05094 | OID05095 | OID05096 | OID05097 | OID05098 |
| IPC1 | 8.70110 | 6.37240 | 5.87490 | 10.70436 | 8.81050 | 9.62055 | 7.24854 | 7.88637 | 9.06224 |
| IPC2 | 8.79279 | 6.41101 | 5.95100 | 10.76818 | 8.89201 | 9.76195 | 7.33720 | 7.96311 | 9.16855 |
| IPC3 | 8.69628 | 6.10396 | 5.96139 | 10.72894 | 8.87138 | 9.78566 | 7.32584 | 7.94400 | 9.10995 |
| NC1 | 0.08952 | −0.06152 | −0.36742 | −1.16915 | −0.19480 | −1.59529 | −0.37281 | 0.34192 | −0.08716 |
| NC2 | −0.25950 | −0.15711 | −0.15029 | −1.40342 | −0.24600 | −1.37362 | 0.04413 | 0.48032 | −0.04372 |
| NC3 | 0.02393 | 0.10429 | 0.25445 | −1.25085 | 0.11727 | −1.60366 | 0.29681 | 0.70513 | 0.08668 |
| Sample 1 | 1.22059 | 3.60101 | 0.27483 | 4.69631 | 6.82328 | −1.13175 | −0.15953 | 0.79327 | 8.91851 |
| Sample 7 | 4.36658 | 3.59475 | 1.75088 | 11.64329 | 6.56848 | −0.72957 | −0.06575 | 2.37855 | 9.27710 |
| Sample 3 | 1.65592 | 3.56826 | 0.27006 | 7.19299 | 7.09946 | −0.67550 | −0.14356 | 1.24596 | 9.00199 |
| Sample 11 | 3.83146 | 4.05529 | 1.86804 | 11.15480 | 6.20954 | −0.60676 | −0.15718 | 2.23091 | 9.35073 |
| Sample 5 | 1.58627 | 3.65390 | 0.34993 | 6.91183 | 7.13504 | −0.72980 | −0.17609 | 1.33508 | 9.12122 |
| Sample 8 | 3.95306 | 3.78011 | 1.24350 | 10.72518 | 6.45326 | −0.85413 | −0.10558 | 1.93374 | 9.25617 |
| Sample 9 | 4.59146 | 3.99960 | 2.40027 | 11.92525 | 6.59661 | −1.17109 | −0.07789 | 2.87049 | 9.38119 |
| Sample 12 | 1.59000 | 3.36158 | 0.53150 | 6.97014 | 6.85675 | −0.79560 | −0.04019 | 2.15254 | 9.24658 |
| Sample 13 | 1.94551 | 3.54555 | 0.51674 | 6.69450 | 6.99321 | −1.14273 | −0.25538 | 2.11399 | 9.18139 |
| Sample 14 | 1.56919 | 3.66052 | 0.51442 | 6.01787 | 7.00949 | −1.05543 | 0.41595 | 0.93986 | 9.13843 |
| Sample 15 | 1.09839 | 3.38377 | 0.21295 | 5.57297 | 6.81661 | −1.87303 | 0.09844 | 0.89178 | 8.73472 |
| Sample 16 | 5.12081 | 3.94453 | 2.78114 | 11.49674 | 6.65906 | −0.67657 | −0.29594 | 4.02337 | 9.32589 |
| Sample 17 | 5.16720 | 4.24364 | 2.44088 | 11.47916 | 6.70465 | −0.60100 | −0.01938 | 3.84670 | 9.46209 |
| Sample 18 | 1.65993 | 3.78288 | 0.46061 | 6.47941 | 7.26560 | −0.98371 | −0.06643 | 1.40892 | 9.25075 |
| Sample 19 | 1.92136 | 3.65345 | 0.38858 | 7.11098 | 7.17916 | −1.07429 | 0.07562 | 1.10639 | 9.19200 |
| LOD | 0.53346 | 0.34604 | 0.40550 | −0.46929 | 0.38150 | −0.86686 | 0.36964 | 0.94715 | 0.96300 |
| Missing | 5% | 5% | 39% | 5% | 5% | 56% | 91% | 21% | 5% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Apbb1ip | Wisp1 | Cdh6 | Pdgfb | Igsf3 | Tgfbr3 | Cxcl1 | Pak4 | Cntn4 |
| Uniprot ID | Q8R5A3 | O54775 | P97326 | P31240 | Q6ZQA6 | O88393 | P12850 | Q8BTW9 | Q69Z26 |
| OlinkID | OID05099 | OID05100 | OID05101 | OID05102 | OID05103 | OID05104 | OID05105 | OID05106 | OID05107 |
| IPC1 | 7.01156 | 7.47905 | 7.89979 | 6.98242 | 8.72112 | 8.21074 | 7.32556 | 9.44560 | 8.37844 |
| IPC2 | 7.12576 | 7.60494 | 7.94508 | 7.12884 | 8.78322 | 8.30174 | 7.46723 | 9.53702 | 8.48147 |
| IPC3 | 7.08163 | 7.72659 | 7.81919 | 7.10501 | 8.75196 | 8.19316 | 7.53899 | 9.58313 | 8.50265 |
| NC1 | −0.28800 | −0.21373 | −0.89823 | 0.31890 | −0.02018 | −0.46880 | −0.58504 | −1.28418 | −0.30201 |
| NC2 | −0.02580 | −0.48906 | −1.10304 | 0.27968 | 0.39496 | −0.32186 | −0.00414 | −1.38565 | −0.01314 |
| NC3 | −0.14955 | −0.19241 | −1.08727 | 0.36854 | 0.65002 | 0.06922 | 0.15123 | −0.60835 | 0.14954 |
| Sample 1 | 2.46524 | 1.61863 | −0.43627 | 3.82189 | 2.86043 | 1.84440 | 3.07343 | 4.50382 | 0.30734 |
| Sample 7 | 4.94661 | 0.62096 | −0.60695 | 4.28536 | 2.48490 | 0.98585 | 6.35971 | 6.11372 | 0.52749 |
| Sample 3 | 2.58838 | 1.34621 | −0.66650 | 3.47981 | 2.59780 | 1.13355 | 3.06945 | 4.99440 | −0.01439 |
| Sample 11 | 5.08365 | 0.31906 | −0.81576 | 4.00555 | 2.23636 | 1.29782 | 6.29375 | 6.12640 | 0.28619 |
| Sample 5 | 2.86624 | 1.20024 | −0.64786 | 3.39891 | 3.01804 | 1.27089 | 3.15562 | 5.39762 | 0.62161 |
| Sample 8 | 4.46073 | 0.60324 | −0.56076 | 3.76129 | 2.41285 | 1.06044 | 5.63475 | 5.90923 | 0.36291 |
| Sample 9 | 5.25618 | 0.50905 | −0.39187 | 4.50508 | 2.21427 | 0.70436 | 6.97974 | 6.20722 | 0.35430 |
| Sample 12 | 2.88361 | 1.68870 | −0.36237 | 4.00568 | 3.29084 | 1.75418 | 8.60256 | 5.62497 | 0.13208 |
| Sample 13 | 2.69457 | 1.52160 | −0.89715 | 4.03819 | 2.86991 | 1.68778 | 9.00658 | 5.61561 | 0.37957 |
| Sample 14 | 2.08028 | 1.79907 | −0.55652 | 4.60660 | 2.81147 | 1.84438 | 3.27832 | 5.30389 | 0.21715 |
| Sample 15 | 1.69676 | 1.24938 | −0.51177 | 2.73019 | 2.25321 | 1.19997 | 2.93316 | 4.36953 | −0.02903 |
| Sample 16 | 5.23531 | 1.23828 | −0.49433 | 4.40551 | 2.38375 | 0.34539 | 6.31453 | 5.57636 | 0.00790 |
| Sample 17 | 5.38761 | 1.18734 | −0.30440 | 4.29092 | 2.49679 | 0.75805 | 6.06713 | 5.90501 | 0.45712 |
| Sample 18 | 2.64042 | 1.84929 | −0.46360 | 4.51490 | 3.25812 | 1.90098 | 3.97796 | 5.77116 | 0.67146 |
| Sample 19 | 2.73114 | 1.78876 | −0.45582 | 3.95759 | 3.10853 | 1.74093 | 6.27653 | 5.93247 | 0.34691 |
| LOD | 0.30688 | 0.22974 | −0.63451 | 0.70646 | 0.86625 | 0.16778 | 0.45402 | −0.28238 | 0.31929 |
| Missing | 5% | 5% | 44% | 5% | 5% | 5% | 5% | 5% | 51% |
| Data freq. | | | | | | | | | |
| | Olink ® | Olink ® | Olink ® | Olink ® | Olink ® | Olink ® | Olink ® | Olink ® | Olink ® |
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Ghrl | Lpl | Fstl3 | Dctn2 | Il5 | Eda2r | Ntf3 | Tnfsf12 | Ccl20 |
| Uniprot ID | Q9EQX0 | P11152 | Q9EQC7 | Q99KJ8 | P04401 | Q8BX35 | P20181 | O54907 | O89093 |
| OlinkID | OID05108 | OID05109 | OID05110 | OID05111 | OID05112 | OID05113 | OID05114 | OID05115 | OID05116 |
| IPC1 | 8.23565 | 8.49470 | 6.17858 | 7.67081 | 11.59456 | 7.80389 | 8.16763 | 9.97459 | 9.24278 |
| IPC2 | 8.41232 | 8.57093 | 6.26093 | 7.82006 | 11.73041 | 7.87461 | 8.29585 | 10.17400 | 9.37389 |
| PC3 | 8.44237 | 8.55238 | 6.31827 | 7.88000 | 11.73434 | 7.92958 | 8.36905 | 10.17402 | 9.40816 |
| NC1 | 0.22678 | −1.17933 | 1.45503 | −0.42698 | −0.49692 | 1.42200 | −0.46501 | −2.12288 | −1.28976 |
| NC2 | 0.48346 | −0.74207 | 1.36375 | 0.17523 | −0.49137 | 1.52027 | 0.13093 | −1.13226 | −0.85437 |
| NC3 | 0.70993 | −0.66734 | 1.40833 | 0.18495 | 0.03190 | 1.56707 | −0.07858 | −0.82596 | −0.52778 |
| Sample 1 | 0.25186 | 3.62828 | 1.55139 | 8.04694 | −0.87611 | 2.03261 | 1.90395 | 1.96609 | 5.55529 |
| Sample 7 | 0.28237 | 1.83760 | 1.88463 | 8.20702 | −0.19042 | 2.45283 | 0.46828 | 0.63245 | 5.98936 |
| Sample 3 | 0.54732 | 3.30368 | 1.41481 | 7.62710 | −0.96755 | 1.71858 | 2.11675 | 1.18220 | 5.35309 |
| Sample 11 | 0.21071 | 1.29447 | 1.88287 | 8.40075 | 0.98943 | 2.03411 | 0.65778 | 0.87821 | 5.25599 |
| Sample 5 | 0.37508 | 5.35924 | 1.62521 | 7.70584 | −0.82830 | 1.95948 | 2.12560 | 1.53165 | 5.47145 |
| Sample 8 | 0.49663 | 1.74881 | 1.79895 | 8.18463 | −0.04881 | 2.21322 | 0.44574 | 0.60799 | 5.87630 |
| Sample 9 | 0.44735 | 1.99514 | 2.32596 | 8.26466 | 0.34940 | 2.29913 | −0.08665 | 0.65019 | 4.69067 |
| Sample 12 | 0.45720 | 4.47225 | 1.61541 | 8.25650 | −0.03006 | 2.25439 | 2.71542 | 1.87328 | 5.30187 |
| Sample 13 | 0.34194 | 2.61065 | 1.50775 | 7.99401 | −0.69811 | 2.19273 | 2.31214 | 1.61095 | 5.63924 |
| Sample 14 | 0.71535 | 3.03231 | 1.58654 | 8.07014 | 0.80785 | 1.92105 | 2.16919 | 1.82731 | 6.38311 |
| Sample 15 | 0.38192 | 2.20855 | 1.31059 | 7.77472 | −0.16778 | 1.71927 | 1.71828 | 1.31742 | 5.85822 |
| Sample 16 | 0.06826 | 2.66607 | 2.17096 | 8.09157 | −0.06900 | 2.81615 | 0.43693 | 0.57128 | 5.53727 |
| Sample 17 | 0.47548 | 2.50067 | 1.73219 | 8.11771 | 0.32981 | 3.07811 | 0.02636 | 0.97644 | 6.38872 |
| Sample 18 | 0.35123 | 2.93713 | 1.57061 | 7.94874 | 0.25094 | 1.79178 | 2.49454 | 1.72064 | 6.58365 |
| Sample 19 | 0.59981 | 3.14888 | 1.57181 | 7.85057 | −1.48122 | 2.09741 | 2.71805 | 1.89747 | 6.48455 |
| LOD | 0.97644 | −0.33241 | 1.77677 | 0.45556 | 1.18988 | 1.86738 | 0.43327 | −0.40346 | −0.10289 |
| Missing | 95% | 6% | 68% | 5% | 96% | 17% | 9% | 5% | 5% |
| Data freq. | | | | | | | | | |

| | Olink ® | Olink ® | Olink ® | Olink ® | Olink ® | Olink ® | Olink ® | Olink ® | Olink ® |
|---|---|---|---|---|---|---|---|---|---|
| | MOUSE | MOUSE | MOUSE | MOUSE | MOUSE | MOUSE | MOUSE | MOUSE | MOUSE |
| | EXPLOR- | EXPLOR- | EXPLOR- | EXPLOR- | EXPLOR- | EXPLOR- | EXPLOR- | EXPLOR- | EXPLOR- |
| | ATORY | ATORY | ATORY | ATORY | ATORY | ATORY | ATORY | ATORY | ATORY |
| Panel | (v.3801) | (v.3801) | (v.3801) | (v.3801) | (v.3801) | (v.3801) | (v.3801) | (v.3801) | (v.3801) |
| Assay | Fli1 | Tpp1 | Tnr | Vegfd | Parp1 | Tnf | Plate ID | QC | QC |
| | | | | | | | | Warning | Deviation |
| | | | | | | | | | from median |
| Uniprot ID | P26323 | O89023 | Q8BY19 | P97946 | P11103 | P06804 | — | — | Inc Ctrl 2 |
| OlinkID | OID05117 | OID05118 | OID05119 | OID05120 | OID05121 | OID05122 | — | — | — |
| IPC1 | 7.62219 | 6.88196 | 7.70882 | 8.35294 | 7.88903 | 6.43173 | HIMC_Mouse_Lee_1204 | Pass | 0.27332 |
| | | | | | | | 2019 | | |
| IPC2 | 7.77449 | 6.97671 | 7.83919 | 8.50147 | 7.98436 | 6.53268 | HIMC_Mouse_Lee_1204 | Pass | 0.23737 |
| | | | | | | | 2019 | | |
| IPC3 | 7.91444 | 7.10988 | 7.93573 | 8.43607 | 8.00497 | 6.64606 | HIMC_Mouse_Lee_1204 | Pass | 0.21549 |
| | | | | | | | 2019 | | |
| NC1 | −0.45994 | −0.00959 | −0.33142 | −0.36761 | −1.12010 | −1.29445 | HIMC_Mouse_Lee_1204 | Pass | 0.24750 |
| | | | | | | | 2019 | | |
| NC2 | 0.34695 | 0.58819 | −0.10585 | −0.12314 | −0.54101 | −1.66215 | HIMC_Mouse_Lee_1204 | Pass | 0.03533 |
| | | | | | | | 2019 | | |
| NC3 | 0.26559 | 0.49780 | 0.13978 | −0.08842 | −0.65735 | −1.37652 | HIMC_Mouse_Lee_1204 | Pass | 0.25475 |
| | | | | | | | 2019 | | |
| Sample 1 | 2.67647 | 3.73193 | −0.11925 | 0.10399 | 9.78744 | −1.00723 | HIMC_Mouse_Lee_1204 | Pass | −0.08932 |
| | | | | | | | 2019 | | |
| Sample 7 | 5.46173 | 3.00523 | −0.02982 | 0.10660 | 9.92729 | −0.80092 | HIMC_Mouse_Lee_1204 | Pass | 0.05165 |
| | | | | | | | 2019 | | |
| Sample 3 | 3.24148 | 2.70659 | −0.31042 | 0.04130 | 10.40390 | −0.60476 | HIMC_Mouse_Lee_1204 | Pass | 0.06460 |
| | | | | | | | 2019 | | |
| Sample 11 | 5.92464 | 3.45976 | 0.31606 | −0.35392 | 9.75386 | −0.65938 | HIMC_Mouse_Lee_1204 | Pass | −0.10833 |
| | | | | | | | 2019 | | |
| Sample 5 | 3.53217 | 3.28431 | −0.13022 | 0.18161 | 10.38260 | −0.70690 | HIMC_Mouse_Lee_1204 | Pass | −0.05582 |
| | | | | | | | 2019 | | |
| Sample 8 | 5.56630 | 3.02685 | 0.20771 | 0.22142 | 10.24351 | −1.02699 | HIMC_Mouse_Lee_1204 | Pass | 0.03847 |
| | | | | | | | 2019 | | |
| Sample 9 | 6.19365 | 3.72332 | 0.04156 | 0.17107 | 10.24990 | −0.10685 | HIMC_Mouse_Lee_1204 | Pass | 0.12632 |
| | | | | | | | 2019 | | |
| Sample 12 | 3.49100 | 3.94787 | 0.12890 | −0.22094 | 10.00200 | −1.33945 | HIMC_Mouse_Lee_1204 | Pass | −0.16670 |
| | | | | | | | 2019 | | |
| Sample 13 | 3.05236 | 3.82080 | 0.13145 | 0.16118 | 10.37796 | −1.04202 | HIMC_Mouse_Lee_1204 | Pass | −0.03251 |
| | | | | | | | 2019 | | |
| Sample 14 | 2.93818 | 4.10761 | 0.10771 | 0.04100 | 10.21362 | −0.78042 | HIMC_Mouse_Lee_1204 | Pass | 0.05048 |
| | | | | | | | 2019 | | |
| Sample 15 | 2.23649 | 3.16642 | 0.00832 | 0.13614 | 9.89764 | −0.96091 | HIMC_Mouse_Lee_1204 | Pass | −0.10398 |
| | | | | | | | 2019 | | |
| Sample 16 | 5.65023 | 3.12745 | −0.35537 | 0.17644 | 10.09898 | −0.11605 | HIMC_Mouse_Lee_1204 | Pass | −0.18315 |
| | | | | | | | 2019 | | |
| Sample 17 | 6.45885 | 3.85709 | −0.12033 | 0.44364 | 10.37092 | −0.10562 | HIMC_Mouse_Lee_1204 | Pass | 0.04634 |
| | | | | | | | 2019 | | |
| Sample 18 | 3.17116 | 3.94763 | 0.20153 | 0.43116 | 10.64561 | −1.44166 | HIMC_Mouse_Lee_1204 | Pass | 0.05135 |
| | | | | | | | 2019 | | |
| Sample 19 | 3.20037 | 4.07127 | 0.19251 | 0.16311 | 10.63985 | −0.85278 | HIMC_Mouse_Lee_1204 | Pass | −0.03682 |
| | | | | | | | 2019 | | |
| LOD | 0.70915 | 0.88752 | 0.37063 | 0.37784 | −0.00544 | −0.88059 | | | |
| Missing | 5% | 5% | 91% | 83% | 5% | 64% | | | |
| Data freq. | | | | | | | | | |

| Panel | Olink ® MOUSE EXPLORATORY(v.3801) |
|---|---|
| Assay | QC Deviation from median |
| Uniprot ID | Det Ctrl |
| OlinkID | |
| IPC1 | 0.00000 |
| IPC2 | 0.00000 |
| IPC3 | 0.00000 |
| NC1 | −0.10899 |
| NC2 | −0.21521 |
| NC3 | −0.06525 |
| Sample 1 | −0.09156 |
| Sample 7 | 0.08801 |
| Sample 3 | 0.04662 |
| Sample 11 | 0.08995 |
| Sample 5 | −0.03608 |
| Sample 8 | 0.06034 |
| Sample 9 | 0.25789 |
| Sample 12 | −0.05181 |
| Sample 13 | 0.08545 |
| Sample 14 | 0.07498 |
| Sample 15 | −0.06985 |
| Sample 16 | 0.02928 |
| Sample 17 | 0.18906 |
| Sample 18 | 0.10329 |
| Sample 19 | 0.05821 |

The mature NTF3 protein is highly conserved in vertebrates, with the human and mouse orthologs sharing 96% amino acid identity. Therefore, freshly isolated primary mouse hepatocytes were incubated in serum-free (FIG. 8F) and in serum-containing media (FIG. 9D) supplemented with recombinant NTF3 or vehicle control. It was found herein that cell proliferation was significantly higher in NTF3-treated primary hepatocytes than in control-treated hepatocytes (FIG. 8F; FIG. 9D). Similarly, NTF3 treatment of the mouse hepatocyte AML12 cell line (FIGS. 9E-F) and the human HepG2 cell line (FIGS. 8G-H) greatly stimulated cell proliferation. NTF3 binds and activates the tropomyosin receptor kinases (TRKs) TRKA, TRKB, and TRKC, which are encoded by NTRK1-3. Binding of NTF3 to a TRK induces autophosphorylation of the TRK and activation of downstream signaling pathways. Cancers with NTRK genes fusions express proteins that have constitutive receptor activity and are oncogenic drivers, rendering them therapeutically responsive to TRK inhibitors. Thus, whether the highly specific TRKA and TRKC inhibitor, Loxo-195 (selectritinib) inhibited NTF3-induced hepatocyte proliferation was investigated. Indeed, Loxo-195 (final concentration 5 nM) significantly inhibited proliferation in AML12 and HepG2 cells, indicating that NTF3 induces cell proliferation through TRK signaling (FIGS. 8G-H and FIGS. 9E-F).

Because HSC depletion decreased hepatocyte CCND1 expression (FIGS. 5I-J), whether NTF3 affected CCND1 abundance in culture was investigated. Incubation of HepG2 cells with NTF3 increased CCND1 abundance, which was inhibited by the addition of Loxo-195 (FIGS. 8I-J). Increased TRK phosphorylation was observed at tyrosine residues in the activation loop of TRKA or TRKB (P-TRKA^{Tyr674/675}, P-TRKB^{Tyr706/707}) which were decreased by addition of Loxo-195 (FIGS. 9G-H). These results were consistent using NTF3 from three different vendors; in all cases, CCND1 amounts increased in both AML 12 and HepG2 cells (FIGS. 9I-L). HGF is a potent hepatocyte mitogen that is secreted by HSCs and liver sinusoidal endothelial cells. Therefore, proliferative effects of NTF3 were compared to HGF in primary mouse hepatocytes (FIG. 9M). HGF and NTF3 increased primary hepatocyte proliferation by 2.6 and 1.5-fold, respectively, indicating that HGF is a more potent mitogen than NTF3 at the tested concentrations. To assess if NTF3 is an additional factor that synergizes with HGF, primary hepatocytes were incubated with either NTF3, HGF, or both growth factors, and found no synergistic effects at the tested concentrations (FIG. 9N).

### Example 5

### Exogenous NTF3 Rescues CCND1 Expression and Increases Liver Mass in HSC-Depleted Mice

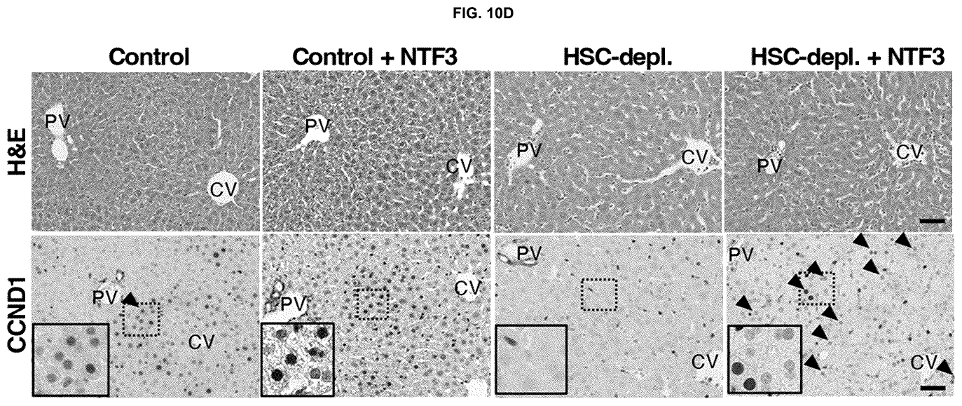

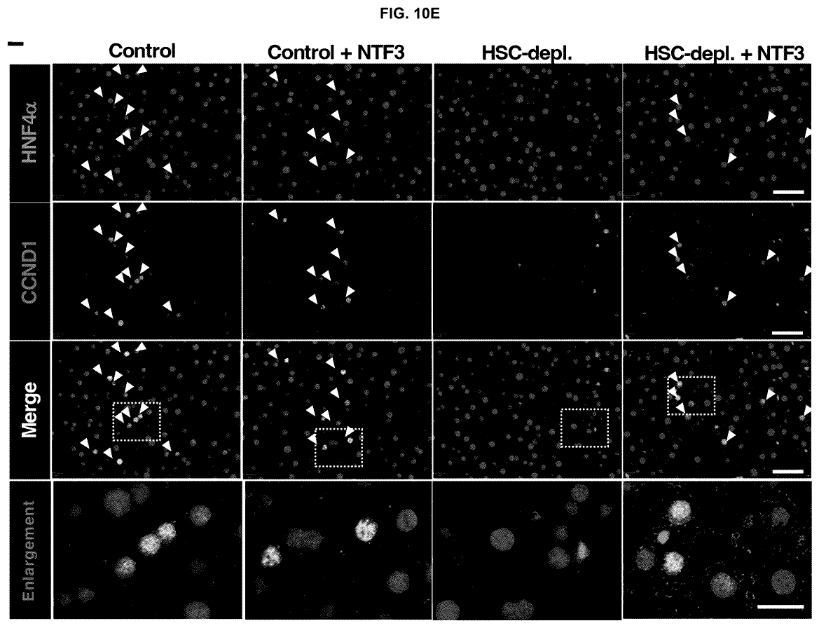

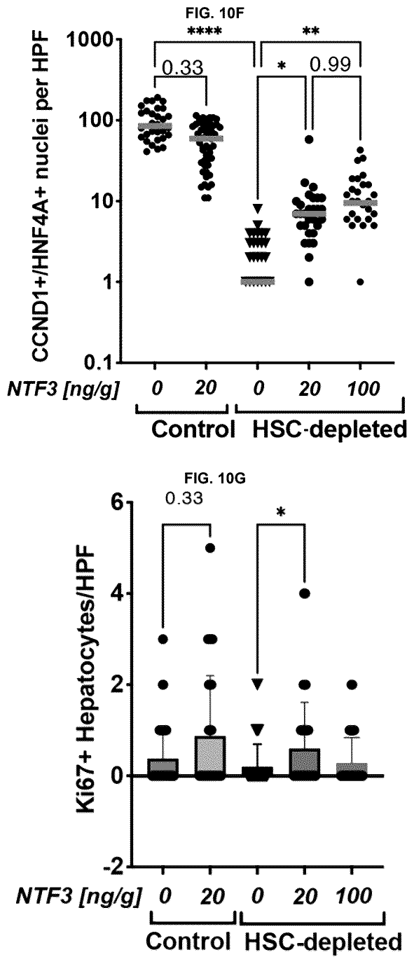

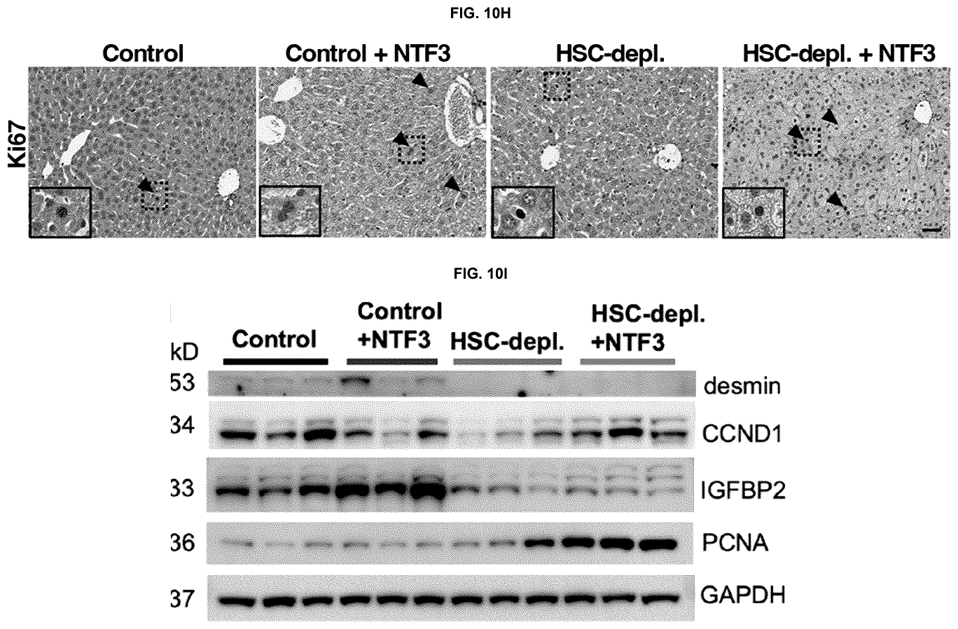

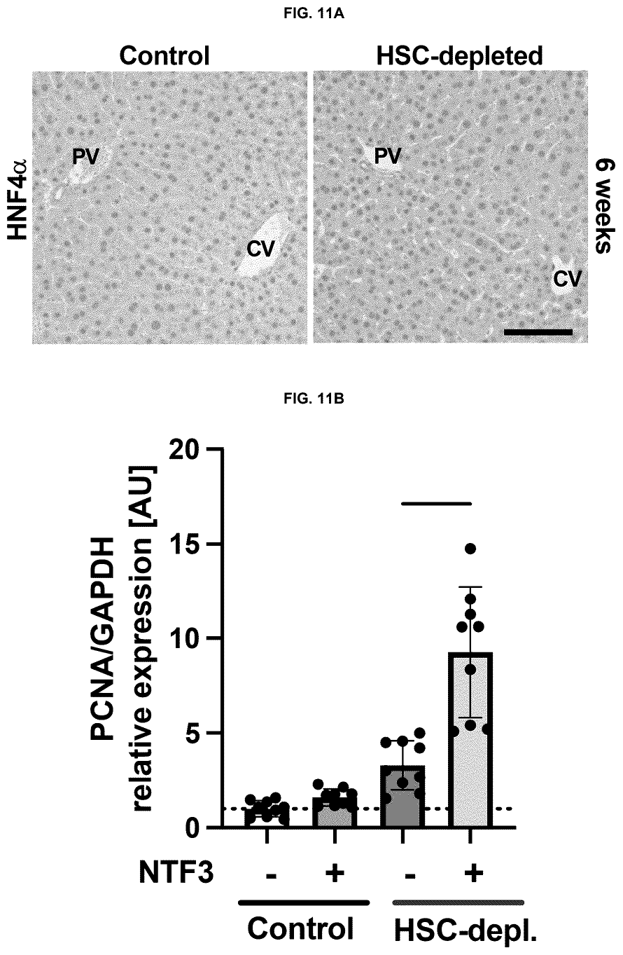

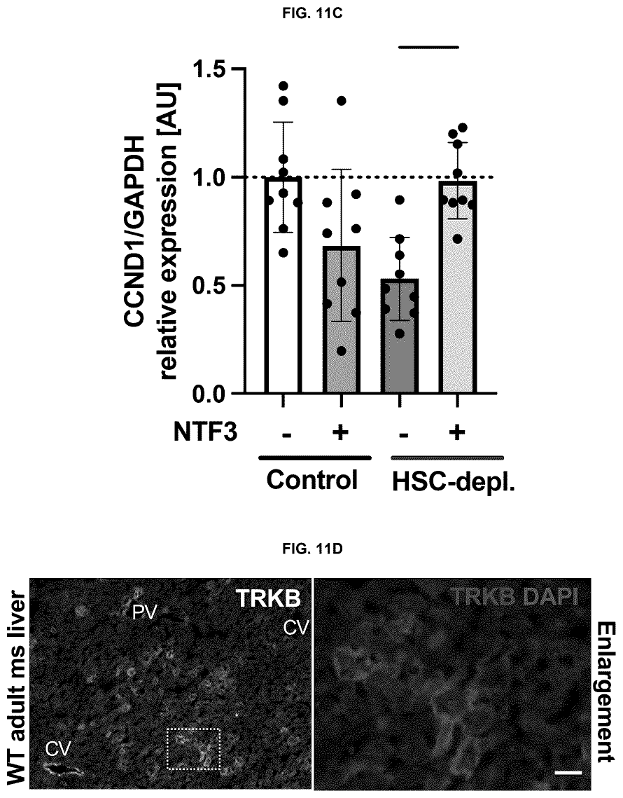

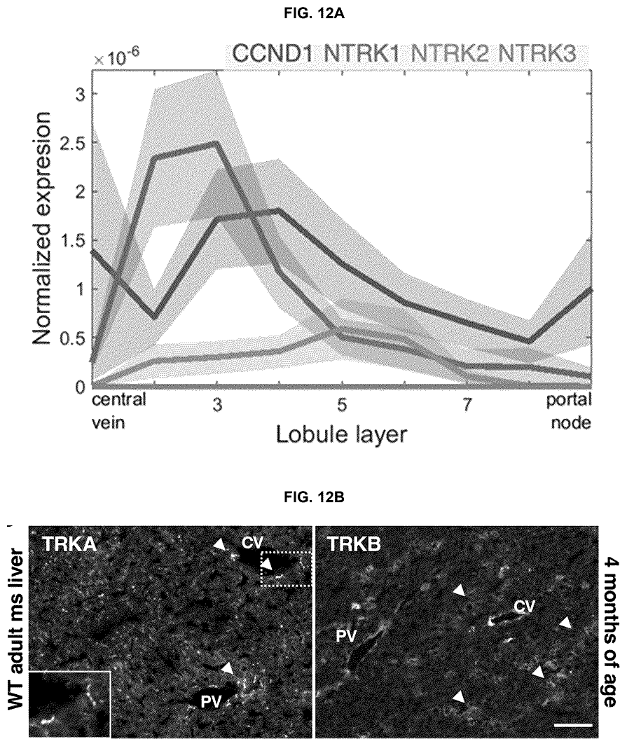

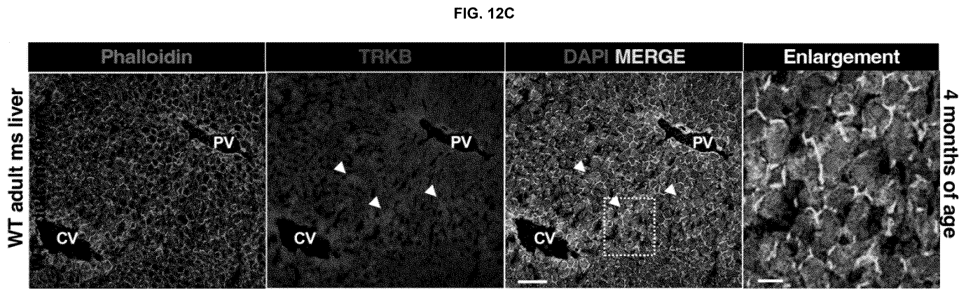

In the murine model herein, ablation of HSCs decreased nuclear CCND1 in midlobular hepatocytes (FIGS. 5I-J). To determine whether NTF3 is required for midlobular CCND1 expression, whether in vivo administration of NTF3 increased hepatocyte proliferation and CCND1 expression in HSC-depleted mice was assessed. HSC depletion was induced and mice were injected with NTF3 at 20 ng/g or 100 ng/g body weight or with vehicle control for 4 consecutive days, then the mice were sacrificed after the last injection (FIGS. 10A-B). The liver-to-body weight ratio of mice treated with NTF3 dose-dependently increased with a significant increase in liver-to-body weight ratio in the higher dose group (FIG. 10C). As expected, in HSC-depleted mice treated with vehicle only, CCND1 was decreased in hepatocytes and there was some scattered CCND1 expression in non-parenchymal cells (FIG. 10D). NTF3 treatment of HSC-depleted mice increased nuclear CCND1 in hepatocytes in the midlobular region (FIG. 10D). To quantify CCND1 expression in hepatocytes, livers were assessed by multiplex immunohistochemistry for CCND1 and hepatocyte nuclear factor 4a (HNF4a), which is ubiquitously expressed in the nuclei of hepatocytes across the liver lobule and was retained in HSC-depleted mice (FIG. 11A). Treatment with NTF3 dose-dependently increased the number of CCND1+HNF4α+ nuclei (FIGS. 10E-F). Similarly, an increase in Ki67+ hepatocytes was observed, consistent with increased regenerative activity in HSC-depleted mice (FIGS. 10G-H). Western blotting of whole liver lysates also confirmed that NTF3 treatment increased CCND1 and proliferating cell nuclear antigen (PCNA) in HSC-depleted mice, further indicating increased regenerative activity (FIG. 10I; FIGS. 11B-D).

### Example 6

### NTF3 Signals Through TRKB in Mouse Hepatocytes

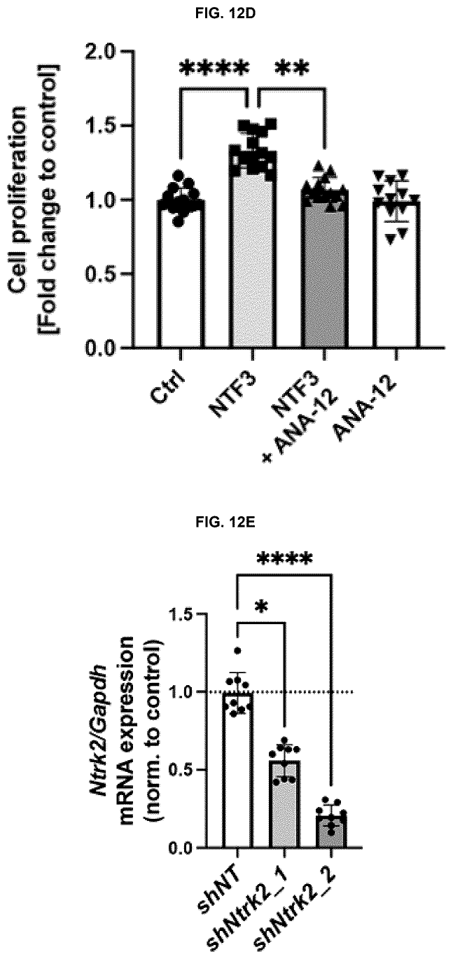

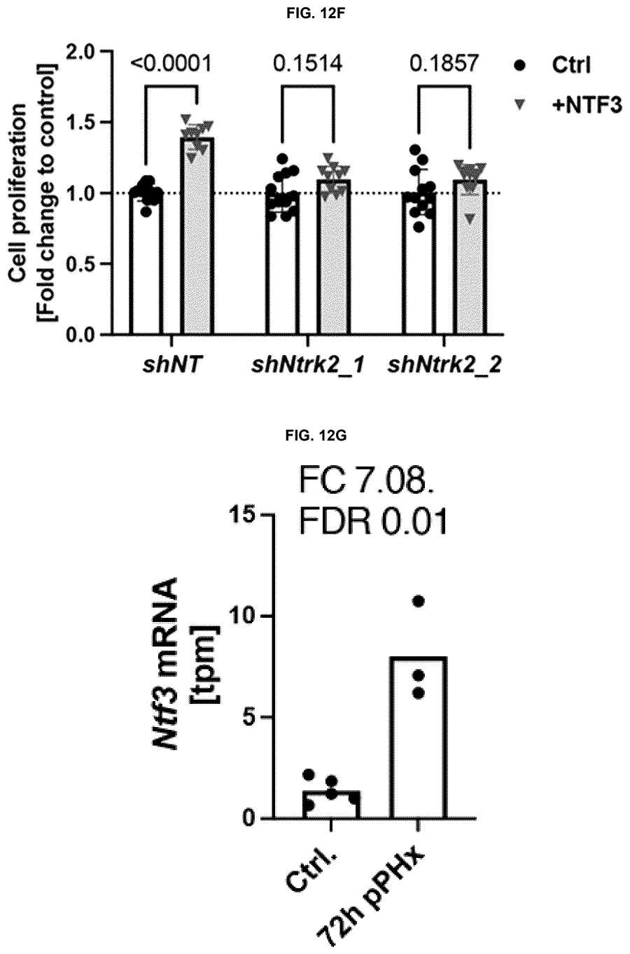

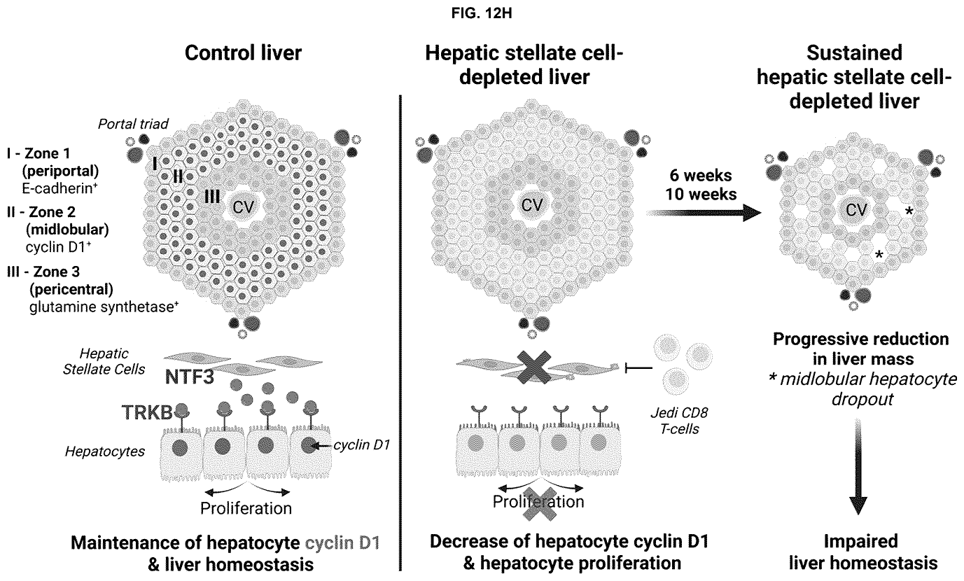

Analysis of a published mouse liver dataset that allows for zone-specific gene expression analysis of hepatocytes (lobule layers 1 through 9) (Halpern et al., Nature. 2017; 542(7641): 352-356) showed expression of NTRK1 and NTRK2 in midlobular hepatocyte layers 2 through 7 (FIG. 12A), whereas NTRK3 was absent. Immunofluorescence analysis for TRKA (NTRK1) showed non-parenchymal TRKA staining around the portal tracts and central veins (FIG. 12B). TRKB (NTRK2) staining was observed in midlobular hepatocytes with decreased staining around portal tracts and central veins, suggesting that TRKB mediates the effects of HSC-derived NTF3 (FIG. 12B and FIG. 11D). Costaining for filamentous actin and TRKB indicated TRKB-positive hepatocytes in zone 2 (FIG. 12C). To assess if NTF3 signals though TRKB in primary mouse hepatocytes, these cells were incubated with ANA-12, a highly specific TRKB antagonist that does not alter TRKA or TRKC function. ANA-12 significantly inhibited NTF3-induced cell proliferation when used at 10 nM (FIG. 12D). To confirm that TRKB mediated NTF-3 signaling in hepatocytes, knockdown experiments were performed in primary mouse hepatocytes using lentiviral vectors expressing two different shRNAs targeting Ntrk2 (shNtrk2_1 and shNtrk2_2) and a control lentiviral vector expressing a non-targeting shRNA (shNT). Knockdown of Ntrk2 mRNA was confirmed by qRT-PCR and showed decreases of 45% for shNtrk2_1 and 80% shNtrk2_1 (FIG. 12E). Knockdown of Ntrk2, which encodes TRKB, blocked NTF3-induced cell proliferation (FIG. 12F). It has been reported that hepatocyte proliferation after partial hepatectomy initiates in midlobular hepatocytes before proceeding to pericentral and periportal areas. By gene expression analysis, Ntf3 was significantly increased in mouse livers at 72 hours after partial hepatectomy, further supporting a pro-proliferative role of NTF3 (FIG. 12G), though further studies are required to address the role of NTF3 in liver regeneration. Overall, the findings herein indicate that HSCs maintain liver homeostasis by secreting NTF3 that stimulates the proliferation of zone 2 hepatocytes by activating TRKB.

### Example 7

### Discussion for Examples 1-6

A highly efficient HSC ablation model was developed herein that enabled for uncovering the role of quiescent HSCs in adult mouse livers during homeostasis. Previous HSC ablation models using transgenic mice expressing diphtheria toxin receptor or thymidine kinase or HSC ablation by gliotoxin administration achieved only partial (50%-65%) ablation of HSCs. Although the HSC-hepatocyte interaction was focused on, this ablation model might also be useful for investigating the interaction of HSCs with other liver cells and to explore how HSCs modulate the immune microenvironment or the effects of HSCs on liver sinusoidal endothelial cell biology. Although GFP was highly expressed in HSCs within the liver in the model herein, extrahepatic expression of GFP in Pdgfrb+ cells in other organs such as the lung, kidney, or pancreas, could be used to study pericyte biology in other organ systems.

Signaling pathways that regulate liver zonation have been best characterized for zone 1 and zone 3, which was facilitated by the longstanding tradition of binary classification of hepatocytes and well-established markers for both zone 1 (E-cadherin) and zone 3 (glutamine synthetase). Much less is known about signals determining midlobular hepatocyte zonation. Unique genes expressed at higher amounts in midlobular hepatocytes have only been identified using advanced scRNA-seq and spatial sorting and transcriptomic analyses and fate-labeling methods. These include hepcidin antimicrobial peptide (Hamp2) and Igfbp2 suggesting that midlobular hepatocytes play important roles in iron metabolism and insulin growth factor signaling. Midlobular hepatocytes also express higher amounts of cytochrome P450 xenobiotic metabolism genes. Genetic fate-labeling in Hamp2-CreER transgenic mice confirm Hamp2 as a primarily midlobular marker and identify IGFBP2-mTOR-CCND1 signaling as a critical pathway to maintain midlobular hepatocyte proliferation. Deletion of either Igfbp2 or inhibition of mechanistic target of rapamycin and 2 (mTORC1/2) leads to decreased CCND1 expression and hepatocyte proliferation in vivo. Whereas our results demonstrate that HSCs were essential to maintain midlobular CCND1 expression and liver homeostasis, NTF3 only increased CCND1 but not IGFBP2 expression in hepatocytes of HSC-ablated mice, suggesting that NTF3-TRKB signaling may regulate CCND1 separately from the IGFBP2-mTOR-CCND1 axis in vivo.

Fate-labeling studies indicate that midlobular hepatocytes have a higher proliferative capacity than periportal or pericentral hepatocytes in the homeostatic liver. The results herein indicate that HSC-derived NTF3 acts as a full-fledged hepatocyte mitogen in the midlobular niche. In liver regeneration, signals inducing hepatocyte proliferation may be classified as ‘complete mitogens’ and ‘auxiliary hepatocyte mitogens’ depending on their ability to induce proliferation in cultured primary hepatocytes and to induce hepatocyte DNA synthesis and liver enlargement in vivo. In contrast to complete mitogens, auxiliary mitogens cannot induce hepatocyte proliferation in culture or if injected into rodents. Whereas auxiliary mitogens include several different types of molecules, complete mitogens include only HGF and ligands of epidermal growth factor receptor (EGFR). Other complex signaling pathways can also promote hepatocyte proliferation, but disruption of these (Wnt-β-catenin and hedgehog signaling) delays liver regeneration without abolishing regeneration. Although the role of NTF3 in liver regeneration remains to be clarified, gene expression analysis of mouse livers 72 hours after partial hepatectomy demonstrated increased Ntf3 mRNA amounts consistent with a pro-proliferative role for NTF3 during liver regeneration (FIG. 12G). The results herein demonstrated that NTF3 increased proliferation of cultured primary hepatocytes through TRKB, and induced hepatocyte proliferation and enlargement of liver size when injected into mice (FIG. 10C, FIG. 10G, FIG. 10H, FIG. 12D, and FIG. 12F). Of note, NTF3 induced hepatocyte proliferation in HSC-depleted mice despite decreased amounts of HGF in these mice, underscoring the effectiveness of NTF3 in inducing proliferation (FIG. 8A). Although it is possible that NTF3 synergizes with HGF to promote proliferative effects, the studies herein did not detect synergism at the tested concentrations. Further studies are required to clarify synergistic effects of NTF3 with HGF, EGF or catecholamines, which can enhance effects of HGF and EGF on hepatocyte proliferation.

The loss in liver mass of ˜22% and ˜27% after 6 weeks and 10 weeks of sustained HSC-depletion, respectively, is substantial but can be explained by considering the dynamics of normal hepatocyte turnover. Mouse hepatocytes have a reported life span of 200 days. Cells reaching the end of their life are thought to undergo mostly caspase-dependent apoptosis to maintain homeostatic cell turnover. The number of apoptotic cells was similar in HSC-depleted and control livers, but the number of proliferating hepatocytes was markedly decreased, resulting in a net negative balance in cell numbers that, in turn, led to a progressive decrease in liver mass over time. Although a life span of ˜200 days may seem long, it would mean that after 100 days 50% of hepatocytes are replaced, and 25% after ˜50 days, and so on. In the HSC-depletion model herein, liver mass was reduced by ˜22% and ˜27% after 42 days and 70 days, respectively, which would be within the theoretically expected range if hepatocyte proliferation were abrogated. Some studies suggest that hepatocyte life spans can vary depending on the cell's location within the liver lobule, with zone 1 hepatocytes having shorter life spans (<1 year) than either zone 3 or zone 2 hepatocytes. It is possible that in addition to decreased hepatocyte proliferation, HSC-depletion also leads to a shortened life span of hepatocyte populations, which will need to be evaluated in more suitable fate-labeling models.

At this point, two question arise: How does NTF3 signal to hepatocytes in vivo? And why does NTF3 have a greater effect on midlobular hepatocytes than on zone 1 or zone 2 hepatocytes? Analysis of a published mouse liver scRNA-seq dataset that allows for expression analysis of hepatocytes according to their localization in the liver lobule (Massalha et al., Mol Syst Biol. 2020; 16(12): e9682) indicated that NTRK1 and NTRK2, but not NTRK3, were expressed in midlobular hepatocytes (FIG. 12A). Immunofluorescence staining for TRKB (NTRK2) showed TRKB+ midlobular hepatocytes, possibly explaining why NTF3 has the largest effect on midlobular hepatocytes (FIGS. 12B-C); however, further studies characterizing TRK expression in the liver are required. Overall, NTRK gene expression seems very low in liver in queries of publicly accessible datasets such as TCGA, Gtex, and the bulk RNA-seq dataset of control and HSC-depleted livers herein. However, low expression of NTRKs in healthy livers might not preclude high activity of specific receptors, as exemplified by the efficiency of HER2-targeting antibody drug conjugates in breast cancers with low HER2 expression. Furthermore, besides hepatocytes, other cells-namely HSCs themselves-express high amounts of NTRK2, NTRK3, and NGFR. Thus, autocrine neurotrophin signaling in HSCs could be biologically relevant, as recently confirmed in the context of fibrosis. HSCs are highly pleiotropic; therefore, other signaling pathways besides NTF3-TRKB might also be affected and contribute to the phenotypes observed in HSC-depleted livers. Furthermore, although how NTF3 affects CCND1 was focused on, NTF3 might also affect other proteins and signaling pathways in either HSC-depleted or control livers.

In summary, HSCs have important physiological functions, and their roles in liver homeostasis must be further elucidated to deepen understanding of pathobiological processes. Although activated HSCs are the principal fibrogenic cells in chronic liver injury driving fibrosis towards end-stage liver disease, therapeutic strategies targeting HSCs in liver cirrhosis analogous to T cell-targeted immunotherapy for cardiac fibrosis should be developed carefully and investigated in detail. NTF3 is a complete hepatocyte mitogen whose signaling pathway is well characterized in the nervous system and in cancer and for which signaling pathway agonists and antagonists are available. Future studies may well reveal how these pathways could be exploited therapeutically to promote liver regeneration and/or inhibit proliferation in hepatic malignancies.

### Example 8

### Materials and Methods for Examples 9-13

Mice. Jedi mice were obtained from the Jackson Laboratories (Jax 028062) and are homozygous for CD45.1 and the H-2Kd haplotype. Pdgfrb-BAC-eGFP cryopreserved sperm was obtained from MMRRC (031796-UCD), and mice were rederived in C57Bl/6, substrain B10.D2 females (Jax 000463) that express CD45.2 and the H-2Kd haplotype. Rederived Pdgfrb-GFP mice were backcrossed into B10.D2 and were maintained heterozygous for GFP, homozygous for CD45.2, and homozygous for the H-2K^{d} haplotype. GFAP-HSV-thymidine kinase (GFAP-TK) were obtained from Jackson Laboratories (Jax 005698). Transgenic (Tg) mice express the herpes simplex thymidine kinase gene driven by the mouse glial fibrillary acidic protein (GFAP) promoter. HSV-Tk-negative littermates served as controls. Hepatic stellate cell (HSC) depletion in proliferating HSCs was induced by administration of ganciclovir in combination with allyl alcohol and carbon tetrachloride induced panlobular injury as previously described (Puche et al., Hepatology. 2013; 57(1): 339-50). C57Bl/6, substrain B10.D2 (Jax 000463) were used for experiments in WT animals. All experiments were performed when mice were 6 weeks to 10 months old, and both males and females were used. All animal procedures were performed according to protocols approved by the Institutional Animal Care and Use Committees at Icahn School of Medicine at Mount Sinai and the Institutional Animal Care and Use Committees at Vanderbilt University Medical Center.

Genotyping. Jedi mice were genotyped by PCR genotyping using the following primers: TCRα-For: GAGGAGCCAGCAGAAGGT (SEQ ID NO: 1); TCRα-Rev: TCCCACCCTACACTCACTACA (SEQ ID NO: 2); TCRβ-For: TCAAGTCGCTTCCAACCTCAA (SEQ ID NO: 3); TCRβ-Rev: TGTCACAGTGAGCCGGGTG (SEQ ID NO: 4). Pdgfrb-GFP mice were genotyped by PCR using the following primers: GFP-For: GTGGAAGCAGAGAGGAGAGCATTTG (SEQ ID NO: 5), GFP-Rev: GGTCGGGGTAGCGGCTGAA (SEQ ID NO: 6). H-2K^{d} haplotype, CD45.1, and CD45.2 status were determined by flow analysis of peripheral blood mononuclear cells using the following antibodies purchased from ebiosciences (Thermo Fisher Scientific, Waltham, MA): CD45.1-eFluor-450 (48-0453-80), CD45.2-PE (12-0454-82), and H-2K^{d}-APC (17-5957-80). GFAP-HSV-TK mice were genotyped by PCR using the following primers: TK-For: GGGGCCTCG GTCCTAGT (SEQ ID NO: 17), TK-Rev: GATGGCAGGGGTACGAAG (SEQ ID NO: 18).

T cell isolation and adoptive transfer. T cell isolation and adoptive transfer were performed as previously described (Trinh et al., Sci Signal. 2023; 16(787): eadf6696). CD8+ T cells were isolated from the spleens and lymphnodes of Jedi and control mice. Spleens and lymph nodes were collected in ice-cold T cell medium [RPMI 1640 (Gibco, Waltham, MA; 11875-093)] supplemented with 10% fetal bovine serum (FBS), 2 mM L-glutamine (Thermo Fisher Scientific, Waltham, MA; 25030-081), 1% MEM (minimum essential medium) non-essential amino acids (Thermo Fisher Scientific, Waltham, MA; 11140050), and 1 mM sodium pyruvate (Thermo Fisher Scientific, Waltham, MA; 11360-070). The spleens and lymph nodes were mechanically homogenized and filtered through a 70-μm cell strainer (Falcon®). The cell suspension was centrifuged for 10 min at 4° C., 300 g. Red blood cell (RBC) lysis was performed with RBC buffer (Thermo Fisher Scientific, Waltham, MA; A10492). Cells were then washed with T cell medium, filtered through a 70-μm cell strainer, and centrifuged for 10 min at 4° C., 300 g. The cell pellet was then resuspended in flow buffer [phosphate-buffered saline (PBS) (pH 7.4), 2 mM EDTA, and 0.05% bovine serum albumin (BSA)], and CD8+ T cells were enriched using the mouse CD8+ T cell isolation kit (STEMCELL Technologies, Vancouver, Canada; 19853A) according to the manufacturer's instructions. Enriched CD8 T cells were centrifuged for 10 min at 4° C., 300 g, and resuspended in PBS. Mice with less than 25-g body weight were injected with up to 12 million Jedi T cells, and mice with greater than 25-g body weight were injected by tail vein injection with 18 million JedidT cells. Control mice were injected with either T cells from H-2K mice or with vehicle only.

Partial hepatectomy. All surgeries were performed per the National Institutes of Health recommendations contained in the Guide for the Care and Use of Laboratory. Mice were anesthetized with isoflurane and received preoperative extended-release buprenorphine subcutaneous (s.c.) for analgesia and eye ointment for protection. An incision was made inferior to the xiphoid process. The evisceration of the median and left lateral lobes allowed suture ligation and ˜70% hepatectomy. Skin and abdominal muscles were closed in a two-step procedure. Mice were euthanized 1 h, 24 h, 2 days, 3 days, and 4 days after partial hepatectomy, and tissues were collected for processing. To test if recombinant NTF3 promotes liver regeneration, mice were injected intravenous (i.v.) with recombinant NTF3 (STEMCELL Technologies, Vancouver, Canada; catalog no. 78074, lot nos. 1000070683 and 1000077434) diluted in sterile 0.9% saline (0.9% NaCl) or vehicle only at approximately 1 hour prior to partial hepatectomy. Mice were then injected i.v. once per day with recombinant NTF3 or vehicle at 20 ng/g body weight until sacrifice. Lyophilized NTF3 was solubilized in 0.1% BSA/PBS (Sigma-Aldrich, St. Louis, MO; A3059) and stored at −80° C. until use. Because activity and protein concentration declined significantly at about 3 months, only aliquots stored for 3 months or less were used for experiments, and freeze-thaw cycles were minimized.

Serum chemistry. Whole blood was collected in blood collection tubes supplemented with lithium heparin (BD vacutainer, Franklin Lakes, NJ; Cat.366664). Blood was centrifuged at 4000 g for 15 min and the plasma collected and stored at −80° C. until analysis. Plasma was analyzed on a VetScan® vs2 (Abaxis, Union City, CA) using mammalian liver profile analysis discs (Abaxis, Union City, CA; 500-0040) according to the manufacturer's instructions.

RNA extraction and qRT-PCR. Bulk liver total RNA was isolated using the RNeasy Kit (Qiagen, Hilden, Germany) following the manufacturer's instructions. Five μg of total RNA were transcribed to cDNA using the RNA-to-cDNA ecoDry Premix (Takara Bio, San Jose, CA). IQ SYBR Green Supermix (Bio-Rad Laboratories, Hercules, CA) was used to quantitate on a LightCycler®480 System (Roche, Basel, Switzerland) or CFX96 Real-time System (Bio-Rad Laboratories, Hercules, CA) using the ddCT method and normalizing to TBP expression. Primer sequences used were hm-Albumin: For-CACGCCTTTGGCACAATGAA (SEQ ID NO: 19), Rev-ATCTCGACGAAACACACCCC (SEQ ID NO: 20), hm-KRT19: For-GAAGGATGCTGAAGCCTGGT (SEQ ID NO: 21), Rev-GTCAGTAACCTCGGACCTGC (SEQ ID NO: 22); hm-SOX9: For-GACTTCTGAACGAGAGCGAGA (SEQ ID NO: 23), Rev-CCGTTCTTCACCGACTTCCTC (SEQ ID NO: 24); hm-HNF4A: For-TGCGACTCTCCAAAACCCTC (SEQ ID NO: 25), Rev-ATTGCCCATCGTCAACACCT (SEQ ID NO: 26); Hm-TBP: For-GCATCACTGTTTCTTGGCGT (SEQ ID NO: 27), Rev-AGAGCATCTCCAGCACACTC (SEQ ID NO: 28).

Cell lines. Mouse HSC cell line JS1, and human HSC line LX2 and TWNT4 were cultured in DMEM supplemented with 10% FBS.

Immunofluorescence. For immunofluorescence, 5-μm cryosections from fresh-frozen liver samples embedded in optimal cutting temperature compound (Sakura, Tokyo, Japan) were cut and dried for a minimum of 1 hour at room temperature and stored at −80° C. until use. Frozen sections were thawed and rehydrated in PBS for 5 min at room temperature, fixed in 100% acetone for 10 min at −20° C., and washed in PBS. The primary antibody (TABLE 1) was diluted in antibody diluent (Agilent, Santa Clara, CA; S080983-2) and incubated for 2 hours at room temperature in a wet chamber. Slides were then washed three times for 5 min in PBS. The secondary antibody (TABLE 1) was diluted in antibody diluent (Agilent, Santa Clara, CA; S080983-2 or PBS), and slides were incubated in a wet chamber for 30 to 60 min. Slides were washed three times in PBS, followed by two washes in distilled H₂O. The slides were then mounted with a DAPI-containing medium (Prolong Antifade, Thermo Fisher Scientific, Waltham, MA; P36931). Images were taken with either a Nikon A1R confocal microscope (Nikon, Tokyo, Japan), an Axio Imager M2 fluorescence stereomicroscope, or an Axio Imager 7 (Zeiss, Oberkochen, Germany) microscope.

Immunohistochemistry. For immunohistochemistry, formalin-fixed paraffin-embedded (FFPE) sections were cut at a 4-μm thickness, and slides were incubated at 65° C. for 30 min. Slides were deparaffinized in Histo-Clear® (National Diagnostics, Charlotte, NC; HS2001GLL) and rehydrated by an ethanol gradient. Heat antigen retrieval was performed in 10 mM sodium citrate (pH 6.0) in a pressure cooker (Cuisinart®, CPC-600C), for 20 min at high pressure, natural pressure release for 30 min, and cooled at room temperature for 30 min. Endogenous peroxidase quenching was performed with 1.5% hydrogen peroxide (Sigma-Aldrich, St. Louis, MO; H1009) for 20 min, and slides were washed twice in PBS. Blocking was performed for 30 min with Protein Block, Serum-free (Agilent, Santa Clara, CA; X090930-2). The primary antibody was diluted in antibody diluent (Agilent, Santa Clara, CA; S080983-2) and applied to the slides in a wet chamber overnight at 4° C. After two washes in PBS, slides were incubated with the appropriate secondary antibody for 30 min, followed by two washes in PBS. The antigen was revealed with DAB (3,3′-diaminobenzidine) (Vector Laboratories, Newark, CA; SK-4105) until the appropriate staining on a control slide was obtained. The same reveal time was then used on all experimental slides. Slides were stained with hematoxylin (Sigma-Aldrich, St. Louis, MO; GHS332) for 30 s, washed gently in lukewarm tap water for 5 min, and washed twice in distilled water, followed by two washes in PBS (pH 7.4). Slides were washed in increasing ethanol/water gradients, followed by three washes in Histo-Clear®. Whole-slide scans for subsequent analyses and publishing were obtained using an SCN400 slide scanner (Leica, Wetzlar, Germany). Whole-slide images were processed and annotated in QuPath 0.5.1.

Primary mouse hepatocyte isolation. Mice were anesthetized and cannulated via the portal vein. Livers were perfused with ˜25 ml of prewarmed liver perfusion medium (Gibco, Waltham, MA; 17707-038) followed by ˜50 ml of liver digestion medium (Gibco, Waltham, MA; 17703-034). The liver capsule was gently teased apart with tweezers, and hepatocytes were released. Cells were taken up in Hepatocyte Wash Buffer (Gibco, Waltham, MA; 17704-024) and filtered through a 100-μm mesh. Cells were then centrifuged for 2 min at 100 g, 4° C., and the cell pellet was resuspended in 10 ml of ice-cold DMEM (Gibco, Waltham, MA; 11885-084)/5% FBS. Ten microliters of ice-cold 100% Percoll®/PBS (GE HealthCare, Chicago, IL; GE17-0891-01) were added for gradient purification, and cells were centrifuged for 10 min at 200 g, 4° C. The cell pellet was resuspended with 20 ml of DMEM/5% FBS and centrifuged for 2 min at 100 g, 4° C. A total of 1×10³ to 2×10³ hepatocytes were plated on collagen-coated 96-well plates in DMEM/5% FBS/Pen/Strep. Rat tail collagen I (Gibco, Waltham, MA; A1048301) was used for coating. Cell culture media were changed 3 hours after plating with either serum-free William's E media (Gibco, Waltham, MA) containing Pen/Strep (Gibco, Waltham, MA) or DMEM/5% FBS/Pen/Strep. The following TRK agonists were added 1 day after plating: NTF3 (STEMCELL Technologies, Vancouver, Canada; final concentration 20 ng/ml), BDNF (STEMCELL Technologies, Vancouver, Canada; final concentration 20 ng/ml), mouse recombinant hepatocyte growth factor (Abcam, Cambridge, UK; ab281797, final concentration 20 ng/ml), LM22A-4 (Fisher Scientific, Waltham, MA; 501931913, stock solution 100 mM in water, final concentration 100-500 mM), 7,8-dihydroxiflavone (7,8-DHF, Fisher Scientific, Waltham, MA; 501365939, stock solution 20 mM in DMSO, final concentration 10-50 mM), N-Acetyl-5-hydroxytryptamine (NAS, Sigma Aldrich, St. Louis, MO; A1824, diluted in ethanol, SL 40 mg/ml, final concentration 500 nM). Primary hepatocytes were cultured for 3 days until analysis.

Cell proliferation assays. Primary mouse hepatocytes were grown in opaque-walled 96-well plates. NTF3 (final concentration, 20 ng/ml) (STEMCELL Technologies, Vancouver, Canada; catalog no. 78074) and/or ANA-12 (final concentration, 10 nM) was added into complete growth media without L-glutamine. Primary mouse hepatocytes were also grown in serum-free William's E media without supplements. Cells were analyzed by CellTiter-Glo® (Promega G7570, Madison, WI), which detects the number of viable cells on days 0 to 5 by on-detection systems Glomax (Promega, Madison, WI) or Bioteck Synergy H1 (Agilent, Santa Clara, CA). Mouse recombinant HGF (Abcam, Cambridge, UK; ab281797) was added to cell culture media at a final concentration of 5-10 ng/ml. TRKB inhibitor ANA-12 (Selleckchem, Houston, TX; S7745) was diluted in dimethyl sulfoxide (10 mM stock solution) and added to cell culture media at a final concentration of 10 nM.

Mouse passaged primary human hepatocytes. Mouse-passaged primary human hepatocytes (mpPHHs) were collected as previously described (Michailidis et al., Proc Natl Acad Sci USA. 2020; 117(3): 1678-1688) and cultured in hepatocyte culture media (CC-3198, Lonza, Basel, Switzerland) in collagen-coated chamber slides. Three days before treatment, mpPHH were incubated in William's E media (with L-glutamine). Vehicle, NTF3, BDNF, or HGF (20 ng/ml each) were diluted in William's E (without L-glutamine) and EdU (EdU Cell Proliferation kit, Alexa Flour 555, Thermo Fisher Scientific, Waltham, MA; C10638) was added to each well. mpPHH were incubated for 4 hours and the experiment terminated by fixing the mpPHH in 4% paraformaldehyde/PFA and permeablized with Triton X-100. Click-iT™ EdU staining was then performed according to the manufacturer's instructions. mpPHH were mounted with Flouromount G and imaged with Nikon confocal microscope. 5-15 images per treatment per experiment were randomly taken and EdU positive nuclei quantified.

Human ductal organoids. The normal human liver tissue was obtained through the Cooperative Human Tissue Network (CHTN) at Vanderbilt University Medical Center. The liver was cut into very small pieces in liver digestion medium (Gibco, Waltham, MA; 17703-034). The cell suspension was filtered through a 100-μm cell strainer. The flow through was then centrifuged for 2 min at 100 g, 4° C., and the cell pellet resuspended in ice-cold DMEM (Gibco, Waltham, MA; 11885-084, supplemented by 5% FBS) and centrifuged for 2 min at 300 g. The cell pellet was gently resuspended in Matrigel® (Corning, Corning, NY; 356231) and a 50 ml dome plated in a 24-well plate. The plate was incubated for 10-15 min at 37° C. After solidifying, complete HepatiCult™ Organoid Initiation Medium (OIM) (STEMCELL Technologies, Vancouver, Canada; 100-0384) was added. For passaging, the Matrigel dome was dissociated using TrypLE (Thermo Fisher Scientific, Waltham, MA; 12604013) and incubated at 37° C. for 5 mins. Organoids were washed by resuspending in advanced DMEM/F12 and consecutive resuspension of the organoid pellet. Organoids were resuspended at a 1:10 dilution in Matrigel® and cultured in complete HepatiCult™ Organoid Growth Medium (OGM) (STEMCELL Technologies, Vancouver, Canada; 100-0385) for organoid expansion. For hepatocytic differentiation of hepatic ductal organoids, culture medium was changed to complete HepatiCult™ Organoid Differentiation Medium (ODM) (STEMCELL Technologies, 100-0383) 4 days after passaging. Vehicle, NTF3, BDNF, HGF (20 ng/ml final concentration) or Taletrectinib (Fisher Scientific, Waltham, MA; 502030245, final concentration 10 nM) or torin2 (Fisher Scientific, Waltham, MA; s2817, final concentration 25 nM) were diluted in William's E (without L-glutamine) and added to organoids.

EdU incorporation and imaging in HDO and mpPHH. A chamber slide was pre-chilled on ice and coated with Matrigel®. Chamber slide was then incubated for 3 min at 37° C. to solidify and organoids plated in 0.02% Matrigel®/complete media into coated chamber slides. Organoids were grown in complete organoid growth media (OGM) for approximately 4 days followed by incubation in organoid differentiation media (ODM) for another 4 days. Organoids were changed to William's E medium 1 day prior to experiment. Vehicle or NTF3, BDNF, HGF (20 ng/ml final concentration each) or Taletrectinib (Fisher Scientific, Waltham, MA; 502030245, final concentration 10 nM) were diluted in William's E (without L-glutamine) and added to organoids. CLICK-iT™ EdU (EdU final concentration 10 mM incubated together with the vehicle, NTF3, BDNF or HGF). After 6 hours, organoids were fixed with 4% paraformaldehyde and permeabilized with 0.5% Triton-X 100. Further processing was performed according to CLICK-ITT EdU manufacturer's instructions. mpPHHs were seeded on collagen coated chamber slides and grown in complete hepatocyte medium (Lonza, Basel, Switzerland). Prior to EdU incorporation assays, mpPHHs were cultured in William's E medium. Vehicle or NTF3, BDNF, HGF (20 ng/ml final concentration each) or Taletrectinib (Fisher Scientific, Waltham, MA; 502030245, final concentration 10 nM) were diluted in William's E (without L-glutamine) and added to mpPHHs. Organoids were imaged in PBS in chamber slides, mpHHs were mounted with fluoromount. Five to fifteen images of random high-power fields were obtained per experiment per treatment and EdU+/Hoechst 33342+ nuclei versus all Hoechst 33342+ nuclei quantified.

Immunoblotting. HDOs and mpPHH were homogenized in RIPA buffer containing proteinase inhibitors (Roche, Basel, Switzerland; cOmplete™ Mini protease inhibitor, 2 tab/10 ml buffer) and 1×HALT phosphatase inhibitors (Thermo Fisher Scientific, Waltham, MA; catalog no. 78428). Lysates were sonicated and then centrifuged for 10 min at 18,800 g. Protein concentration of supernatants was determined by Direct Detect Spectrophotometer (MilliporeSigma, Burlington, MA). HDOs were treated with growth factors as described above. Lyophilized NTF3 was solubilized with sterile-filtered PBS supplemented with 0.1% BSA, aliquoted, and stored at −80° C. until use. Only aliquots with <3-month shelf life were used for experiments because activity and protein concentration declined significantly after 3 months. HDOs and mpPHH were collected on ice in PBS and centrifuged for 10 s, and the cell pellet was homogenized in RIPA buffer containing inhibitors (Roche, Basel, Switzerland; cOmplete™ Mini protease inhibitor, 1×HALT phosphatase inhibitors, Thermo Fisher Scientific, Waltham, MA; catalog no. 78428). Cell lysates were sonicated and centrifuged for 10 min at 18,800 g. The protein concentration of supernatants was determined by a Direct Detect® spectrophotometer (MilliporeSigma, Burlington, MA). For immunoblot analysis, 10 to 50 μg of protein diluted in D-lysergic acid diethylamide sample buffer (Invitrogen, Waltham, MA) were analyzed per SDS-polyacrylamide gel electrophoresis (NuPAGE™, Invitrogen, Waltham, MA) using Mops buffer or MES buffer (Invitrogen, Waltham, MA). Gels were stained with Coomassie (Bio-Safe™ Coomassie G-250 Stain, Bio-Rad Laboratories, Hercules, CA) and transferred onto polyvinylidene difluoride membranes using semi-dry blotting (Trans-Blot® SD, Bio-Rad Laboratories, Hercules, CA). Antibodies and dilutions used are indicated below. Images were visualized on an Amersham™ Imager 680 (Cytiva, Marlborough, MA). Semi-quantitative band densitometry was performed using ImageJ software (version 1.53).

Statistical analysis. Data analysis was performed using GraphPad Prism (version 10.4.2). Two groups were compared by unpaired two-tailed Mann-Whitney test. Data from three or more groups were assessed by the Kruskal-Wallis test with post hoc Dunn's test or One-way ANOVA post hoc Tukey's.

Antibodies. Immunohistochemistry or immunofluorescence: Ki67 (Abcam, Cambridge, UK; ab15580, dilution 1:800), Ki67 (Abcam, Cambridge, UK; ab16667, dilution 1:1,000), PCNA (Cell Signaling Technology, Danvers, MA; 13110, dilution 1:5,000), BDNF (Novus Biologicals, Centennial, CO; NB198682, dilution 1:50), Reelin (R&D, Minneapolis, MN; AF3820, dilution 1:50), NTF3 (Alomone Labs, Jerusalem, Isreal; ANT-003, dilution 1:50), NuMA (Abcam, Cambridge, UK; ab86129, dilution 1:50), Anti-mouse secondary HRP-conjugated antibody (Vector Laboratories, Newark, CA; MP-7402), Goat anti-rat (Vector Laboratories, Newark, CA; MP-7404), Goat anti-rabbit (Vector Laboratories, Newark, CA; MP-7451), Donkey anti-goat (Invitrogen, Waltham, MA; A-21447), Goat anti-rabbit (Invitrogen, Waltham, MA; A-21245). Immunoblotting: BDNF (Abcam, Cambridge, UK; ab203573, dilution 1:1,00), NTF3 (Invitrogen, Waltham, MA; PA5-88570, dilution 1:1,500) pS6RP Ser240/Ser244 (Cell Signaling Technology, Danvers, MA; 5364, 1:5,000), pS6RP Ser235/236 (Cell Signaling Technology, Danvers, MA; 4858, 1:5,000), S6RP (Cell Signaling Technology, Danvers, MA; 2217, 1;5,000), P70S6K (Cell Signaling Technology, Danvers, MA; 2708, 1:5,000), P-P70S6K Thr389 (Cell Signaling Technology, Danvers, MA; 9234, 1:5,000), CCND1 (Abcam, Cambridge, UK; ab16663, 1:5,000), PCNA (Cell Signaling Technology, Danvers, MA; 13110, dilution 1:5,000), GAPDH (MilliporeSigma, Burlington, MA; CB1001, dilution 1:5,000).

### Example 9

### Hepatic Stellate Cells are Required for Liver Regeneration

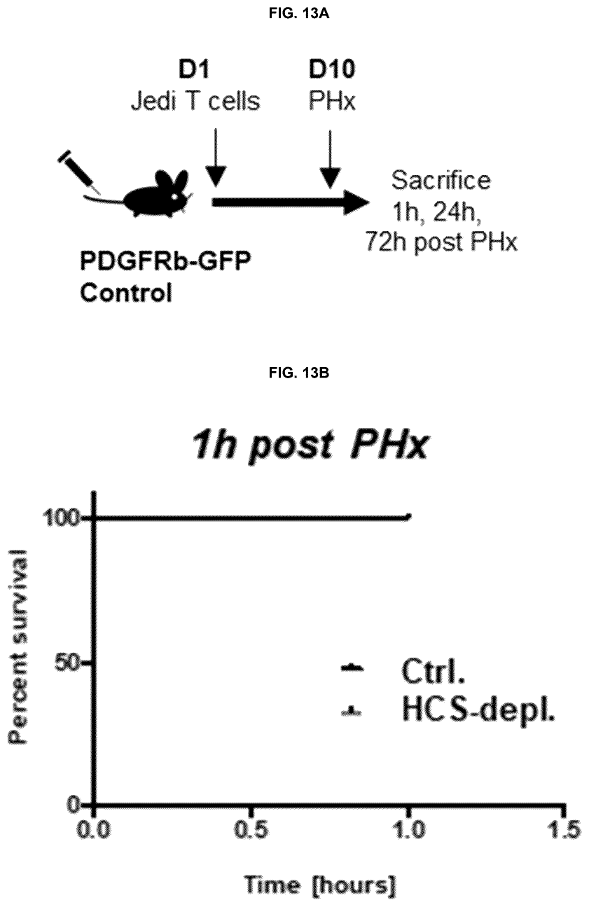

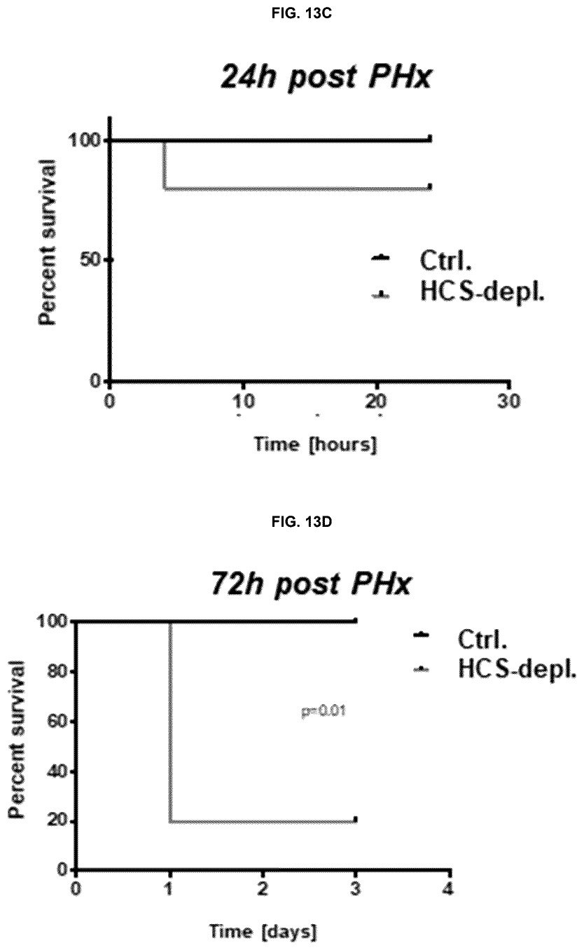

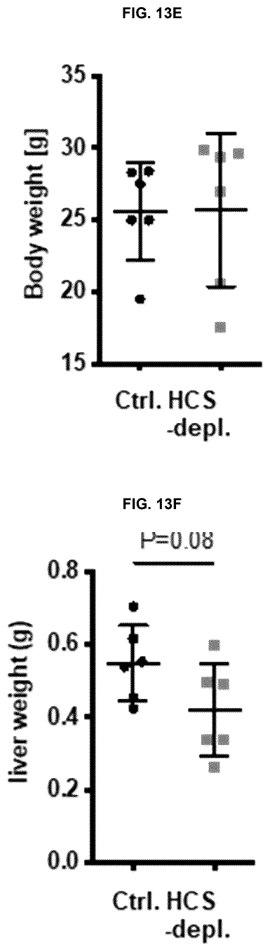

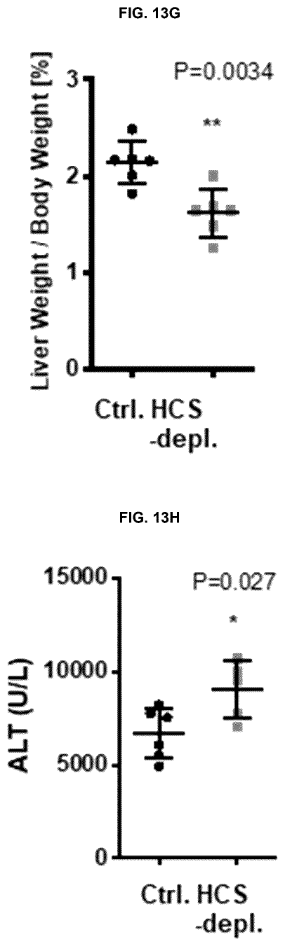

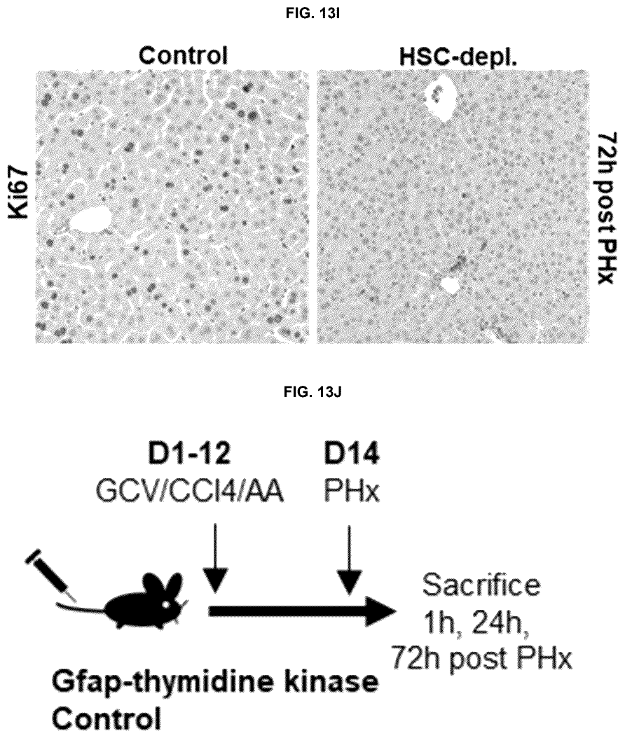

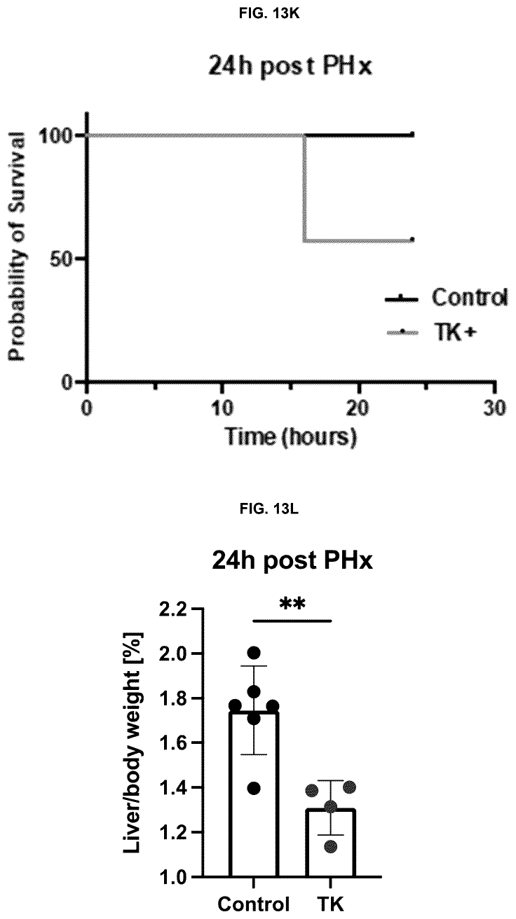

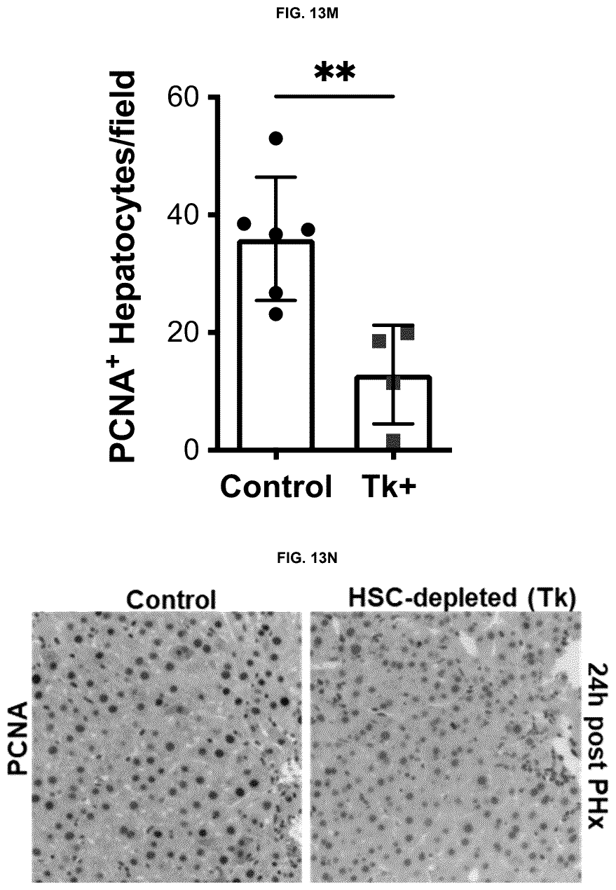

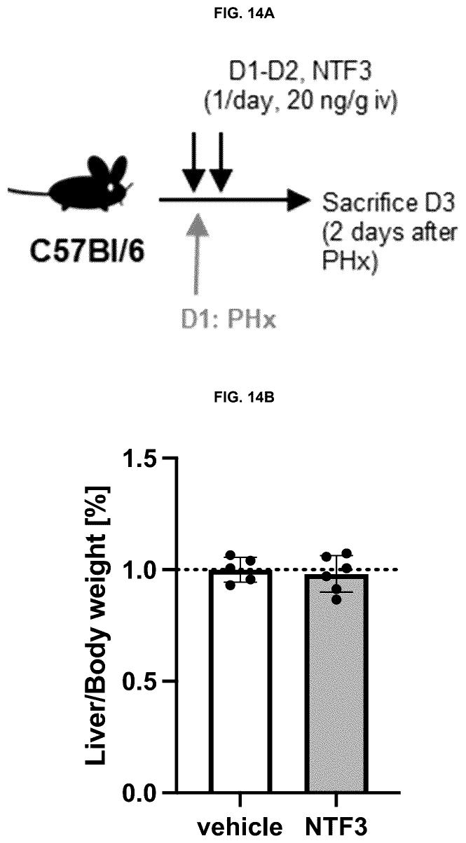

Herein a very efficient murine HSC-depletion method using Just eGFP death inducing (Jedi) T cells was established. Jedi T cells are engineered T cells that specifically target enhanced green fluorescent (eGFP) expressing cells. Adoptive transfer of Jedi T cells into Pdgfrb-BAC-eGFP transgenic mice, which express eGFP within the liver specifically in HSCs, led to very efficient depletion of HSCs. Herein, it was demonstrated that HSCs are essential to maintain liver homeostasis and proliferation of midlobular cyclin D1 positive midlobular hepatocytes in healthy adult livers. To clarify if HSCs are also important for liver regeneration, HSCs were depleted by adoptive transfer of Jedi T cells and partial hepatectomy was performed, a very established murine model in which 2/3 of the livers are removed (Mitchell and Willenbring. Nat Protoc. 2008; 3(7): 1167-70). Mice were sacrificed after 1 hour, 24 hours, and 72 hours after partial hepatectomy (FIG. 13A). Survival analyses showed marked decreased survival in HSC-depleted mice in the 24-hour group and 72-hour group compared to control (FIGS. 13B-D). This is in line with prior studies in which gross inhibition of HGF-cMET and EGFR signaling in murine models of liver regeneration led to higher mortality. Body weights in control and HSC-depleted mice was similar, while liver/body weight ratios were significantly decreased in HSC-depleted mice indicative of impaired liver regeneration (FIGS. 13E-G). Serum ALT levels were also higher in HSC-depleted mice indicative of greater liver impairment after partial hepatectomy (FIG. 13H). Immunostaining for Ki67 demonstrated decreased proliferation (FIG. 13I). As only a small number of mice survived at 72 hours a quantification was not performed. Very similar results in a second HSC-depletion model were observed in which mice expressing the herpes simplex virus thymidine kinase (HSV-Tk) under the mouse glial fibrially acidic protein (Gfap) promoter (henceforth Gfap-Tk) were treated with carbon tetrachloride and allyl alcohol for a total of 10 days to induce liver injury and HSC proliferation. Co-administration of ganciclovir led to cell death in proliferating HSCs with a depletion rate of approximately 65% as described above. Partial hepatectomy in this second model also led to decreased survival in HSC-depleted mice (FIGS. 13J-K) and decreased liver/body weight ratio indicating impaired liver regeneration (FIG. 13L). Immunostaining for PCNA, a proliferation marker, demonstrated a significantly decreased number of proliferating hepatocytes in HSC-depleted Gfap-Tk mice (FIGS. 13M-N).

### Example 10

### Neurotrophin-3 Administration Increases Liver Regeneration after Partial Hepatectomy

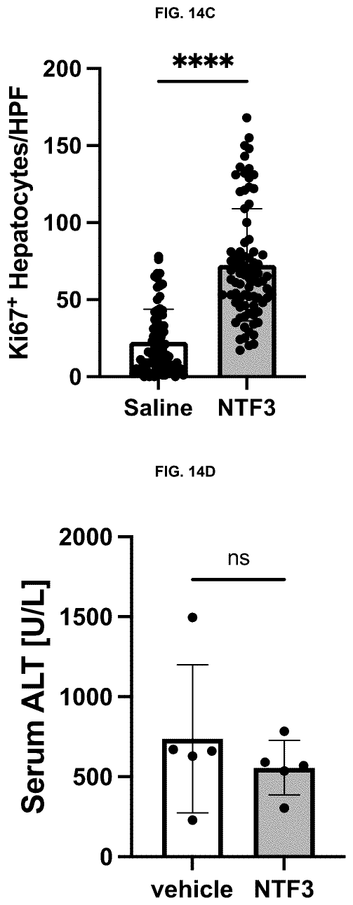

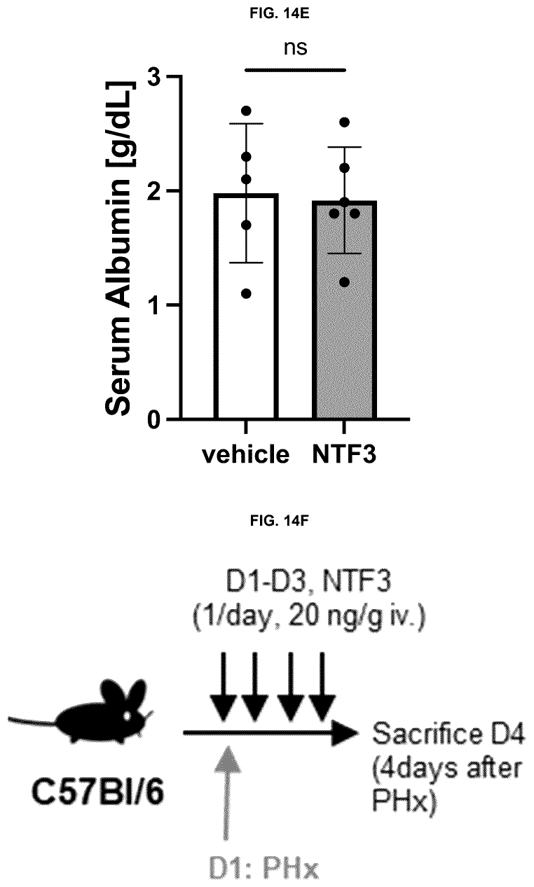

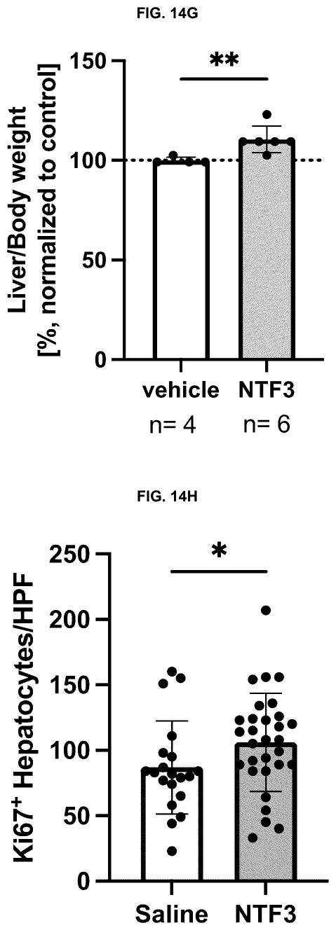

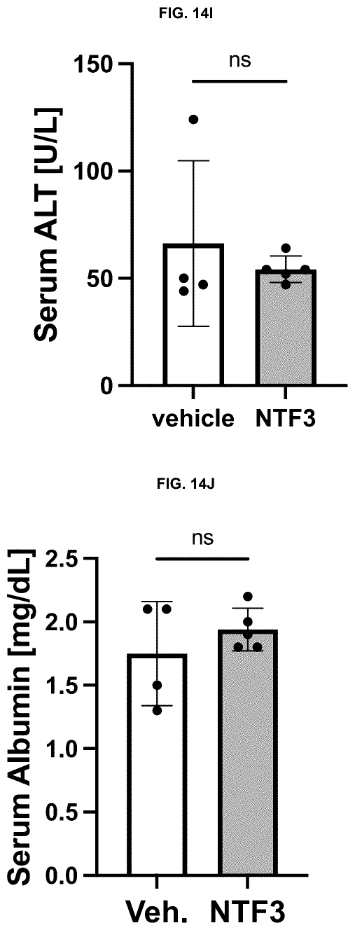

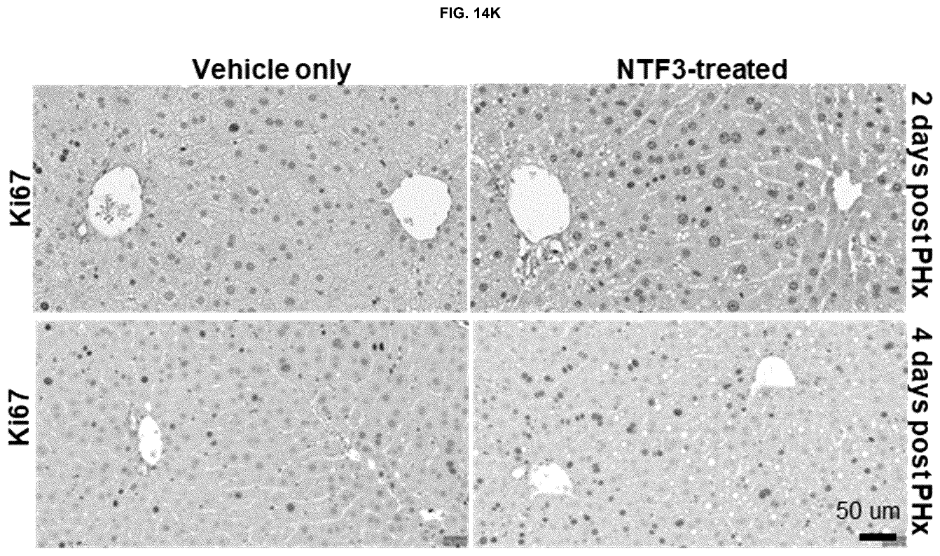

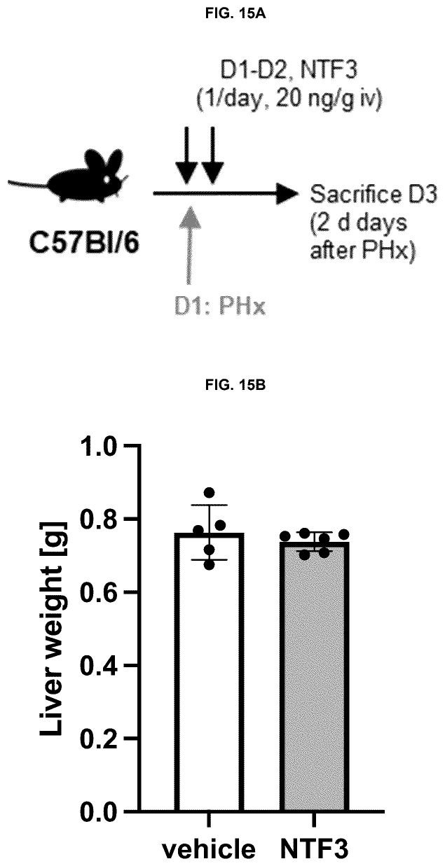

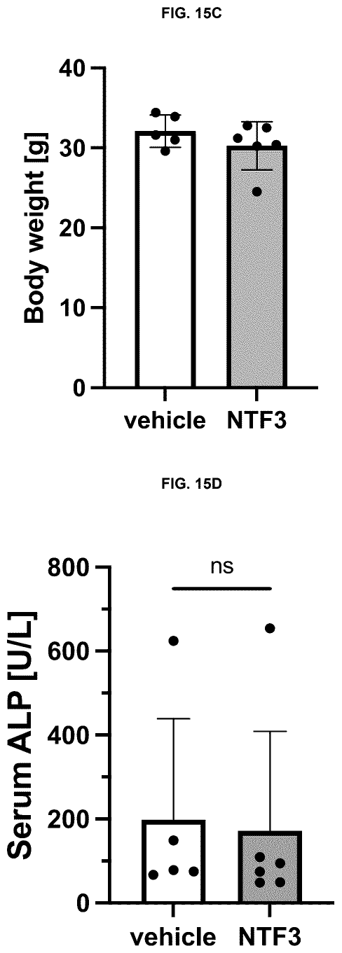

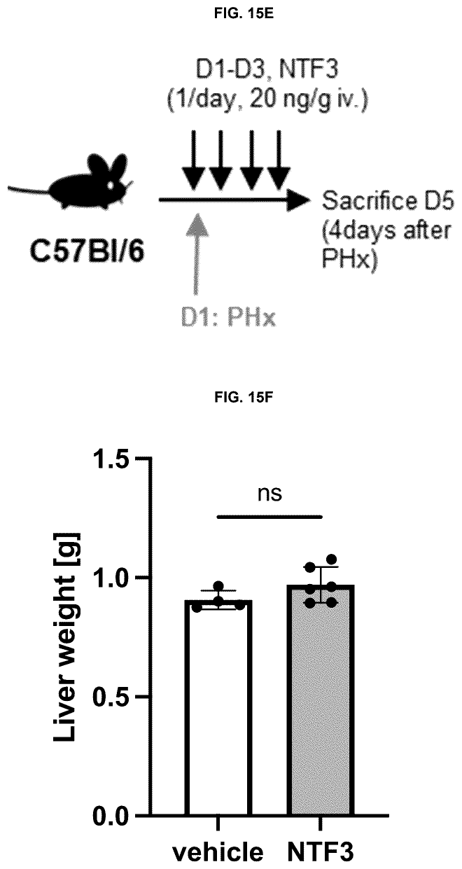

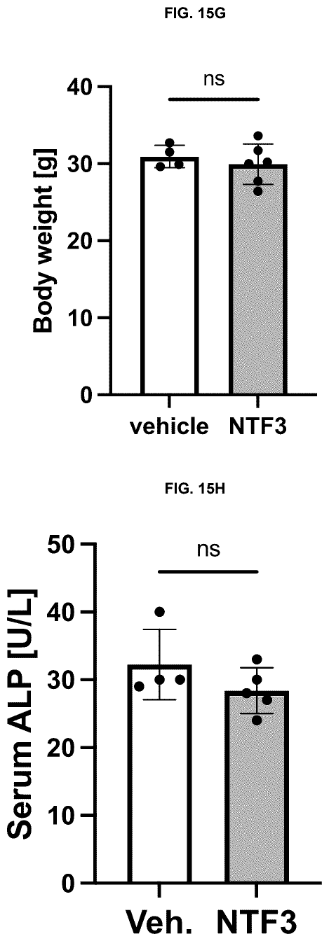

Above, NTF3 was identified as a growth factor that induces hepatocyte proliferation. To assess if administration of NTF3 enhances liver regeneration in wild type mice, mice were injected with NTF3 at 20 ng/g body weight prior to partial hepatectomy. Mice were then treated with NTF3 or vehicle only on consecutive days following partial hepatectomy once per day and were sacrificed after 2 days (FIG. 14A) or 4 days (FIG. 14F). Liver to body weight ratio was similar at 2 days (FIG. 14B, FIGS. 15A-C) but NTF3-treated mice showed significantly increased number of Ki67+ hepatocytes (FIGS. 14C, K). Serum ALT and albumin levels were similar in vehicle and NTF3-treated animals (FIGS. 14D, E) as were ALP (FIG. 15D). In mice sacrificed 4 days after partial hepatectomy, liver to body weight ratios were significantly increased in NTF3-treated mice with relative increase of 10-23% compared to controls (FIG. 14G, FIGS. 15F, G). Ki67 immunostaining demonstrated increased number of Ki67+ hepatocytes in NTF3 treated animals (FIGS. 14H, K). Serum ALT and albumin, as well as ALP were similar in NTF3, and vehicle-only treated animals demonstrating no adverse effects of NTF3 (FIGS. 14I-J, FIGS. 15E-H).

### Example 11

### Brain-Derived Neurotrophic Factor is Expressed and Induces Proliferation in Primary Mouse Hepatocytes

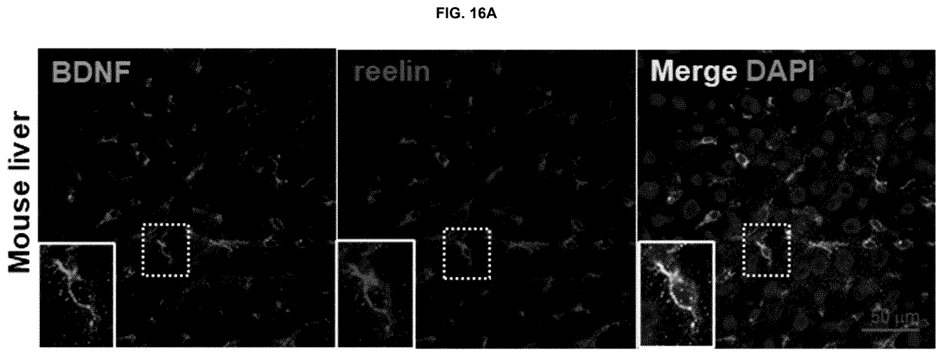

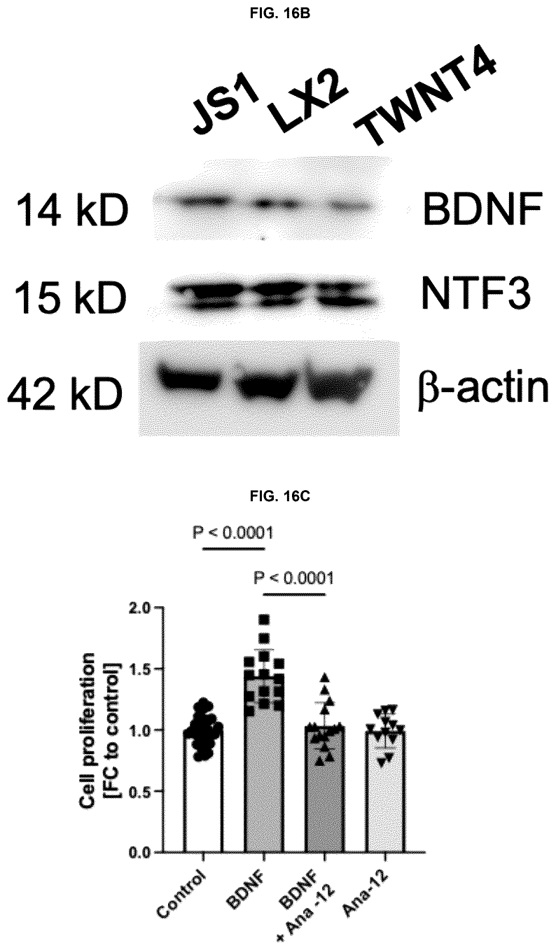

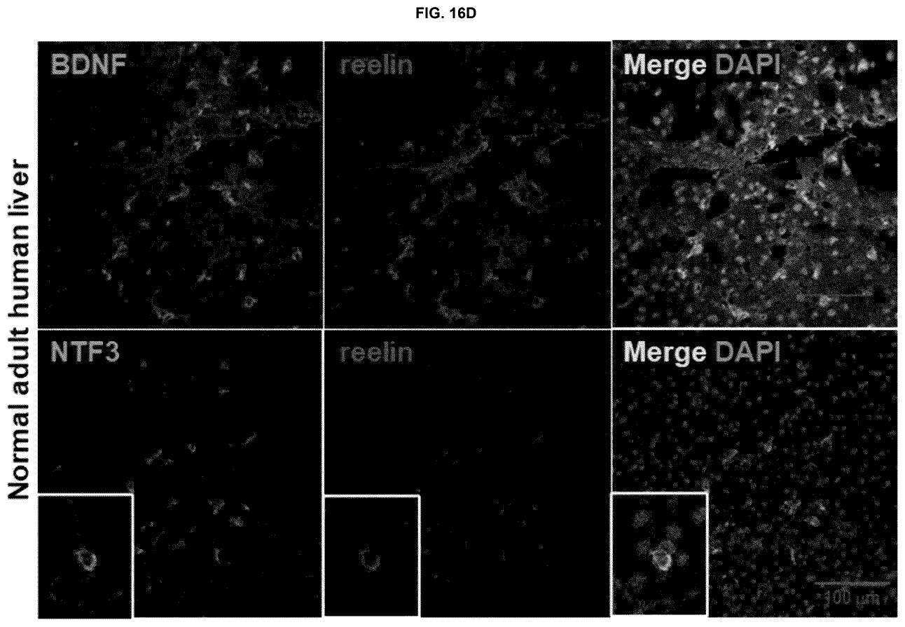

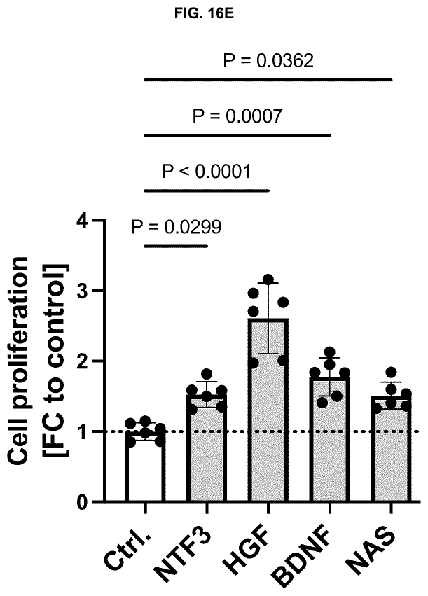

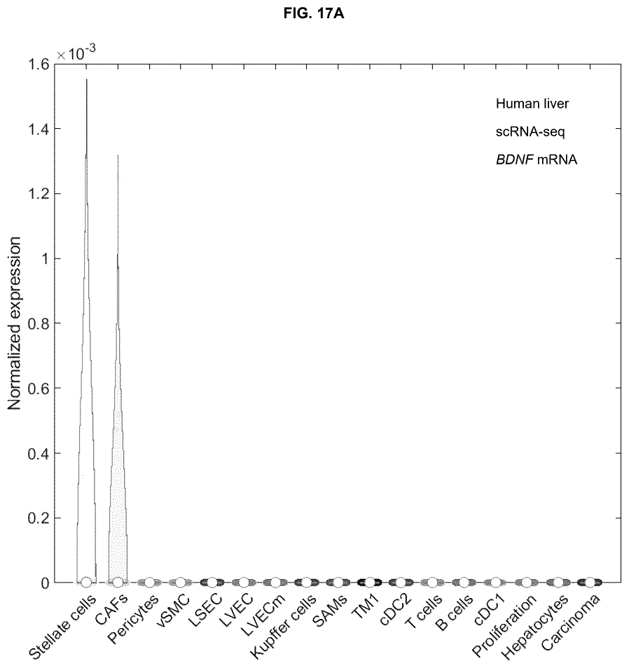

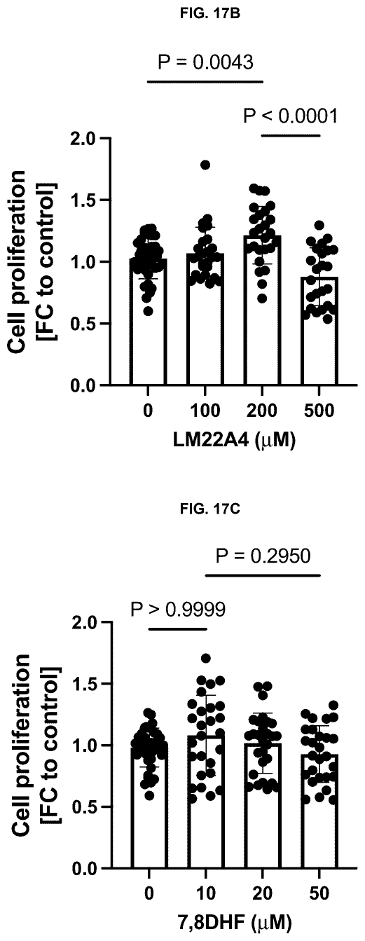

NTF3 signals through tropomyosin receptor kinase b (NTRK2) in murine hepatocytes. As brain-derived neurotrophic factor (BDNF) is the high affinity receptor of TRKB, whether HSCs express BDNF was assessed as well. Indeed, immunofluorescence analyses in adult mouse liver for BDNF and reelin, an HSC-marker, demonstrated that HSCs express BDNF (FIG. 16A). Similarly, immunoblot analyses of cell lysates from JS1, a mouse HSC cell line, LX2 and TWNT4, human HSC and fibroblast cell lines demonstrated that these cell lines express both BDNF and NTF3 (FIG. 16B). To assess if BDNF and NTF3 are also expressed in HSCs of human adult liver, liver sections was assessed by immunofluorescence analyses for BDNF and NTF3 with reelin respectively demonstrated that HSCs in human adult livers express BDNF, underscoring a NTF3/BDNF-TRK signaling in human and mouse livers (FIG. 16D). Analysis of a published human scRNA-seq dataset (Massalha et al., Mol Syst Biol. 2020; 16(12): e9682) demonstrated high BDNF levels in HSCs and pericytes only (FIG. 17A). To determine if BDNF induces proliferation in primary mouse hepatocytes, primary hepatocytes were incubated with BDNF, or a TRKB antagonist (ANA-12) to assess Trkb-dependent cell proliferation. BDNF significantly induced proliferation which was inhibited by co-incubation with ANA-12, suggesting that BDNF induces proliferation through Trkb (FIG. 16C). Incubation of primary mouse hepatocytes with NTF3, HGF and BDNF, or N-acetyl serotonin (NAS), a TRKB agonist, demonstrated that BDNF and NAS induce proliferation to a similar extent though HGF was more potent (FIG. 16E). Of note, other reported TRKB analogs LM22A-4 or 7,8-DHF did not significantly increase hepatocyte proliferation or seemed to have toxic effects in higher dosages (FIGS. 17B-C). Indeed, other reports disputed that these compounds are TRKB agonists.

### Example 12

### BDNF and NTF3 Induce Proliferation in Human Hepatic Organoids and Mouse Passaged Human Hepatocytes

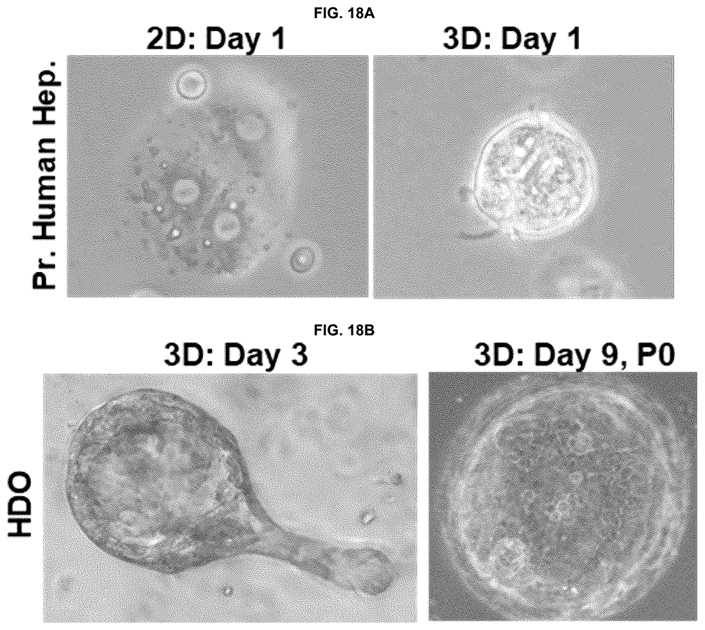

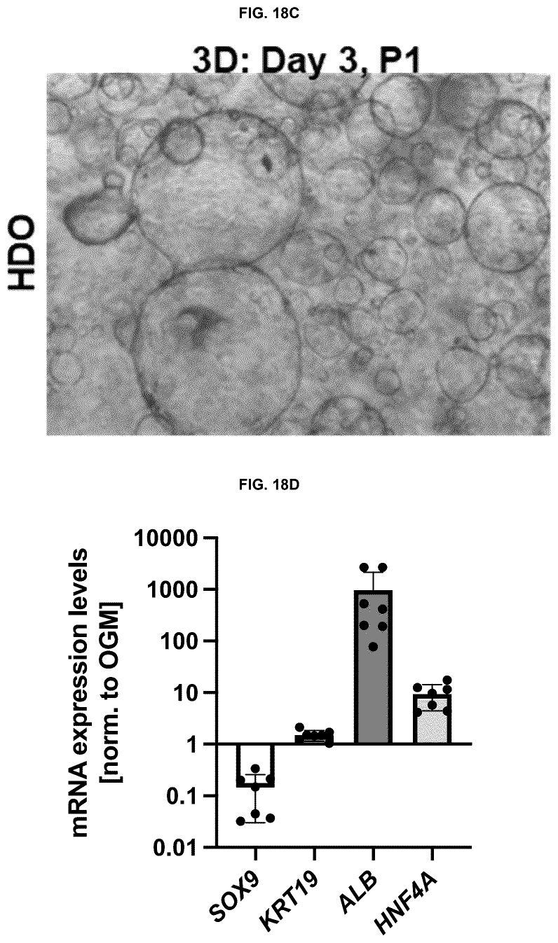

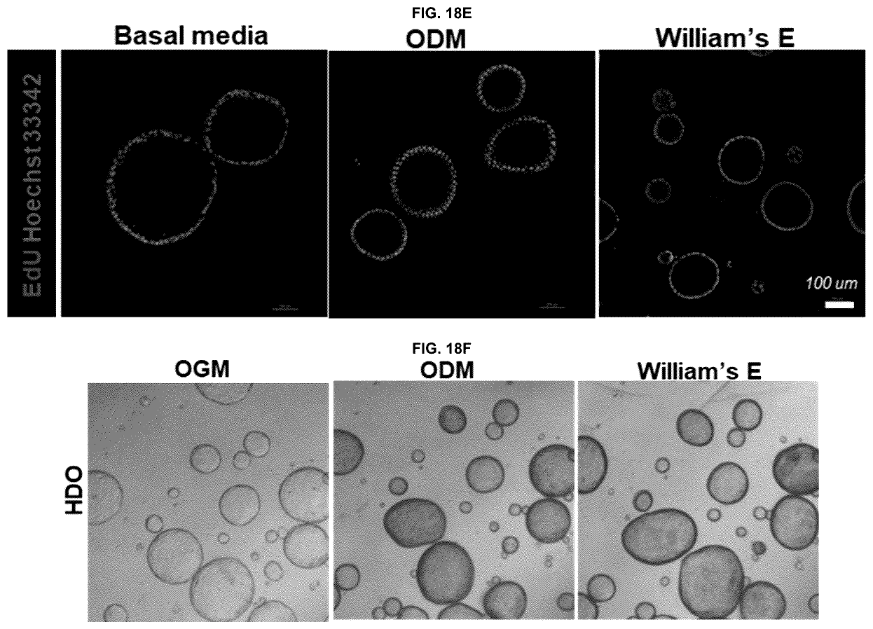

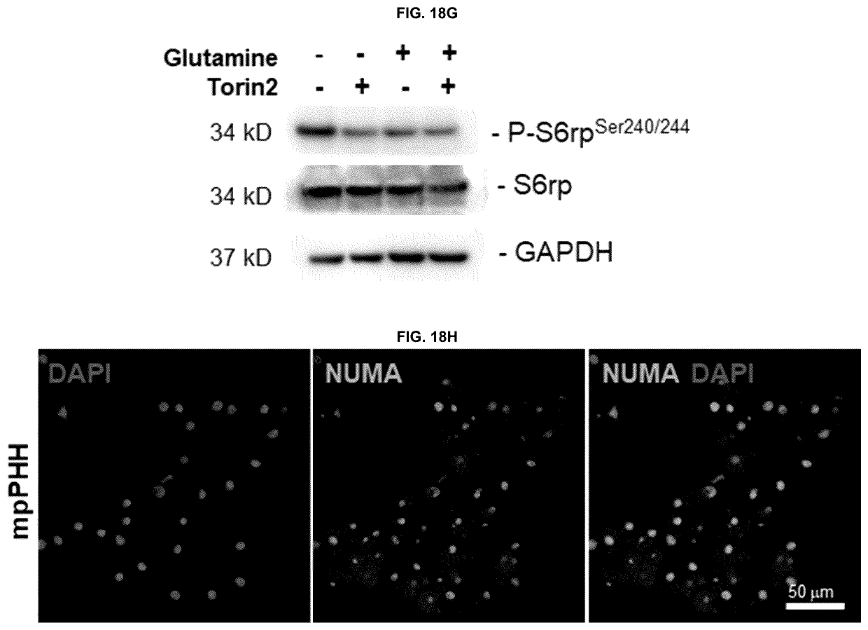

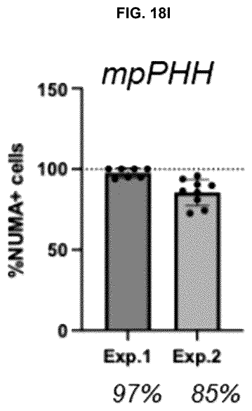

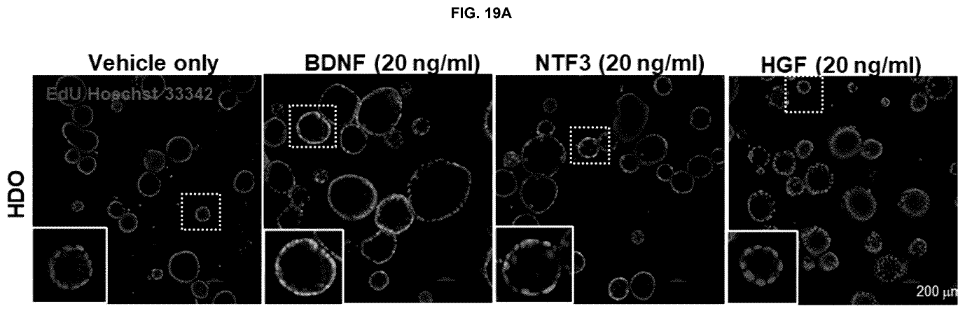

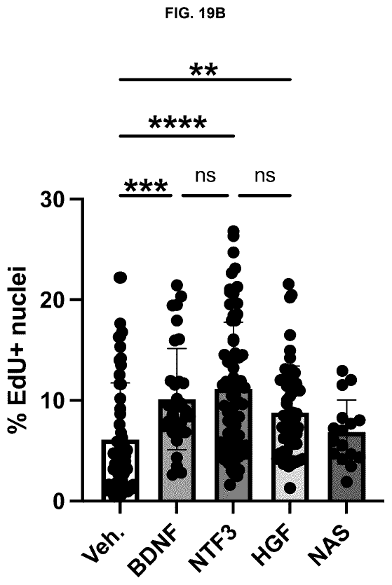

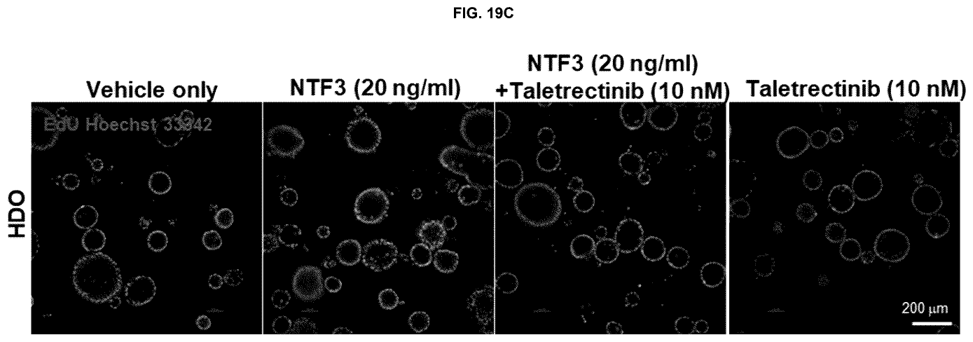

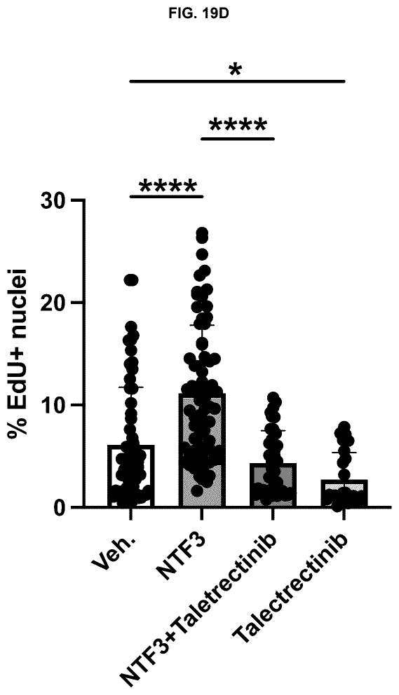

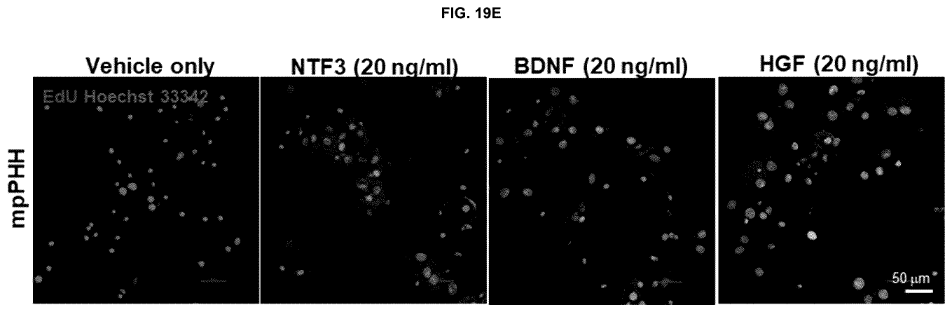

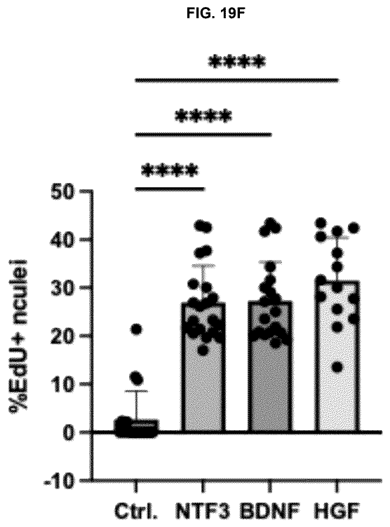

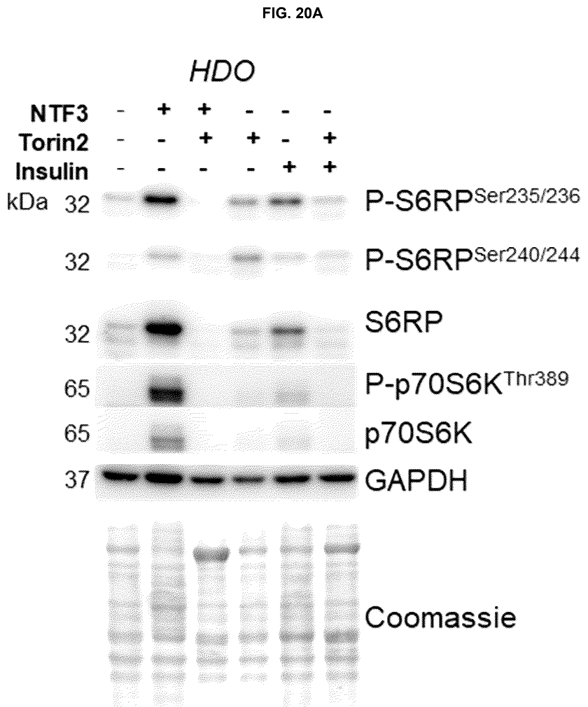

To evaluate if BDNF or NTF3 could induce hepatocyte proliferation in human livers, human ductal organoids (HDO) which may be differentiated to a hepatic phenotype using commercially available media based on the Clever's protocol (Broutier et al., Nat Protoc. 2016; 11(9): 1724-43) were assessed. Primary human liver cells were isolated using a modified hepatocyte isolation protocol and cultured cells on collagen-coated plates to observe their phenotype in 2D and in 3D using Matrigel® (FIGS. 18A-C). HDOs were expanded in organoid growth media (OGM) for approximately two weeks and were passaged up to 8 times to ensure stability. Experiments were performed with cryopreserved HDO and results assessed from at least 2 different lines. To induce hepatocytic differentiation, HDOs were incubated in organoid differentiation medium (ODM) for 5 days. qPCR analyses demonstrated low or decreased levels for cholangiocyte differentiation genes SOX9, KRT19 but marked increased gene expression for ALB and HNF4A indicative of hepatocytic differentiation (FIG. 18D). For proliferation analyses, hepatocytic differentiated HDO were incubated in serum free William's E medium to ensure defined culturing conditions free of possible growth factors, as incubation with commercial basal media without growth factors suggested presence of unknown factors conducive to proliferation (FIG. 18E). HDO cultured in OGM were lighter in agreement with previous descriptions while HDOs cultured in ODM were darker. HDOs incubated in ODM and William's E were similar in appearance (FIG. 18F). HDOs were incubated with either vehicle, BDNF, NTF3, or HGF and proliferation assessed by EdU labeling. Quantification of Edu+ nuclei indicated significantly increased EdU+ cells compared to vehicle only treated HDO, suggesting increased proliferation (FIGS. 19A-B). As NTF3 is more readily commercially available than BDNF, NTF3 was used for further studies. To assess if NTF3-induced proliferation is mediated by TRK signaling, proliferation was tested in response to co-incubation with taletrectinib, a specific Pan-TRK inhibitor. Quantification of Edu+ cells showed significant increase of EdU+ cells with NTF3 treatment compared to vehicle alone, which was decreased by co-incubation with taletrectinib (FIGS. 19C-D). Primary human hepatocytes (PHH) that had been expanded in vivo (mouse passaged primary human hepatocytes, mpPHH) (Michailidis et al., Proc Natl Acad Sci USA. 2020; 117(3): 1678-1688) were also tested. Incubation of mpPHHs with NTF3, BDNF, or HGF increased EdU+ nuclei compared to vehicle only treated mpPHHs (FIGS. 19E-F). As reported previously, the vast majority of hepatocytes were human (85-97%) as assessed by human nuclear mitotic apparatus protein (NuMA) staining (FIGS. 18H-I).

### Example 13

### NTF3 Activates mTOR in Hepatocytic Differentiated Hepatic Ductal Organoids and Mouse Passaged Primary Human Hepatocytes

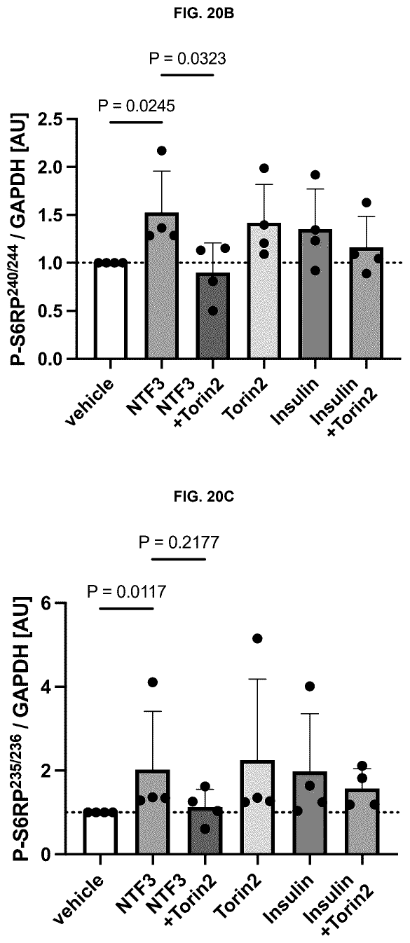

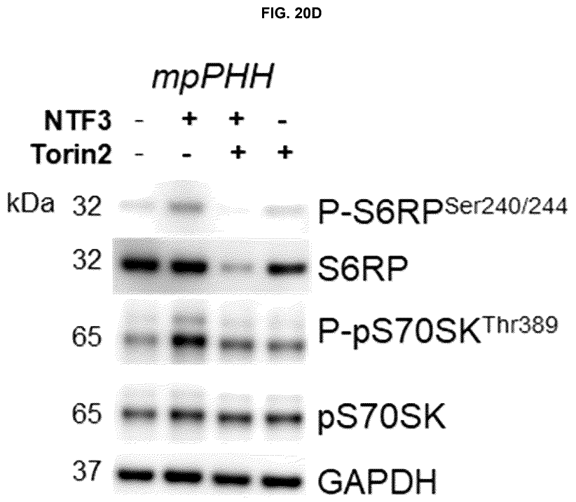

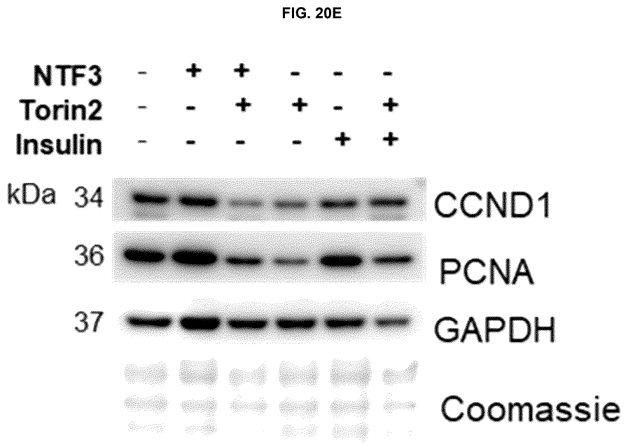

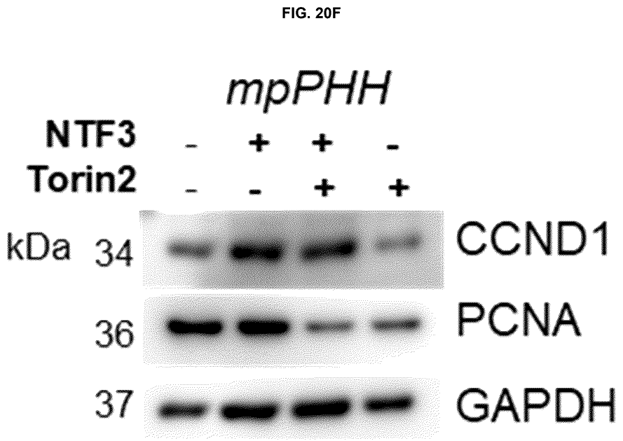

In the central nervous system, NTF3 binds and activates downstream signaling pathways including PI3K-mTOR to promote survival, proliferation and differentiation of neurons and neuronal progenitors. TOR is a major growth signaling pathway that is activated by nutrient availability, growth hormones and oxygen and is known to be inhibited by amino acids such as glutamine. Herein it was confirmed that HDOs cultured in glutamine free media decreased phosphorylated S6rp (Ser240/244) upon coincubation with either mTORC1 inhibitor Torin2, glutamine or both (FIG. 18G), therefore the following experiments were carried out in glutamine free media. To assess if NTF3 activates mTORC1 in HDO, HDO were incubated with serum free and glutamine free William's E medium containing either vehicle, NTF3, Torin2, or insulin, a known activator of mTORC1. Immunoblot analysis for P-p70S6 kinase and P-S6 ribosomal protein (S6RP), showed significantly increased phosphorylation with NTF3 and insulin, which was decreased by co-incubation of NTF3 or Insulin with Torin2. These results suggest that NTF3 activates mTORC1 in HDOs (FIGS. 20A-C). Similar results were obtained by immunoblotting of mpPHHs that had been incubated with NTF3, torin2, or torin2 only. Incubation with NTF3 increased levels of phosphorylated P-S6RP^{Ser240/244} and P-pS70SK^{Thr389} which was inhibited co-incubation with Torin2 thereby suggesting that NTF3 activates mTOR in mpPHHs (FIG. 19E). The above experiments demonstrated that HSCs are essential for CCND1 expression in liver. To test if NTF3 induces CCND1 and proliferation downstream of mTOR, HDOs and mpPHHs lysates were assessed by immunoblotting. Incubation of HDOs and mpPHHs with NTF3 induced CCND1 and PCNA, a proliferation marker, which was inhibited by co-incubation with torin2 (FIGS. 19F-G).

In conclusion, the results herein suggest that NTF3 and BDNF are released by mouse and human HSCs. Administration of NTF3 accelerates liver regeneration after partial hepatectomy in mice. BDNF and NTF3 induce proliferation in human hepatocytic differentiated HDOs and mpPHHs that is TRK dependent. The results herein clarified NTF3 downstream signaling and demonstrated that it activates mTOR to promote growth and proliferation. Given the paucity of available therapies for patients with acute liver failure due to small-for size livers after either liver transplantation or in living liver donor patients who donated part of their liver for transplantation, NTF3, BDNF, and their analogon NAS merit further studies to evaluate their possible therapeutic potential.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

| | SEQUENCES |
|---|---|
| | TCRα-Forward Primer |
| SEQ ID NO: 1 | |
| | GAGGAGCCAGCAGAAGGT |
| | TCRα-Reverse Primer |
| SEQ ID NO: 2 | |
| | TCCCACCCTACACTCACTACA |
| | TCRβ-Forward Primer |
| SEQ ID NO: 3 | |
| | TCAAGTCGCTTCCAACCTCAA |
| | TCRβ-Reverse Primer |
| SEQ ID NO: 4 | |
| | TGTCACAGTGAGCCGGGTG |
| | GFP-Forward Primer |
| SEQ ID NO: 5 | |
| | GTGGAAGCAGAGAGGAGAGCATTTG |
| | GFP-Reverse Primer |
| SEQ ID NO: 6 | |
| | GGTCGGGGTAGCGGCTGAA |
| | Desmin Forward Primer |
| SEQ ID NO: 7 | |
| | GCCACCATGAGCCAGGCCTACT |
| | Desmin Reverse Primer |
| SEQ ID NO: 8 | |
| | TGCTCGAGGGAAC-ACGGGAGA |
| | Pdgfrb Forward Primer |
| SEQ ID NO: 9 | |
| | ACTACATCTCCAAAGGCAGCACCT |
| | Pdgfrb Reverse Primer |
| SEQ ID NO: 10 | |
| | TGTAGAACTGGTCGTTCATGGGCA |
| | Ntrk2 Forward Primer |
| SEQ ID NO: 11 | |
| | GTCTGGAGGGTGCTATGCTA |
| | Ntrk2 Reverse Primer |
| SEQ ID NO: 12 | |
| | CAGGGGCAGAAACTCCAGAA |
| | GAPDH Forward Primer |
| SEQ ID NO: 13 | |
| | CAATGACCCCTTCATTGACC |
| | GAPDH Reverse Primer |
| SEQ ID NO: 14 | |
| | GATCTCGCTCCTGGAAGATG |
| | shNtrk2_1 |
| SEQ ID NO: 15 | |
| | CATTCCAAGTTTGGCATGAAA |
| | shNtrk2_2 |
| SEQ ID NO: 16 | |
| | CAGCAACCTGCGGCACATAAA |
| | TK-Forward Primer |
| SEQ ID NO: 17 | |
| | GGGGCCTCGGTCCTAGT |
| | TK-Reverse Primer |
| SEQ ID NO: 18 | |
| | GATGGCAGGGGTACGAAG |
| | hm-Albumin Forward Primer |
| SEQ ID NO: 19 | |
| | CACGCCTTTGGCACAATGAA |
| | hm-Albumin Reverse Primer |
| SEQ ID NO: 20 | |
| | ATCTCGACGAAACACACCCC |
| | hm-KRT19 Forward Primer |
| SEQ ID NO: 21 | |
| | GAAGGATGCTGAAGCCTGGT |
| | hm-KRT19 Reverse Primer |
| SEQ ID NO: 22 | |
| | GTCAGTAACCTCGGACCTGC |
| | hm-SOX9 Forward Primer |
| SEQ ID NO: 23 | |
| | GACTTCTGAACGAGAGCGAGA |
| | hm-SOX9 Reverse Primer |
| SEQ ID NO: 24 | |
| | CCGTTCTTCACCGACTTCCTC |
| | hm-HNF4A Forward Primer |
| SEQ ID NO: 25 | |
| | TGCGACTCTCCAAAACCCTC |
| | hm-HNF4A Reverse Primer |
| SEQ ID NO: 26 | |
| | ATTGCCCATCGTCAACACCT |
| | Hm-TBP Forward Primer |
| SEQ ID NO: 27 | |
| | GCATCACTGTTTCTTGGCGT |
| | Hm-TBP Reverse Primer |
| SEQ ID NO: 28 | |
| | AGAGCATCTCCAGCACACTC |

What is claimed is:

1. A method of stimulating proliferation of a liver cell, comprising contacting the liver cell with a tropomyosin kinase receptor (TRK) ligand in an amount effective to stimulate proliferation of the liver cell.

2. The method of claim 1, wherein the liver cell is a hepatocyte, a cholangiocyte, or a combination thereof.

3. The method of claim 1, wherein the TRK ligand is a neurotrophin, a melatonin precursor, a neurotrophin mimetic, a flavone, or a combination thereof.

4. The method of claim 3, wherein the neurotrophin is one or more of neurotrophin-3 (NTF3) and brain-derived neurotrophic factor (BDNF);
wherein the melatonin precursor is N-acetyl serotonin (NAS);
wherein the neurotrophin mimetic is LM22A-4, GSB-106, BNN-20, tricyclic dimeric peptide 6 (TDP6), LM11A-31, or a combination thereof; and
wherein the flavone is 7.8-dihydroxyflavone (DHF).

5. The method of claim 4, wherein the NTF3 and the BDNF are recombinant proteins.

6-8. (canceled)

9. The method of claim 1, wherein the effective amount of the TRK ligand is about 10 ng/mL to about 100 μg/mL.

10. The method of claim 1, wherein the liver cell is contacted with the TRK ligand daily for about 1 day to about 14 days; or
wherein the liver cell is contacted with the TRK ligand every other day for about 1 day to about 14 days.

11. The method of claim 1, wherein the TRK ligand is administered to a subject in need thereof.

12. The method of claim 11, wherein the TRK ligand is administered intravenously, subcutaneously, or intramuscularly.

13. The method of claim 11, wherein the TRK ligand is administered following acute or chronic liver damage.

14. (canceled)

15. The method of claim 13, wherein the liver damage is a result of hepatectomy, partial liver resection, post liver transplantation, cirrhosis of the liver, hepatic malignancies, exposure to alcohol, hepatotoxic drugs, infectious agents, side effects of gene therapy, exposure to anti-tuberculosis agents, exposure to chemotherapeutic agents, acetaminophen overdoses, mushroom poisoning, infarction, latrogenic injury, cardiogenic shock, other kind of shock with decrease in blood pressure and resulting hypoperfusion of the liver, ischemia-reperfusion injury, or combinations thereof.

16. The method of claim 11, wherein the subject is a human, a monkey, a pig, a mouse, a dog, a guinea pig, or a rat.

17. The method of claim 1, wherein the TRK ligand is in a composition comprising a pharmaceutically acceptable excipient or a culture medium composition.

18. The method of claim 17, wherein the pharmaceutically acceptable excipient or the culture medium comprises saline, water, albumin-supplemented ringer solution, methyl cellulose, polyethylene glycol 500 (PEG500), or a combination thereof.

19. The method of claim 1, wherein the liver cell is further contacted with a growth factor other than a neurotrophin.

20. The method of claim 19, wherein the growth factor other than a neurotrophin is one or more of insulin, fibroblast growth factor (FGF), vascular endothelial growth factor (VEGF), epidermal growth factor (EGF), platelet-derived growth factor (PDGF), nerve growth factor (NGF), insulin-like growth factor (IGF), transforming growth factor (TGF), hepatocyte growth factor (HGF), bone morphogenetic proteins (BMPs), erythropoietin (EPO), colony-stimulating factors (CSFs), and hepatoma-derived growth factor (HDGF).

21. The method of claim 1, wherein contacting the liver cell with the TRK ligand increases liver cell proliferation at least 2 days following contact.

22. A method of stimulating liver regeneration in a subject in need thereof, comprising administering to the subject a tropomyosin kinase receptor (TRK) ligand in an amount effective to stimulate liver regeneration.

23-28. (canceled)

29. The method of claim 11, wherein the effective amount of the TRK ligand is about 10 ng/kg to about 100 μg/kg.

30-60. (canceled)

61. A cell culture medium composition comprising:
a base cell culture medium comprising one or more amino acids, salts, buffers, trace minerals, lipids, nucleic acids, additives, and proteins;
one or more tropomyosin kinase receptor (TRK) ligands;
one or more growth factors other than a neurotrophin;
one or more additional additives.
